(19) United States
(12) Patent Application Publication
(10) Pub. No.: US 2017/0166555 A1
(43) Pub. Date: Jun. 15, 2017

(54) FUNCTIONALISED AND SUBSTITUTED INDOLES AS ANTI-CANCER AGENTS

(71) Applicant: Novogen Limited, Hornsby, NSW (AU)

(72) Inventors: Ian James, Hornsby (AU); Ian Dixon, Hornsby (AU); John Feutrill, Hornsby (AU); Anthony Cuzzupe, Hornsby (AU); Herbert Treutlein, Scoresby (AU); Jun Zeng, Scoresby (AU); Tracy Nero, St. Albans (AU); Peter Gunning, Sydney (AU)

(21) Appl. No.: 15/039,017

(22) PCT Filed: Nov. 25, 2014
(86) PCT No.: PCT/AU2014/050372

§ 371 Date: May 24, 2016

Related U.S. Application Data
(60) Provisional application No. 62/035,671, filed on Aug. 11, 2014.
(60) Provisional application No. 61/908,285, filed on Nov. 25, 2013.

(51) Int. Cl. C07D 405/14; C07D 403/04; C07D 401/14; C07D 405/12; C07D 403/06; C07D 401/06; C07D 403/14; C07D 209/08; C07D 401/12

(52) U.S. Cl. CPC C07D 405/14; C07D 209/08; C07D 403/04; C07D 401/14; C07D 401/12; C07D 403/06; C07D 401/06; C07D 403/14; C07D 405/12

(57) ABSTRACT

The present invention relates to anti-tropomyosin compounds, processes for their preparation, and methods for treating or preventing a proliferative disease, preferably cancer, using compounds of the invention.

### FIELD OF THE INVENTION

The present invention relates broadly to pharmaceutical agents as treatments for proliferative disease such as cancer and a range of degenerative diseases such as osteoarthritis, atherosclerosis, heart disease and inflammatory bowel disease. In particular, the present invention relates to pharmaceutical agents which comprise aryl and/or alkyl substituted indole compounds. The invention further relates to methods for treating or preventing a proliferative disease, preferably cancer. The invention also relates to processes for preparing the compounds.

### BACKGROUND OF THE INVENTION

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

Cancer kills many thousands of people and is the second largest cause of death in the USA. There have been significant breakthroughs made in treating or preventing a wide variety of cancers. For example patients with breast cancer have benefited from early screening programs as well as a variety of surgical techniques. However, these often prove physically and emotionally debilitating. Moreover, patients who have undergone surgery and subsequent chemotherapy often experience a recurrence in their disease.

A potential new method of specifically attacking cancer cells is through disruption of cancer cells' cellular skeletal system comprised predominantly of actin. The actin cytoskeleton is intimately involved in cell division and cell migration. However, actin plays a ubiquitous role as the cytoskeleton of tumor cells and the actin filaments of the muscle sarcomere. The differing roles but similarity in structure make actin a hard target for drug development, due to unwanted off-target side effects.

### SUMMARY OF THE INVENTION

The invention seeks to address one or more of the above mentioned problems, and/or to provide improvements in cancer therapy and in one embodiment provides an anti-tropomyosin compound.

In a first aspect of the invention there is provided a compound of general formula (I), or a pharmaceutically acceptable drug or prodrug thereof, wherein:

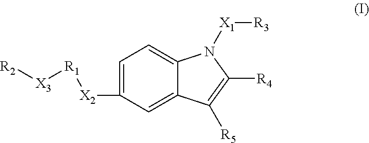

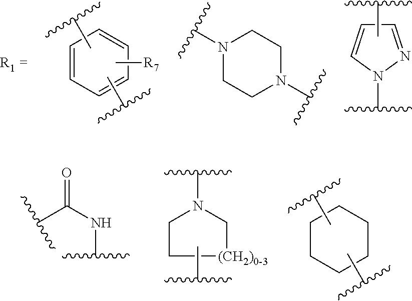

X₁═(CH₂)₀₋₅
X₂ and X₃═O, NH, NHR_{5′}, SO₂, C(O), C(O)NH, (CH₂)₀₋₅, C(O)(CH₂)₁₋₅, NH(CH₂)₁₋₅, CHR_{5′}, CHR(R_{5′})C(O), pyrazole, isooxazole
X₄═O, NH, NR₆

R₂═N(R₆)₂, CH(R₆)₂, indole

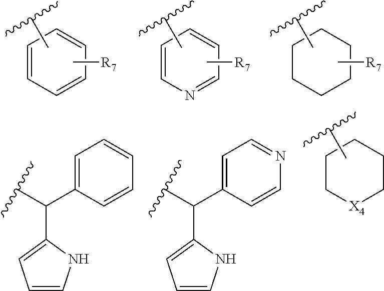

R₄, R₅ and R_{5′}═H, CH₃, CH₂CH₃
R₆═H, CH₃, (CH₂)₁₋₅CH₃, (CH₂)₁₋₅OCH₃, CF₃, CN, OCF₃
R₇═H, OH, alkyl, halo, alkoxy, hydroxyalkyl, amino, aminoalkyl, diaminoalkyl, or a dioxolane ring fused to 2 adjacent carbon atoms of R₁ of R₂

R₃═H, NH₂, N(R₆)₂, OR₄,

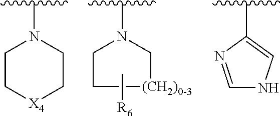

In one embodiment, X₁ is (CH₂)₃. In one embodiment, R₃ is N(R₆)₂. In one embodiment, R₆ is CH₃.

In one embodiment, X₁ is CH₂. In one embodiment, R₃ is H.

In one embodiment, X₁ is (CH₂)₂. In one embodiment R₃ is N(R₆)₂. In one embodiment, R₆ is CH₃.

In one embodiment, R₃ is

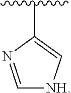 [SMILES: *c1c[nH]cn1]

In one embodiment, R₃ is

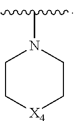

In one embodiment, X₄ is NR₅. In one embodiment, R₅ is CH₃.

In one embodiment, R₄ is CH₃ or H.

In one embodiment, R₅ is CH₃ or H.

In one embodiment, X₂ is CH₂, O, (CH₂)₀, NH or C(O).

In one embodiment, R₁ is

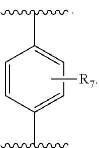

In one embodiment, R₇ is H.

In one embodiment, R₁ is

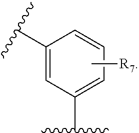

In one embodiment, R₇ is H.

In one embodiment, R₁ is

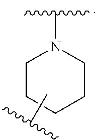

In one embodiment, X₃ is (CH₂)₂, C(O)NH, CH₂, (CH₂)₀, O or CHR_{5′}. In one embodiment, R_{5′} is CH₃.

In one embodiment, R₂ is

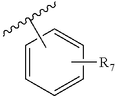

and R₇ is H, OH, halo, alkoxy, or the dioxolane ring. In one embodiment, halo is F. In one embodiment, alkoxy is OCH₃.

In one embodiment, R₂ is

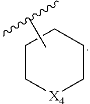

In one embodiment, X₄ is O. In one embodiment, X₄ is NR₆. In one embodiment, R₆ is CH₃.

In one embodiment, R₂ is

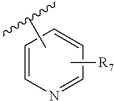

### and R₇ is H.

In one embodiment, R₂ is CH(R₆)₂. In one embodiment, R₆ is CH₃.

In one embodiment, R₂ is N(R₆)₂. In one embodiment, R₆ is CH₃.

In one embodiment, R₂ is

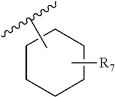

and R₇ is hydroxyalkyl. In one embodiment, hydroxyalkyl is CH₂OH.

Preferably, the compounds of the first aspect of the invention are exemplified in the following structures:

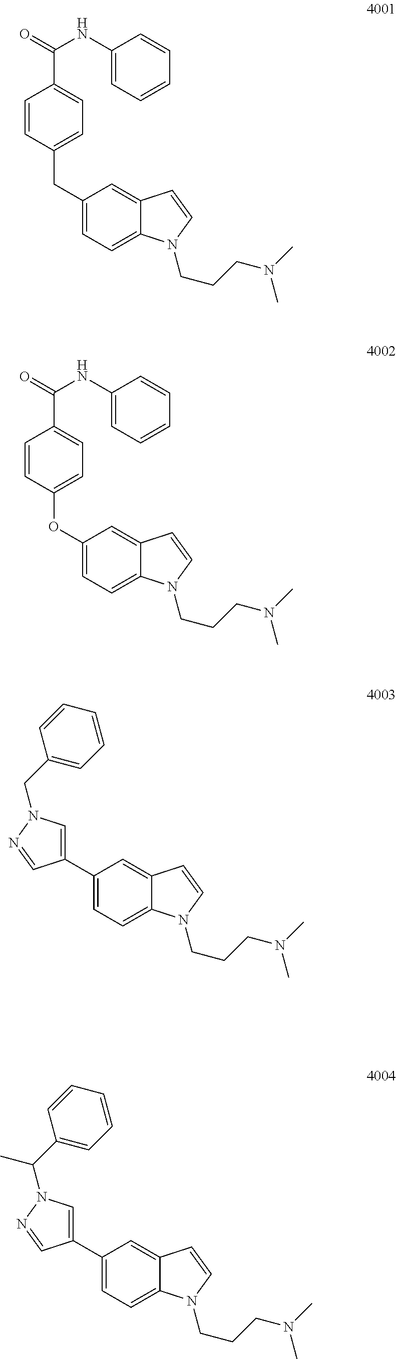

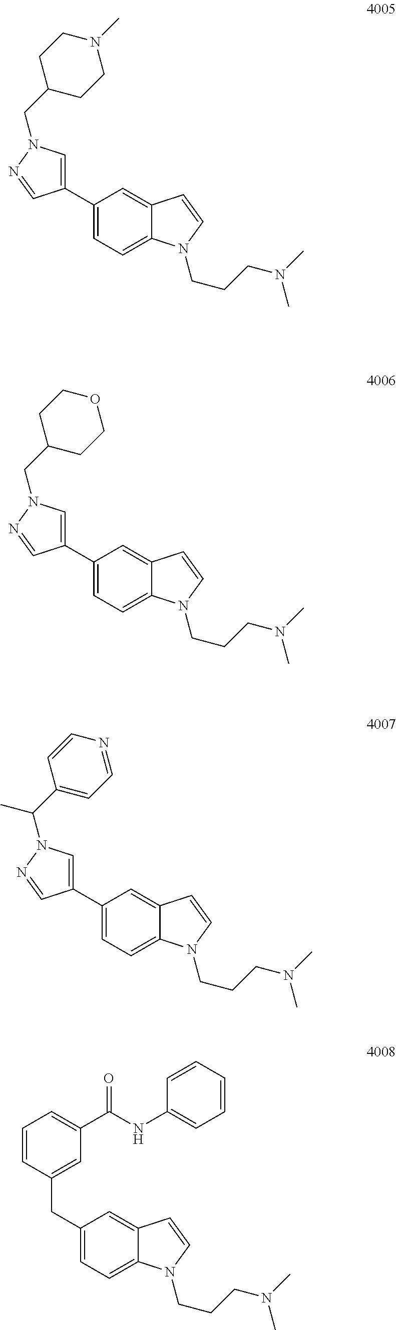

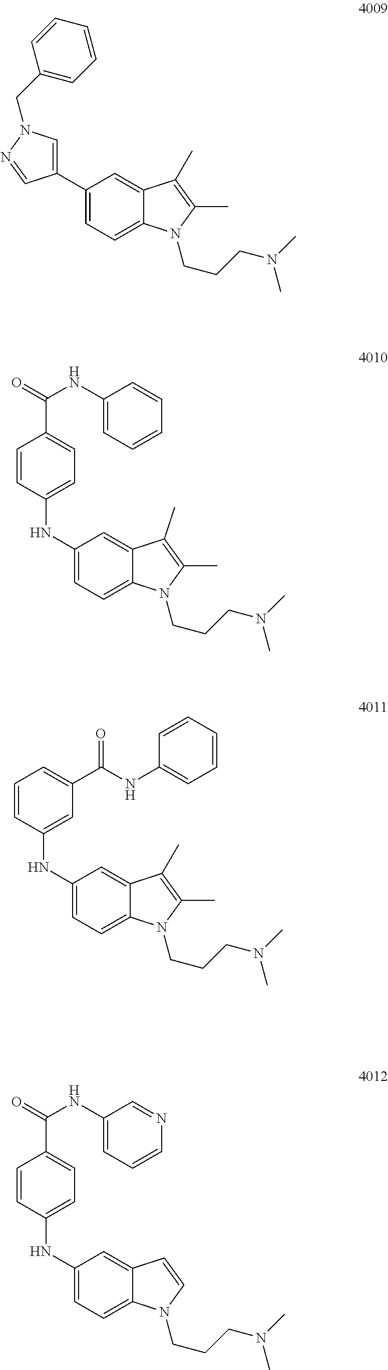 [SMILES: Cc1c(C)n(CCCN(C)C)c2ccc(-c3cnn(Cc4ccccc4)c3)cc12 ; Cc1c(C)n(CCCN(C)C)c2ccc(Nc3ccc(C(=O)Nc4ccccc4)cc3)cc12 ; Cc1c(C)n(CCCN(C)C)c2ccc(Nc3cccc(C(=O)Nc4ccccc4)c3)cc12 ; CN(C)CCCn1ccc2cc(Nc3ccc(C(=O)Nc4cccnc4)cc3)ccc21]

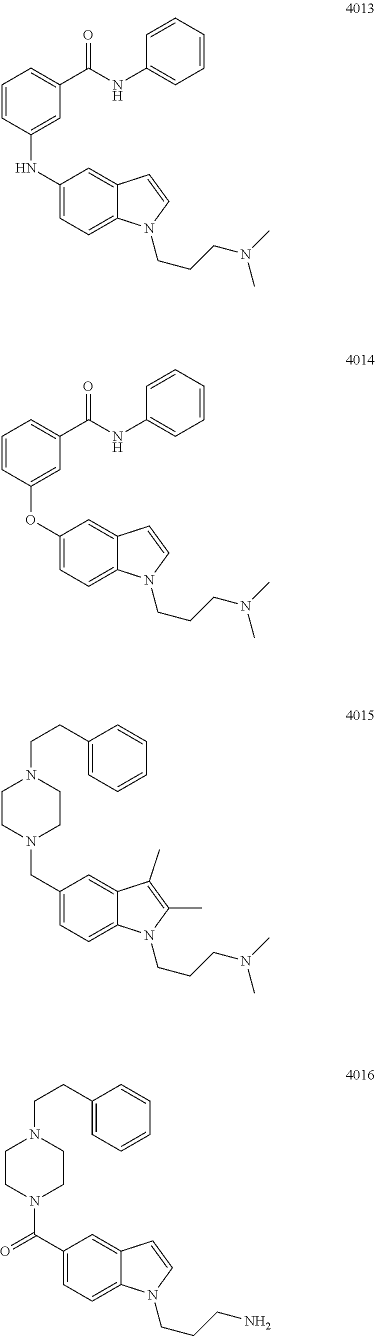 [SMILES: CN(C)CCCn1ccc2cc(Nc3cccc(C(=O)Nc4ccccc4)c3)ccc21 ; CN(C)CCCn1ccc2cc(Oc3cccc(C(=O)Nc4ccccc4)c3)ccc21 ; Cc1c(C)n(CCCN(C)C)c2ccc(CN3CCN(CCc4ccccc4)CC3)cc12 ; NCCCn1ccc2cc(C(=O)N3CCN(CCc4ccccc4)CC3)ccc21]

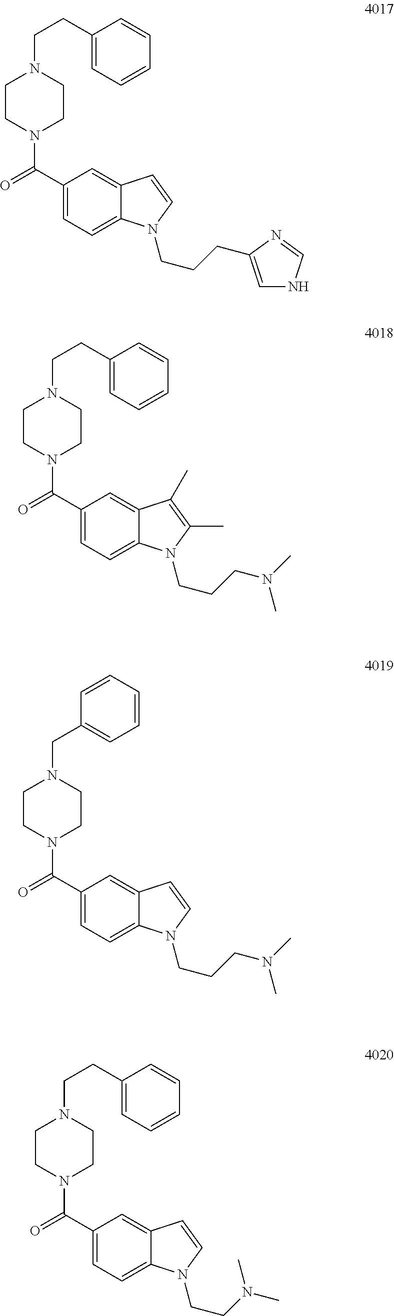 [SMILES: O=C(c1ccc2c(ccn2CCCc2c[nH]cn2)c1)N1CCN(CCc2ccccc2)CC1 ; Cc1c(C)n(CCCN(C)C)c2ccc(C(=O)N3CCN(CCc4ccccc4)CC3)cc12 ; CN(C)CCCn1ccc2cc(C(=O)N3CCN(Cc4ccccc4)CC3)ccc21 ; CN(C)CCn1ccc2cc(C(=O)N3CCN(CCc4ccccc4)CC3)ccc21]

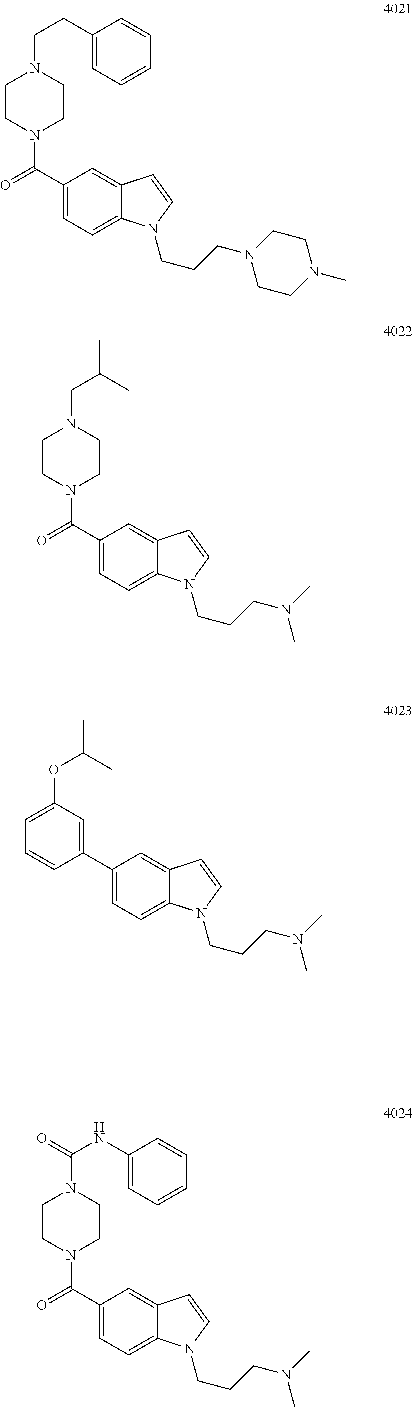 [SMILES: CN1CCN(CCCn2ccc3cc(C(=O)N4CCN(CCc5ccccc5)CC4)ccc32)CC1 ; CC(C)CN1CCN(C(=O)c2ccc3c(ccn3CCCN(C)C)c2)CC1 ; CC(C)Oc1cccc(-c2ccc3c(ccn3CCCN(C)C)c2)c1 ; CN(C)CCCn1ccc2cc(C(=O)N3CCN(C(=O)Nc4ccccc4)CC3)ccc21]

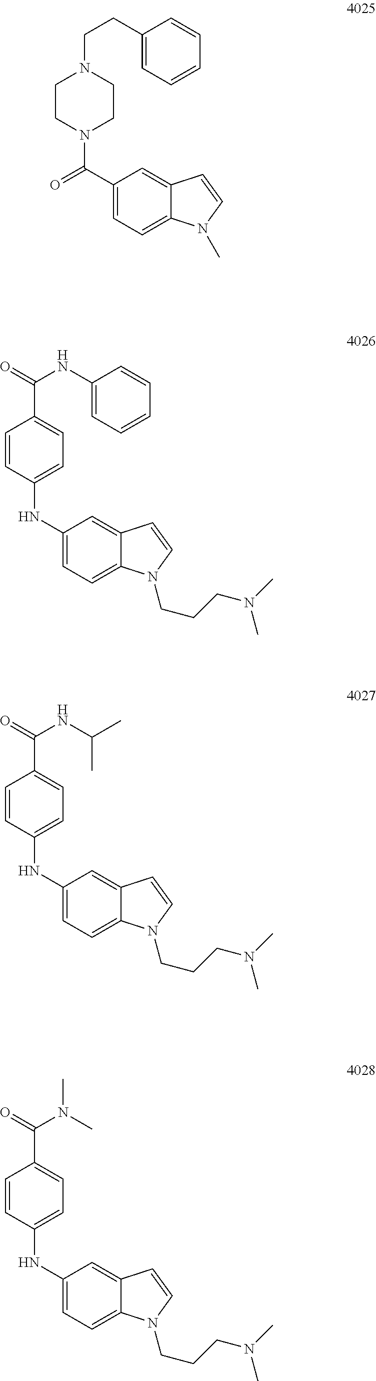 [SMILES: Cn1ccc2cc(C(=O)N3CCN(CCc4ccccc4)CC3)ccc21 ; CN(C)CCCn1ccc2cc(Nc3ccc(C(=O)Nc4ccccc4)cc3)ccc21 ; CC(C)NC(=O)c1ccc(Nc2ccc3c(ccn3CCCN(C)C)c2)cc1 ; CN(C)CCCn1ccc2cc(Nc3ccc(C(=O)N(C)C)cc3)ccc21]

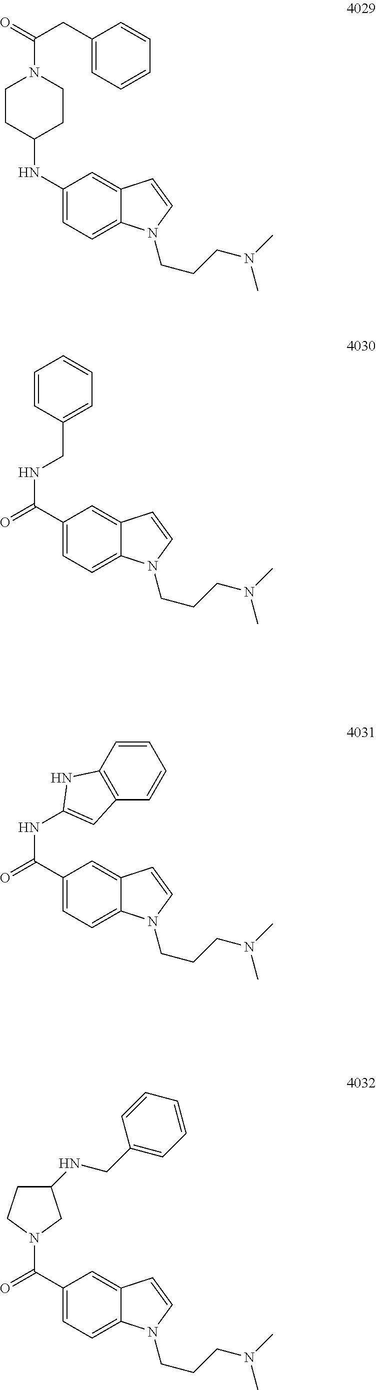 [SMILES: CN(C)CCCn1ccc2cc(NC3CCN(C(=O)Cc4ccccc4)CC3)ccc21 ; CN(C)CCCn1ccc2cc(C(=O)NCc3ccccc3)ccc21 ; CN(C)CCCn1ccc2cc(C(=O)Nc3cc4ccccc4[nH]3)ccc21 ; CN(C)CCCn1ccc2cc(C(=O)N3CCC(NCc4ccccc4)C3)ccc21]

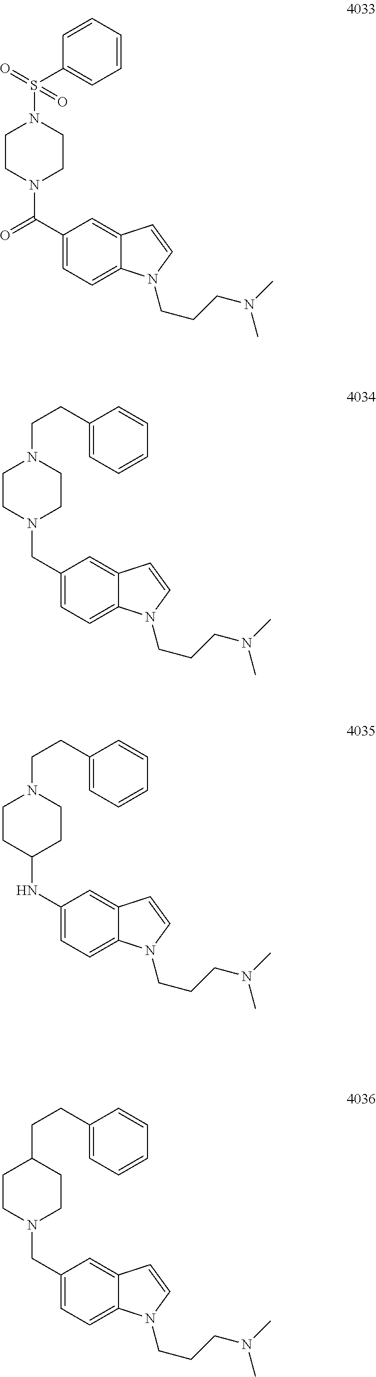 [SMILES: CN(C)CCCn1ccc2cc(C(=O)N3CCN(S(=O)(=O)c4ccccc4)CC3)ccc21 ; CN(C)CCCn1ccc2cc(CN3CCN(CCc4ccccc4)CC3)ccc21 ; CN(C)CCCn1ccc2cc(NC3CCN(CCc4ccccc4)CC3)ccc21 ; CN(C)CCCn1ccc2cc(CN3CCC(CCc4ccccc4)CC3)ccc21]

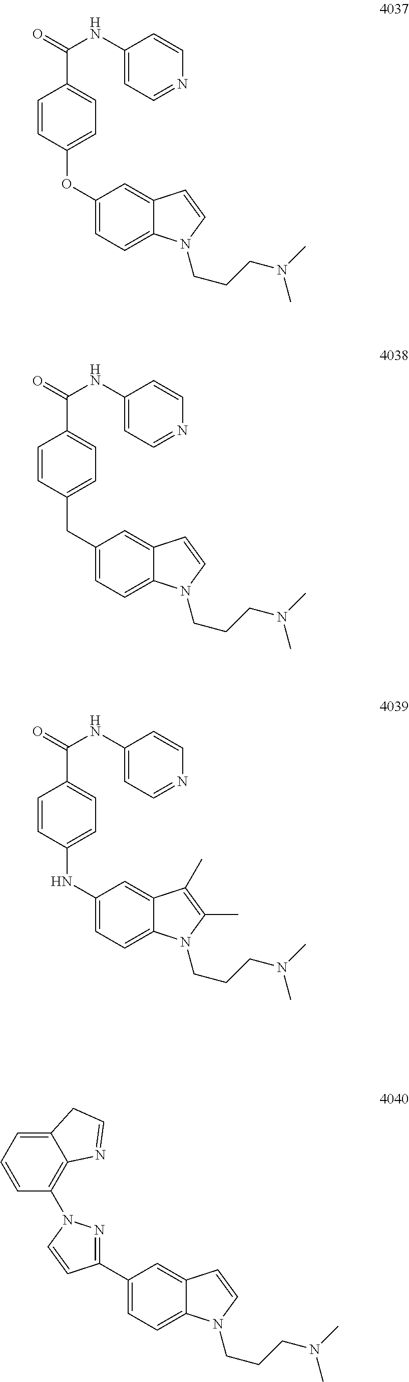 [SMILES: CN(C)CCCn1ccc2cc(Oc3ccc(C(=O)Nc4ccncc4)cc3)ccc21 ; CN(C)CCCn1ccc2cc(Cc3ccc(C(=O)Nc4ccncc4)cc3)ccc21 ; Cc1c(C)n(CCCN(C)C)c2ccc(Nc3ccc(C(=O)Nc4ccncc4)cc3)cc12 ; CN(C)CCCn1ccc2cc(-c3ccn(-c4cccc5c4N=CC5)n3)ccc21]

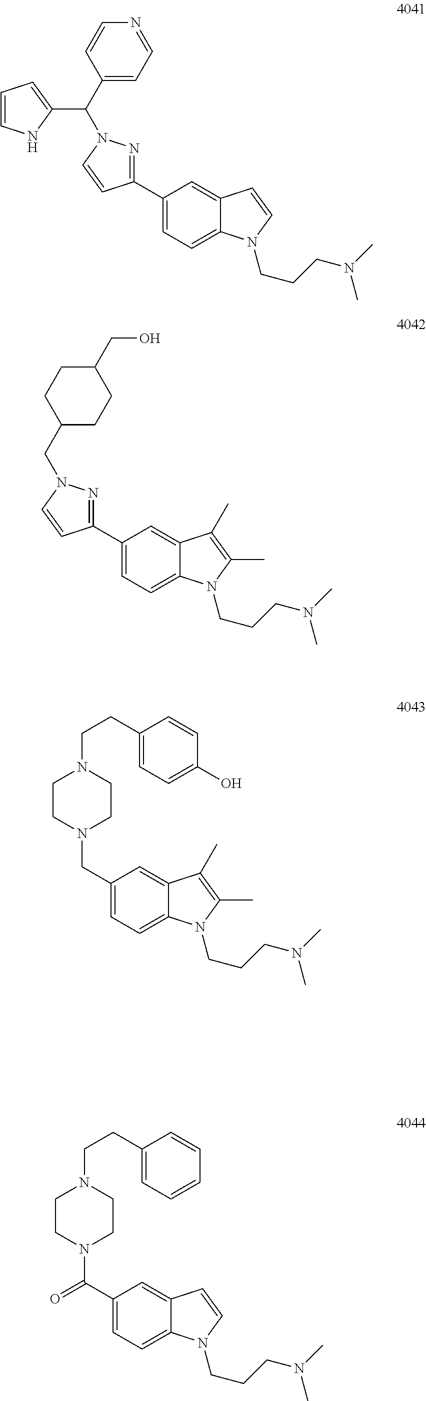 [SMILES: CN(C)CCCn1ccc2cc(-c3ccn(C(c4ccncc4)c4ccc[nH]4)n3)ccc21 ; Cc1c(C)n(CCCN(C)C)c2ccc(-c3ccn(CC4CCC(CO)CC4)n3)cc12 ; Cc1c(C)n(CCCN(C)C)c2ccc(CN3CCN(CCc4ccc(O)cc4)CC3)cc12 ; CN(C)CCCn1ccc2cc(C(=O)N3CCN(CCc4ccccc4)CC3)ccc21]

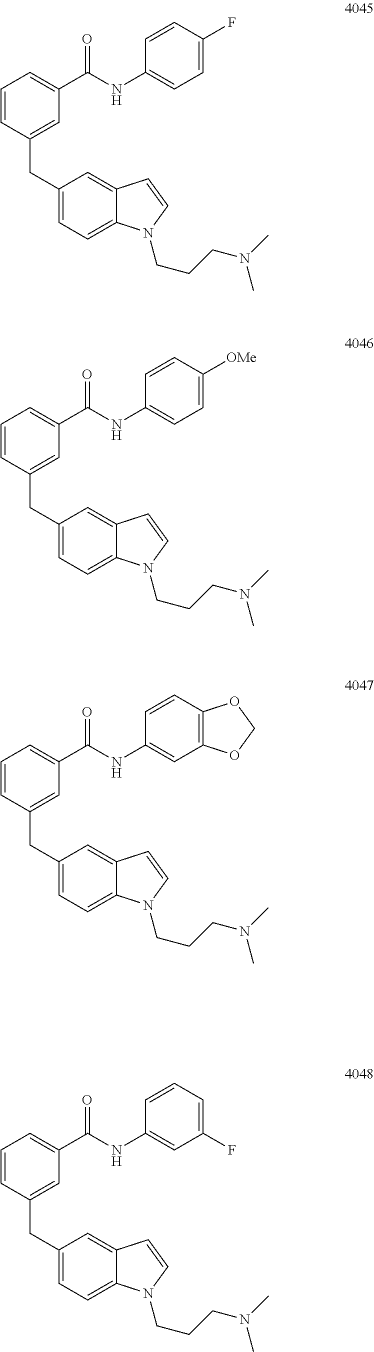 [SMILES: CN(C)CCCn1ccc2cc(Cc3cccc(C(=O)Nc4ccc(F)cc4)c3)ccc21 ; COc1ccc(NC(=O)c2cccc(Cc3ccc4c(ccn4CCCN(C)C)c3)c2)cc1 ; CN(C)CCCn1ccc2cc(Cc3cccc(C(=O)Nc4ccc5c(c4)OCO5)c3)ccc21 ; CN(C)CCCn1ccc2cc(Cc3cccc(C(=O)Nc4cccc(F)c4)c3)ccc21]

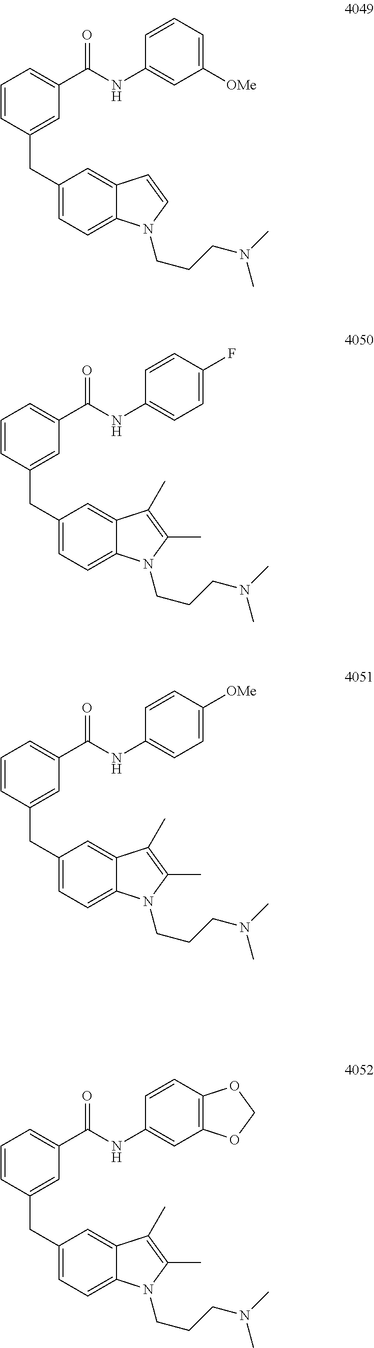 [SMILES: COc1cccc(NC(=O)c2cccc(Cc3ccc4c(ccn4CCCN(C)C)c3)c2)c1 ; Cc1c(C)n(CCCN(C)C)c2ccc(Cc3cccc(C(=O)Nc4ccc(F)cc4)c3)cc12 ; COc1ccc(NC(=O)c2cccc(Cc3ccc4c(c3)c(C)c(C)n4CCCN(C)C)c2)cc1 ; Cc1c(C)n(CCCN(C)C)c2ccc(Cc3cccc(C(=O)Nc4ccc5c(c4)OCO5)c3)cc12]

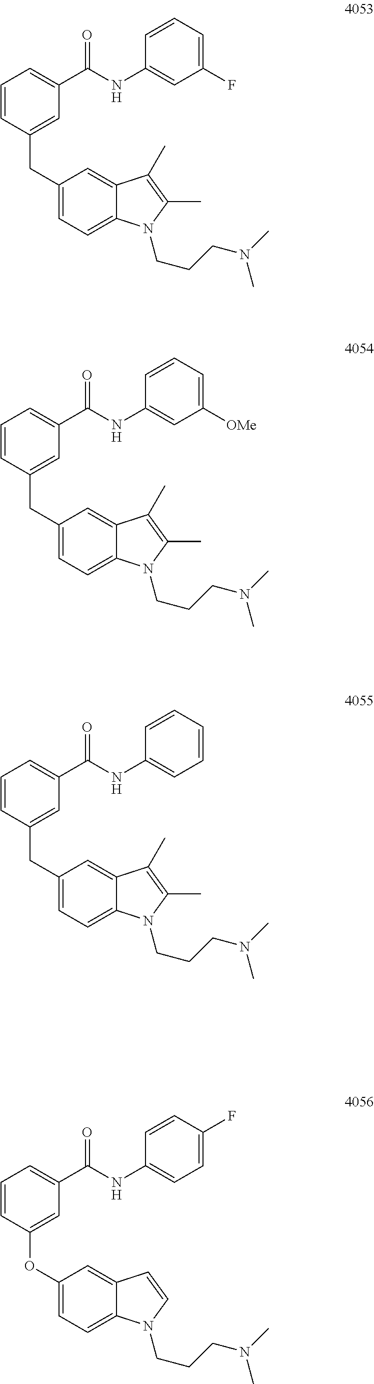 [SMILES: Cc1c(C)n(CCCN(C)C)c2ccc(Cc3cccc(C(=O)Nc4cccc(F)c4)c3)cc12 ; COc1cccc(NC(=O)c2cccc(Cc3ccc4c(c3)c(C)c(C)n4CCCN(C)C)c2)c1 ; Cc1c(C)n(CCCN(C)C)c2ccc(Cc3cccc(C(=O)Nc4ccccc4)c3)cc12 ; CN(C)CCCn1ccc2cc(Oc3cccc(C(=O)Nc4ccc(F)cc4)c3)ccc21]

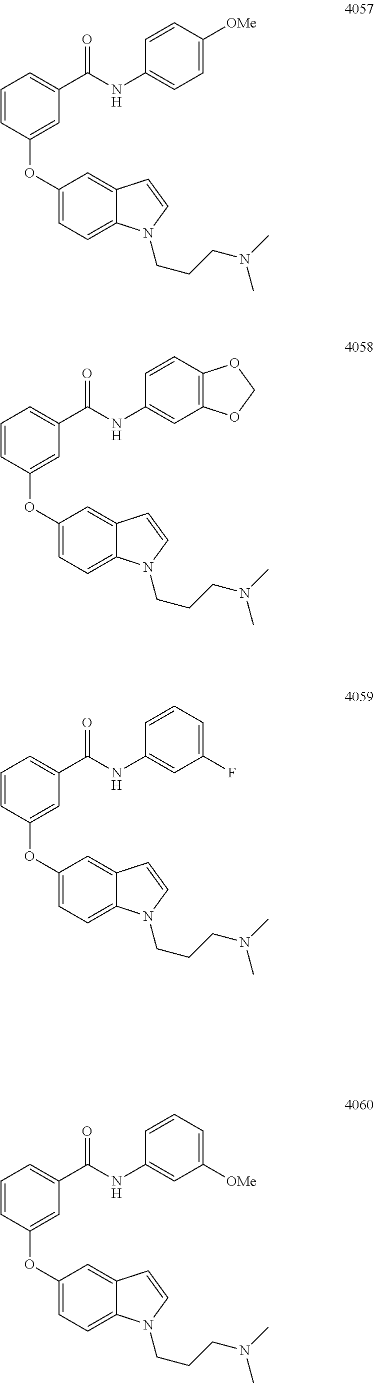 [SMILES: COc1ccc(NC(=O)c2cccc(Oc3ccc4c(ccn4CCCN(C)C)c3)c2)cc1 ; CN(C)CCCn1ccc2cc(Oc3cccc(C(=O)Nc4ccc5c(c4)OCO5)c3)ccc21 ; CN(C)CCCn1ccc2cc(Oc3cccc(C(=O)Nc4cccc(F)c4)c3)ccc21 ; COc1cccc(NC(=O)c2cccc(Oc3ccc4c(ccn4CCCN(C)C)c3)c2)c1]

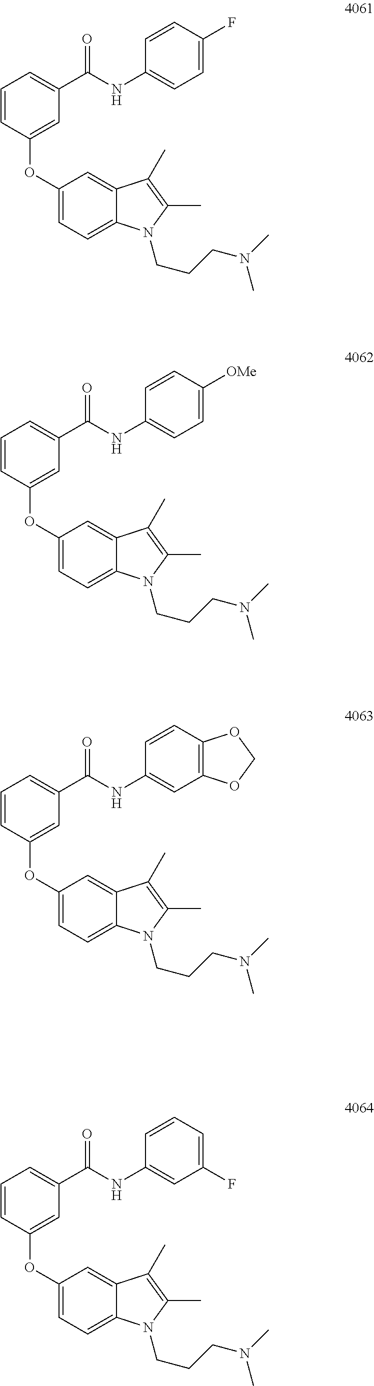 [SMILES: Cc1c(C)n(CCCN(C)C)c2ccc(Oc3cccc(C(=O)Nc4ccc(F)cc4)c3)cc12 ; COc1ccc(NC(=O)c2cccc(Oc3ccc4c(c3)c(C)c(C)n4CCCN(C)C)c2)cc1 ; Cc1c(C)n(CCCN(C)C)c2ccc(Oc3cccc(C(=O)Nc4ccc5c(c4)OCO5)c3)cc12 ; Cc1c(C)n(CCCN(C)C)c2ccc(Oc3cccc(C(=O)Nc4cccc(F)c4)c3)cc12]

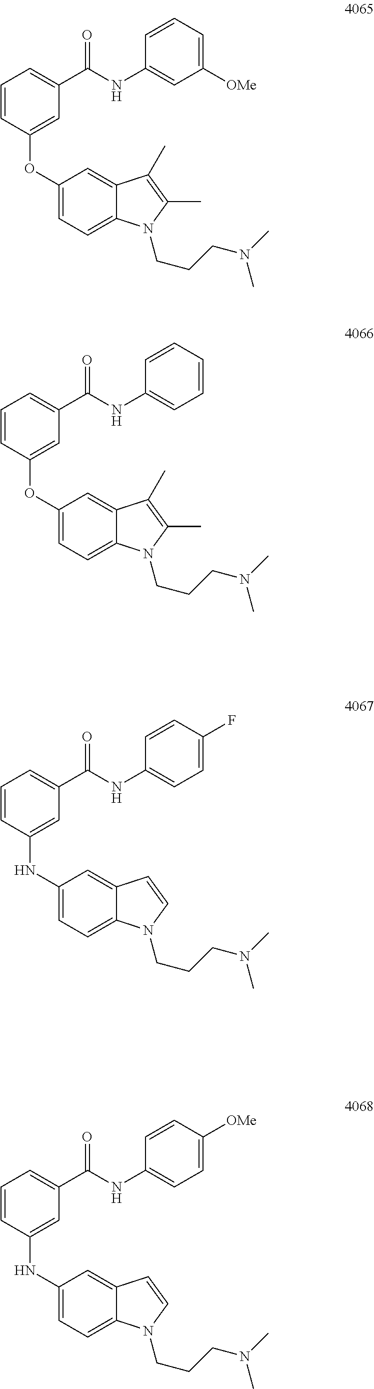 [SMILES: COc1cccc(NC(=O)c2cccc(Oc3ccc4c(c3)c(C)c(C)n4CCCN(C)C)c2)c1 ; Cc1c(C)n(CCCN(C)C)c2ccc(Oc3cccc(C(=O)Nc4ccccc4)c3)cc12 ; CN(C)CCCn1ccc2cc(Nc3cccc(C(=O)Nc4ccc(F)cc4)c3)ccc21 ; COc1ccc(NC(=O)c2cccc(Nc3ccc4c(ccn4CCCN(C)C)c3)c2)cc1]

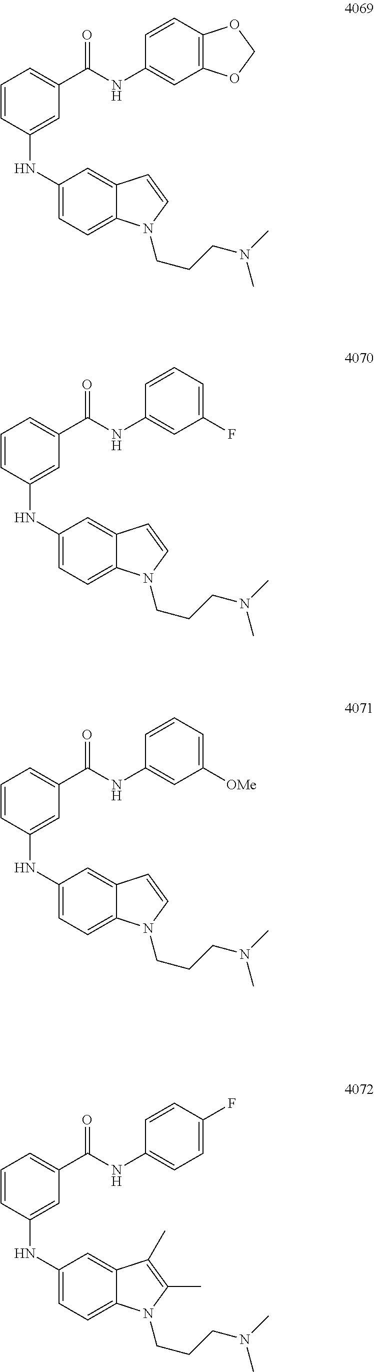 [SMILES: CN(C)CCCn1ccc2cc(Nc3cccc(C(=O)Nc4ccc5c(c4)OCO5)c3)ccc21 ; CN(C)CCCn1ccc2cc(Nc3cccc(C(=O)Nc4cccc(F)c4)c3)ccc21 ; COc1cccc(NC(=O)c2cccc(Nc3ccc4c(ccn4CCCN(C)C)c3)c2)c1 ; Cc1c(C)n(CCCN(C)C)c2ccc(Nc3cccc(C(=O)Nc4ccc(F)cc4)c3)cc12]

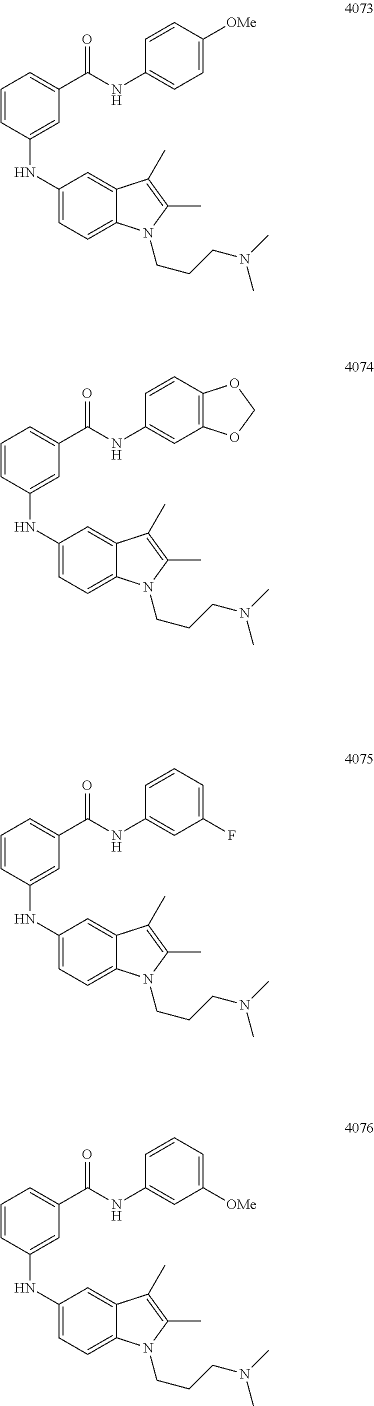 [SMILES: COc1ccc(NC(=O)c2cccc(Nc3ccc4c(c3)c(C)c(C)n4CCCN(C)C)c2)cc1 ; Cc1c(C)n(CCCN(C)C)c2ccc(Nc3cccc(C(=O)Nc4ccc5c(c4)OCO5)c3)cc12 ; Cc1c(C)n(CCCN(C)C)c2ccc(Nc3cccc(C(=O)Nc4cccc(F)c4)c3)cc12 ; COc1cccc(NC(=O)c2cccc(Nc3ccc4c(c3)c(C)c(C)n4CCCN(C)C)c2)c1]

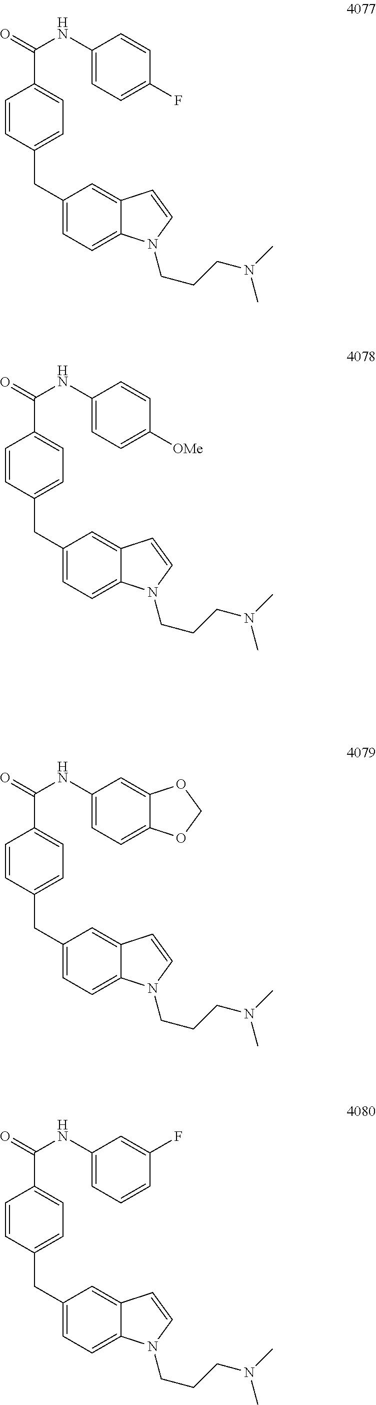 [SMILES: CN(C)CCCn1ccc2cc(Cc3ccc(C(=O)Nc4ccc(F)cc4)cc3)ccc21 ; COc1ccc(NC(=O)c2ccc(Cc3ccc4c(ccn4CCCN(C)C)c3)cc2)cc1 ; CN(C)CCCn1ccc2cc(Cc3ccc(C(=O)Nc4ccc5c(c4)OCO5)cc3)ccc21 ; CN(C)CCCn1ccc2cc(Cc3ccc(C(=O)Nc4cccc(F)c4)cc3)ccc21]

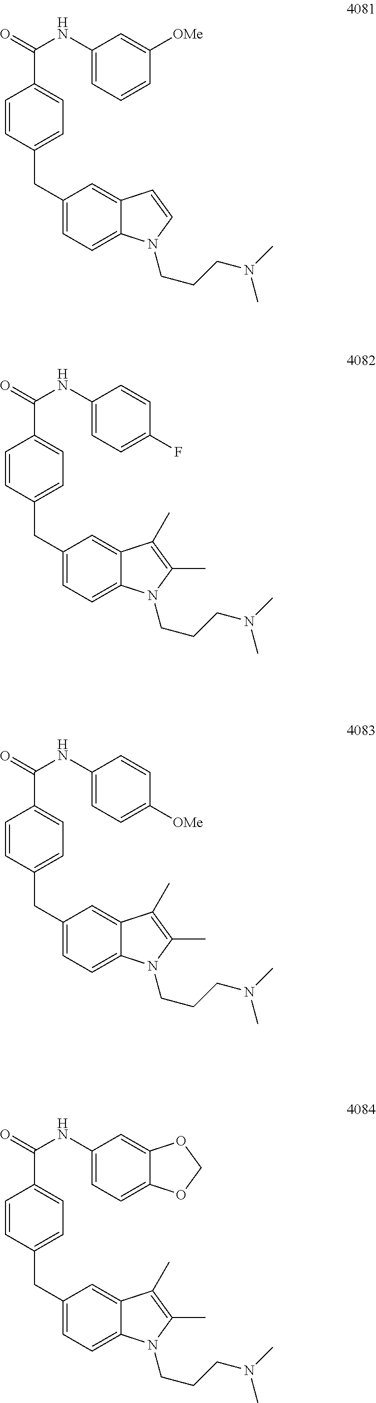 [SMILES: COc1cccc(NC(=O)c2ccc(Cc3ccc4c(ccn4CCCN(C)C)c3)cc2)c1 ; Cc1c(C)n(CCCN(C)C)c2ccc(Cc3ccc(C(=O)Nc4ccc(F)cc4)cc3)cc12 ; COc1ccc(NC(=O)c2ccc(Cc3ccc4c(c3)c(C)c(C)n4CCCN(C)C)cc2)cc1 ; Cc1c(C)n(CCCN(C)C)c2ccc(Cc3ccc(C(=O)Nc4ccc5c(c4)OCO5)cc3)cc12]

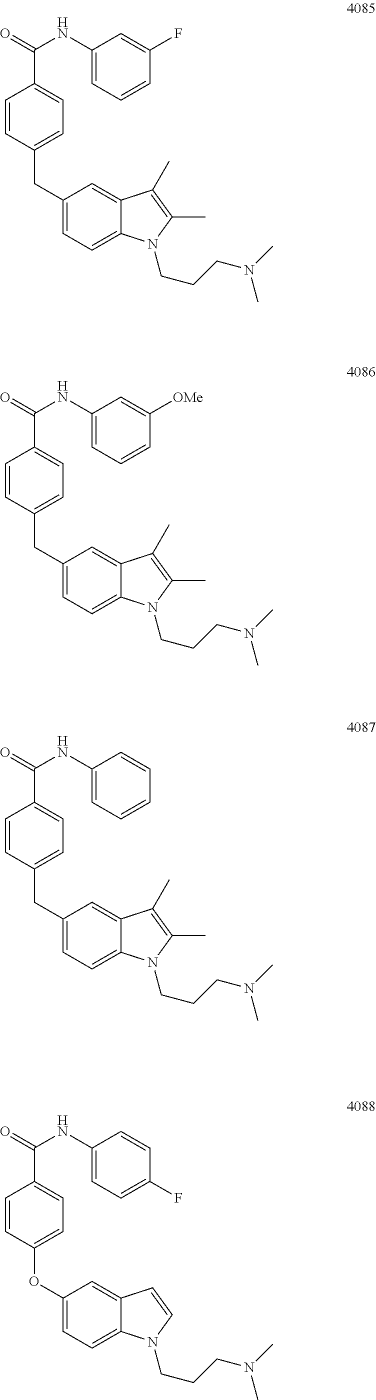 [SMILES: Cc1c(C)n(CCCN(C)C)c2ccc(Cc3ccc(C(=O)Nc4cccc(F)c4)cc3)cc12 ; COc1cccc(NC(=O)c2ccc(Cc3ccc4c(c3)c(C)c(C)n4CCCN(C)C)cc2)c1 ; Cc1c(C)n(CCCN(C)C)c2ccc(Cc3ccc(C(=O)Nc4ccccc4)cc3)cc12 ; CN(C)CCCn1ccc2cc(Oc3ccc(C(=O)Nc4ccc(F)cc4)cc3)ccc21]

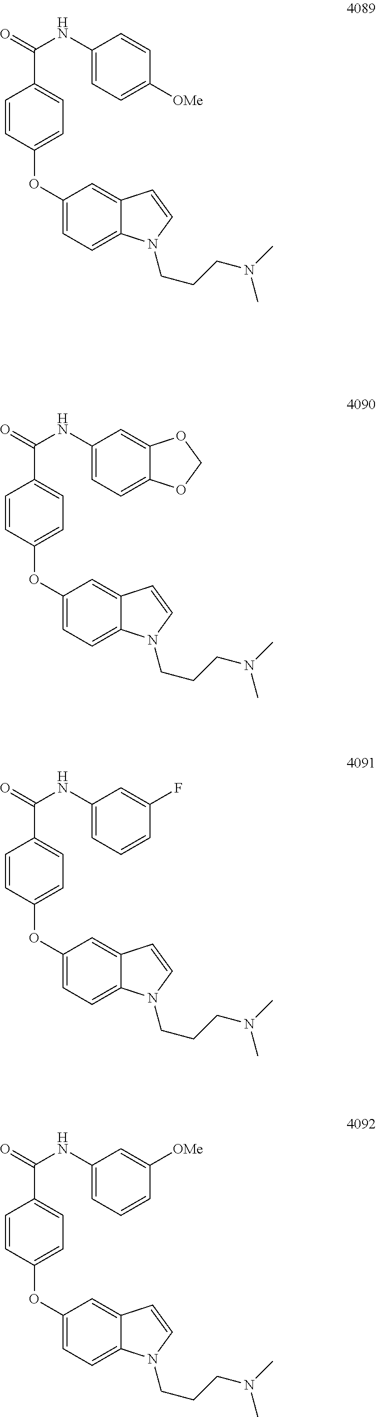 [SMILES: COc1ccc(NC(=O)c2ccc(Oc3ccc4c(ccn4CCCN(C)C)c3)cc2)cc1 ; CN(C)CCCn1ccc2cc(Oc3ccc(C(=O)Nc4ccc5c(c4)OCO5)cc3)ccc21 ; CN(C)CCCn1ccc2cc(Oc3ccc(C(=O)Nc4cccc(F)c4)cc3)ccc21 ; COc1cccc(NC(=O)c2ccc(Oc3ccc4c(ccn4CCCN(C)C)c3)cc2)c1]

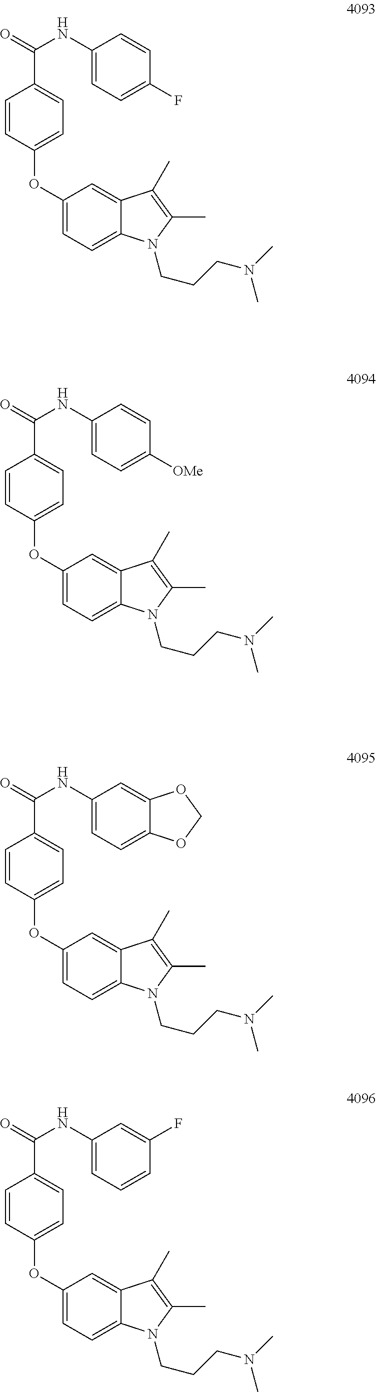 [SMILES: Cc1c(C)n(CCCN(C)C)c2ccc(Oc3ccc(C(=O)Nc4ccc(F)cc4)cc3)cc12 ; COc1ccc(NC(=O)c2ccc(Oc3ccc4c(c3)c(C)c(C)n4CCCN(C)C)cc2)cc1 ; Cc1c(C)n(CCCN(C)C)c2ccc(Oc3ccc(C(=O)Nc4ccc5c(c4)OCO5)cc3)cc12 ; Cc1c(C)n(CCCN(C)C)c2ccc(Oc3ccc(C(=O)Nc4cccc(F)c4)cc3)cc12]

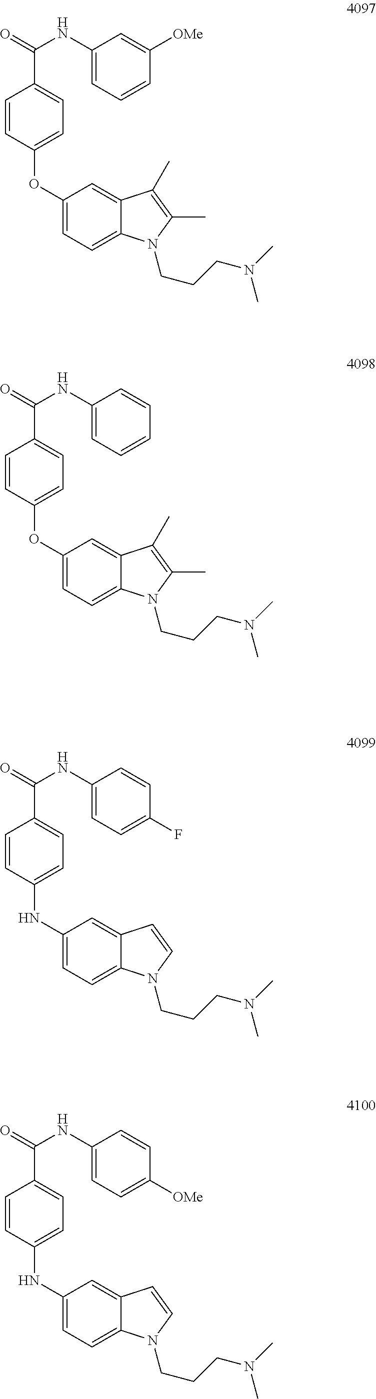 [SMILES: COc1cccc(NC(=O)c2ccc(Oc3ccc4c(c3)c(C)c(C)n4CCCN(C)C)cc2)c1 ; Cc1c(C)n(CCCN(C)C)c2ccc(Oc3ccc(C(=O)Nc4ccccc4)cc3)cc12 ; CN(C)CCCn1ccc2cc(Nc3ccc(C(=O)Nc4ccc(F)cc4)cc3)ccc21 ; COc1ccc(NC(=O)c2ccc(Nc3ccc4c(ccn4CCCN(C)C)c3)cc2)cc1]

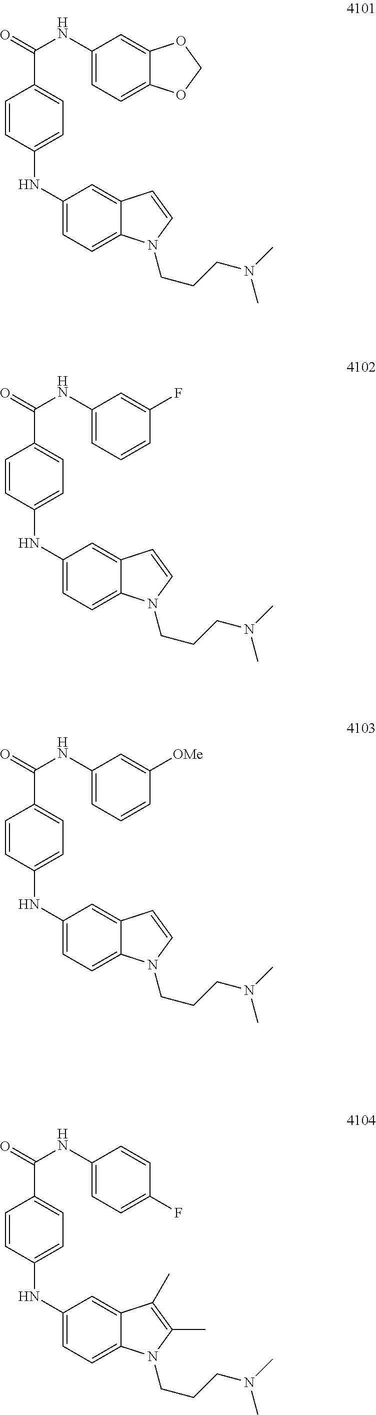 [SMILES: CN(C)CCCn1ccc2cc(Nc3ccc(C(=O)Nc4ccc5c(c4)OCO5)cc3)ccc21 ; CN(C)CCCn1ccc2cc(Nc3ccc(C(=O)Nc4cccc(F)c4)cc3)ccc21 ; COc1cccc(NC(=O)c2ccc(Nc3ccc4c(ccn4CCCN(C)C)c3)cc2)c1 ; Cc1c(C)n(CCCN(C)C)c2ccc(Nc3ccc(C(=O)Nc4ccc(F)cc4)cc3)cc12]

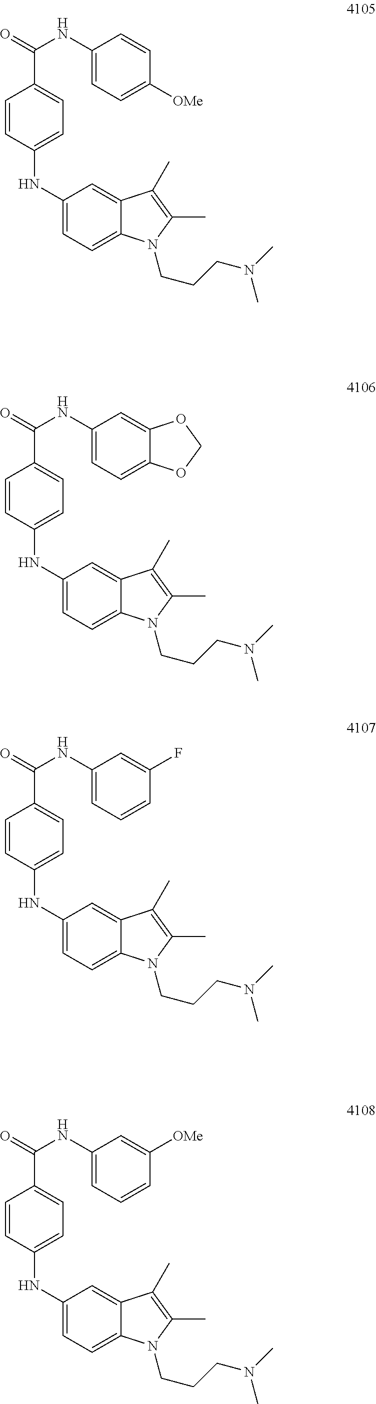 [SMILES: COc1ccc(NC(=O)c2ccc(Nc3ccc4c(c3)c(C)c(C)n4CCCN(C)C)cc2)cc1 ; Cc1c(C)n(CCCN(C)C)c2ccc(Nc3ccc(C(=O)Nc4ccc5c(c4)OCO5)cc3)cc12 ; Cc1c(C)n(CCCN(C)C)c2ccc(Nc3ccc(C(=O)Nc4cccc(F)c4)cc3)cc12 ; COc1cccc(NC(=O)c2ccc(Nc3ccc4c(c3)c(C)c(C)n4CCCN(C)C)cc2)c1]

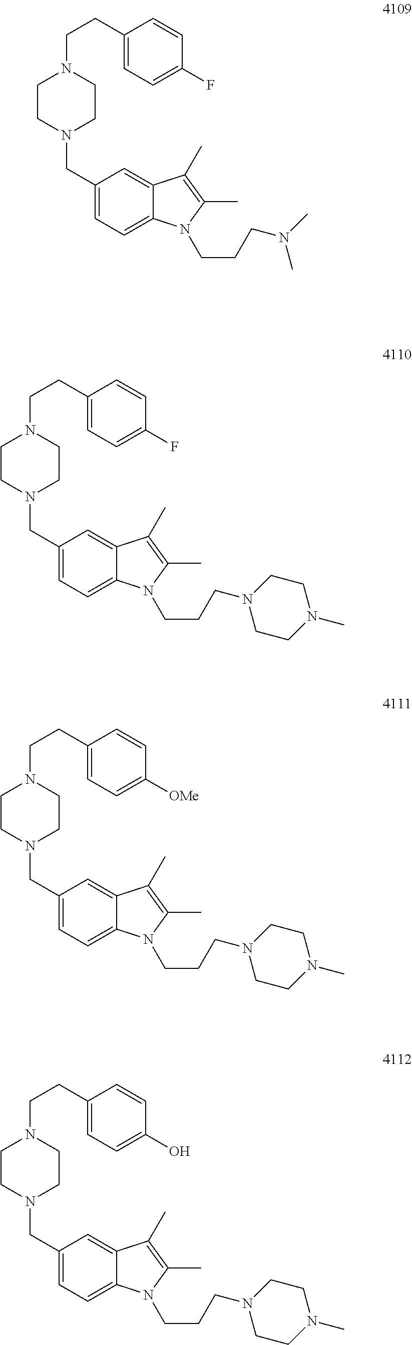 [SMILES: Cc1c(C)n(CCCN(C)C)c2ccc(CN3CCN(CCc4ccc(F)cc4)CC3)cc12 ; Cc1c(C)n(CCCN2CCN(C)CC2)c2ccc(CN3CCN(CCc4ccc(F)cc4)CC3)cc12 ; COc1ccc(CCN2CCN(Cc3ccc4c(c3)c(C)c(C)n4CCCN3CCN(C)CC3)CC2)cc1 ; Cc1c(C)n(CCCN2CCN(C)CC2)c2ccc(CN3CCN(CCc4ccc(O)cc4)CC3)cc12]

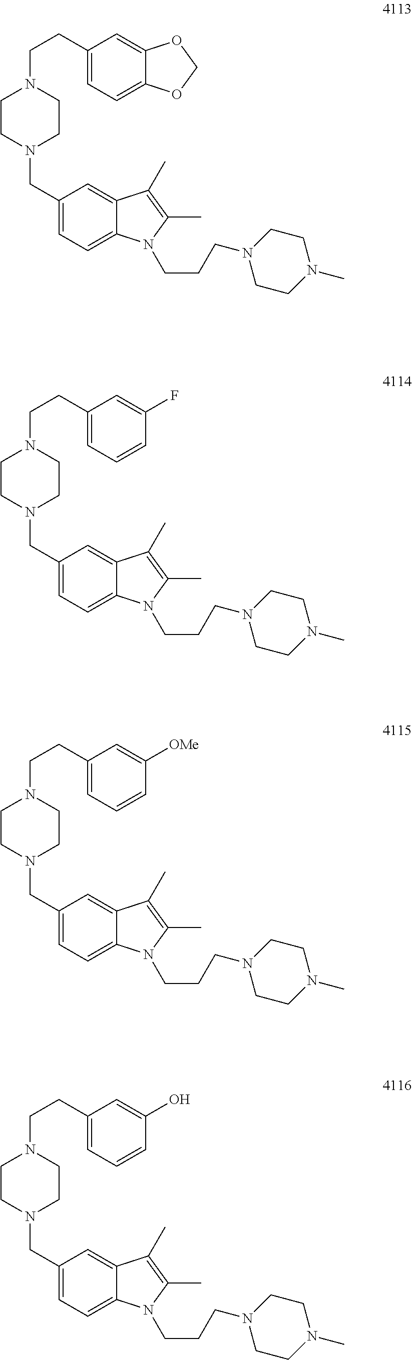 [SMILES: Cc1c(C)n(CCCN2CCN(C)CC2)c2ccc(CN3CCN(CCc4ccc5c(c4)OCO5)CC3)cc12 ; Cc1c(C)n(CCCN2CCN(C)CC2)c2ccc(CN3CCN(CCc4cccc(F)c4)CC3)cc12 ; COc1cccc(CCN2CCN(Cc3ccc4c(c3)c(C)c(C)n4CCCN3CCN(C)CC3)CC2)c1 ; Cc1c(C)n(CCCN2CCN(C)CC2)c2ccc(CN3CCN(CCc4cccc(O)c4)CC3)cc12]

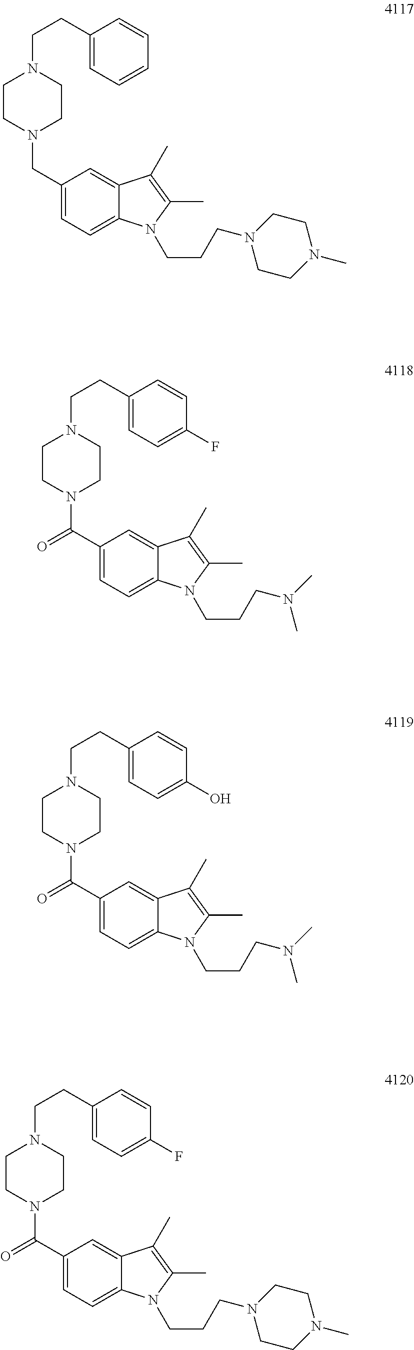 [SMILES: Cc1c(C)n(CCCN2CCN(C)CC2)c2ccc(CN3CCN(CCc4ccccc4)CC3)cc12 ; Cc1c(C)n(CCCN(C)C)c2ccc(C(=O)N3CCN(CCc4ccc(F)cc4)CC3)cc12 ; Cc1c(C)n(CCCN(C)C)c2ccc(C(=O)N3CCN(CCc4ccc(O)cc4)CC3)cc12 ; Cc1c(C)n(CCCN2CCN(C)CC2)c2ccc(C(=O)N3CCN(CCc4ccc(F)cc4)CC3)cc12]

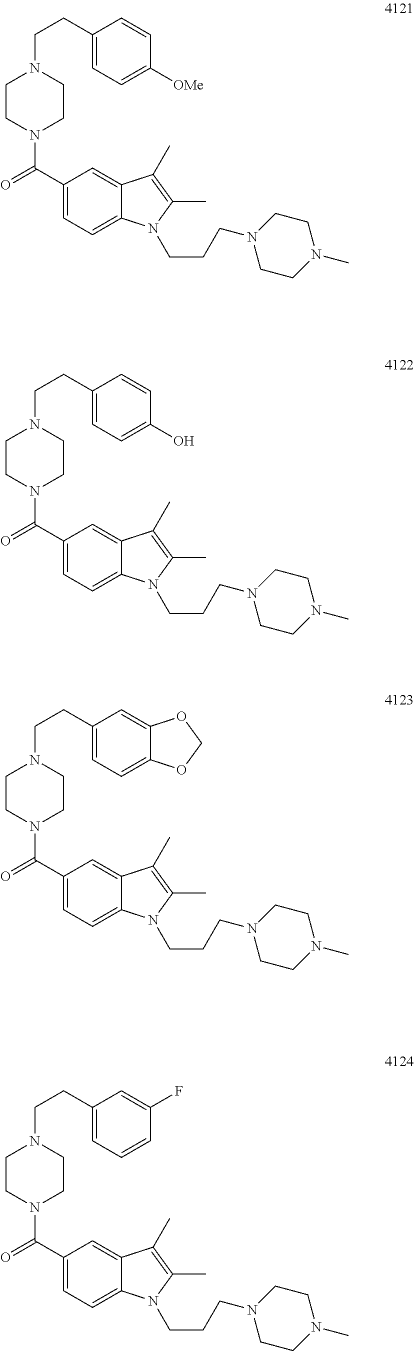 [SMILES: COc1ccc(CCN2CCN(C(=O)c3ccc4c(c3)c(C)c(C)n4CCCN3CCN(C)CC3)CC2)cc1 ; Cc1c(C)n(CCCN2CCN(C)CC2)c2ccc(C(=O)N3CCN(CCc4ccc(O)cc4)CC3)cc12 ; Cc1c(C)n(CCCN2CCN(C)CC2)c2ccc(C(=O)N3CCN(CCc4ccc5c(c4)OCO5)CC3)cc12 ; Cc1c(C)n(CCCN2CCN(C)CC2)c2ccc(C(=O)N3CCN(CCc4cccc(F)c4)CC3)cc12]

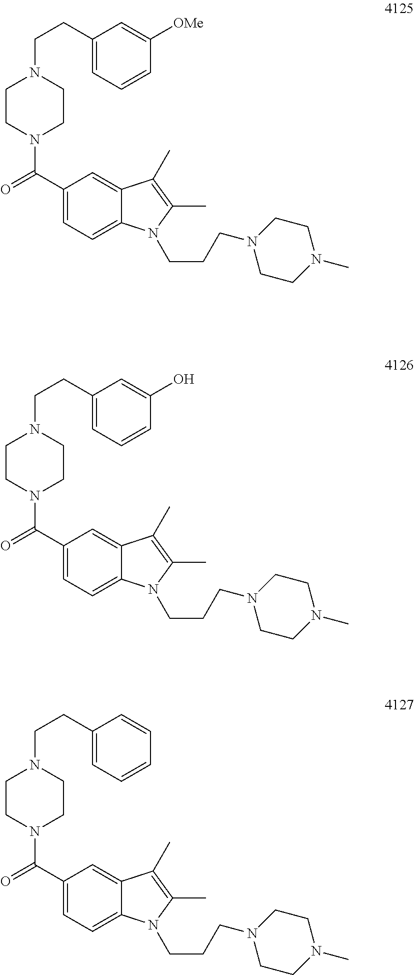 [SMILES: COc1cccc(CCN2CCN(C(=O)c3ccc4c(c3)c(C)c(C)n4CCCN3CCN(C)CC3)CC2)c1 ; Cc1c(C)n(CCCN2CCN(C)CC2)c2ccc(C(=O)N3CCN(CCc4cccc(O)c4)CC3)cc12 ; Cc1c(C)n(CCCN2CCN(C)CC2)c2ccc(C(=O)N3CCN(CCc4ccccc4)CC3)cc12]

In one embodiment, the compounds are:

4-((1-(3-(dimethylamino)propyl)-1H-indol-5-yl)methyl)-N-phenylbenzamide

4-((1-(3-(dimethylamino)propyl)-1H-indol-5-yl)oxy)-N-phenylbenzamide

3-(5-(1-benzyl-1H-pyrazol-4-yl)-1H-indol-1-yl)-N,N-dimethylpropan-1-amine

N,N-dimethyl-3-(5-(1-(1-phenylethyl)-1H-pyrazol-4-yl)-1H-indol-1-yl)propan-1-amine

N,N-dimethyl-3-(5-(1-(1-phenylethyl)-1H-pyrazol-4-yl)-1H-indol-1-yl)propan-1-amine

N,N-dimethyl-3-(5-(1-((tetrahydro-2H-pyran-4-yl)methyl)-1H-pyrazol-4-yl)-1H-indol-1-yl)propan-1-amine

N,N-dimethyl-3-(5-(1-(1-(pyridin-4-yl)ethyl)-1H-pyrazol-4-yl)-1H-indol -1-yl)propan-1-amine

3-((1-(3-(dimethylamino)propyl)-1H-indol-5-yl)methyl)-N-phenylbenzamide

3-(5-(1-benzyl-1H-pyrazol-4-yl)-2,3-dimethyl-1H-indol-1-yl)-N,N-dimethylpropan-1-amine

4-((1-(3-(dimethylamino)propyl)-2,3-dimethyl-1H-indol-5-yl)amino)-N-phenylbenzamide

3-((1-(3-(dimethylamino)propyl)-2,3-dimethyl-1H-indol-5-yl)amino)-N-phenylbenzamide

4-((1-(3-(dimethylamino)propyl)-1H-indol-5-yl)amino)-N-(pyridin-3-yl)benzamide

3-((1-(3-(dimethylamino)propyl)-1H-indol-5-yl)amino)-N-phenylbenzamide

3-((1-(3-(dimethylamino)propyl)-1H-indol-5-yl)oxy)-N-phenylbenzamide

3-(2,3-dimethyl-5-((4-phenethylpiperazin-1-yl)methyl)-1H-indol-1-yl)-N,N-dimethylpropan-1-amine

(1-(3-aminopropyl)-1H-indol-5-yl)(4-phenethylpiperazin-1-yl)methanone

(1-(3-(1H-imidazol-4-yl)propyl)-1H-indol-5-yl)(4-phenethylpiperazin-1-yl)methanone

(1-(3-(dimethylamino)propyl)-2,3-dimethyl-1H-indol-5-yl)(4-phenethylpiperazin-1-yl)methanone

(4-benzylpiperazin-1-yl)(1-(3-(dimethylamino)propyl)-1H-indol-5-yl)methanone

(1-(2-(dimethylamino)ethyl)-1H-indol-5-yl)(4-phenethylpiperazin-1-yl)methanone

(1-(3-(4-methylpiperazin-1-yl)propyl)-1H-indol-5-yl)(4-phenethylpiperazin-1-yl)methanone

(1-(3-(dimethylamino)propyl)-1H-indol-5-yl)(4-isobutylpiperazin-1-yl)methanone

3-(5-(3-isopropoxyphenyl)-1H-indol-1-yl)-N,N-dimethylpropan-1-amine

1-(1-(3-(dimethylamino)propyl)-1H-indole-5-carbonyl)-N-phenylpiperidine-4-carboxamide

(1-methyl-1H-indol-5-yl)(4-phenethylpiperazin-1-yl)methanone

4-((1-(3-(dimethylamino)propyl)-1H-indol-5-yl)amino)-N-phenylbenzamide

4-((1-(3-(dimethylamino)propyl)-1H-indol-5-yl)amino)-N-isopropylbenzamide

4-((1-(3-(dimethylamino)propyl)-1H-indol-5-yl)amino)-N,N-dimethylbenzamide

1-(4-((1-(3-(dimethylamino)propyl)-1H-indol-5-yl)amino)piperidin-1-yl)-2-phenylethan-1-one

N-benzyl-1-(3-(dimethylamino)propyl)-1H-indole-5-carboxamide

1-(3-(dimethylamino)propyl)-N-(1H-indol-2-yl)-1H-indole-5-carboxamide

(3-(benzylamino)pyrrolidin-1-yl)(1-(3-(dimethylamino)propyl)-1H-indol-5-yl)methanone

(1-(3-(dimethylamino)propyl)-1H-indol-5-yl)(4-(phenylsulfonyl)piperazin-1-yl)methanone

N,N-dimethyl-3-(5-((4-phenethylpiperazin-1-yl)methyl)-1H-indol-1-yl)propan-1-amine

1-(3-(dimethylamino)propyl)-N-(1-phenethylpiperidin-4-yl)-1H-indol-5-amine

N,N-dimethyl-3-(5-((4-phenethylpiperidin-1-yl)methyl)-1H-indol-1-yl)propan-1-amine

4-((1-(3-(dimethylamino)propyl)-1H-indol-5-yl)oxy)-N-(pyridin-4-yl)benzamide

4-((1-(3-(dimethylamino)propyl)-1H-indol-5-yl)methyl)-N-(pyridin-4-yl)benzamide

4-((1-(3-(dimethylamino)propyl)-2,3-dimethyl-1H-indol-5-yl)amino)-N-(pyridin-4-yl)benzamide

3-(5-(1-(3H-indol-7-yl)-1H-pyrazol-3-yl)-1H-indol-1-yl)-N,N-dimethylpropan-1-amine

N,N-dimethyl-3-(5-(1-(pyridin-4-yl(1H-pyrrol-2-yl)methyl)-1H-pyrazol-3-yl)-1H-indol-1-yl)propan-1-amine

(4-((4-(1-(3-(dimethylamino)propyl)-2,3-dimethyl-1H-indol-5-yl)-1H-pyrazol-1-yl)methyl)cyclohexyl)methanol

4-(2-(4-((1-(3-(dimethylamino)propyl)-2,3-dimethyl-1H-indol-5-yl)methyl)piperazin-1-yl)ethyl)phenol

(1-(3-(dimethylamino)propyl)-1H-indol-5-yl)(4-phenethylpiperazin-1-yl)methanone

3-((1-(3-(dimethylamino)propyl)-1H-indol-5-yl)methyl)-N-(4-fluorophenyl)benzamide

3-((1-(3-(dimethylamino)propyl)-1H-indol-5-yl)methyl)-N-(4-methoxyphenyl)benzamide

N-(benzo[d][1,3]dioxol-5-yl)-3-((1-(3-(dimethylamino)propyl)-1H-indol-5-yl)methyl)benzamide

3-((1-(3-(dimethylamino)propyl)-1H-indol-5-yl)methyl)-N-(3-fluorophenyl)benzamide

3-((1-(3-(dimethylamino)propyl)-1H-indol-5-yl)methyl)-N-(3-methoxyphenyl)benzamide

3-((1-(3-(dimethylamino)propyl)-2,3-dimethyl-1H-indol-5-yl)methyl)-N-(4-fluorophenyl)benzamide

3-((1-(3-(dimethylamino)propyl)-2,3-dimethyl-1H-indol-5-yl)methyl)-N-(4-methoxyphenyl)benzamide

N-(benzo[d][1,3]dioxol-5-yl)-3-((1-(3-(dimethylamino)propyl)-2,3-dimethyl-1H-indol-5-yl)methyl)benzamide

3-((1-(3-(dimethylamino)propyl)-2,3-dimethyl-1H-indol-5-yl)methyl)-N-(3-fluorophenyl)benzamide

3-((1-(3-(dimethylamino)propyl)-2,3-dimethyl-1H-indol-5-yl)methyl)-N-(3-methoxyphenyl)benzamide

3-((1-(3-(dimethylamino)propyl)-2,3-dimethyl-1H-indol-5-yl)methyl)-N-phenylbenzamide

3-((1-(3-(dimethylamino)propyl)-1H-indol-5-yl)oxy)-N-(4-fluorophenyl)benzamide

3-((1-(3-(dimethylamino)propyl)-1H-indol-5-yl)oxy)-N-(4-methoxyphenyl)benzamide

N-(benzo[d][1,3]dioxol-5-yl)-3-((1-(3-(dimethylamino)propyl)-1H-indol-5-yl)oxy)benzamide

3-((1-(3-(dimethylamino)propyl)-1H-indol-5-yl)oxy)-N-(3-fluorophenyl)benzamide

3-((1-(3-(dimethylamino)propyl)-1H-indol-5-yl)oxy)-N-(3-methoxyphenyl)benzamide

3-((1-(3-(dimethylamino)propyl)-2,3-dimethyl-1H-indol-5-yl)oxy)-N-(4-fluorophenyl)benzamide

3-((1-(3-(dimethylamino)propyl)-2,3-dimethyl-1H-indol-5-yl)oxy)-N-(4-methoxyphenyl)benzamide

N-(benzo[d][1,3]dioxol-5-yl)-3-((1-(3-(dimethylamino)propyl)-2,3-dimethyl-1H-indol-5-yl)oxy)benzamide

3-((1-(3-(dimethylamino)propyl)-2,3-dimethyl-1H-indol-5-yl)oxy)-N-(3-fluorophenyl)benzamide

3-((1-(3-(dimethylamino)propyl)-2,3-dimethyl-1H-indol-5-yl)oxy)-N-(3-methoxyphenyl)benzamide

3-((1-(3-(dimethylamino)propyl)-2,3-dimethyl-1H-indol-5-yl)oxy)-N-phenylbenzamide

3-((1-(3-(dimethylamino)propyl)-1H-indol-5-yl)amino)-N-(4-fluorophenyl)benzamide

3-((1-(3-(dimethylamino)propyl)-1H-indol-5-yl)amino)-N-(4-methoxyphenyl)benzamide

N-(benzo[d][1,3]dioxol-5-yl)-3-((1-(3-(dimethylamino)propyl)-1H-indol-5-yl)amino)benzamide

3-((1-(3-(dimethylamino)propyl)-1H-indol-5-yl)amino)-N-(3-fluorophenyl)benzamide

3-((1-(3-(dimethylamino)propyl)-1H-indol-5-yl)amino)-N-(3-methoxyphenyl)benzamide

3-((1-(3-(dimethylamino)propyl)-2,3-dimethyl-1H-indol-5-yl)amino)-N-(4-fluorophenyl)benzamide

3-((1-(3-(dimethylamino)propyl)-2,3-dimethyl-1H-indol-5-yl)amino)-N-(4-methoxyphenyl)benzamide

N-(benzo[d][1,3]dioxol-5-yl)-3-((1-(3-(dimethylamino)propyl)-2,3-dimethyl-1H-indol-5-yl)amino)benzamide

3-((1-(3-(dimethylamino)propyl)-2,3-dimethyl-1H-indol-5-yl)amino)-N-(3-fluorophenyl)benzamide

3-((1-(3-(dimethylamino)propyl)-2,3-dimethyl-1H-indol-5-yl)amino)-N-(3-methoxyphenyl)benzamide

4-((1-(3-(dimethylamino)propyl)-1H-indol-5-yl)methyl)-N-(4-fluorophenyl)benzamide

4-((1-(3-(dimethylamino)propyl)-1H-indol-5-yl)methyl)-N-(4-methoxyphenyl)benzamide

N-(benzo[d][1,3]dioxol-5-yl)-4-((1-(3-(dimethylamino)propyl)-1H-indol-5-yl)methyl)benzamide

4-((1-(3-(dimethylamino)propyl)-1H-indol-5-yl)methyl)-N-(3-fluorophenyl)benzamide

4-((1-(3-(dimethylamino)propyl)-1H-indol-5-yl)methyl)-N-(3-methoxyphenyl)benzamide

4-((1-(3-(dimethylamino)propyl)-2,3-dimethyl-1H-indol-5-yl)methyl)-N-(4-fluorophenyl)benzamide

4-((1-(3-(dimethylamino)propyl)-2,3-dimethyl-1H-indol-5-yl)methyl)-N-(4-methoxyphenyl)benzamide

N-(benzo[d][1,3]dioxol-5-yl)-4-((1-(3-(dimethylamino)propyl)-2,3-dimethyl-1H-indol-5-yl)methyl)benzamide

4-((1-(3-(dimethylamino)propyl)-2,3-dimethyl-1H-indol-5-yl)methyl)-N-(3-fluorophenyl)benzamide

4-((1-(3-(dimethylamino)propyl)-2,3-dimethyl-1H-indol-5-yl)methyl)-N-(3-methoxyphenyl)benzamide

4-((1-(3-(dimethylamino)propyl)-2,3-dimethyl-1H-indol-5-yl)methyl)-N-phenylbenzamide

4-((1-(3-(dimethylamino)propyl)-1H-indol-5-yl)oxy)-N-(4-fluorophenyl)benzamide

4-((1-(3-(dimethylamino)propyl)-1H-indol-5-yl)oxy)-N-(4-methoxyphenyl)benzamide

N-(benzo[d][1,3]dioxol-5-yl)-4-((1-(3-(dimethylamino)propyl)-1H-indol-5-yl)oxy)benzamide

4-((1-(3-(dimethylamino)propyl)-1H-indol-5-yl)oxy)-N-(3-fluorophenyl)benzamide

4-((1-(3-(dimethylamino)propyl)-1H-indol-5-yl)oxy)-N-(3-methoxyphenyl)benzamide

4-((1-(3-(dimethylamino)propyl)-2,3-dimethyl-1H-indol-5-yl)oxy)-N-(4-fluorophenyl)benzamide

4-((1-(3-(dimethylamino)propyl)-2,3-dimethyl-1H-indol-5-yl)oxy)-N-(4-methoxyphenyl)benzamide

N-(benzo[d][1,3]dioxol-5-yl)-4-((1-(3-(dimethylamino)propyl)-2,3-dimethyl-1H-indol-5-yl)oxy)benzamide

4-((1-(3-(dimethylamino)propyl)-2,3-dimethyl-1H-indol-5-yl)oxy)-N-(3-fluorophenyl)benzamide

4-((1-(3-(dimethylamino)propyl)-2,3-dimethyl-1H-indol-5-yl)oxy)-N-(3-methoxyphenyl)benzamide

4-((1-(3-(dimethylamino)propyl)-2,3-dimethyl-1H-indol-5-yl)oxy)-N-phenylbenzamide

4-((1-(3-(dimethylamino)propyl)-1H-indol-5-yl)amino)-N-(4-fluorophenyl)benzamide

4-((1-(3-(dimethylamino)propyl)-1H-indol-5-yl)amino)-N-(4-methoxyphenyl)benzamide

N-(benzo[d][1,3]dioxol-5-yl)-4-((1-(3-(dimethylamino)propyl)-1H-indol-5-yl)amino)benzamide

4-((1-(3-(dimethylamino)propyl)-1H-indol-5-yl)amino)-N-(3-fluorophenyl)benzamide

4-((1-(3-(dimethylamino)propyl)-1H-indol-5-yl)amino)-N-(3-methoxyphenyl)benzamide

4-((1-(3-(dimethylamino)propyl)-2,3-dimethyl-1H-indol-5-yl)amino)-N-(4-fluorophenyl)benzamide

4-((1-(3-(dimethylamino)propyl)-2,3-dimethyl-1H-indol-5-yl)amino)-N-(4-methoxyphenyl)benzamide

N-(benzo[d][1,3]dioxol-5-yl)-4-((1-(3-(dimethylamino)propyl)-2,3-dimethyl-1H-indol-5-yl)amino)benzamide

4-((1-(3-(dimethylamino)propyl)-2,3-dimethyl-1H-indol-5-yl)amino)-N-(3-fluorophenyl)benzamide

4-((1-(3-(dimethylamino)propyl)-2,3-dimethyl-1H-indol-5-yl)amino)-N-(3-methoxyphenyl)benzamide

3-(5-((4-(4-fluorophenethyl)piperazin-1-yl)methyl)-2,3-dimethyl-1H-indol-1-yl)-N,N-dimethylpropan-1-amine

5-((4-(4-fluorophenethyl)piperazin-1-yl)methyl)-2,3-dimethyl-1-(3-(4-methylpiperazin-1-yl)propyl)-1H-indole

5-((4-(4-methoxyphenethyl)piperazin-1-yl)methyl)-2,3-dimethyl-1-(3-(4-methylpiperazin-1-yl)propyl)-1H-indole

4-(2-(4-((2, 3-dimethyl-1-(3-(4-methylpiperazin-1-yl)propyl)-1H-indol-5-yl)methyl)piperazin-1-yl)ethyl)phenol

5-((4-(2-(benzo[d][1,3]dioxol-5-yl)ethyl)piperazin-1yl)methyl)-2,3-dimethyl-1-(3-(4-methylpiperazin-1-yl)propyl)-1H-indole

5-((4-(3-fluorophenethyl)piperazin-1-yl)methyl)-2,3-dimethyl-1-(3-(4-methylpiperazin-1-yl)propyl)-1H-indole

5-((4-(3-methoxyphenethyl)piperazin-1-yl)methyl)-2,3-dimethyl-1-(3-(4-methylpiperazin-1-yl)propyl)-1H-indole

3-(2-(4-((2,3-dimethyl-1-(3-(4-methylpiperazin-1-yl)propyl)-1H-indol-5-yl)methyl)piperazin-1-yl)ethyl)phenol

2,3-dimethyl-1-(3-(4-methylpiperazin-1-yl)propyl)-5-((4-phenethylpiperazin-1-yl)methyl)-1H-indole

(1-(3-(dimethylamino)propyl)-2, 3-dimethyl-1H-indol-5-yl)(4-(4-fluorophenethyl)piperazin-1-yl)methanone

(1-(3-(dimethylamino)propyl)-2,3-dimethyl-1H-indol-5-yl)(4-(4-hydroxyphenethyl)piperazin-1-yl)methanone

(2,3-dimethyl-1-(3-(4-methylpiperazin-1-yl)propyl)-1H-indol-5-yl)(4-(4-fluorophenethyl)piperazin-1-yl)methanone

(2,3-dimethyl-1-(3-(4-methylpiperazin-1-yl)propyl)-1H-indol-5-yl)(4-(4-methoxyphenethyl)piperazin-1-yl)methanone

(2,3-dimethyl-1-(3-(4-methylpiperazin-1-yl)propyl)-1H-indol-5-yl)(4-(4-hydroxyphenethyl)piperazin-1-yl)methanone

(4-(2-(benzo[d][1,3]dioxol-5-yl)ethyl)piperazin-1-yl)(2,3-dimethyl-1-(3-(4-methylpiperazin-1-yl)propyl)-1H-indol-5-yl)methanone

(2,3-dimethyl-1-(3-(4-methylpiperazin-1-yl)propyl)-1H-indol-5-yl)(4-(3-fluorophenethyl)piperazin-1-yl)methanone

(2,3-dimethyl-1-(3-(4-methylpiperazin-1-yl)propyl)-1H-indol-5-yl)(4-(3-methoxyphenethyl)piperazin-1-yl)methanone

(2,3-dimethyl-1-(3-(4-methylpiperazin-1-yl)propyl)-1H-indol-5-yl)(4-(3-hydroxyphenethyl)piperazin-1-yl)methanone

(2,3-dimethyl-1-(3-(4-methylpiperazin-1-yl)propyl)-1H-indol-5-yl)(4-phenethylpiperazin-1-yl)methanone

In a second aspect, the invention relates to a pharmaceutical composition comprising a compound of formula (I) together with a pharmaceutically acceptable carrier, diluent or excipient.

Compounds and pharmaceutical compositions according to the present invention may be suitable for the treatment or prevention of a proliferative disease. Accordingly, in another aspect, the invention relates to a method of treating or preventing a proliferative disease in a subject, the method comprising administering to the subject an effective amount of a compound of formula (I) according to the first aspect of the invention or a pharmaceutical composition according to the second aspect of the invention.

In a further aspect, the present invention relates to the use of a compound of formula (I) according to the first aspect of the invention or a pharmaceutical composition according to the second aspect of the invention in the manufacture of a medicament for treating or preventing a proliferative disease.

In a further aspect, the present invention relates to the use of a compound of formula (I) according to the first aspect of the invention or a pharmaceutical composition according to the second aspect of the invention for the treatment or prevention of a proliferative disease in a subject.

In a further aspect, the present invention relates to a compound of formula (I) according to the first aspect of the invention or a pharmaceutical composition according to the second aspect of the invention for use in the treatment or prevention of a proliferative disease in a subject.

In a further aspect, the present invention relates to a pharmaceutical composition for use in the treatment or prevention of a proliferative disease in a subject, in any of the embodiments described in the specification.

In a further aspect, the present invention relates to a compound of formula (I) according to the first aspect of the invention or a pharmaceutical composition according to the second aspect of the invention when used in a method of treating or preventing a proliferative disease in a subject.

In a further aspect, the present- invention relates to a composition having an active ingredient for use in a method of treating or preventing a proliferative disease in a subject, wherein the active ingredient is a compound of formula (I) according to the first aspect of the invention.

In a further aspect, the present invention relates to the use of a compound of formula (I) according to the first aspect of the invention or a pharmaceutical composition according to the second aspect of the invention in treating or preventing a proliferative disease in a subject, such as described herein.

In one embodiment, a compound of formula (I) according to the first aspect of the invention is the only active administered to the subject. In one embodiment, a compound of formula (I) according to the first aspect of the invention is the only active in the pharmaceutical composition.

In one or more preferred embodiments, the proliferative disease is cancer, preferably a solid tumour. In various preferred embodiments, the cancer is selected from the group consisting of breast cancer, lung cancer, prostate cancer, ovarian cancer, uterine cancer, brain cancer, skin cancer, colon cancer and bladder cancer.

Those skilled in the art will understand that in the context of the present invention an ‘effective amount’ is an amount sufficient to produce a desired therapeutic or pharmacological effect in the subject being treated.

In a further aspect, the present invention relates to a method of completely or partially preventing the recurrence of a solid tumor in a subject, the method comprising administering to the subject an effective amount of a compound of formula (I) according to the first aspect of the invention or a pharmaceutical composition according to the second aspect of the invention.

In another aspect, the present invention relates to the use of a compound according to the first aspect of the invention or the pharmaceutical composition according to the second aspect of the invention in the manufacture of a medicament for completely or partially preventing the recurrence of a solid tumor.

In a further aspect, the present invention relates to the use of a compound of formula (I) according to the first aspect of the invention or a pharmaceutical composition according to the second aspect of the invention for completely or partially preventing the recurrence of a solid tumor in a subject.

In a further aspect, the present invention relates to a compound of formula (I) according to the first aspect of the invention or a pharmaceutical composition according to the second aspect of the invention for use in completely or partially preventing the recurrence of a solid tumor in a subject.

In a further aspect, the present invention relates to a pharmaceutical composition for use in completely or partially preventing the recurrence of a solid tumor in a subject, in any of the embodiments described in the specification.

In a further aspect, the present invention relates to a compound of formula (I) according to the first aspect of the invention or a pharmaceutical composition according to the second aspect of the invention when used in a method of completely or partially preventing the recurrence of a solid tumor in a subject.

In a further aspect, the present invention relates to a composition having an active ingredient for use in a method of completely or partially preventing the recurrence of a solid tumor, wherein the active ingredient is a compound of formula (I) according to the first aspect of the invention.

In a further aspect, the present invention relates to the use of a compound of formula (I) according to the first aspect of the invention or a pharmaceutical composition according to the second aspect of the invention in completely or partially preventing the recurrence of a solid tumor, such as described herein.

In one embodiment, a compound of formula (I) according to the first aspect of the invention is the only active administered to the subject. In one embodiment, a compound of formula (I) according to the first aspect of the invention is the only active in the pharmaceutical composition.

The compounds of formula (I) may be used in therapy alone or in combination with one or more other chemotherapeutic agents, for example, as part of a combination therapy.

In another aspect the present invention relates to a process for preparing a compound of formula (I) comprising the step of:

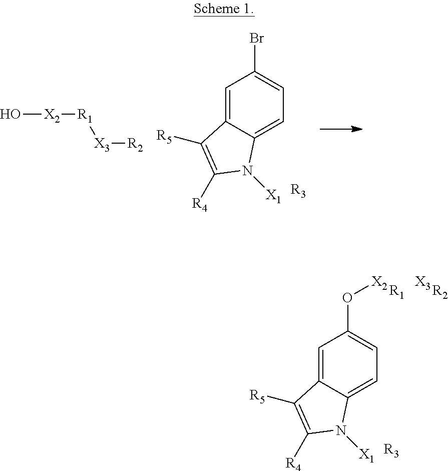

In another aspect, the present invention relates to a process for preparing a compound of formula (I) comprising the step of:

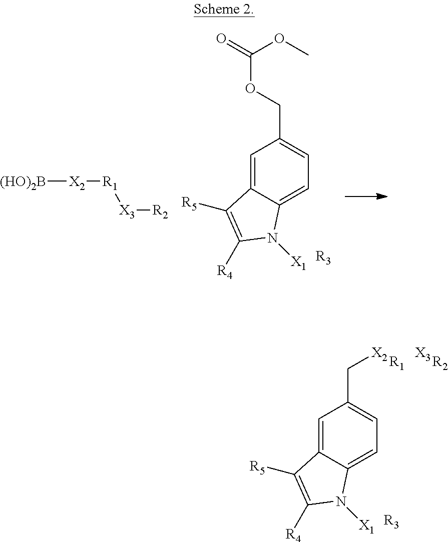

In another aspect, the present invention relates to a process for preparing a compound of formula (I) comprising the step of:

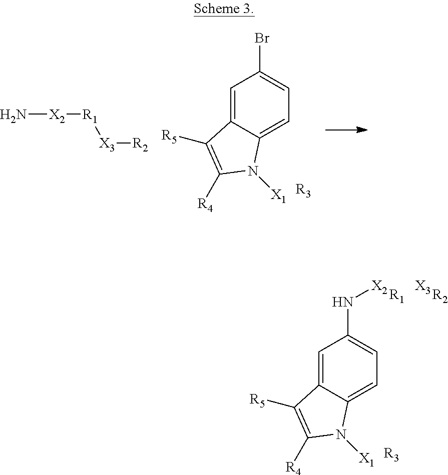

In another aspect, the present invention relates to a process for preparing a compound of formula (I) comprising the step of:

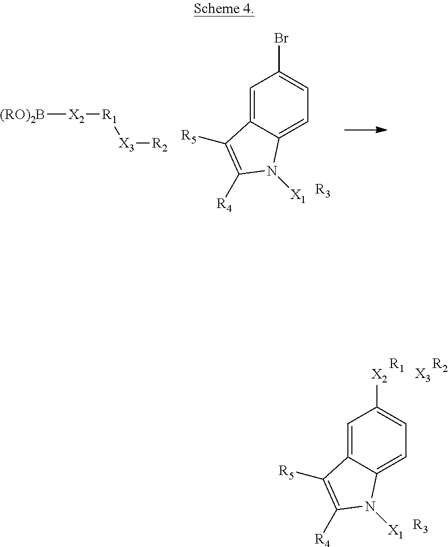

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Activity of selected compounds against Tm5NM1/2 transfected mouse embryonic fibroblasts versus non-transfected MEFs.

FIG. 2: Imaging and quantitation of actin filaments in SK—N—SH neuroblastoma cells treated with compound (A) 4093 and (B) 4113. Cells were stained with 488-Atto-Phallodin and DAPI to visualize the actin filament bundles and the nucleus, respectively. Shown in the top panel is a representative grey scale immunofluorescent image from control (vehicle alone), 5 μM and 10 μM treated cells. The middle panel (with enlarged inset shown in the bottom panel) shows the overlay of the cell image with the linear feature quantitation. The coloured lines indicate the detected actin filaments. Also shown is the quantitation of cell number, filament number/cell and filament number/cell area (μM²). Statistical analysis was performed using a one way ANNOVA-multiple comparison where each drug treated group was compared to the control. **** p<0.001, *** p<0.01, ** p<0.1, *p<0.5.

FIG. 3: Imaging and quantitation of actin filaments in SK—N—SH neuroblastoma cells treated with compound (A) 4093 and (B) 4113. Cells were stained with γ9d (sheep polycolonal, 1:100) followed by 488-conjugated secondary (1:1000) and DAPI to visualize the Tm5NM1 containing filament bundles and the nucleus, respectively. Shown in the top panel is a representative grey scale immunofluorescent image from control (vehicle alone), 5 μM and 10 μM treated cells. The middle panel (with enlarged inset shown in the bottom panel) shows the overlay of the cell image with the linear feature quantitation. The coloured lines indicate the detected actin filaments. Also shown is the quantitation of cell number, filament number/cell and filament number/cell area (μM²). Statistical analysis was performed using a one way ANNOVA-multiple comparison where each drug treated group was compared to the control. **** p<0.001, *** p<0.01, ** p<0.1, *p<0.5.

FIG. 4: Impact of compounds 4015 and 4093 on Tm5NM1-regulated actin-filament depolymerization kinetics. (A, C and E) Depolymerization time course of 6 μM actin filaments (35% pyrene labelled) diluted 12-fold into F-actin buffer (100 mM NaCl, 10 mM Tris-HCl pH 7.0, 2 mM MgCl₂, 1 mM EGTA, 0.2 mM CaCl₂, 0.2 mM ATP, 0.5 mM DTT, 0.01% (v/v) NaN3) in the presence or absence of saturating amounts (10 μM) of Tm5NM1. Final concentration of F-actin and Tm5NM1 was 0.5 μM and 0.83 μM respectively. Tm5NM1 was pre-incubated with 50 μM 4015, 4093 or 1% (v/v) DMSO prior to mixing with F-actin. Depolymerization data is normalized to the initial fluorescence value. (B, D and F) Initial rates (V₀) of depolymerization for F-actin alone or Tm5NM1/F-actin, in the presence of compound 4015 or 4093. Initial rates of depolymerization were determined from the first 3600 s, fitted to a linear regression model. Data represents mean ±SEM, averaged from n>8 replicates. **** p<0.0001, ** p<0.005.

FIG. 5: Average body weight measurement ±SEM (grams) of animals in Control (Carbonate vehicle) and compound 4015 (20 mg/kg, I.V., QD) treated animals.

FIG. 6: Tumor volumes ±SEM of animals treated with Control (Carbonate vehicle) or compound 4015 (20 mg/kg, I.V., QD).

### DETAILED DESCRIPTION OF THE EMBODIMENTS

The invention is based on the surprising finding that compounds of general formula (I) effectively inhibit tropomyosin, which results in unexpected improvement in the treatment of proliferative diseases, particularly cancer. The development of the actin cytoskeleton involves a number of ancillary control and regulatory proteins. Identification and specific targeting of actin regulatory proteins associated with the cytoskeleton of cancer cells offers the opportunity to develop cancer specific drugs without unwanted side effects.

Actin filaments are constructed through the polymerisation of globular actin protein monomers. The actin monomer is polar with one end bearing a positive charge and the other end a negative charge. The actin filaments thus have all the actin proteins aligned in one direction. These filaments have secondary coiled proteins (tropomyosins) associated with them. The tropomyosins play an integral role in regulating the function of actin filaments. Structurally the actin filaments are made up of polymeric actin monomers with tropomyosin dimers sitting in the alpha helical groove of the actin filament to form a homopolymer. There are more than 40 mammalian tropomyosin isoforms each of which regulates specific actin filaments. There are specific isoforms of tropomyosins that regulate the cytoskeleton of cancer cells, disruption of this interaction offers a basis to specifically treat cancer cells.

### I. Definitions

The following are some definitions of terms used in the art that may be helpful in understanding the description of the present invention. These are intended as general definitions and should in no way limit the scope of the present invention to those terms alone, but are put forth for a better understanding of the following description.

Unless the context requires otherwise or specifically states to the contrary, integers, steps, or elements of the invention recited herein as singular integers, steps or elements clearly encompass both singular and plural forms of the recited integers, steps or elements.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps, features, compositions and compounds.

The terms “comprising” and “including” are used herein in their open-ended and non- limiting sense unless otherwise noted.

The term “optionally substituted” as used throughout the specification denotes that the group may or may not be further substituted or fused (so as to form a polycyclic system), with one or more non-hydrogen substituent groups. Suitable chemically viable optional substituents for a particular functional, group will be apparent to those skilled in the art. Typical optional substituents include C₁-C₄ alkyl, C₂-C₄ alkenyl, OH, halogen, O(C₁-C₄ alkyl), NR^{a}R^{b} wherein R^{a} and R^{b} are independently selected from H, C₁-C₃ alkyl, CONH₂, SH, S(C₁-C₃ alkyl), —CH₂—O(C₁₋₃ alkyl), C₆₋₁₀ aryl, —CH₂-phenyl, hydroxyl-(C₁₋₃ alkyl), and halo-(C₁₋₃ alkyl). Presently preferred optional substituents include C₁₋₃ alkyl, C₁₋₃ alkoxy, —CH₂-(C₁₋₃)alkoxy, C₆₋₁₀ aryl, —CH₂-phenyl, halogen, OH, hydroxy-(C₁₋₃)alkyl, and halo-(C₁₋₃)alkyl, e.g, CF₃, CH₂CF₃.

“Acyl” means an alkyl-CO- group in which the alkyl group is as described herein. Examples of acyl include acetyl and benzoyl. The alkyl group may be a C₁-C₆ alkyl, C₁-C₄ alkyl, or C₁-C₃ alkyl group. The group may be a terminal group or a bridging group.

“Alkyl” as a group or part of a group refers to a straight or branched aliphatic hydrocarbon group having 1-12 carbon atoms, or 1-10 carbon atoms, or 1-6 carbon atoms, or 1-4 carbon atoms, or 1-3 carbon atoms. Thus, for example, the term alkyl includes, but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethyl pentyl, 1,2,3-trimethylbutyl, 1,1,2-trimethylbutyl, 1,1,3-trimethylbutyl, 5-methylheptyl, 1-methylheptyl, octyl, nonyl, decyl, and the like. The group may be a terminal group or a bridging group.

“Alkenyl” as a group or part of a group denotes an aliphatic hydrocarbon group containing at least one carbon-carbon double bond and which may be straight or branched such as a group having 2-12 carbon atoms, or 2-6 carbon atoms, or 2-4 carbon atoms, in the normal chain. The group may contain a plurality of double bonds in the normal chain and the orientation about each double bond is independently cis or trans, E or Z. Exemplary alkenyl groups include, but are not limited to, ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 1-nonenyl, 1-decenyl, and the like. The group may be a terminal group or a bridging group.

“Alkenyloxy” refers to an —O— alkenyl group in which alkenyl is as defined herein. Preferred alkenyloxy groups are C₂-C₁₂ alkenyloxy groups. The group may be a terminal group or a bridging group.

The terms “alkyloxy” and “alkoxy” are synonymous and refer to an —O-alkyl group in which alkyl is as defined herein. Presently preferred alkoxy groups are C₁₋₆ alkoxy or C₁₋₄ alkoxy or C₁₋₃ alkoxy. Examples include, but are not limited to, methoxy, ethoxy, n-propoxy, iso-propoxy, sec-butoxy, tert-butoxy, and the like. The group may be a terminal group or a bridging group.

“Alkylamino” (or “aminoalkyl”) includes both mono-alkylamino and dialkylamino, unless specified. “Mono-alkylamino” means a —NH-alkyl group, in which alkyl is as defined above. “Dialkylamino” means a —N(alkyl)₂ group, in which each alkyl may be the same or different and are each as defined herein for alkyl. The alkyl group may be a C₁-C₆ alkyl group. The group may be a terminal group or a bridging group.

“Alkynyl” as a group or part of a group means an aliphatic hydrocarbon group containing a carbon-carbon triple bond and which may be straight or branched and may have from 2-12 carbon atoms or 2-6 carbon atoms or 2-4 carbon atoms in the normal chain. Exemplary structures include, but are not limited to, ethynyl and propynyl. The group may be a terminal group or a bridging group.

“Alkynyloxy” refers to an —O-alkynyl group in which alkynyl is as defined herein. Presently preferred alkynyloxy groups are C₂-C₆ alkynyloxy groups, C₂-C₄ alkynyloxy. The group may be a terminal group or a bridging group.

“Aryl” as a group or part of a group denotes (i) an optionally substituted monocyclic, or fused polycyclic, aromatic carbocycle (ring structure having ring atoms that are all carbon) that may have from 5-18 atoms per ring. Presently preferred aryl groups have 6-14 atoms per ring, or more preferably 6-10 atoms per ring. Examples of aryl groups include phenyl, naphthyl, phenanthryl and the like; (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. The group may be a terminal group or a bridging group.

“Cycloalkenyl” means a non-aromatic monocyclic or multicyclic ring system containing at least one carbon-carbon double bond and may have from 5-10 carbon atoms per ring. Exemplary monocyclic cycloalkenyl rings include cyclopentenyl, cyclohexenyl or cycloheptenyl. The cycloalkenyl group may be substituted by one or more substituent groups. The group may be a terminal group or a bridging group.

“Cycloalkyl” refers to a saturated or partially saturated, monocyclic or fused or spiro polycyclic, carbocycle that may contain from 3 to 9 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. The group may be a terminal group or a bridging group.

The terms “halogen” or “halo” are synonymous and refer to fluorine, chlorine, bromine or iodine.

“Heteroaryl” either alone or as part of a group refers to groups containing an aromatic ring (such as a 5- or 6-membered aromatic ring) having one or more heteroatoms as ring atoms in the aromatic ring with the remainder of the ring atoms being carbon atoms. Suitable heteroatoms include nitrogen, oxygen and sulphur. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl. The group may be a terminal group or a bridging group.

The term “heteroatom” or variants such as “hetero-” as used herein refers to O, N, NH and S.

Certain compounds of the disclosed embodiments may exist as single stereoisomers, racemates, and/or mixtures of enantiomers and/or diastereomers. All such single stereoisomers, racemates and mixtures thereof, are intended to be within the scope of the subject matter described and claimed.

Additionally, formula (I) is intended to cover, where applicable, solvated as well as unsolvated forms of the compounds. Thus, formula (I) includes compounds having the indicated structure, including the hydrated or solvated form, as well as the non-hydrated and non-solvated forms.

The term “pharmaceutically acceptable salt” refers to those salts which, within the scope of sound medical judgement, are suitable for use in contact with the tissues of humans and animals without undue toxicity, irritation, allergic response and the like, and are commensurate with a reasonable benefit/risk ratio. Pharmaceutically acceptable salts are well known in the art. S. M. Berge et al. describe pharmaceutically acceptable salts in detail in J. Pharmaceutical Sciences, 1977, 66:1-19. The salts can be prepared in situ during the final isolation and purification of the compounds of the invention, or separately by reacting the free base function with a suitable organic acid. Suitable pharmaceutically acceptable acid addition salts of the compounds of the present invention may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, ascorbic, glucoronic, fumaric, maleic, pyruvic, alkyl sulfonic, arylsulfonic, aspartic, glutamic, benzoic, anthranilic, mesylic, salicylic, p-hydroxybenzoic, phenylacetic, mandelic, ambonic, pamoic, pantothenic, sulfanilic, cyclohexylaminosulfonic, stearic, algenic, β-hydroxybutyric, galactaric, and galacturonic acids. Suitable pharmaceutically acceptable base addition salts of the compounds of the present invention include metallic salts made from lithium, sodium, potassium, magnesium, calcium, aluminium, and zinc, and organic salts made from organic bases such as choline, diethanolamine, morpholine. Alternatively, organic salts made from N,N′-dibenzylethylenediamine, chloroprocaine, choline, diethanolamine, ethylenediamine, meglumine (N-methylglucamine), procaine, ammonium salts, quaternary salts such as tetramethylammonium salt, amino acid addition salts such as salts with glycine and arginine. In the case of compounds that are solids, it will be understood by those skilled in the art that the inventive compounds, agents and salts may exist in different crystalline or polymorphic forms, all of which are intended to be within the scope of the present invention and specified formulae.

“Prodrug” means a compound which is convertible in vivo by metabolic means (e.g. by hydrolysis, reduction or oxidation) to a compound of the present invention. For example an ester prodrug of a compound of the present invention containing a hydroxyl group may be convertible by hydrolysis in vivo to the parent molecule. Suitable esters are for example, acetates, citrates, lactates, tartrates, malonates, oxalates, salicylates, propionates, succinates, fumarates, maleates, methylene-bis-β-hydroxynaphthoates, gestisates, isethionates, di-ρ-toluoyltartrates, methanesulphonates, ethanesulphonates, benzenesulphonates, ρ-toluenesulphonates, cyclohexylsulphamates and quinates.

The terms “treating”, “treatment” and “therapy” are used herein to refer to curative therapy, prophylactic therapy and preventative therapy. Thus, in the context of the present disclosure the term “treating” encompasses curing, ameliorating or tempering the severity of cancer or its associated symptoms.

“Preventing” or “prevention” means preventing the occurrence of the cancer or tempering the severity of the cancer if it develops subsequent to the administration of the compounds or pharmaceutical compositions of the present invention. This prevents the onset of clinically evident unwanted cell proliferation altogether or the onset of a preclinically evident stage of unwanted rapid cell proliferation in individuals at risk. Also intended to be encompassed by this definition is the prevention of metastases of malignant cells or the arrest or reversal of the progression of malignant cells.

The terms “therapeutically effective” or “pharmacologically effective” are intended to qualify the amount of each agent which will achieve the goal of improvement in disease severity and the frequency of incidence over treatment of each agent by itself while avoiding adverse side effects typically associated with other therapies.

A “pharmaceutical carrier, diluent or excipient” includes, but is not limited to, any physiological buffered (i.e., about pH 7.0 to 7.4) medium comprising a suitable water soluble organic carrier, conventional solvents, dispersion media, fillers, solid carriers, coatings, antibacterial and antifungal agents, isotonic and absorption delaying agents. Suitable water soluble organic carriers include, but are not limited to saline, dextrose, corn oil, dimethylsulfoxide, and gelatin capsules. Other conventional additives include lactose, mannitol, corn starch, potato starch, binders such as crystalline cellulose, cellulose derivatives, acacia, gelatins, disintegrators such as sodium carboxymethyl-cellulose, and lubricants such as talc or magnesium stearate.

“Subject” includes any human or non-human animal. Thus, in addition to being useful for human treatment, the compounds of the present invention may also be useful for veterinary treatment of mammals, including companion animals and farm animals, such as, but not limited to dogs, cats, horses, cows, sheep, and pigs.

In the context of this specification the term “administering” and variations of that term including “administer” and “administration”, includes contacting, applying, delivering or providing a compound or composition of the invention to an organism, or a surface by any appropriate means.

### II. Synthesis of Compounds of the Invention

The present invention relates to functionalized indole compounds of general formula (I) as defined herein, and to the use of such compounds as anticancer agents.

Compounds of general formula (I), or salts, hydrates or solvates thereof, may be prepared by methods known to those skilled in the art. The general synthetic schemes for preparing compounds of formula (I) are described below.

Compounds belonging to the indole families are prepared as shown below. The first step of a presently preferred synthetic route for preparing compounds of formula (I) is the preparation of a suitably N-alkylated intermediate as shown in Scheme 5.

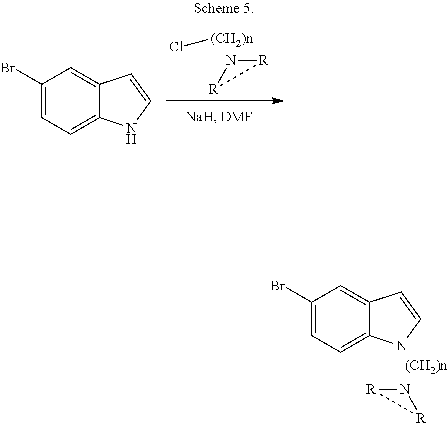

These compounds can then be further ligated with a number of linking groups, specific conditions being used for compounds linked with C, O, N groups as shown in Scheme 6.

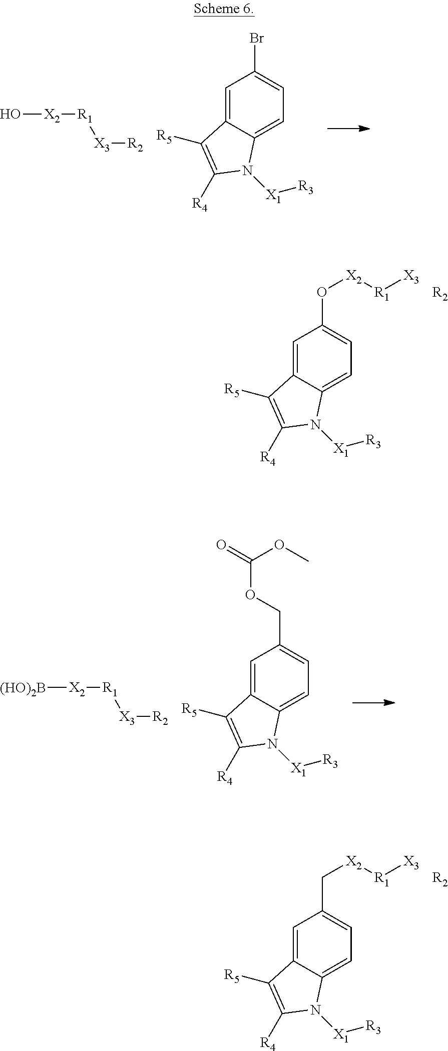

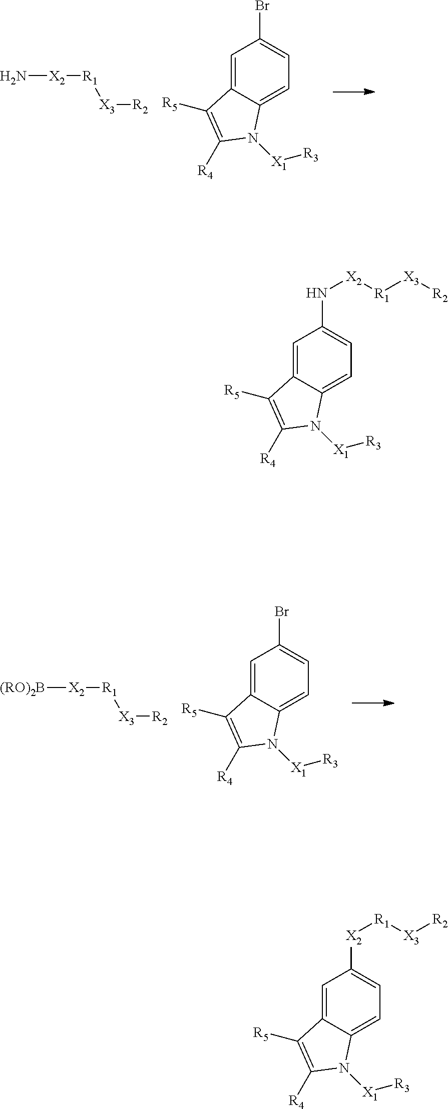

The methods described above in Schemes 5-6 may offer one or more advantages including high yields, control of stereochemistry, few synthetic steps and reaction conditions that are amenable to large scale manufacture. The methods described above are merely representative and routine modifications and variations that would be apparent to persons skilled in the art fall within the broad scope and ambit of the invention disclosed herein.

### Methods of Treatment Using Compounds of the Invention

The compounds of general formula (I) according to the present invention, and pharmaceutical compositions thereof, may be used in the treatment or prevention of proliferative diseases, preferably cancer. The compounds and compositions of the invention may be useful for the treatment of a wide variety of cancers (tumours), including but not limited to, solid tumours, such as for example, breast cancer, lung cancer, prostate cancer, ovarian cancer, uterine cancer brain cancer, skin cancer, colon cancer and bladder cancer.

Advantageously, compounds of the present invention may possess superior pharmaceutical properties, such as improved resistance to conjugation via glucuronyl transferases and other water solubilizing transferases such as sulfases, which may be over-expressed on proliferative cells such as cancer cells. This may advantageously confer superior pharmaceutical properties, such as an enhanced pharmacokinetic profile through reduced conjugation and elimination.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

The compounds or pharmaceutical compositions of the present invention may be administered orally, intravenously, intranasally, rectally, parenterally, subcutaneously, intramuscularly, topically or by any means which delivers an effective amount of the active agent to the tissue or site to be treated. It will be appreciated that different dosages may be required for treating different disorders. An effective amount of an agent is that amount which causes a statistically significant decrease in neoplastic cell count, growth, or size. Neoplastic disorders responsive to the agents of the present invention include, but are not limited to, breast cancer.

The dosage form and amount of the compounds or pharmaceutical compositions of the present invention can be readily established by reference to known treatment or prophylactic regimens.

For example, the compounds and pharmaceutical compositions may be formulated for oral, injectable, rectal, parenteral, subcutaneous, intravenous or intramuscular delivery. Non-limiting examples of particular formulation types include tablets, capsules, caplets, powders, granules injectables, ampoules, vials, ready-to-use solutions or suspensions, lyophilized materials, suppositories and implants. The solid formulations such as the tablets or capsules may contain any number of suitable pharmaceutically acceptable expients or carriers described above.

For intravenous, intramuscular, subcutaneous, or intraperitoneal administration, one or more compounds may be combined with a sterile aqueous solution which is preferably isotonic with the blood of the recipient. Such formulations may be prepared by dissolving solid active ingredient in water containing physiologically compatible substances such as sodium chloride or glycine, and having a buffered pH compatible with physiological conditions to produce an aqueous solution, and rendering said solution sterile. The formulations may be present in unit or multi-dose containers such as sealed ampoules or vials.

The amount of therapeutically effective compound that is administered and the dosage regimen for treating a disease condition with the compounds and/or pharmaceutical compositions of the invention depends on a variety of factors, including the age, weight, sex, and medical condition of the subject, the severity of the disease, the route and frequency of administration, the particular compound employed, the location of the unwanted proliferating cells, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

The compounds of the present invention may be administered along with a pharmaceutical carrier, diluent or excipient as described above. Alternatively, or in addition to, the compounds may be administered in combination with other agents, for example, chemotherapeutic or immune-stimulating drugs or therapeutic agents.

The terms “combination therapy” or “adjunct therapy” in defining use of a compound of the present invention and one or more other pharmaceutical agents, are intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations of each agent.

In accordance with various embodiments of the present invention one or more compounds of general formula (I) may be formulated or administered in combination with one or more other therapeutic agents. Thus, in accordance with various embodiments of the present invention, one or more compounds of general formula (I) may be included in combination treatment regimens with surgery and/or other known treatments or therapeutic agents, such as other anticancer agents, in particular, chemotherapeutic agents, radiotherapeutic agents, and/or adjuvant or prophylactic agents.

There are large numbers of antineoplastic agents available in commercial use, in clinical evaluation and in pre-clinical development, which could be selected for treatment of cancers or other neoplasias by combination drug chemotherapy. Such anti-neoplastic agents fall into several major categories, namely, antibiotic-type agents, antimetabolite agents, hormonal agents, immunological agents, interferon-type agents and a category of miscellaneous agents. Alternatively, other anti-neoplastic agents, such as metallomatrix proteases inhibitors may be used. Suitable agents which may be used in combination therapy will be recognized by those of skill in the art. Suitable agents are listed, for example, in the Merck Index, An Encyclopaedia of Chemicals, Drugs and Biologicals, 12^{th} Ed., 1996, the entire contents of which are incorporated herein by reference.

Combination regimens may involve the active agents being administered together, sequentially, or spaced apart as appropriate in each case. Combinations of active agents including compounds of the invention may be synergistic.

The co-administration of compounds of the general formula (I) may be effected by a compound of the general formula (I) being in the same unit dose as a chemotherapeutic or other anti-cancer agent, or the compound of the general formula (I) and the chemotherapeutic or other anti-cancer agents may be present in individual and discrete unit doses administered at the same, or at a similar time. Sequential administration may be in any order as required, and may require an ongoing physiological effect of the first or initial compound to be current when the second or later compound is administered, especially where a cumulative or synergistic effect is desired.

Embodiments of the invention will now be discussed in more detail with reference to the examples which are provided for exemplification only and which should not be considered limiting on the scope of the invention in any way.

### EXAMPLES

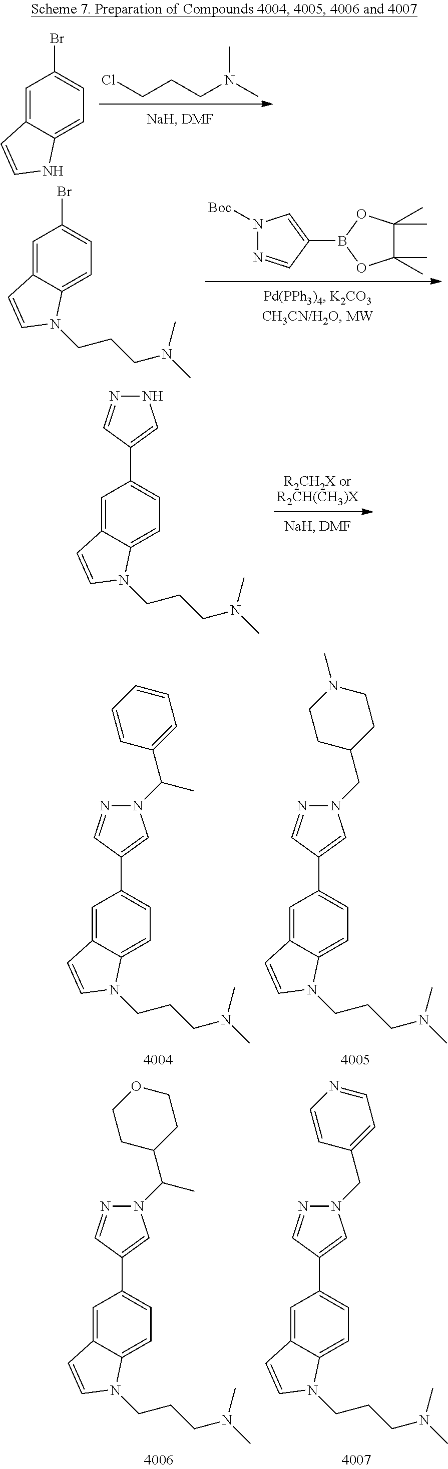

### Preparation of 3-(5-bromo-1H-indol-1-yl)-N,N-dimethylpropan-1-amine

To a suspension of NaH (1.24 g, 30.6 mmol, 60%) in DMF (20 mL) was added 5-bromoindole (2.0 g, 10.2 mmol) at 0° C. The resulting mixture was stirred for 20 minutes. Then a solution of 3-chloro-N,N-dimethylpropan-1-amine (1.77 g, 12.24 mmol) in DMF was added to the reaction mixture at 0° C., and the whole mixture was allowed to warm to 50° C. overnight. TLC showed that all of the starting material had been consumed. The reaction mixture was extracted with EtOAc. The organic layer was washed with water and brine, dried over Na₂SO₄ and concentrated to give the crude product. Column chromatography on silica gel (MeOH/DCM=1/30) afforded the target compound (2.6 g, 91%).

### Preparation of 3-(5-(1H-pyrazol-4-yl)-1H-indol-1-yl)-N,N-dimethylpropan-1-amine

A mixture of 5-bromo-1-N,N-dimethylaminopropylindole (500 mg, 1.78 mmol), Boc-pyrazole-4-pinacolboronate (785.3 mg, 2.67 mmol), Pd[PPh₃]₄ (108 mg, 0.09 mmol), and K₂CO₃ (491.2 mg, 3.56 mmol) in CH₃CN/H₂O (5.5 mL, 10:1) was reacted in a microwave at 110° C. for 2 hours. The solvents were removed under vacuum and ethyl acetate was added to the residue. The solution was washed with brine and dried over Na₂SO₄. After concentration, the crude product was purified by silica column chromatography to afford the target compound (240 mg, 50%).

¹H NMR (400 MHz, d₆-DMSO): δ 11.90 (br s, 1H), 8.05 (br s, 1H), 7.95 (br s, 1H), 7.45 (s, 1H), 7.45-7.38 (m, 2H), 7.33 (d, J=3.2 Hz, 1H), 6.40 (d, J=3.2 Hz, 1H), 4.18 (t, J=7.0 Hz, 2H), 2.18-2.16 (m, 2H), 2.14 (s, 6H), 1.88 (quintet, J=7.0 Hz, 2H).

### Preparation of Compound 4004, N,N-dimethyl-3-(5-(1-(1-phenylethyl)-1H-pyrazol-4-yl)-1H-indol-1-yl)propan-1-amine

To a mixture of NaH (40 mg, 1 mmol, 60%) in DMF was added 3-(5-(1H-pyrazol-4-yl)-1H-indol-1-yl)-N,N-dimethylpropan-1-amine (134.2 mg, 0.5 mmol) at 0° C. After 20 minutes, a solution of 2-bromoethylbenzene (111 mg, 0.6 mmol) in DMF was added slowly. The mixture was stirred at 50° C. overnight. The reaction mixture was poured into ice water and extracted with ethyl acetate. The organic phase was washed with brine, dried over Na₂SO₄, and filtered. After concentration, the residue was purified by preparative TLC to give the desired product (110 mg, 59%).

¹H NMR (400 MHz, d₆-DMSO): δ 8.26 (br s, 1H), 7.87 (br s, 1H), 7.75 (br s, 1H), 7.46 (d, J=8.8 Hz, 1H), 7.40-7.27 (m, 7H), 6.41 (d, J=2.8 Hz, 1H), 5.61 (q, J=7.2 Hz, 1H), 4.20 (t, J=6.8 Hz, 2H), 2.38-2.34 (m, 2H), 2.27 (s, 6H), 1.94 (quintet, J=6.8 Hz, 2H), 1.86 (d, J=7.2 Hz, 3H). LCMS: m/z 373.2 [M+H]⁺.

### Preparation of Compound 4005, N,N-dimethyl-3-(5-(1-((1-methylpiperidin-4-yl)methyl)-1H-pyrazol-4-yl)-1H-indol-1-yl)propan-1-amine

To a mixture of 1-methyl-4-hydroxymethylpiperidine (1 g, 7.7 mmol) in dichloromethane (20 mL) was added Et₃N (860.1 mg, 1.1 mmol) and DMAP (47 mg, 0.385 mmol). MsCl (973.6 mg, 8.5 mmol) was added slowly at 0° C. The mixture was stirred at room temperature for two hours. Water (30 mL) was added. The water phase was extracted with DCM, and the DCM layer was washed with brine and dried over Na₂SO₄. After filtration and concentration, the target (1-methylpiperidin-4-yl)methyl methanesulfonate was obtained (1.5 g, 94%).

Next, to a mixture of NaH (26.8 mg, 0.67 mmol, 60%) in DMF (3 mL) was added 3-(5-(1H-pyrazol-4-yl)-1H-indol-1-yl)-N,N-dimethylpropan-1-amine (90 mg, 0.355 mmol) at 0° C. After 20 minutes, (1-methylpiperidin-4-yl)methyl methanesulfonate (82.8 mg, 0.40 mmol) was added portionwise. The solution was then heated to 100° C. and stirred overnight. The reaction mixture was poured into ice water and extracted with ethyl acetate. The organic phase was washed with brine, dried over Na₂SO₄, filtered, concentrated, and purified by preparative HPLC to give the product (14 mg, 11%).

¹H NMR (400 MHz, CD₃OD): δ 7.95 (s, 1H), 7.82 (s, 1H). 7.74 (s, 1H), 7.42-7.36 (m, 2H), 7.21 (d, J=3.2 Hz, 1H), 6.45 (d, J=3.2 Hz, 1H), 4.21-4.03 (m, 4H), 3.37-3.32 (m, 1H), 2.88-2.78 (m, 1H), 2.69-2.46 (m, 1H), 2.33-2.13 (m, 12H), 2.08-1.89 (m, 5H), 1.65-1.31 (m, 2H). LCMS: m/z 380.2 [M+H]⁺.

### Preparation of Compound 4006, N,N-dimethyl-3-(5-(1-((tetrahydro-2H-pyran-4-yl)methyl)-1H-pyrazol-4-yl)-1H-indol-1-yl)propan-1-amine

To a mixture of NaH (40 mg, 1 mmol, 60%) in DMF (3 mL) at 0° C. was added 3-(5-(1H-pyrazol-4-yl)-1H-indol-1-yl)-N,N-dimethylpropan-1-amine (134.2 mg, 0.5 mmol). After 20 minutes, 4-bromomethyltetrahydropyran (108 mg, 0.6 mmol) was added slowly. The mixture was stirred at 50° C. overnight. The mixture was poured into ice water and extracted with ethyl acetate. The combined ethyl acetate phases were washed with brine, dried over Na₂SO₄, filtered, and concentrated to give the product (80 mg, 44%).

¹H NMR (400 MHz, CDCl₃): δ 7.83 (s, 1H), 7.75 (s, 1H), 7.62 (s, 1H), 7.40-7.34 (m, 2H), 7.14 (d, J=3.2 Hz, 1H), 6.50 (d, J=3.2 Hz, 1H), 4.22 (t, J=7.0 Hz, 2H), 4.05-3.98 (m, 4H), 3.44-3.37 (m, 2H), 2.28-2.19 (m, 9H), 2.03-1.97 (m, 2H), 1.59-1.56 (m, 2H), 1.47-1.38 (m, 2H). LCMS: m/z 367.3 [M+H]⁺.

### Preparation of Compound 4007, N,N-dimethyl-3-(5-(1-(1-(pyridin-4-yl)ethyl)-1H-pyrazol-4-yl)-1H-indol-1-yl)propan-1-amine

To a mixture of 1-(pyridin-4-yl)ethan-1-ol (1.0 g, 8.1 mmol) in DCM (20 mL) were added Et₃N (980 mg, 9.7 mmol) and DMAP (50 mg, 0.40 mmol). MsCl (1.12 g, 9.7 mmol) was added slowly at 0° C. The mixture was stirred at room temperature for two hours. Water (20 mL) was added. The water phase was extracted with DCM and the combined organics were washed with brine and dried over Na₂SO₄. After filtration and concentration, 1-(pyridin-4-yl)ethyl methanesulfonate was obtained (1.5 g, 93%).

To a mixture of NaH (29.6 mg, 0.74 mmol, 60%) in DMF was added 3-(5-(1H-pyrazol-4-yl)-1H-indol-1-yl)-N,N-dimethylpropan-1-amine (100 mg, 0.37 mmol) at 0° C. After 20 minutes, 4-(2-mesyloxy)pyridine (88.4 mg, 0.44 mmol) was added slowly. The mixture was stirred at 50° C. overnight. The reaction mixture was poured into ice water and extracted with ethyl acetate. The organic phase was washed with brine, dried over Na₂SO₄, and filtered. After concentration the residue was purified by preparative HPLC to give the product (100 mg, 72%).

¹H NMR, (400 MHz, d₅-DMSO): δ 8.48 (d, J=5.6 Hz, 2H), 8.31 (s, 1H), 7.88 (s, 1H), 7.73 (s, 1H), 7.44 (d, J=8.8 Hz, 1H), 7.37-7.32 (m, 2H), 7.17 (d, J=5.6 Hz, 2H), 6.37 (d, J=3.2 Hz, 1H), 5.64 (q, J=7.2 Hz, 1H), 4.17 (t, J=6.8 Hz, 2H), 2.38-2.34 (m, 2H), 2.25 (s, 6H), 1.92 (quintet, J=6.8 Hz, 2H), 1.81 (d, J=7.2 Hz, 3H). LCMS: m/z 374.2 [m+H]⁺.

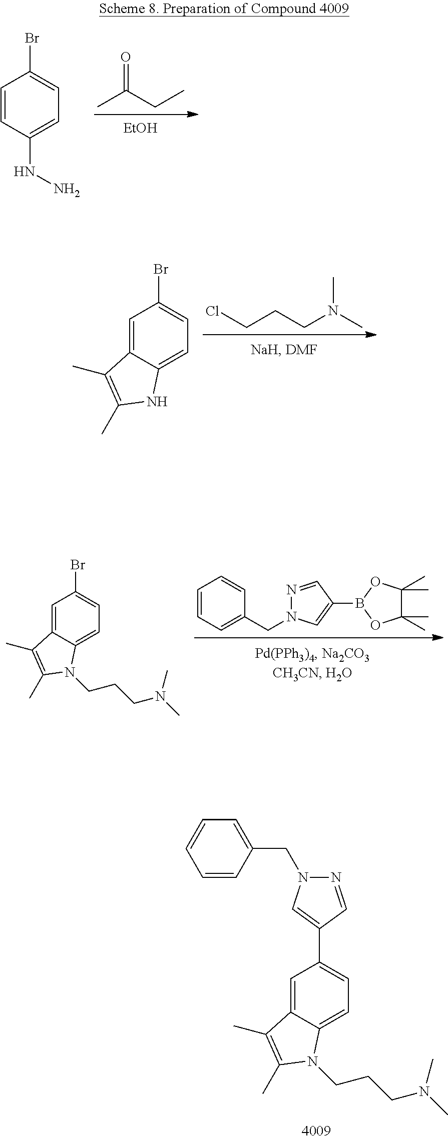

### Preparation of 5-bromo-2,3-dimethylindole

A mixture of 4-bromophenylhydrazine (5.0 g, 22.3 mmol) and methyl ethyl ketone (1.6 g, 22.3 mmol) was heated in ethanol (60 mL) at reflux overnight. The solvent was removed under vacuum and the residue recrystallized from ethanol/water, to give the target compound (3.6 g, 73%).

¹H NMR (400 MHz, d₆-DMSO): δ 10.87 (s, 1H), 7.51 (d, J=1.2 Hz, 1H), 7.18 (d, J=6.3 Hz, 1H), 7.07 (dd, J=6.3 Hz, 1.2 Hz, 1H), 2.31 (s, 3H), 2.13 (s, 3H).

### Preparation of 3-(5-bromo-2,3-dimethyl-1H-indol-1-yl)-N,N-dimethylpropan-1-amine

To a mixture of NaH (1.4 g, 35.6 mmol) in DMF (30 mL) was added 5-bromo-2,3-dimethylindole (2.0 g, 8.9 mmol) at 0° C. After 20 minutes, 3-chloro-N,N-dimethylpropan-1-amine (1.98 g, 12.5 mmol) was added slowly. The solution was stirred at 50° C. overnight. The reaction mixture was poured into ice water, extracted with ethyl acetate, washed with brine, dried over Na₂SO₄, filtered, and concentrated to give of the desired intermediate (2.5 g, 91%). LCMS: m/z 309.1, 311.1 [M+H]⁺.

### Preparation of Compound 4009, 3-(5-(1-benzyl-1H-pyrazol-4-yl)-2,3-dimethyl-1H-indol-1-yl)-N,N-dimethylpropan-1-amine

A mixture of 3-(5-bromo-2,3-dimethyl-1H-indol-1-yl)-N,N-dimethylpropan-1-amine (154 mg, 0.5 mmol), 1-benzylpyrazole-4-pinacolboronate (142.1 mg, 0.5 mmol), Pd(PPh₃)₄, (31.1 mg, 0.025 mmol), and Na₂CO₃ (106 mg, 1 mmol) in CH₃CN/H₂O (3.5 mL, 6:1) was allowed to react under microwave conditions at 110° C. for 1 hour. The mixture was poured into water and ethyl acetate. The organic phase was washed with brine and dried. After concentration the residue was purified by preparative HPLC to give the desired product (12.0 mg, 6%).

¹H NMR (400 MHz, CD₃OD): δ 8.01 (br s, 1H), 7.86 (s, 1H), 7.61 (br s, 1H), 7.40-7.27 (m, 7H), 5.39 (s, 2H), 4.27-4.24 (m, 2H), 3.15-3.11 (m, 2H), 2.85 (s, 6H), 2.41 (s, 3H), 2.26 (s, 3H), 2.21-2.15 (m, 2H). LCMS: m/z 387.3 [M+H]⁺.

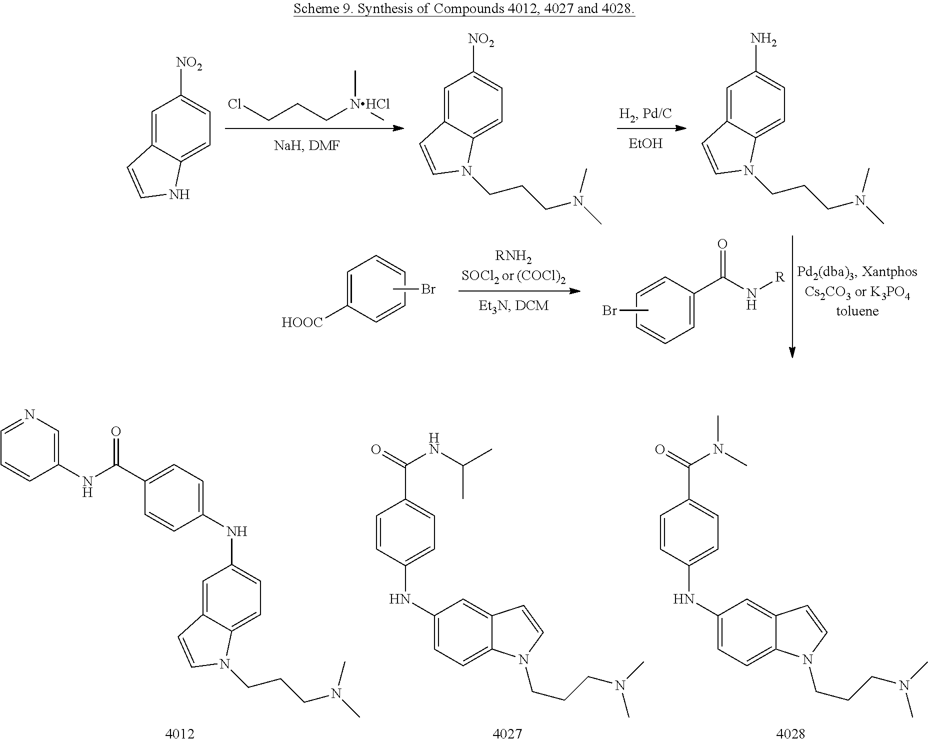

### Preparation of N,N-dimethyl-3-(5-nitro-1H-indol-1-yl)propan-1-amine

To a solution of NaH (1.60 g, 40.0 mmol) in DMF (10 mL), 5-nitro-indole (3.24 g, 20.0 mmol) in DMF (10 mL) was added dropwise at 0-5° C. The mixture was stirred for 15 minutes. Then, a solution of 3-chloro-N,N-dimethylpropan-1-amine hydrochloride (3.48 g, 22.0 mmol) in DMF (10 mL) was added dropwise (suspension) and the mixture was stirred for 15 minutes, then heated to 50° C. for 20 hours. The reaction was diluted with ethyl acetate and washed with water and brine. The organic layer was dried (Na₂SO₄), evaporated in vacuo and purified by chromatography (DCM/MeOH, 20:1 to 10:1) to afford the product (4.10 g, 83%).

¹H NMR (400 MHz, DMSO-d₆): δ 8.56 (d, J=2.4 Hz, 1H), 8.02 (dd, J=9.2 Hz, 2.4 Hz, 1H), 7.66 (d, J=8.8 Hz, 1H), 7.63 (d, J=3.2 Hz, 1H), 6.75 (d, J=3.2 Hz, 1H), 4.27 (t, J=6.8 Hz, 2H), 2.11-2.07 (m, 8H), 1.89-1.88 (m, 2H). LCMS: m/z 248.1 [M+H]⁺.

### Preparation of 1-(3-(dimethylamino)propyl)-1H-indol-5-amine

To a solution of 1-N,N-dimethylaminopropyl-5-nitroindole (1.0 g, 4.0 mmol) in ethanol (20 mL), was added Pd/C (0.2 g, 20%). The mixture was stirred under a hydrogen atmosphere at 40° C. overnight. After filtration the solution was concentrated to give the product (0.8 g, 91%).

¹H NMR (400 MHz, CDCl₃): δ 7.20 (d, J=8.8 Hz, 1H), 7.06 (d, J=3.2 Hz, 1H), 6.95 (d, J=2.4 Hz, 1H), 6.70 (dd, J=8.8 Hz, 2.4 Hz, 1H), 6.32 (d, J=2.8 Hz, 1H), 4.15 (t, J=6.8 Hz, 2H), 3.48 (br s, 2H), 2.45 (m, 8H), 2.01-1.94 (m, 2H). LCMS: m/z 218.2 [M+H]⁺.

### Preparation of 4-bromo-N-(pyridin-3-yl)benzamide

4-Bromobenzoic acid (2.0 g, 10.0 mmol) was dissolved in SOCl₂ (20 mL) and was heated at reflux for 3 hours. The excess SOCl₂ was removed under vacuum. The residue was dissolved in dry DCM and added dropwise to a solution of 3-aminopyridine (1.12 g, 12.0 mmol) and Et₃N (1.5 g, 15 mmol) in DCM (30 mL) at 0° C., then stirred at room temperature for 3 hours. The mixture was poured into water and extracted with DCM. The combined organic phases were washed with 1 N HCl and NaHCO₃, then brine, and dried over Na₂SO₄. Filtration, concentration and recrystallization from EtOH/H₂O gave the target intermediate (1.6 g, 58%).

### Preparation of Compound 4012, 44(1-(3-(dimethylamino)propyl)-1H-indol-5-yl)amino)-N-(pyridin-3-yl)benzamide

4-Bromo-N-(pyridin-3-yl)benzamide (277 mg, 1.0 mmol) was combined with 1-(3-(dimethylamino)propyl)-1H-indol-5-amine (217.6 mg, 1.0 mmol), Pd₂(dba)₃ (45.78 mg, 0.05 mmol), Xantphos (28.93 mg, 0.05 mmol), and Cs₂CO₃ (651.6 mg, 2.0 mmol) in toluene and allowed to react under microwave conditions at 140° C. for 5 hours. The toluene was removed under reduced pressure, ethyl acetate was added, and the solid was filtered. The organic phase was washed with brine and concentrated, before being purified by preparative HPLC to give the desired product (10 mg, 2.4%).

¹H NMR (400 MHz, CD₃OD): δ 9.47 (br s, 1H), 8.63 (dt, J=7.6 Hz, 1.2 Hz, 1H), 8.51 (d, J=5.2 Hz, 1H), 7.95 (dd, J=7.6 Hz, 5.2 Hz, 1H), 7.87 (d, J=8.8 Hz, 2H), 7.49 (d, J=8.8 Hz, 1H), 7.45 (d, J=2.0 Hz, 1H), 7.30 (d, J=3.2 Hz, 1H), 7.12 (dd, J=8.4 Hz, 2.0 Hz, 1H), 6.98 (d, J=8.8 Hz, 2H), 6.48 (d, J=3.2 Hz, 1H), 4.35 (t, J=6.8 Hz, 2H), 3.15-3.12 (m, 2H), 2.88 (s, 6H), 2.33-2.25 (m, 2H). LCMS: miz 414.2 [M+H]⁺.

### Preparation of 4-bromo-N-isopropylbenzamide

4-Bromobenzoic acid (630 mg, 3.00 mmol) was added to CH₂Cl₂ (10 mL). To this suspension was added oxalyl chloride (766 mg, 9.00 mmol) and 2 drops of DMF. The reaction was stirred until clear (approximately 1 hour). Then more oxalyl chloride (655 mg, 5.16 mmol) was added. No gas was liberated. To the reaction was added Et₃N (3.4 mL, 24.0 mmol) followed by isopropylamine (1.02 mL, 12.0 mmol). The reaction was stirred for 10 minutes before it was quenched with 2 N HCl, extracted with DCM, dried and evaporated under reduced pressure. The crude product was purified by chromatography eluting with petrolum/EtOAc from 5/1 to give an off-white solid (670 mg, 92%).

¹H NMR (400 MHz, CDCl₃): δ 7.64 (d, J=8.8 Hz, 2H), 7.56 (d, J=8.8 Hz, 2H), 5.92 (br s, 1H), 4.32-4.27 (m, 1H), 1.28 (d, J=6.8 Hz, 6H).

### Preparation of Compound 4027, 4-((1-(3-(dimethylamino)propyl)-1H-indol-5-yl)amino)-N-isopropylbenzamide

A solution of 1-(3-(dimethylamino)propyl)-1H-indol-5-amine (115 mg, 0.529 mmol), 4-bromo-N-isopropylbenzamide (128 mg, 0.529 mmol), K₃PO₄ (112 mg, 0.529 mmol), Pd₂(dba)₃ (7.3 mg, 0.00794 mmol) and Xantphos (9.2 mg, 0.0159 mmol) in DMF (2 mL) was heated to 80° C. for 16 hours. No product was observed by LCMS. DMF (2 mL) was added to the mixture. Then the mixture was heated to 80° C. for another 16 hours. The solvent was removed under reduced pressure. The crude product was purified by chromatography eluting with DCM/MeOH from 30/1 to 12/1 to afford a brown solid (90 mg, 45%).

¹H NMR (400 MHz, CDCl₃): δ 7.63 (d, J=8.8 Hz, 1H), 7.46 (d, J=2.0 Hz, 1H), 7.37 (d, J=8.4 Hz, 1H), 7.17 (d, J=3.2 Hz, 1H), 7.07 (dd, J=8.4 Hz, 2.0 Hz, 1H), 6.88 (d, J=8.8 Hz, 2H), 6.46 (d, J=3.2 Hz, 1H), 5.92 (br s, 1H), 5.79 (d, J=7.6 Hz, 1H), 4.34-4.27 (m, 1H), 4.23 (t, J=7.2 Hz, 2H), 2.32 (t, J=6.8 Hz, 2H), 2.29 (s, 6H), 2.08-2.01 (m, 2H), 1.26 (d, J=6.4 Hz, 6H). LCMS: m/z 397.2 [M+H]⁺.

### Preparation of 4-bromo-N,N-dimethylbenzamide

4-Bromobenzoic acid (1.01 g, 4.98 mmol) was added to CH₂Cl₂ (40 mL). To this suspension was added oxalyl chloride (0.64 ml, 7.46 mmol) and 4 drops of DMF. The reaction was stirred until clear (approximately 1 hour). No gas was liberated. To the reaction was added dimethylamine (2.84 ml, 42.8 mmol). The reaction was stirred for 10 minutes before it was quenched with saturated sodium bicarbonate (50 mL). The organic layer was separated. The aqueous layer was extracted with DCM. The organic layers were combined, dried and evaporated under reduced pressure to give a white solid (1.09 g, 96%).

¹H NMR (400 MHz, CDCl₃): δ 7.55 (d, J=8.4 Hz, 2H), 7.32 (d, J=8.4 Hz, 2H), 3.12 (s, 3H), 2.99 (s, 3H). LCMS: m/z 230.0 [M+H]⁺.

### Preparation of Compound 4028, 4-((1-(3-(dimethylamino)propyl)-1H-indol-5-yl)amino)-N,N-dimethylbenzamide

A solution of N,N-dimethyl-3-(5-nitro-1 H-indol-1-yl)propan-1-amine (200 mg, 0.92 mmol), 4-bromo-N,N-dimethylbenzamide (210 mg, 0.92 mmol), K₃PO₄ (195 mg, 0.92 mmol), Pd₂(dba)₃ (12.6 mg, 0.0138 mmol) and Xantphos (16.0 mg, 0.0276 mmol) are heated to 80° C. for 18 hours. LCMS indicated that the starting material had not been consumed. The mixture was then heated to 100° C. After 5 hours, the reaction appeared to be incomplete by TLC. The temperature was increased to 120° C. for 24 hours. The solvent was removed under reduced pressure. The crude product was purified by chromatography eluting with DCM/MeOH (15/1) to afford an oil (40 mg, 12%).

¹H NMR (400 MHz, CDCl₃): δ 7.46 (d, J=2.0 Hz, 1H), 7.37-7.33 (m, 3H), 7.15 (d, J=3.2 Hz, 1H), 7.07 (dd, J=8.8 Hz, 2.0 Hz, 1H), 6.88 (d, J=8.4 Hz, 2H), 6.45 (d, J=2.8 Hz, 1H), 5.83 (br s, 1H), 4.23 (t, J=7.2 Hz, 2H), 3.09 (s, 6H), 2.35-2.30 (m, 8H), 2.07-2.04 (m, 2H). LCMS: m/z 365.2 [M+H]⁺.

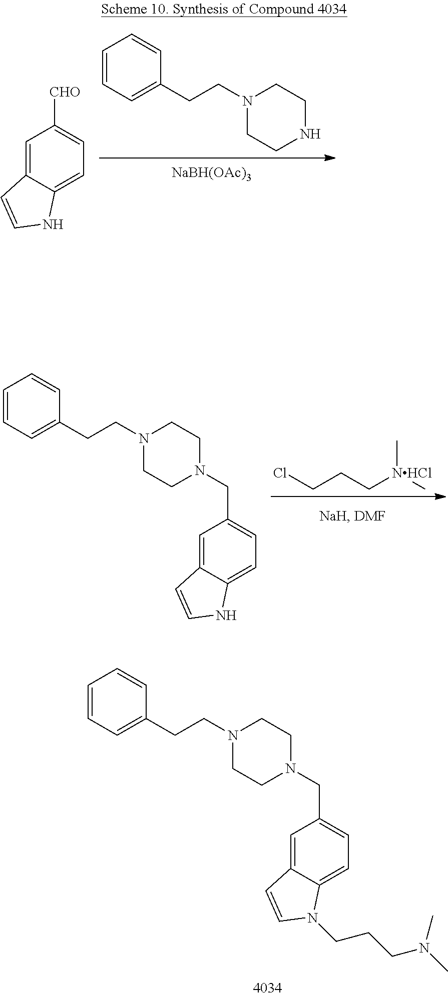

### Preparation of 5-((4-phenethylpiperazin-1-yl)methyl)-1H-indole

A solution of 1H-indole-5-carbaldehyde (500 mg, 3.45 mmol) and 1-phenethylpiperazine (650 mg, 3.45 mmol) in EtOH was heated at reflux for 4 hours. NaBH₃CN (1.08 g, 17.2 mmol) was added and the whole mixture was stirred at reflux overnight. The reaction liquid was partitioned between EtOAc and H₂O, and the organic layer was washed with water and brine, dried, concentrated and purified by silica gel column (MeOH/DCM=1/20) to obtain a solid (300 mg, 27%). LCMS: m/z 320.2 [M+H]⁺.

### Preparation of Compound 4034, N,N-dimethyl-3-(5-((4-phenethylpiperazin-1-yl)methyl)-1H-indol-1-yl)propan-1-amine

To a suspension of NaH (45 mg, 1.1 mmol) in DMF (5 mL) was added compound 5-((4-phenethylpiperazin-1-yl)methyl)-1H-indole (160 mg, 0.5 mmol) at 0° C. The resulting mixture was stirred for 20 minutes. Then the solution of 3-chloro-N,N-dimethylpropan-1-amine (95 mg, 0.6 mmol) in DMF was added to the reaction mixture at 0° C., and the whole mixture was allowed to warm to 50° C. overnight. When TLC indicated that all of the starting material had been consumed, the reaction liquid was partitioned between EtOAc and water. The organic layer was washed with water and brine, dried over Na₂SO₄, concentrated to give the crude product. The crude compound was purified by prep-TLC (MeOH/DCM=1/20) to afford the product (80 mg, 40%).

¹H NMR (400 MHz, CDCl₃): δ 7.59 (br s, 1H), 7.36-7.22 (m, 7H), 7.14 (d, J=2.8 Hz, 1H), 6.49 (d, J=2.8 Hz, 1H), 4.21 (t, J=7.2 Hz, 2H), 3.68 (s, 2H), 2.87-2.83 (m, 2H), 2.71-2.32 (m, 10H), 2.31-2.27 (m, 8H), 2.03-2.00 (m, 2H). LCMS: m/z 405.3 [M+H]⁺.

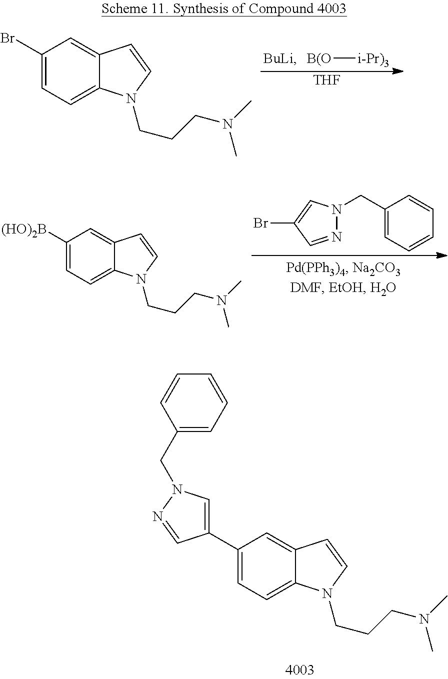

### Preparation of (1-(3-(dimethylamino)propyl)-1H-indol-5-yl)boronic acid

To a solution of 3-(5-bromo-1H-indol-1-yl)-N,N-dimethylpropan-1-amine (284 mg, 1.0 mmol) in THF (0.5 mL), n-BuLi (2.5 M, 0.48 mL, 1.2 mmol) was added dropwise at −68° C. The mixture was stirred at this temperature for 1 hour, after which B(O-i-Pr)₃ (0.345 mL, 1.5 mmol) was added dropwise and the mixture was slowly allowed to warm to room temperature overnight. 1 N HCl (6 mL) was added slowly. The mixture was stirred at room temperature for 1 hour and then extracted with DCM. To the aqueous solution, saturated NaHCO₃ was added until a precipitate was observed. The mixture was then extracted with DCM and the organic layer was dried with Na₂SO₄ and concentrated to give the product (190 mg, 77%), which was used without further purification.

### Preparation of Compound 4003, 3-(5-(1-benzyl-1H-pyrazol-4-yl)-1H-indol-1-yl)-N,N-dimethylpropan-1-amine

1-Benzyl-4-bromo-1H-pyrazole (237 mg, 1.0 mmol), 1-(3-(dimethylamino)propyl)-1H-indol-5-ylboronic acid (180 mg, 0.73 mmol) and Na₂CO₃ (315 mg, 3.0 mmol) were dissolved in a mixture of DMF (8 mL), EtOH (2 mL) and water (2 mL). Pd(PPh₃)₄ (115 mg, 0.1 mmol) was added under a nitrogen atmosphere and the mixture was heated at 100° C. for 2 hours. Water (40 mL) was added and the resulting precipitate was collected, redissolved in EtOAc, dried over Na₂SO₄, and concentrated. The residue was purified by column chromatography (silica gel, DCM:MeOH=15:1), followed by prep-TLC to afford the product (11 mg, 4%).

¹H NMR (400 MHz, CDCl₃): δ 7.84 (s, 1H), 7.71 (s, 1H), 7.62 (s, 1H), 7.36-7.26 (m, 7H), 7.10 (d, J=3.2 Hz, 1H), 6.46 (d, J=2.8 Hz, 1H), 5.35 (s, 2H), 4.19 (t, J=7.0 Hz, 2H), 2.23-2.20 (m, 8H), 2.00-1.98 (m, 2H).

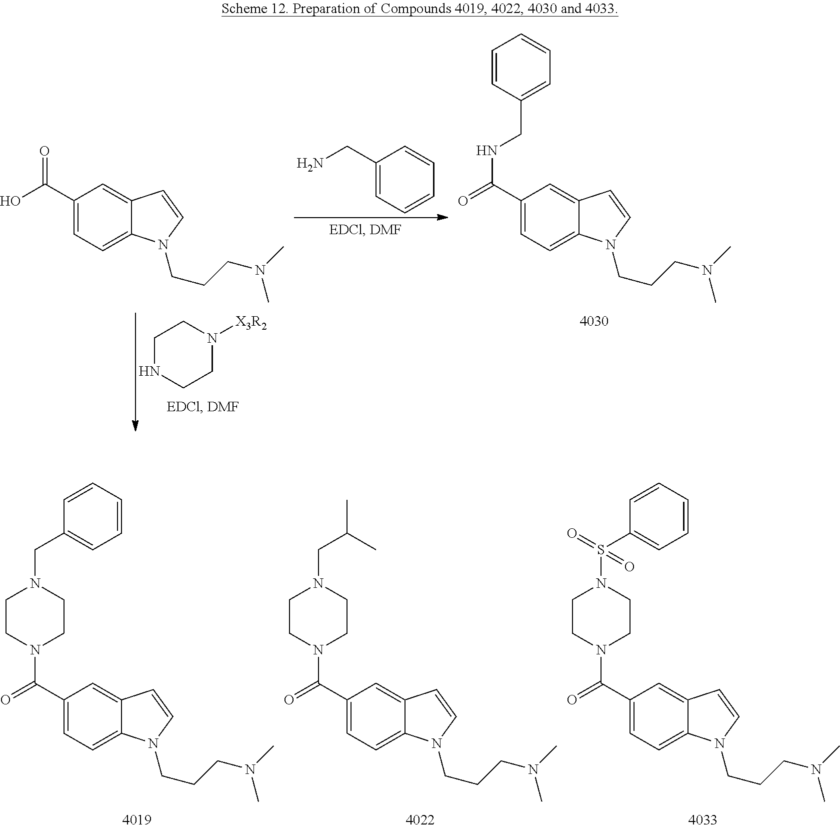

### Preparation of Compound 4019, (4-benzylpiperazin-1-yl)(1-(3-(dimethylamino)propyl)-1H-indol-5-yl)methanone

To a solution of 1-(3-(dimethylamino)propyl)-1H-indole-5-carboxylic acid (123 mg, 0.5 mmol) and 1-benzylpiperazine (88 mg, 0.5 mmol) in DMF (5 mL), was added EDCl (192 mg, 1 mmol). The mixture was stirred at 40° C. overnight. The reaction mixture was then partitioned between EtOAc and water. The organic layer was washed with water and brine, then dried, concentrated and purified by prep-TLC (MeOH/DCM=1/20) to afford the target compound (50 mg, 24%).

¹H NMR (400 MHz, CDCl₃): δ 7.71 (br s, 1H), 7.39-7.29 (m, 7H), 7.19 (d, J=3.2 Hz, 1H), 6.54 (d, J=3.2 Hz, 1H), 4.24 (t, J=6.8 Hz, 2H), 3.81 (br s, 4H), 3.56 (s, 2H), 2.50 (br s, 4H), 2.31-2.28 (m, 8H), 2.03 (quintet, J=6.8 Hz, 2H). LCMS: m/z 405.3 [M+H]⁺.

### Preparation of Compound 4022, (1-(3-(dimethylamino)propyl)-1H-indol-5-yl)(4-isobutylpiperazin-1-yl)methanone

To a solution of 1-(3-(dimethylamino)propyl)-1H-indole-5-carboxylic acid (246 mg, 1.0 mmol) and 1-isobutylpiperazine (142 mg, 1.0 mmol) in DMF (10 mL) was added EDCl (384 mg, 2.0 mmol). The mixture was stirred at 40° C. overnight. The reaction mixture was then partitioned between EtOAc and water. The organic layer was washed with water and brine, then dried, concentrated and purified by rep-TLC (MeOH/DCM=1/20) to afford the target product (48 mg, 13%).

¹H NMR (400 MHz, CDCl₃): δ 7.71 (br s, 1H), 7.38 (d, J=8.8 Hz, 1H), 7.29 (dd, J=8.4 Hz, 1.2 Hz, 1H), 7.18 (d, J=3.2 Hz, 1H), 6.53 (d, J=3.2 Hz, 1H), 4.22 (t, J=7.2 Hz, 2H), 3.66 (br s, 4H), 2.42 (br s, 4H), 2.28-2.25 (m, 8H), 2.12 (d, J=7.6 Hz, 2H), 2.00 (quintet, J=7.6 Hz, 2H), 1.80-1.70 (m, 1H), 0.92 (d, J=6.4 Hz, 6H). LCMS: m/z 371.3 [M+H]⁺.

### Preparation of Compound 4030, N-benzyl-1-(3-(dimethylamino)propyl)-1H-indole-5-carboxamide

To a solution of 1-(3-(dimethylamino)propyl)-1H-indole-5-carboxylic acid (246 mg, 1.0 mmol) and phenylmethanamine (107 mg, 1.0 mmol). in DMF (10 mL) was added EDCl (384 mg, 2.0 mmol). The mixture was stirred at 40° C. overnight. The reaction mixture was then partitioned between EtOAc and water. The organic layer was washed with water and brine, then dried, concentrated and purified by prep-TLC (MeOH/DCM=1/20) to afford the target product (40 mg, 12%).

¹H NMR (400 MHz, CDCl₃): δ 8.13 (d, J=1.6 Hz, 1H), 7.71 (dd, J=8.8 Hz, 1.6 Hz, 1H), 7.40-7.29 (m, 6H), 7.19 (d, J=3.2 Hz, 1H), 6.63 (br s, 1H), 6.56 (d, J=2.8 Hz, 1H), 4.69 (d, J=6.0 Hz, 2H), 4.22 (t, J=6.8 Hz, 2H), 2.28-2.25 (m, 8H), 2.00 (quintet, J=6.8 Hz, 2H). LCMS: m/z 336.2 [M+H]⁺.

### Preparation of Compound 4033, (1-(3-(dimethylamino)propyl)-1H-indol-5-yl)(4-(phenyisulfonyl)piperazin-1-yl)methanone

To a solution of 1-(3-(dimethylamino)propyl)-1H-indole-5-carboxylic acid (87 mg, 0.35 mmol) and 1-(phenylsulfonyl)piperazine (80 mg, 0.35 mmol) in DMF (5 mL) was added EDCl (140 mg, 0.7 mmol). The mixture was stirred at 40° C. overnight. The reaction mixture was then partitioned between EtOAc and water. The organic layer was washed with water and brine, then dried, concentrated and purified by prep-TLC (MeOH/DCM=1/20) to afford the target product (52 mg, 33%).

¹H NMR (400 MHz, CDCl₃): δ 7.75 (d, J=7.6 Hz, 2H), 7.65-7.55 (m, 4H), 7.36 (d, J=8.4 Hz, 1H), 7.20-7.18 (m, 2H), 6.50 (d, J=3.2 Hz, 1H), 4.22 (t, J=6.8 Hz, 2H), 3.77 (br s, 4H), 3.04 (br s, 4H), 2.33-2.29 (m, 8H), 2.02 (quintet, J=6.8 Hz, 2H). LCMS: m/z 455.2 [M+H]⁺.

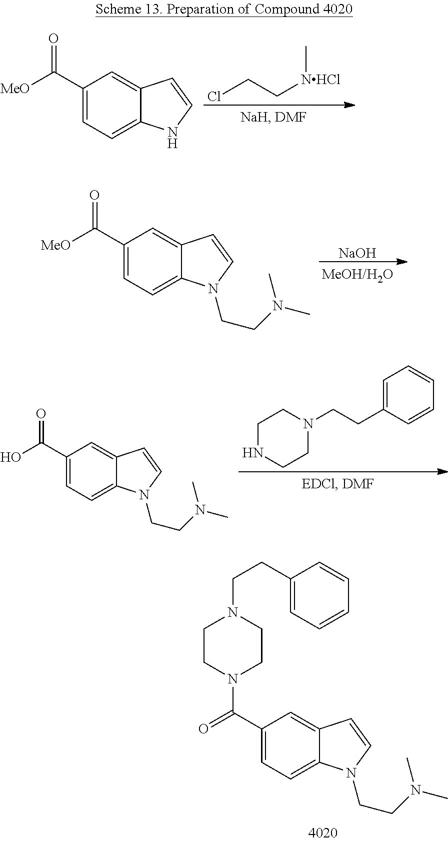

### Preparation of methyl 1-(2-(dimethylamino)ethyl)-1H-indole-5-carboxylate

To a suspension of NaH (880 mg, 22 mmol) in DMF (20 mL) was added methyl 1H-indole-5-carboxylate (1.75 g, 10 mmol) at 0° C. The resulting mixture was stirred for 20 minutes. A solution of 2-chloro-N,N-dimethylethan-1-amine hydrochloride (1.71 g, 12 mmol) in DMF was added at 0° C., and the reaction mixture was allowed to warm to 50° C. overnight. When TLC indicated that all of the starting material had been consumed, the reaction liquid was partitioned between EtOAc and water. The organic layer was washed with water and brine, dried over Na₂SO₄, concentrated to give the crude product. Purification by silica gel column (MeOH/DCM=1/20) gave the pure target compound (1.95 g, 79%).

### Preparation of 1-(2-(dimethylamino)ethyl)-1H-indole-5-carboxylic acid

To a solution of methyl 1-(2-(dimethylamino)ethyl)-1H-indole-5-carboxylate (1.95 g, 7.9 mmol) in MeOH/H₂O (10 mL/10 mL) was added NaOH (0.95 g, 23.8 mmol). The mixture was stirred at 40° C. for 4 hours. The reaction mixture was acidified with HCl (conc.) to pH 2-3 and then concentrated in vacuo to obtain the target (3.75 g, with NaCl).

### Preparation of Compound 4020, (1-(2-(dimethylamino)ethyl)-1H-indol-5-yl)(4-phenethylpiperazin-1-yl)methanone

To a solution of 1(2-(dimethylamino)ethyl)-1H-indole-5-carboxylic acid (470 mg, 1.0 mmol) and 1-phenethylpiperazine (190 mg, 1.0 mmol) in DMF (10 mL) was added EDCl (380 mg, 2.0 mmol). The mixture was stirred at 40° C. overnight. When TLC indicated that the reaction was complete, the mixture was poured into water and extracted with EtOAc. The organic layer was washed with brine, dried with Na₂SO₄ and concentrated in vacuo. The residue oil was purified by prep-TLC to obtain the target as a colorless oil (30 mg, 8%).

¹H NMR (400 MHz, CD₃OD): δ 7.70 (d, J=1.2 Hz, 1H), 7.56 (d, J=8.4 Hz, 1H), 7.40 (d, J=3.2 Hz, 1H), 7.30-7.18 (m, 6H), 6.60 (d, J=3.2 Hz, 1H), 4.44 (t, J=7.2 Hz, 2H), 3.72 (br s, 4H), 2.99 (t, J=7.2 Hz, 2H), 2.86 (t, J=8.8 Hz, 2H), 2.71-2.50 (m, 6H), 2.47 (s, 6H). LCMS: m/z 405.3 [M+H]⁺.

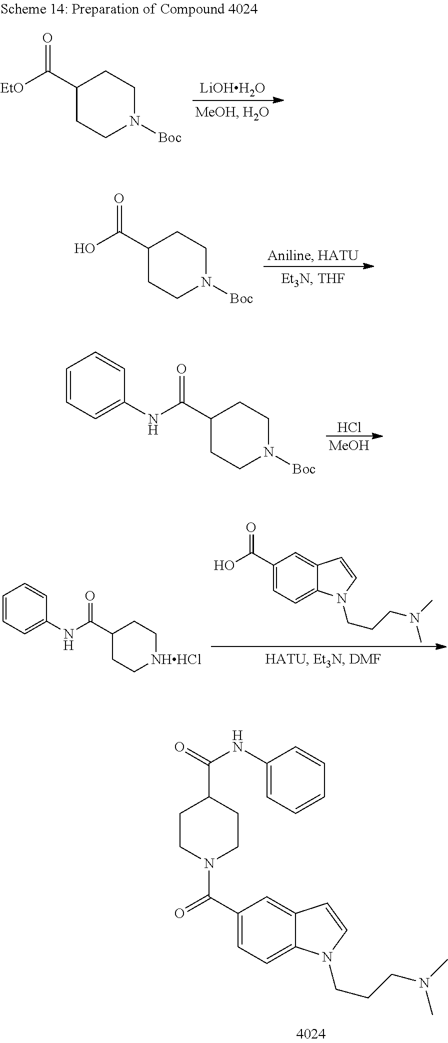

### Preparation of 1-(tert-butoxycarbonyl)piperidine-4-carboxylic acid

A solution of 1-(tert-butyl) 4-ethyl piperidine-1,4-dicarboxylate (5.0 g, 0.02 mol) and LiOH.H₂O (4.3 g, 0.1 mol) in MeOH/H₂O (10 mL/10 mL) was stirred at room temperature for 1 hour. When TLC indicated that the reaction was complete, the solution was acidified (pH 6) with 2 N HCl and filtered. The residue was washed with water and dried in vacuo to give a white solid (2.9 g, 62%).

¹H NMR (400 MHz, CDCl₃): δ 4.07-4.02 (m, 2H), 2.93-2.82 (m, 2H), 2.54-2.50 (m, 1H), 1.95-1.92 (m, 2H), 1.69-1.64 (m, 2H), 1.48 (s, 9H).

### Preparation of tert-butyl 4-(phenylcarbamoyl)piperidine-1-carboxylate

To a solution of 1-(tert-butoxycarbonyl)piperidine-4-carboxylic acid (7.0 g, 0.03 mol) in THF (70 mL) was added HATU (23.0 g, 0.06 mol). The mixture was stirred at room temperature for 1 hour. Aniline (4.3 g, 0.05 mol) and Et₃N (15.4 g, 0.15 mol) were added dropwise at room temperature. The reaction mixture was stirred at 45° C. overnight. When TLC indicated that the reaction was complete, the solution was concentrated in vacuo. The residue was dissolved in EtOAc, washed with 0.5 N HCl, aqueous Na₂CO₃ and brine, and dried with Na₂SO₄. The organic layer was concentrated in vacuo. The residue oil was purified by flash chromatography (Petroleum etherEtOAc=2:1) on silica gel to give a white solid (7.3 g, 69%).

¹H NMR (400 MHz, CDCl₃): δ 7.54 (d, J=7.6 Hz, 2H), 7.35 (t, J=7.6 Hz, 2H), 7.23 (br s, 1H), 7.14 (t, J=7.6 Hz, 1H), 4.22 (br s, 2H), 2.85-2.78 (m, 2H), 2.40 (quintet, J=8.0 Hz, 1H), 1.94-1.91 (m, 2H), 1.81-1.72 (m, 2H), 1.48 (s, 9H).

### Preparation of N-phenylpiperidine-4-carboxamide hydrochloride

To a solution of tert-butyl 4-(phenylcarbamoyl)piperidine-1-carboxylate (3.0 g, 9.9 mmol) in MeOH (10 mL) was added HCl/MeOH (10 mL) dropwise at room temperature. The solution was stirred at room temperature overnight. When TLC indicated that the reaction was complete, the mixture was concentrated in vacuo to give an off-white solid (2.3 g, 96%).

¹H NMR (400 MHz, CD₃OD): δ 7.58 (d, J=7.6 Hz, 2H), 7.33 (t, J=7.6 Hz, 2H), 7.12 (t, J=7.6 Hz, 1H), 3.53-3.48 (m, 2H), 3.19-3.08 (m, 2H), 2.78 (quintet, J=6.8 Hz, 1H), 2.15-2.10 (m, 2H), 2.05-1.98 (m, 2H).

### Preparation of Compound 4024, 1-(1-(3-(dimethylamino)propyl)-1H-indole-5-carbonyl)-N-phenylpiperidine-4-carboxamide

A mixture of 1-(2-(dimethylamino)ethyl)-1H-indole-5-carboxylic acid (2.0 g, 4.1 mmol) and HATU (2.3 g, 6.1 mmol) in DMF (10 mL) was stirred at room temperature for 1 hour. N-phenylpiperidine-4-carboxamide hydrochloride (1.2 g, 4.9 mmol) and Et₃N (2.1 g, 20.3 mmol) were added to the mixture, which was then stirred at 35° C. overnight. When TLC showed that the reaction was complete, the mixture was concentrated in vacuo. The residue was purified by prep-TLC to give the target compound, (50 mg, 14%).

¹H NMR (400 MHz, CD₃OD): δ 7.70 (br s, 1H), 7.64-7.56 (m, 3H), 7.39 (d, J=3.2 Hz, 1H), 7.34-7.29 (m, 3H), 7.11 (t, J=7.2 Hz, 1H), 6.62 (d, J=3.2 Hz, 1H), 4.91-4.70 (m, 1H), 4.37 (t, J=6.8 Hz, 2H), 4.07-3.95 (m, 1H), 3.30-3.06 (m, 4H), 2.83 (s, 6H), 2.76-2.71 (m, 1H), 2.30-2.25 (m, 2H), 2.05-1.78 (m, 4H). LCMS: m/z 433.2 [M+H]⁺.

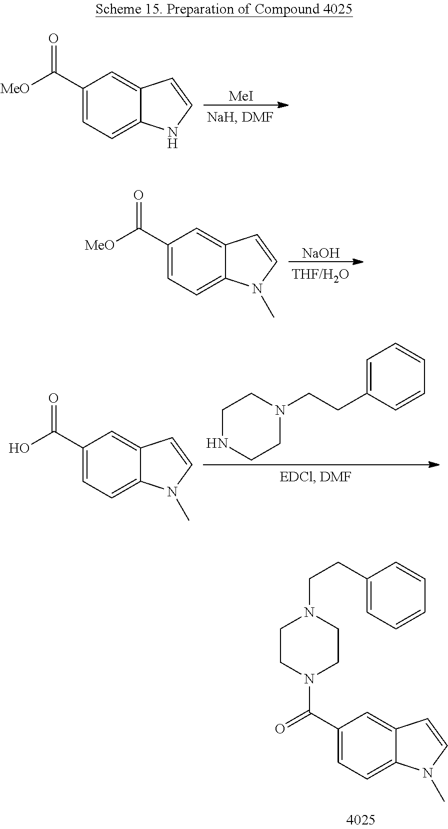

### Preparation of methyl 1-methyl-1H-indole-5-carboxylate

To a suspension of NaH (264 mg, 1.1 mmol) in DMF (20 mL) was added methyl 1H-indole-5-carboxylate (1.05 g, 6 mmol) at 0° C. and the resulting mixture was stirred for 20 minutes. MeI (1.70 g, 2.2 mmol) was added to the reaction mixture, which was then allowed to warm to 50° C. overnight. The reaction liquid was partitioned between EtOAc and water. The organic layer was washed with water and brine, dried over Na₂SO₄ and concentrated to give the crude product, which was purified by silica gel column (EtOAc/petroleum ether=1/10) to give the target compound (860 mg, 76%). LCMS: m/z 190.1 [M+H]⁺.

### Preparation of 1-methyl-1H-indole-5-carboxylic acid

To a solution of methyl 1-methyl-1H-indole-5-carboxylate (500 mg, 2.64 mmol) in THF/H₂O (2 mL/2 mL) was added NaOH (635 mg, 15.9 mmol). The mixture was stirred at 40° C. for 4 hours. Then the reaction liquid was concentrated in vacuo to remove most of organic solvent, and H₂O (10 mL) was added to the residue. The mixture was acidified (pH 5-6) with HCl (conc.) and the resulting precipitate was collected as the target (450 mg, 97%). LCMS: m/z 176.1 [M+H]⁺.

### Preparation of Compound 4025, (1-methyl-1H-indol-5-yl)(4-phenethylpiperazin-1-yl)methanone

To a solution of 1-methyl-1H-indole-5-carboxylic acid (88 mg, 0.5 mmol) and 1-phenethylpiperazine (95 mg, 0.5 mmol) in DMF (5 mL) was added EDCl (192 mg, 1 mmol). The mixture was stirred at 40° C. overnight. The reaction mixture was partitioned between EtOAc and water. The organic layer was washed with water and brine, then dried, concentrated and purified by prep-TLC (MeOH/DCM=1/20) to afford a solid (105 mg, 63%).

¹H NMR (400 MHz, CDCl₃): δ 7.75 (br s, 1H), 7.35-7.29 (m, 4H), 7.25-7.23 (m, 3H), 7.13 (d, J=3.2 Hz, 1H), 6.55 (d, J=3.2 Hz, 1H), 3.82 (s, 3H), 3.71 (br s, 4H), 2.87-2.82 (m, 2H), 2.69-2.65 (m, 2H), 2.58 (br s, 4H). LCMS: m/z 348.2 [M+H]⁺.

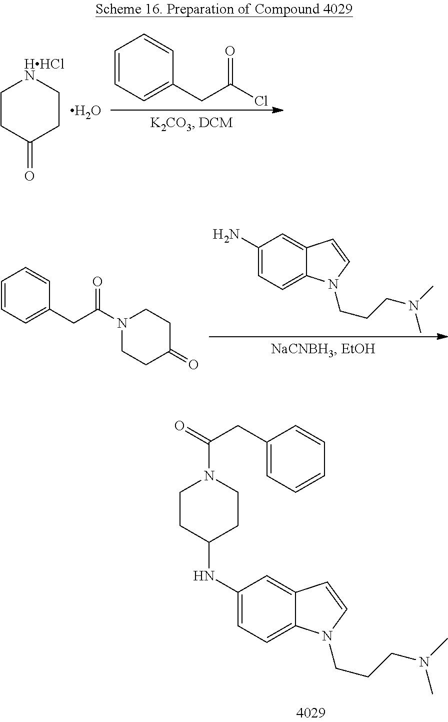

### Preparation of 1-(2-phenylacetyl)piperidin-4-one

To a solution of piperidin-4-one hydrochloride hydrate (1.50 g, 9.76 mmol) in DCM was added K₂CO₃ (4.04 g, 19.5 mmol). After stirring for 5 minutes, the addition of phenylacetyl chloride (2.6 mL, 19.5 mmol) proceeded at room temperature. Stirring continued for a further 18 hours. The reaction was quenched with 1 N NaOH (30 mL) and extracted with DCM. The organic layers were dried (Na₂SO₄), filtered and concentrated in vacuo. The crude product was purified with chromatography eluting with DCM/MeOH from 30/1 to 15/1 to give an oil (1.50 g, 71%).

¹H NMR (400 MHz, CDCl₃): δ 7.37-7.32 (m, 2H), 7.30-7.27 (m, 3H), 3.89 (t, J=6.4 Hz, 2H), 3.83 (s, 2H), 3.71 (t, J=6.4 Hz, 2H), 2.42 (t, J=6.4 Hz, 2H), 2.14 (t, J=6.4 Hz, 2H). LCMS: m/z 218.1 [M+H]⁺.

### Preparation of Compound 4029, 1-(4-((1-(3-(dimethylamino)propyl)-1H-indol-5-yl)amino)piperidin-1-yl)-2-phenylethan-1-one

A mixture of 1-(3-(dimethylamino)propyl)-1H-indol-5-amine (200 mg, 0.92 mmol) and 1-(2-phenylacetyl)piperidin-4-one (440 mg, 2.02 mmol) in EtOH (10 mL) was stirred for 30 minutes at room temperature. Sodium cyonoborohydride (75 mg, 1.20 mmol) was added to the reaction mixture. After stirring for 17 hours, the solvent was evaporated under reduced pressure. Water (15 mL) was added and the mixture was extracted with EtOAc. The combined organic layers were dried (Na₂SO₄) and concentrated to give a red oil. The crude product was purified by chromatography eluting with DCM/MeOH 15/1, followed by preparative TLC to give the pure target compound (40 mg, 10%).

¹ H NMR (400 MHz, CDCl₃): δ 7.39-7.29 (m, 2H), 7.28-7.24 (m, 3H), 7.18 (d, J=8.8 Hz, 1H), 7.05 (d, J=2.0 Hz, 1H), 6.83 (d, J=2.8 Hz, 1H), 6.62 (dd, J=8.8 Hz, 2.0 Hz, 1H), 6.33 (d, J=2.8 Hz, 1H), 4.54 (dd, J=13.2 Hz, 1.6 Hz, 1H), 4.21 (t, J=6.4 Hz, 2H), 3.90-3.85 (m, 1H), 3.78 (s, 2H), 3.51-3.44 (m, 1H), 3.18-3.11 (m, 1H), 2.94-2.87 (m, 1H), 2.57 (t, J=7.2 Hz, 2H), 2.48 (s, 6H), 2.25-2.17 (m, 2H), 2.16-2.09 (m, 1H), 2.07-1.99 (m, 1H), 1.40-1.26 (m, 1H), 1.15-1.05 (m, 1H). LCMS: m/z 419.3 [M+H]⁺.

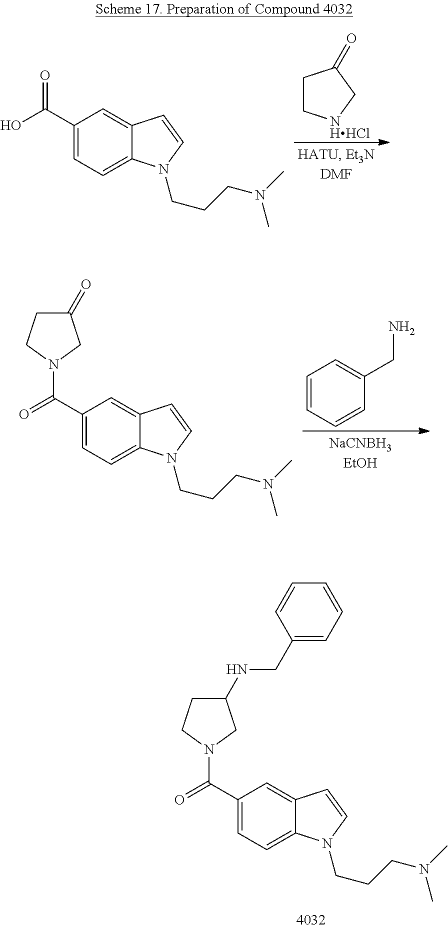

### Preparation of 1-(1-(3-(dimethylamino)propyl)-1H-indole-5-carbonyl)pyrrolidin-3-one

A mixture of 1-(3-(dimethylamino)propyl)-1H-indole-5-carboxylic acid (3.5 g, 7.1 mmol) and HATU (4.1 g, 10.8 mmol) in DMF (12 mL) was stirred at room temperature for 1 hour. A mixture of pyrrolidin-3-one hydrochloride (1.0 g, 8.5 mmol) in DMF (12 mL) and Et₃N (2.1 g, 20.3 mmol) was added to the reaction mixture, which was then stirred at 35° C. overnight. When TLC showed that the reaction was complete, the mixture was filtered and the filtrate was concentrated in vacuo. The residue was purified by flash chromatography (CH₂Cl₂:MeOH=10:1) on silica gel to give a pale yellow solid (1.0 g, 45%).

### Preparation of Compound 4032, (3-(benzylamino)pyrrolidin-1-yl)(1-(3-(dimethylamino)propyl)-1H-indol-5-yl)methanone

A mixture of 1-(1-(3-(dimethylamino)propyl)-1H-indole-5-carbonyl)pyrrolidin-3-one (300 mg, 0.96 mmol), benzylamine (1.0 g, 2.1 mmol) and NaCNBH₃ (78 mg, 1.25 mmol) in EtOH (5.0 mL) was stirred at room temperature overnight. When TLC showed that the reaction was complete, the mixture was concentrated in vacuo. The residue was purified by prep-TLC to give the target as a colorless oil (18 mg, 5%).

¹H NMR (400 MHz, CD₃OD): δ 7.79 (d, J=18.4 Hz, 1H), 7.51 (d, J=8.8 Hz, 1H), 7.40-7.19 (m, 7H), 6.56 (d, J=2.8 Hz, 1H), 4.28 (t, J=6.8 Hz, 2H), 3.85-3.49 (m, 6H), 3.32-3.31 (m, 1H), 2.48 (t, J=7.6 Hz, 2H), 2.36 (s, 6H), 2.24-2.06 (m, 3H), 2.00-1.89 (m, 1H). LCMS: m/z 405.3 [M+H]⁺.

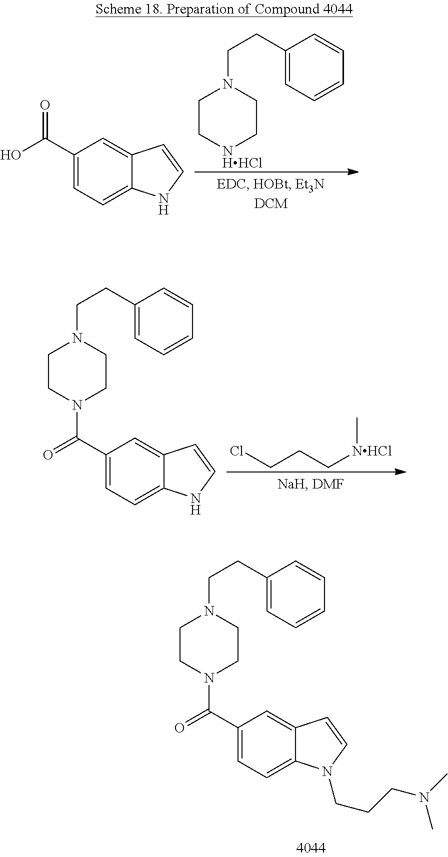

### Preparation of (1H-indol-5-yl)(4-phenethylpiperazin-1-yl)methanone

To a solution of 1-phenethylpiperazine hydrochloride (210 mg, 1.24 mmol) in DCM (10 mL) was added Et₃N (125 mg, 1.24 mmol), 1H-indole-5-carboxylic acid (100 mg, 0.62 mmol), EDC (238 mg, 1.24 mmol) and HOBt (167 mg, 1.24 mmol) over an ice bath. The reaction mixture was warmed to room temperature and stirred overnight. To the resultant was added DCM. The mixture was washed with NH₄Cl solution, NaHCO₃ solution and brine. The DCM phase was concentrated and purified via a short silica gel column to give a pale solid (144 mg, 70%).

### Preparation of Compound 4044, (1-(3-(dimethylamino)propyl)-1H-indol-5-yl)(4-phenethylpiperazin-1-yl)methanone

To a solution of (1H-indol-5-yl)(4-phenethylpiperazin-1-yl)methanone (100 mg, 0.3 mmol) in DMF (10 mL) was added NaH (18 mg, 0.75 mmol) portion wise over an ice bath. The reaction mixture was stirred at 0° C. for 0.5 hours followed by 3-chloro-N,N-dimethylpropan-1-amine hydrochloride (65 mg, 0.45 mmol) added. The mixture was warmed to 80° C. and stirred for 1.5 hours. The resultant was poured into water and extracted with EtOAc. The organic layer was concentrated and purified by gel column to give a colorless oil, (65 mg, 50%).

¹H NMR (400 MHz, DMSO-d₆): δ 7.62 (br s, 1H), 7.57 (d, J=9.6 Hz, 1H), 7.50 (d, J=2.4 Hz, 1H), 7.30-7.16 (m, 6H), 7.53 (d, J=3.2 Hz, 1H), 4.28 (t, J=8.0 Hz, 2H), 3.53-3.37 (m, 4H), 2.75-2.73 (m, 4H), 2.56-2.50 (m, 12H), 2.09 (t, J=8.0 Hz, 2H).

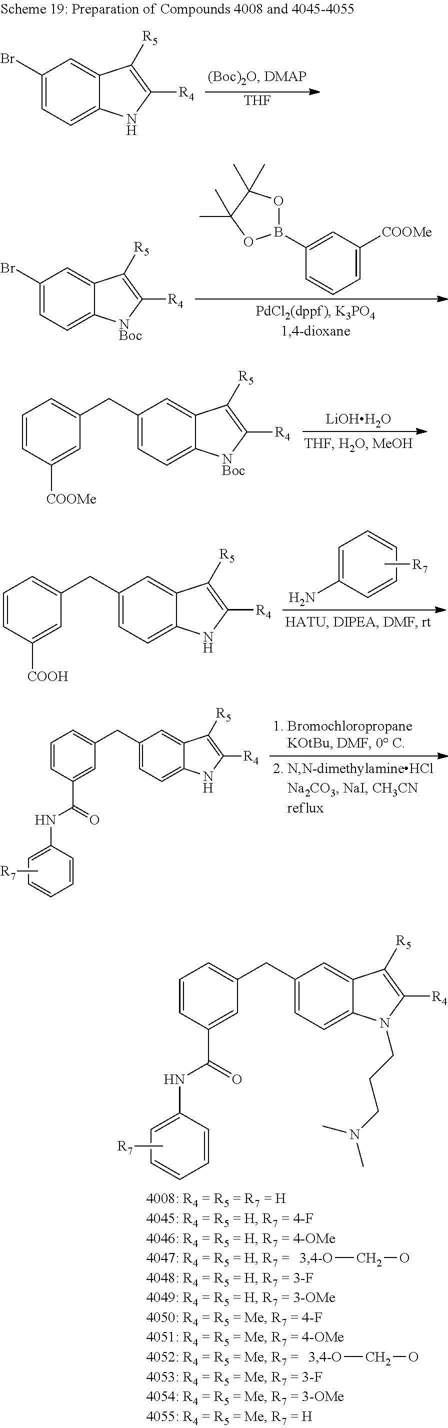

### Preparation of tert-butyl 5-bromo-1H-indole-1-carboxylate

Boc-anhydride (12.8 g, 58.65 mmol) was added to a stirred solution of 5-bromo-1H-indole (5.0 g, 25.50 mmol) in THF (100 mL) at room temperature. DMAP (1.24 g, 10.20 mmol) was added portionwise. The reaction mass was stirred at room temperature for 16 hours. After complete consumption of the starting material THF was evaporated under vacuum. The residue was dissolved in EtOAc, washed with water followed by brine solution and dried over anhydrous Na₂SO₄. The organic layer was concentrated under reduced pressure to afford the crude product. The crude compd was purified on 100-200 mesh silica-gel eluting with 10% EtOAc in petroleum ether to obtain an white solid (6.5 g, 86%).

¹H NMR (400 MHz, CDCl₃): δ 8.02 (br d, J=8.4 Hz, 1H), 7.69 (d, J=2.0 Hz, 1H), 7.58 (d, J=4.0 Hz, 1H), 7.39 (dd, J=8.8 Hz, 2.0 Hz, 1H), 6.50 (d, J=3.6 Hz, 1H), 1.67 (s, 9H). LCMS: m/z 297.0 [M+H]⁺.

Other analogues prepared by this method:

tert-butyl 5-bromo-2,3-dimethyl-1H-indole-1-carboxylate (76%).

¹H NMR (400 MH_{z}, CDCl₃): δ 7.97 (d, J=8.4 Hz, 1H), 7.52 (d, J=2.0 Hz, 1H), 7.28 (dd, J=8.4 Hz, 2.0 Hz, 1H), 2.52 (s, 3H), 2.16 (s, 3H), 1.65 (s, 9H).

### Preparation of tert-butyl 5-(3-(methoxycarbonyl)benzyl)-1H-indole-1-carboxylate

K₃PO₄ (8.06 g, 37.98 mmol) was added to a stirred solution of tert-butyl 5-bromo-1H-indole-1-carboxylate (4.5 g, 15.19 mmol) and methyl 3-(4,4,5,5-tetramethyl-1,3,2-dioxaborolan-2-yl)benzoate (5.03 g, 18.23 mmol) in dry 1, 4-dioxane (50 mL) at room temperature. Argon was purged into the reaction mixture for 10 minutes. PdCl₂(dppf).DCM (0.745 g, 0.91 mmol) was added into reaction mixture, which was subsequently purged with argon for another 10 minutes. The reaction mixture was heated to 100° C. and stirred for 16 hours. After complete consumption of the starting material, the reaction mixture was cooled to room temperature, diluted with EtOAc and filtered through a bed of celite. The filtrate was concentrated under reduced pressure in order to afford the crude product. The crude compound was purified by flash column chromatography using 5% EtOAc in petroleum ether as an eluent, to afford a yellow liquid (2.9 g, 54%).

¹H NMR (400 MHz, CDCl₃): δ 8.04 (br d, J=8.8 Hz, 1H), 7.92 (d, J=1.6 Hz, 1H), 7.87 (d, J=8.4 Hz, 1H), 7.57 (d, J=3.2 Hz, 1H), 7.40-7.27 (m, 3H), 7.16 (dd, J=8.4 Hz, 1.6 Hz, 1H), 6.48 (d, J=3.2 Hz, 1H), 4.13 (s, 2H), 3.89 (s, 3H), 1.66 (s, 9H). LCMS: m/z 365.9 [M+H]⁺.

Other analogues prepared by this method:

tert-butyl 5-(3-(methoxycarbonyl)benzyl)-2,3-dimethyl-1H-indole-1-carboxylate (27%)

¹H NMR (400 MHz, DMSO-d₆): δ 7.93 (d, J=8.4 Hz, 1H), 7.80 (br s, 1H), 7.78 (d, J=8.8 Hz, 1H), 7.54 (br d, J=8.0 Hz, 1H), 7.43 (t, J=8.0 Hz, 1H), 7.36 (br s, 1H), 7.08 (dd, J=8.8 Hz, 2.0 Hz, 1H), 4.12 (s, 2H), 3.82 (s, 3H), 2.46 (s, 3H), 2.14 (s, 3H), 1.60 (s, 9H).

### Preparation of 3-((1H-indol-5-yl)methyl)benzoic acid

tert-Butyl 5-(3-(methoxycarbonyl)benzyl)-1H-indole-1-carboxylate (2.5 g, 6.8 mmol) was dissolved in a THF:MeOH (1:1) mixture (40 mL). A solution of LiOH.H₂O (1.14 g, 27.20 mmol) in water (20 mL) was added at 0° C. The reaction mixture was allowed to stir at room temperature for 40 hours. After complete consumption of the starting material, the reaction mass was concentrated and then partitioned between ethyl acetate and water. The aqueous layer was collected, acidified with 2 N HCl and extracted with ethyl acetate. The organic layer was washed with brine solution, dried over anhydrous Na₂SO₄ and concentrated under reduced pressure to afford an off white solid (1.1 g, 64%).

¹ H NMR (400 MHz, DMSO-d₆): δ 12.80 (br s, 1H), 10.98 (br s, 1H), 7.79 (br s, 1H), 7.75 (d, J=8.4 Hz, 1H), 7.47 (d, J=7.6 Hz, 1H), 7.40-7.34 (m, 2H), 7.32-7.28 (m, 2H), 6.94 (dd, J=8.4 Hz, 1.6 Hz, 1H), 6.38 (d, J=3.2 Hz, 1H), 4.07 (s, 2H). LCMS: m/z 250.28 [M−H]⁻.

Other analogues prepared by this method:

3-((2,3-dimethyl-1H-indol-5-yl)methyl)benzoic acid (77%).

¹H NMR (400 MHz, DMSO-d₆): δ 12.82 (br s, 1H), 10.52 (br s, 1H), 7.77 (br s, 1H), 7.73 (d, J=7.6 Hz, 1H), 7.48 (d, J=7.6 Hz, 1H), 7.38 (t, J=7.6 Hz, 1H), 7.21 (br s, 1H), 7.12 (d, J=8.4 Hz, 1H), 6.83 (dd, J=8.4 Hz, 1.6 Hz, 1H), 4.04 (s, 2H), 2.28 (s, 3H), 2.11 (s, 3H). LCMS: m/z 280.39 [M+H]⁺.

### Preparation of 3-((1H-indol-5-yl)methyl)-N-(4-methoxyphenyl)benzamide

To a stirred solution of 3-((1H-indol-5-yl)methyl)benzoic acid (230 mg, 0.913 mmol) in DMF (5 mL), DIPEA (0.45 mL) was added. The mixture was stirred for 10 minutes, followed by the addtion of HATU (696 mg, 1.83 mmol), and stirring for a further 30 minutes. The reaction mass was cooled to 0° C. and 4-methoxyaniline (124 mg, 1.01 mmol) was added. The mixture was then stirred at room temperature overnight. After complete consumption of the starting material, the reaction mixture was poured into ice water. The resulting precipitate was collected by filtration, dried and purified by flash column chromatography using 20% ethyl acetate in petroleum ether as an eluent to obtain a brown solid (120 mg, 38%).

¹H NMR (400 MHz, DMSO-d₆): δ 10.98 (br s, 1H), 10.22 (br s, 1H), 7.82 (d, J=2.4 Hz, 1H), 7.76 (d, J=8.4 Hz, 1H), 7.64 (d, J=8.0 Hz, 2H), 7.43-7.37 (m, 3H), 7.32-7.28 (m, 2H), 6.96 (dd, J=8.4 Hz, 2.4 Hz, 1H), 6.92 (d, J=8.0 Hz, 2H), 6.36 (d, J=2.8 Hz, 1H), 4.07 (s, 2H), 3.76 (s, 3H). LCMS: m/z 357.16 [M+H]⁺.

Other analogues prepared by this method:

3-((1H-indol-5-yl)methyl)-N-phenylbenzamide (77%).

3-((1H-indol-5-yl)methyl)-N-(4-fluorophenyl)benzamide (70%).

3-((1H-indol-5-yl)methyl)-N-(benzo[d][1,3]dioxol-5-yl)benzamide (36%).

3-((1H-indol-5-yl)methyl)-N-(3-fluorophenyl)benzamide (65%).

3-((1H-indol-5-yl)methyl)-N-(3-methoxyphenyl)benzamide (70%).

3-((2,3-dimethyl-1H-indol-5-yl)methyl)-N-(4-fluorophenyl)benzamide (75%).

3-(2,3-dimethyl-1H-indol-5-yl)methyl)-N-(4-methoxyphenyl)benzamide (73%).

N-(benzo[d][1,3]dioxol-5-yl)-3-((2,3-dimethyl-1H-indol-5-yl)methyl)benzamide (70%).

3-((2,3-dimethyl-1H-indol-5-yl)methyl)-N-(3-fluorophenyl)benzamide (75%).

3-((2,3-dimethyl-1H-indol-5-yl)methyl)-N-(3-methoxyphenyl)benzamide (73%).

3((2,3-dimethyl-1H-indol-5-yl)methyl)-N-phenylbenzamide (78%).

### Preparation of Compound 4045, 3-((1-(3-chloropropyl)-1H-indol-5-yl)methyl)-N-(4-fluorophenyl)benzamide

Potassium tert-butoxide (157 mg, 1.39 mmol) was added portionwise to a stirred solution of 3-((1H-indol-5-yl)methyl)-N-(4-fluorophenyl)benzamide (240 mg, 0.699 mmol) in dry DMF (5 mL) at 0° C. The reaction mixture was allowed to warm to room temperature for 30 minutes. Bromochloropropane (0.095 mL, 1.04 mmol) was added dropwise at 0° C. and the mixture was allowed to stir at room temperature for 3 hours.

After complete consumption of the starting material, ice cold water was added into the reaction mixture, which was then extracted with ethyl acetate. The organic layer was washed with brine solution, dried over anhydrous Na₂SO₄ and concentrated under reduced pressure to afford a brown solid (150 mg, 51%).

¹H NMR (400 MHz, DMSO-d₆): δ 10.22 (br s, 1H), 7.82 (d, J=2.0 Hz, 1H), 7.79-7.74 (m, 2H), 7.47-7.37 (m, 3H), 7.33 (d, J=2.8 Hz, 1H), 7.17 (t, J=8.8 Hz, 2H), 7.04 (br d, J=8.4 Hz, 1H), 6.80 (t, J=7.6 Hz, 1H), 6.55-6.50 (m, 1H), 6.36 (d, J=2.8 Hz, 1H), 4.13 (t, J=7.2 Hz, 2H), 4.07 (s, 2H), 3.52 (t, J=7.2 Hz, 2H), 2.18 (quintet, J=7.2 Hz, 2H). LCMS: m/z 433.29 [M+H]⁺.

Other analogues prepared by this method:

3-((1-(3-chloropropyl)-1H-indol-5-yl)methyl)-N-phenylbenzamide (77%).

3-((1-(3-chloropropyl)-1H-indol-5-yl)methyl)-N-(4-methoxyphenyl)benzamide (50%).

N-(benzo[d][1,3]dioxol-5-yl)-3-((1-(3-chloropropyl)-1H-indol-5-yl)methyl)benzamide (51%).

3-((1-(3-chloropropyl)-1H-indol-5-yl)methyl)-N-(3-fluorophenyl)benzamide (65%).

3-((1-(3-chloropropyl)-1H-indol-5-yl)methyl)-N-(3-methoxyphenyl)benzamide (70%).

3-((1-(3-chloropropyl)-2,3-dimethyl-1H-indol-5-yl)methyl)-N-(4-fluorophenyl)benzamide (75%).

3-((1-(3-chloropropyl)-2,3-dimethyl-1H-indol-5-yl)methyl)-N-(4-methoxyphenyl)benzamide (73%).

N-(benzo[d][1, 3]dioxol-5-yl)-3-((1-(3-chloropropyl)-2,3-dimethyl-1H-indol-5-yl)methyl)benzamide (70%).

3-((1-(3-chloropropyl)-2,3-dimethyl-1H-indol-5-yl)methyl)-N-(3-fluorophenyl)benzamide (75%).

3-((1-(3-chloropropyl)-2,3-dimethyl-1H-indol-5-yl)methyl)-N-(3-methoxyphenyl)benzamide (73%).

3-((1-(3-chloropropyl)-2,3-dimethyl-1H-indol-5-yl)methyl)-N-phenylbenzamide (78%).

### Preparation of Compound 4045, 3-((1-(3-(dimethylamino)propyl)-1H-indol-5-yl)methyl)-N-(4-fluorophenyl)benzamide

To a stirred solution of the intermediate 3-((1-(3-chloropropyl)-1H-indol-5-yl)methyl)-N-(4-fluorophenyl)benzamide (150 mg, 0.357 mmol) in acetonitrile (5 mL) at room temperature, sodium iodide (151 mg, 0.694 mmol) and sodium carbonate (151 mg, 1.42 mmol) were added, followed by N,N-dimethylamine hydrochloride (72 mg, 0.892 mmol). The reaction mixture was heated to 75° C. for 16 hours. After complete consumption of the starting material, the reaction mixture was cooled to room temperature, diluted with EtOAc, washed with water and brine solution, dried over anhydrous Na₂SO₄ and concentrated under reduced pressure to afford the crude product. The crude product was purified by flash column chromatography using 5% MeOH-DCM as an eluent to afford an off-white solid (24 mg, 16%).

¹H NMR (400 MHz, CD₃OD): δ 7.79 (br s, 1H), 7.73 (br d, J=7.6 Hz, 1H), 7.65 (dd, J=8.8 Hz, 4.8 Hz, 2H), 7.46-7.33 (m, 4H), 7.20 (d, J=3.2 Hz, 1H), 7.12-7.03 (m, 3H), 6.41 (d, J=2.8 Hz, 1H), 4.26 (t, J=6.8 Hz, 2H), 4.13 (s, 2H), 2.84-2.76 (m, 2H), 2.62 (s, 6H), 2.19-2.12 (m, 2H). LCMS: m/z 430.33 [M+H]⁺.

Other analogues prepared by this method:

Compound 4008, 34(1-(3-(dimethylamino)propyl)-1H-indol-5-yl)methyl)-N-phenylbenzamide (20%).

¹H NMR (300 MHz, DMSO-d₆): δ 10.20 (br s, 1H), 7.81-7.70 (m, 4H), 7.50-7.40 (m, 4H), 7.44-7.35 (m, 3H), 7.13-7.02 (m, 2H), 6.39 (d, J=2.7 Hz, 1H), 4.20 (t, J=6.6 Hz, 2H), 4.09 (s, 2H), 2.86-2.70 (m, 2H), 2.57 (br s, 6H), 2.11-1.97 (m, 2H). LCMS: m/z 412.50 [M+H]⁺.

Compound 4046, 3-((1-(3-(dimethylamino)propyl)-1H-indol-5-yl)methyl)-N-(4-methoxyphenyl)benzamide (24%).

¹ H NMR (300 MHz, DMSO-d₆): δ 10.07 (br s, 1H), 7.81 (br s, 1H), 7.75 (br d, J=6.9 Hz, 1H), 7.64 (d, J=9.3 Hz, 2H), 7.45-7.33 (m, 4H), 7.31 (d, J=2.7 Hz, 1H), 7.04 (br d, J=8.1 Hz, 1H), 6.91 (d, J=9.0 Hz, 2H), 6.36 (d, J=3.0 Hz, 1H), 4.15 (t, J=7.2 Hz, 2H), 4.08 (s, 2H), 3.74 (s, 3H), 2.30-2.18 (m, 8H), 1.93-1.82 (m, 2H). LCMS: m/z 442.52 [M+H]⁺.

Compound 4047, N-(benzo[d][1,3]dioxol-5-yl)-3-((1-(3-(dimethylamino)propyl)-1H-indol-5-yl)methyl)benzamide (18%).

¹H NMR (400 MHz, DMSO-d₆): δ 10.06 (br s, 1H), 7.80 (br s, 1H), 7.73 (br d, J=6.8 Hz, 1H), 7.47-7.34 (m, 5H), 7.30 (d, J=3.2 Hz, 1H), 7.16 (dd, J=8.4 Hz, 2.0 Hz, 1H), 7.03 (dd, J=8.4 Hz, 1.6 Hz, 1H), 6.88 (d, J=8.4 Hz, 1H), 6.35 (d, J=3.2 Hz, 1H), 5.99 (s, 2H), 4.14 (t, J=6.8 Hz, 2H), 4.08 (s, 2H), 2.15-2.08 (m, 8H), 1.84 (quintet, J=6.8 Hz, 2H). LCMS: m/z 456.41 [M+H]⁺.

Compound 4048, 3-((1-(3-(dimethylamino)propyl)-1H-indol-5-yl)methyl)-N-(3-fluorophenyl)benzamide (6%).

¹H NMR (400 MHz, DMSO-d₆): δ 10.36 (br s, 1H), 7.82 (br s, 1H), 7.77-7.70 (m, 2H), 7.54 (br d, J=9.2 Hz, 1H), 7.48-7.34 (m, 5H), 7.30 (d, J=2.8 Hz, 1H), 7.03 (br d, J=7.2 Hz, 1H), 6.92 (td, J=8.8 Hz, 2.8 Hz, 1H), 6.35 (d, J=2.8 Hz, 1H), 4.15 (t, J=6.8 Hz, 2H), 4.09 (s, 2H), 2.18-2.09 (m, 8H), 1.84 (quintet, J=6.8 Hz, 2H). LCMS: m/z 430.40 [M+H]⁺.

Compound 4049, 3-((1-(3-(dimethylamino)propyl)-1H-indol-5-yl)methyl)-N-(3-methoxyphenyl)benzamide (29%).

¹H NMR (300 MHz, DMSO-d₆): δ 10.16 (br s, 1H), 7.81 (br s, 1H), 7.76 (br d, J=6.9 Hz, 1H), 7.50-7.29 (m, 7H), 7.23 (t, J=8.1 Hz, 1H), 7.05 (br d, J=8.7 Hz, 1H), 6.67 (dd, J=8.1 Hz, 2.4 Hz, 1H), 6.37 (d, J=3.3 Hz, 1H), 4.18 (t, J=6.9 Hz, 2H), 4.09 (s, 2H), 3.74 (s, 3H), 2.52-2.32 (m, 8H), 2.02-1.91 (m, 2H). LCMS: m/z 442.50 [M+H]⁺.

Compound 4050, 34(1-(3-(dimethylamino)propyl)-2,3-dimethyl-1H-indol-5-yl)methyl)-N-(4-fluorophenyl)benzamide (21%).

¹H NMR (300 MHz, CD₃OD): δ 7.79 (br s, 1H), 7.72 (br d, J=6.9 Hz, 1H), 7.65 (dd, J=8.7 Hz, 4.8 Hz, 2H), 7.46-7.35 (m, 2H), 7.27 (br s, 1H), 7.22 (d, J=8.4 Hz, 1H), 7.07 (t, J=8.7 Hz, 2H), 6.95 (br d, J=8.4 Hz, 1H), 4.15-4.10 (m, 4H), 2.41-2.31 (m, 5H), 2.25 (s, 6H), 2.19 (s, 3H), 1.90 (quintet, J=7.8 Hz, 2H). LCMS: m/z 458.59 [M+H]⁺.

Compound 4051, 34(1-(3-(dimethylamino)propyl)-2,3-dimethyl-1H-indol-5-yl)methyl)-N-(4-methoxyphenyl)benzamide (40%).

¹H NMR (300 MHz, CD₃OD): δ 7.79 (br s, 1H), 7.71 (br d, J=6.9 Hz, 1H), 7.53 (d, J=9.0 Hz, 2H), 7.45-7.34 (m, 2H), 7.27 (br s, 1H), 7.22 (d, J=8.4 Hz, 1H), 6.95 (dd, J=8.4 Hz, 1.5 Hz, 1H), 6.90 (d, J=9.3 Hz, 2H), 4.16-4.10 (m, 4H), 3.79 (s, 3H), 2.41-2.33 (m, 5H), 2.24 (s, 6H), 2.19 (s, 3H), 1.89 (quintet, J=7.5 Hz, 2H). LCMS: m/z 470.57 [M+H]⁺.

Compound 4052, N-(benzo[d][1,3]dioxol-5-yl)-3-((1-(3-(dimethylamino)propyl)-2,3-dimethyl-1H-indol-5-yl)methyl)benzamide (22%).

¹H NMR (300 MHz, CD₃OD): δ 7.77 (br s, 1H), 7.69 (br d, J=7.5 Hz, 1H), 7.46-7.34 (m, 2H), 7.32-7.24 (m, 2H), 7.21 (d, J=8.4 Hz, 1H), 7.03 (dd, J=8.4 Hz, 1.8 Hz, 1H), 6.95 (dd, J=8.4 Hz, 1.5 Hz, 1H), 6.78 (d, J=8.4 Hz, 1H), 5.94 (s, 2H), 4.16-4.10 (m, 4H), 2.41-2.33 (m, 5H), 2.25 (s, 6H), 2.19 (s, 3H), 1.90 (quintet, J=7.2 Hz, 2H). LCMS: m/z 484.60 [M+H]⁺.

Compound 4053, 34(1-(3-(dimethylamino)propyl)-2,3-dimethyl-1H-indol-5-yl)methyl)-N-(3-fluorophenyl)benzamide (31%).

¹H NMR (300 MHz, CD₃OD): δ 7.80 (br s, 1H), 7.72 (br d, J=6.9 Hz, 1H), 7.62 (dt, J=11.4 Hz, 2.1 Hz, 1H), 7.48-7.29 (m, 4H), 7.27 (br s, 1H), 7.22 (d, J=8.4 Hz, 1H), 6.96 (dd, J=8.4 Hz, 1.5 Hz, 1H), 6.86 (td, J=8.1 Hz, 1.5 Hz, 1H), 4.15-4.10 (m, 4H), 2.39-2.31 (m, 5H), 2.23 (s, 6H), 2.19 (s, 3H), 1.89 (quintet, J=7.5 Hz, 2H). LCMS: m/z 458.59 [M+H]⁺.

Compound 4054, 34(1-(3-(dimethylamino)propyl)-2,3-dimethyl-1H-indol-5-yl)methyl)-N-(3-methoxyphenyl)benzamide (26%).

¹H NMR (300 MHz, CD₃OD): δ 7.79 (br s, 1H), 7.71 (d, J=6.9 Hz, 1H), 7.47-7.34 (m, 3H), 7.27 (br s, 1H), 7.24-7.16 (m, 3H), 6.95 (dd, J=8.4 Hz, 1.5 Hz, 1H), 6.71 (dt, J=6.9 Hz, 2.7 Hz, 1H), 4.16-4.10 (m, 4H), 3.79 (s, 3H), 2.42-2.32 (m, 5H), 2.24 (s, 6H), 2.19 (s, 3H), 1.89 (quintet, J=7.5 Hz, 2H). LCMS: m/z 470.60 [M+H]⁺.

Compound 4055, 34(1-(3-(dimethylamino)propyl)-2,3-dimethyl-1H-indol-5-yl)methyl)-N-phenylbenzamide (12%).

¹H NMR (300 MHz, CD₃OD): δ 7.80 (br s, 1H), 7.73 (dt, J=7.2 Hz, 1.5 Hz, 1H), 7.65 (d, J=8.4 Hz, 2H), 7.47-7.30 (m, 5H), 7.28 (br s, 1H), 7.13 (t, J=7.2 Hz, 1H), 6.96 (dd, J=8.7 Hz, 1.5 Hz, 1H), 4.16-4.10 (m, 4H), 2.41-2.32 (m, 5H), 2.25 (s, 6H), 2.19 (s, 3H), 1.90 (quintet, J=7.2 Hz, 2H). LCMS: m/z 440.54 [M+H]⁺.

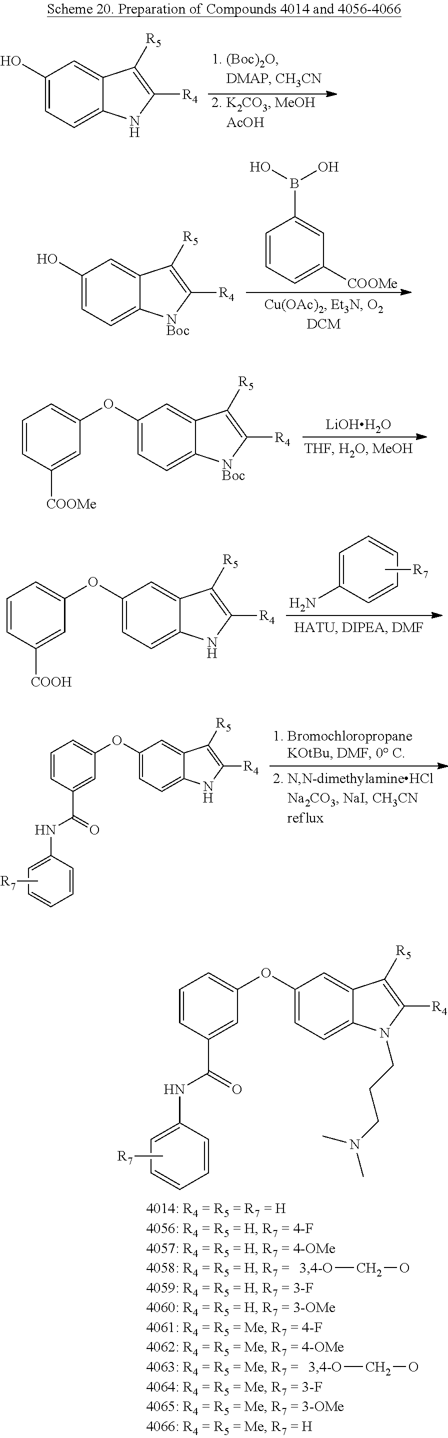

### Preparation of tent-butyl 5-hydroxy-2,3-dimethyl-1H-indole-1-carboxylate

To a stirred solution of 2,3-dimethyl-1H-indol-5-ol (7.2 g, 44.72 mmol), in acetonitrile (72 mL) was added Boc-anhydride (29.2 g, 134.16 mmol) and DMAP (0.55 g, 4.472 mmol) at room temperature. The reaction mass was stirred at room temperature overnight. After complete consumption of the starting material, acetonitrile was evaporated under reduced pressure to yield a crude mixture of the desired 5-hydroxy indole product and its 5-O-Boc protected analogue (8.2 g, 51.42 mmol). The mixture was re-dissolved in MeOH (828 mL), K₂CO₃ (21.3 g, 154.2 mmol) was added and the resulting mixture was stirred at room temperature for 2 hours. After completion of the reaction, the mixture was cooled to 0° C., acetic acid was added (10 mL) and the mixture was stirred for 10 minutes. The reaction mass was extracted with EtOAc. The organic layer was washed with water and brine solution, dried over anhydrous Na₂SO₄ and concentrated under reduced pressure to afford the crude product. The crude product was purified on 100-200 mesh silica gel eluting with 20% EtOAc in pet ether to afford the desired compound as a brown liquid, (9.5 g, 72%). LCMS: m/z 262.40 [M+H]⁺.

Other analogues prepared by this method:

tert-butyl 5-hydroxy-1H-indole-1-carboxylate (64%).

### Preparation of tert-butyl 5-(3-(methoxycarbonyl)phenoxy)-2,3-dimethyl-1H-indole-1-carboxylate

To a stirred solution of tert-butyl 5-hydroxy-2,3-dimethyl-1H-indole-1-carboxylate (7.0 g, 26.79 mmol) in DCM (100 mL) was added (3-(methoxycarbonyl)phenyl)boronic acid (14.46 g, 80.36 mmol). Then Cu(OAc)₂ (12.16 g, 66.97 mmol) was added, followed by Et₃N (18.5 mL, 133.9 mmol) and the system was purged with oxygen gas for 4 hours. The whole reaction mass was stirred under an oxygen atmosphere overnight. After complete consumption of the starting material, the reaction mass was filtered through a bed of Celite. The filtrate was diluted with water and extracted with DCM. The organic layer was washed with brine, dried over anhydrous Na₂SO₄ and concentrated under reduced pressure to afford the crude product. The crude compound was purified on 100-200 mesh silica gel eluting with 10% EtOAc in petroleum ether to afford a brown solid (8.2 g, 77%). LCMS: m/z 396.0 [M+H]⁺.

Other analogues prepared by this method:

tent-butyl 5-(3-(methoxycarbonyl)phenoxy)-1H-indole-1-carboxylate (70%).

### Preparation of 3-((2,3-dimethyl-1 H-indol-5-yl)oxy)benzoic acid

To a stirred solution of tert-butyl 5-(3-(methoxycarbonyl)phenoxy)-2,3-dimethyl-1H-indole-1-carboxylate (8.2 g, 20.8 mmol) in THF (100 mL) methanol (100 mL) and water (100 mL), was added LiOH.H₂O (17.43 g, 415.2 mmol). The mixture was stirred at room temperature for 4 hours. After complete consumption of the starting material, THF was evaporated under reduced pressure and the reaction mass was cooled to 0° C., acidified (pH 1) with 1 N HCl, and then extracted with EtOAc. The organic layers were dried over anhydrous Na₂SO₄ and concentrated under reduced pressure to afford the crude product. Trituration with n-pentane afforded an off-white solid (5.0 g, 86%).

¹H NMR (300 MHz, DMSO-d₆): δ 12.99 (br s, 1H), 10.77 (br s, 1H), 7.58 (d, J=8.4 Hz, 1H), 7.44 (t, J=7.5 Hz, 1H), 7.38-7.17 (m, 3H), 7.08 (d, J=1.8 Hz, 1H), 6.76 (dd, J=8.4 Hz, 1.5 Hz, 1H), 2.35 (s, 3H), 2.13 (s, 3H). LCMS: m/z 282.2 [M+H]⁺.

Other analogues prepared by this method:

3-((1H-indol-5-yl)oxy)benzoic acid (77%).

¹H NMR (400 MHz, DMSO-d₆): δ 12.98 (br s, 1H), 11.17 (br s, 1H), 7.59 (d, J=8.4 Hz, 1H), 7.44-7.36 (m, 3H), 7.31 (d, J=3.2 Hz, 1H), 7.29-7.20 (m, 2H), 6.84 (dd, J=8.4 Hz, 1.6 Hz, 1H), 6.42 (d, J=3.2 Hz, 1H). LCMS: m/z 253.98 [M+H]⁺.

### Preparation of 3-((1H-indol-5-yl)oxy)-N-(4-fluorophenyl)benzamide

To a stirred solution of 3-((1H-indol-5-yl)oxy)benzoic acid (220 mg, 0.87 mmol) in DMF (3 mL), DIPEA (0.76 mL, 4.34 mmol) was added. After 10 minutes stirring, HATU (495 mg, 1.3 mmol) was added and the mixture was stirred for a further 30 minutes at room temperature. The reaction mass was cooled to 0° C., 4-fluoroaniline (160 mg, 1.30 mmol) was added and the reaction mixture was stirred at room temperature overnight. After complete consumption of the starting material, the reaction mixture was poured into ice water and extracted with EtOAc. The organic layer was washed with water and brine, dried over anhydrous Na₂SO₄ and concentrated under reduced pressure to afford the crude product. The crude product was purified on 100-200 mesh silica eluting with 30% EtOAc in petroleum ether to obtain the pure compound (244 mg, 80%).

¹H NMR (400 MHz, DMSO-d₆): δ 11.17 (br s, 1H), 10.26 (br s, 1H), 7.78 (dd, J=8.8 Hz, 4.8 Hz, 2H), 7.62 (br d, J=7.6 Hz, 1H), 7.51-7.38 (m, 4H), 7.27 (br s, 1H), 7.22-7.10 (m, 3H), 6.87 (dd, J=8.4 Hz, 1.6 Hz, 1H), 6.38 (d, J=3.2 Hz, 1H). LCMS: m/z 345.37 [M−H]⁻.

Other analogues prepared by this method:

3-((1H-indol-5-yl)oxy)-N-phenylbenzamide (72%).

3-((1H-indol-5-yl)oxy)-N-(4-methoxyphenyl)benzamide (78%).

3-((1H-indol-5-yl)oxy)-N-(benzo[d][1,3]dioxol-5-yl)benzamide (83%).

3-((1H-indol-5-yl)oxy)-N-(3-fluorophenyl)benzamide (85%).

3-((1H-indol-5-yl)oxy)-N-(3-methoxyphenyl)benzamide (87%).

3-((2,3-dimethyl-1H-indol-5-yl)oxy)-N-(4-fluorophenyl)benzamide (100%).

3-((2,3-dimethyl-1H-indol-5-yl)oxy)-N-(4-methoxyphenyl)benzamide (68%).

N-(benzo[d][1,3]dioxol-5-yl)-3-((2,3-dimethyl-1H-indol-5-yl)oxy)benzamide (80%).

3-((2,3-dimethyl-1H-indol-5-yl)oxy)-N-(3-fluorophenyl)benzamide (77%).

3-((2,3-dimethyl-1H-indol-5-yl)oxy)-N-(3-methoxyphenyl)benzamide (92%).

3-((2,3-dimethyl-1H-indol-5-yl)oxy)-N-phenylbenzamide (86%).

### Preparation of 3-((1-(3-chloropropyl)-1H-indol-5-yl)oxy)-N-(4-fluorophenyl)benzamide

Potassium tert-butoxide (143 mg, 1.27 mmol) was added portionwise to a stirred solution of 3-((1H-indol-5-yl)oxy)-N-(4-fluorophenyl)benzamide (294 mg, 0.850 mmol) in dry DMF (5 mL) at 0° C. The reaction mixture was allowed to warm to room temperature for 30 minutes. Bromochloropropane (0.17 mL, 1.7 mmol) was added dropwise at 0° C. and the mixture was allowed to stir at room temperature for 3 hours. After complete consumption of the starting material, ice cold water was added into the reaction mixture, which was then extracted with ethyl acetate. The organic layer was washed with brine solution, dried over anhydrous Na₂SO₄ and concentrated under reduced pressure to afford the crude product. The crude product was purified by flash column chromatography on silica gel using 5% EtOAc in petroleum ether as an eluent to give a brown liquid (259 mg, 72%). LCMS: m/z 423.32 [M+H]⁺.

Other analogues prepared by this method:

3-((1-(3-chloropropyl)-1H-indol-5-yl)oxy)-N-phenylbenzamide (82%).

3-((1-(3-chloropropyl)-1H-indol-5-yl)oxy)-N-(4-methoxyphenyl)benzamide (65%).

N-(benzo[d][1,3]dioxol-5-yl)-3-((1-(3-chloropropyl)-1H-indol-5-yl)oxy)benzamide (60%).

3-((1-(3-chloropropyl)-1H-indol-5-yl)oxy)-N-(3-fluorophenyl)benzamide (64%).

3-((1-(3-chloropropyl)-1H-indol-5-yl)oxy)-N-(3-methoxyphenyl)benzamide (64%).

3-((1-(3-chloropropyl)-2, 3-dimethyl-1H-indol-5-yl)oxy)-N-(4-fluorophenyl)benzamide (26%).

3-((1-(3-chloropropyl)-2, 3-dimethyl-1H-indol-5-yl)oxy)-N-(4-methoxyphenyl)benzamide (36%).

N-(benzo[d][1,3]dioxol-5-yl)-3-((1-(3-chloropropyl)-2,3-dimethyl-1H-indol-5-yl)oxy)benzamide (32%).

3-((1-(3-chloropropyl)-2, 3-dimethyl-1H-indol-5-yl)oxy)-N-(3-fluorophenyl)benzamide (35%).

3-((1-(3-chloropropyl)-2,3-dimethyl-1H-indol-5-yl)oxy)-N-(3-methoxyphenyl)benzamide (37%).

3-((1-(3-chloropropyl)-2,3-dimethyl-1H-indol-5-yl)oxy)-N-phenylbenzamide (25%).

### Preparation of Compound 4056, 3-((1-(3-(dimethylamino)propyl)-1H-indol-5-yl)oxy)-N-(4-fluorophenyl)benzamide

To a stirred solution of the intermediate 3-((1-(3-chloropropyl)-1H-indol-5-yl)oxy)-N-(4-fluorophenyl)benzamide (128 mg, 0.303 mmol) in acetonitrile (5 mL) at room temperature, sodium iodide (114 mg, 0.761 mmol) and sodium carbonate (160 mg, 1.51 mmol) were added, followed by N,N-dimethylamine hydrochloride (99 mg, 1.21 mmol). The reaction mixture was heated to 90° C. for 12 hours. After complete consumption of the starting material, the reaction mixture was cooled to room temperature, diluted with EtOAc, washed with water and brine solution, dried over anhydrous Na₂SO₄ and concentrated under reduced pressure to afford the crude product. The crude product was purified by flash column chromatography using 5% MeOH-DCM as an eluent to afford a pale brown liquid (21 mg, 6%).

¹H NMR (300 MHz, DMSO-d₆): δ 10.23 (br s, 1H), 7.75 (dd, J=9.3 Hz, 5.1 Hz, 2H), 7.64 (br d, J=8.1 Hz, 1H), 7.56-7.42 (m, 3H), 7.42 (d, J=3.0 Hz, 1H), 7.26 (d, J=1.8 Hz, 1H), 7.21-7.12 (m, 3H), 6.93 (dd, J=8.7 Hz, 2.4 Hz, 1H), 6.42 (d, J=3.0 Hz, 1H), 4.21 (t, J=6.9 Hz, 2H), 2.27-2.07 (m, 8H), 1.90 (quintet, J=6.9 Hz, 2H). LCMS: m/z 432.4 [M+H]⁺.

Other analogues prepared by this method:

Compound 4014, 3-((1-(3-(dimethylamino)propyl)-1H-indol-5-yl)oxy)-N-phenylbenzamide (9%).

¹H NMR (300 MHz, DMSO-d₆): δ 10.22 (br s, 1H), 7.73 (d, J=7.8 Hz, 2H), 7.65 (br d, J=8.1 Hz, 1H), 7.54 (d, J=9.0 Hz, 1H), 7.51-7.45 (m, 2H), 7.42 (d, J=3.3 Hz, 1H), 7.33 (t, J=7.8 Hz, 2H), 7.27 (d, J=2.1 Hz, 1H), 7.14 (dd, J=8.1 Hz, 2.7 Hz, 1H), 7.09 (t, J=7.8 Hz, 1H), 6.93 (dd, J=9.0 Hz, 2.1 Hz, 1H), 6.43 (d, J=2.7 Hz, 1H), 4.21 (t, J=6.9 Hz, 2H), 2.30-2.12 (m, 8H), 1.96-1.87 (m, 2H). LCMS: m/z 414.4 [M+H]⁺.

Compound 4057, 3-((1-(3-(dimethylamino)propyl)-1H-indol-5-yl)oxy)-N-(4-methoxyphenyl)benzamide (30%).

¹H NMR (300 MHz, DMSO-d₆): δ 10.11 (br s, 1H), 7.65-7.60 (m, 3H), 7.54-7.43 (m, 3H), 7.42 (d, J=3.0 Hz, 1H), 7.26 (d, J=2.7 Hz, 1H), 7.13 (dd, J=9.0 Hz, 2.1 Hz, 1H), 6.95-6.87 (m, 3H), 6.42 (d, J=2.7 Hz, 1H), 4.21 (t, J=6.9 Hz, 2H), 3.73 (s, 3H), 2.20-2.12 (m, 8H), 1.89 (quintet, J=6.9 Hz, 2H). LCMS: m/z 444.48 [M+H]⁺.

Compound 4058, N-(benzo[d][1,3]dioxol-5-yl)-3-((1-(3-(dimethylamino)propyl)-1H-indol-5-yl)oxy)benzamide (30%).

¹ H NMR (300 MHz, DMSO-d₆): δ 10.12 (br s, 1H), 7.62 (br d, J=7.5 Hz, 1H), 7.53 (d, J=9.0 Hz, 1H), 7.49-7.44 (m, 2H), 7.42 (d, J=3.3 Hz, 1H), 7.40 (d, J=1.8 Hz, 1H), 7.25 (d, J=2.1 Hz, 1H), 7.17-7.09 (m, 2H), 6.92 (dd, J=8.7 Hz, 2.1 Hz, 1H), 6.87 (d, J=8.4 Hz, 1H), 6.42 (d, J=2.7 Hz, 1H), 5.99 (s, 2H), 4.21 (t, J=6.9 Hz, 2H), 2.19-2.10 (m, 8H), 1.89 (quintet, J=6.9 Hz, 2H). LCMS: m/z 458.47 [M+H]⁺.

Compound 4059, 3-((1-(3-(dimethylamino)propyl)-1H-indol-5-yl)oxy)-N-(3-fluorophenyl)benzamide (4%).

¹H NMR (300 MHz, DMSO-d₆): δ 10.41 (br s, 1H), 7.71 (dt, J=11.7 Hz, 2.4 Hz, 1H), 7.64 (br d, J=8.1 Hz, 1H), 7.56-7.32 (m, 6H), 7.26 (d, J=2.1 Hz, 1H), 7.14 (dd, J=7.8 Hz, 1.8 Hz, 1H), 6.96-6.87 (m, 2H), 6.42 (d, J=3.3 Hz, 1H), 4.21 (t, J=6.9 Hz, 2H), 2.21-2.09 (m, 8H), 1.94-1.84 (m, 2H). LCMS: m/z 432.49 [M+H]⁺.

Compound 4060, 34(1-(3-(dimethylamino)propyl)-1H-indol-5-yl)oxy)-N-(3-methoxyphenyl)benzamide (13%).

¹H NMR (300 MHz, DMSO-d₆): δ 10.21 (br s, 1H), 7.64 (br d, J=7.8 Hz, 1H), 7.53 (d, J=9.0 Hz, 1H), 7.50-7.39 (m, 4H), 7.33 (br d, J=8.7 Hz, 1H), 7.27-7.19 (m, 2H), 7.13 (dd, J=7.8 Hz, 1.8 Hz, 1H), 6.93 (dd, J=8.4 Hz, 2.1 Hz, 1H), 6.67 (dd, J=7.8 Hz, 1.8 Hz, 1H), 6.42 (d, J=3.3 Hz, 1H), 4.21 (t, J=6.9 Hz, 2H), 3.73 (s, 3H), 2.20-2.11 (m, 8H), 1.89 (quintet, J=6.9 Hz, 2H). LCMS: m/z 444.4 [M+H]⁺.

Compound 4061, 34(1-(3-(dimethylamino)propyl)-2,3-dimethyl-1H-indol-5-yl)oxy)-N-(4-fluorophenyl)benzamide (1%).

¹H NMR (400 MHz, CD₃OD): δ 7.64 (dd, J=9.2 Hz, 4.8 Hz, 2H), 7.55 (br d, J=7.6 Hz, 1H), 7.46 (t, J=2.0 Hz, 1H), 7.41 (t, J=8.0 Hz, 1H), 7.34 (d, J=8.8 Hz, 1H), 7.12-7.04 (m, 4H), 6.84 (dd, J=8.8 Hz, 2.4 Hz, 1H), 4.19 (t, J=6.8 Hz, 2H), 2.51-2.44 (m, 2H), 2.38 (s, 3H), 2.32 (s, 6H), 2.18 (s, 3H), 1.99-1.92 (m, 2H). LCMS: m/z 460.54 [M+H]⁺.

Compound 4062, 34(1-(3-(dimethylamino)propyl)-2,3-dimethyl-1H-indol-5-yl)oxy)-N-(4-methoxyphenyl)benzamide (29%).

¹H NMR (400 MHz, CD₃OD): δ 7.55 (br d, J=7.2 Hz, 1H), 7.51 (d, J=8.8 Hz, 2H), 7.44-7.39 (m, 2H), 7.37 (d, J=8.8 Hz, 1H), 7.14-7.00 (m, 2H), 6.90 (d, J=8.8 Hz, 2H), 6.86 (dd, J=8.8 Hz, 2.0 Hz, 1H), 4.25 (t, J=6.8 Hz, 2H), 3.78 (s, 3H), 3.05-3.00 (m, 2H), 2.76 (s, 6H), 2.40 (s, 3H), 2.18 (s, 3H), 2.16-2.09 (m, 2H). LCMS: m/z 472.4 [M+H]⁺.

Compound 4063, N-(benzo[d][1,3]dioxol-5-yl)-3-((1-(3₇(dimethylamino)propyl)-2,3-dimethyl-1H-indol-5-yl)oxy)benzamide (10%).

¹H NMR (300 MHz, DMSO-d₆): δ 10.12 (br s, 1H), 7.60 (br d, J=7.5 Hz, 1H), 7.48-7.38 (m, 4H), 7.17-7.07 (m, 3H), 6.89-6.80 (m, 2H), 5.99 (s, 2H), 4.13 (t, J=7.2 Hz, 2H), 2.35-2.10 (m, 14H), 1.90-1.75 (m, 2H). LCMS: m/z 486.49 [M+H]⁺.

Compound 4064, 34(1-(3-(dimethylamino)propyl)-2,3-dimethyl-1H-indol-5-yl)oxy)-N-(3-fluorophenyl)benzamide (25%).

¹H NMR (300 MHz, DMSO-d₆): δ 10.41 (br s, 1H), 7.71 (dt, J=11.7 Hz, 2.1 Hz, 1H), 7.63 (br d, J=8.1 Hz, 1H), 7.52-7.32 (m, 5H), 7.15-7.11 (m, 2H), 6.97-6.83 (m, 2H), 4.15 (t, J=6.9 Hz, 2H), 2.73-2.61 (m, 2H), 2.48 (br s, 6H), 2.35 (s, 3H), 2.15 (s, 3H), 1.96-1.82 (m, 2H). LCMS: m/z 460.54 [M+H]⁺.

Compound 4065, 3-((1-(3-(dimethylamino)propyl)-2,3-dimethyl-1H-indol-5-yl)oxy)-N-(3-methoxyphenyl)benzamide (18%).

¹ H NMR (400 MHz, CD₃OD): δ 7.55 (br d, J=7.6 Hz, 1H), 7.46 (t, J=2.0 Hz, 1H), 7.41 (t, J=8.0 Hz, 1H), 7.36-7.32 (m, 2H), 7.24-7.17 (m, 2H), 7.11-7.08 (m, 2H), 6.83 (dd, J=8.8 Hz, 2.4 Hz, 1H), 6.70 (ddd, J=8.0 Hz, 2.4 Hz, 1.2 Hz, 1H), 4.17 (t, J=6.8 Hz, 2H), 3.79 (s, 3H), 2.39-2.34 (m, 5H), 2.24 (s, 6H), 2.18 (s, 3H), 1.97-1.86 (m, 2H). LCMS: m/z 472.4 [M+H]⁺.

Compound 4066, 3-((1-(3-(dimethylamino)propyl)-2,3-dimethyl-1H-indol-5-yl)oxy)-N-phenylbenzamide (18%).

¹H NMR (400 MHz, CD₃OD): δ 7.63 (br d, J=7.6 Hz, 2H), 7.56 (br d, J=8.0 Hz, 1H), 7.47 (t, J=2.0 Hz, 1H), 7.41 (t, J=8.0 Hz, 1H), 7.36-7.30 (m, 3H), 7.15-7.08 (m, 3H), 6.83 (dd, J=8.8 Hz, 2.4 Hz, 1H), 4.17 (t, J=7.2 Hz, 2H), 2.39-2.33 (m, 5H), 2.23 (s, 6H), 2.18 (s, 3H), 1.96-1.87 (m, 2H). LCMS: m/z 442.4 [M+H]⁺.

Scheme 21. Preparation of Compounds 4067-4076

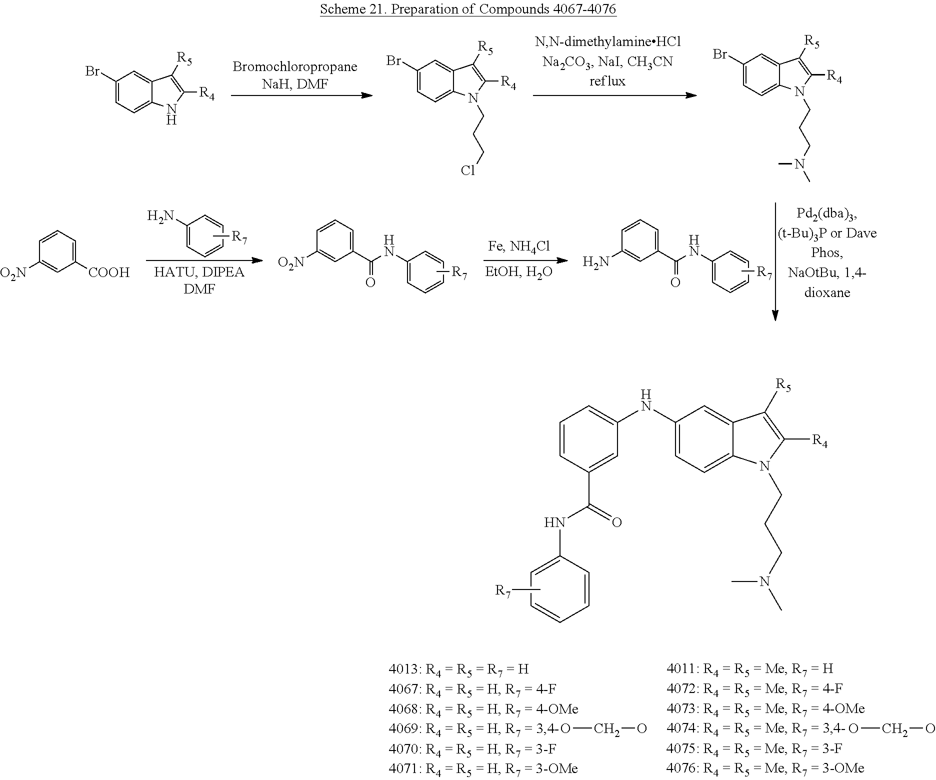

### Preparation of 5-bromo-1-(3-chloropropyl)-1H-indole

NaH (3.88 g, 102 mmol) was added portionwise to a stirred solution of 5-bromo-1H-indole (10 g, 51 mmol) in dry DMF (100 mL) at 0° C. The reaction mixture was allowed to warm to room temperature for 30 minutes. Bromochloropropane (25 mL, 255 mmol) was added drop wise at 0° C. The mixture was allowed to stir at room temperature for 3 hours. After complete consumption of the starting material, NH₄Cl solution was added into the reaction mixture, which was then extracted with ethyl acetate. The organic layer was washed with brine solution, dried over anhydrous Na₂SO₄ and concentrated under reduced pressure to afford a white solid (10.2 g, 74%). LCMS: m/z 271.83, 273.81 [M+H]⁺.

Other analogues prepared by this method:

5-bromo-1-(3-chloropropyl)-2,3-dimethyl-1H-indole (40%). LCMS: m/z 300.09, 302.08 [M+H]⁺.

### Preparation of 3-(5-bromo-2,3-dimethyl-1H-indol-1-yl)-N,N-dimethylpropan-1-amine

To a stirred solution of 5-bromo-1-(3-chloropropyl)-2,3-dimethyl-1H-indole (6.50 g, 21.6 mmol) in acetonitrile (65 mL), sodium iodide (8.10 g, 54.1 mmol), sodium carbonate (11.45 g, 108.1 mmol) and then N,N-dimethylamine hydrochloride (6.96 g, 86.5 mmol) were added at room temperature. The reaction mixture was heated to 75° C. for 16 hours. After complete consumption of the starting material, the reaction mixture was cooled to room temperature, diluted with EtOAc, washed with water and brine solution, dried over anhydrous Na₂SO₄ and concentrated under reduced pressure to afford the crude product. The crude compound was purified by flash column chromatography using 5% MeOH-DCM as an eluent to give a brown semi solid (3.27 g, 49%).

¹H NMR (400 MHz, CDCl₃): δ 7.57 (d, J=2.0 Hz, 1H), 7.18 (dd, J=8.8 Hz, 2.0 Hz, 1H), 7.14 (d, J=8.4 Hz, 1H), 4.11 (t, J=7.2 Hz, 2H), 2.33-2.29 (m, 5H), 2.26 (s, 6H), 2.19 (s, 3H), 1.89 (quintet, J=7.2 Hz, 2H). LCMS: m/z 309.17, 311.16 [M+H]⁺.

Other analogues prepared by this method:

3-(5-bromo-1H-indol-1-yl)-N,N-dimethylpropan-1-amine (50%).

¹H NMR (400 MHz, CDCl₃): δ 7.73 (d, J=1.2 Hz, 1H), 7.29-7.22 (m, 2H), 7.11 (d, J=3.2 Hz, 1H), 6.42 (d, J=3.2 Hz, 1H), 4.18 (t, J=6.8 Hz, 2H), 2.23-1.90 (m, 8H), 1.96 (quintet, J=6.8 Hz, 2H). LCMS: m/z 281.1, 283.1 [M+H]⁺.

### Preparation of N-(4-fluorophenyl)-3-nitrobenzamide

To a stirred solution of 3-nitrobenzoic acid (3.0 g, 17.9 mmol) in DMF (30 mL), DIPEA (7.5 mL, 43 mmol) was added and the mixture was stirred for 10 minutes. HATU (13.6 g, 35.9 mmol) was added and the resulting mixture was stirred for 30 minutes at room temperature. The reaction mass cooled to 0° C., 4-fluoroaniline (1.67 g, 17.9 mmol) was added and the mixture was stirred at room temperature overnight. The progress of the reaction was monitored by TLC. After complete consumption of the starting material, the reaction mixture was poured into ice water. The resulting precipitate was filtered off and dried to obtain the crude product. The crude compound was purified by flash column chromatography using 10% EtOAc in petroleum ether as an eluent to obtain an off white solid (1.3 g, 28%). LCMS: m/z 261.0 [M+H]⁺.

Other analogues prepared by this method:

3-nitro-N-phenylbenzamide (80%).

N-(4-methoxyphenyl)-3-nitrobenzamide (96%).

N-(benzo[d][1,3]dioxol-5-yl)-3-nitrobenzamide (83%).

N-(3-fluorophenyl)-3-nitrobenzamide (73%).

N-(3-methoxyphenyl)-3-nitrobenzamide (66%).

### Preparation of 3-amino-N-(4-fluorophenyl)benzamide

To a stirred solution of compound N-(4-fluorophenyl)-3-nitrobenzamide (1.3 g, 5.0 mmol) in EtOH (15 mL), H₂O (15 mL), Fe powder (0.62 g, 25 mmol) and NH₄Cl (0.53 g, 10 mmol) were added at room temperature. The reaction mixture was heated to 70° C. for 2 hours. The reaction mixture was filtered off and washed with ethyl acetate. The mother liquor concentrated to remove EtOH. The residue was dissolved in ethyl acetate, washed with water and brine and then concentrated under reduced pressure to afford a white solid (0.70 g, 63%).

¹H NMR (400 MHz, DMSO-d₆): δ 10.10 (br s, 1H), 7.78 (dd, J=8.4 Hz, 5.4 Hz, 2H), 7.20-7.12 (m, 3H), 7.09-7.02 (m, 2H), 6.77 (br d, J=8.4 Hz, 1H) 5.26 (br s, 2H). LCMS: m/z 231.06 [M+H]⁺.

Other analogues prepared by this method:

3-amino-N-phenylbenzamide (36%).

3-amino-N-(4-methoxyphenyl)benzamide (80%).

3-amino-N-(benzo[d][1,3]dioxol-5-yl)benzamide (80%).

3-amino-N-(3-fluorophenyl)benzamide (76%).

3-amino-N-(3-methoxyphenyl) benzamide (78%).

### Preparation of Compound 4067, 3-((1-(3-(dimethylamino)propyl)-1H-indol-5-yl)amino)-N-(4-fluorophenyl)benzamide

To a stirred solution of 3-(5-bromo-1H-indol-1-yl)-N,N-dimethylpropan-1-amine (100 mg, 0.356 mmol) in 1,4-dioxane (10 mL) was added NaOtBu (85 mg, 0.88 mmol), Pd₂(dba)₃ (49 mg, 0.054 mmol), (t-Bu)₃P (22 mg, 0.11 mmol) and 3-amino-N-(4-fluorophenyl)benzamide (125 mg, 0.543 mmol) at room temperature. The reaction mixture was heated to 100° C. for 16 hours. After complete consumption of the starting material, the reaction mixture was filtered and extracted with ethyl acetate. The organic layer was washed with water and brine solution, dried over anhydrous Na₂SO₄ and concentrated under reduced pressure to afford the crude product. The crude compound was purified by using prep-TLC, eluting with 5% MeOH in DCM to afford a white solid (71 mg, 47%).

¹H NMR (400 MHz, CDCl₃): δ 7.74 (br s, 1H), 7.56 (dd, J=8.4 Hz, 4.4 Hz, 2H), 7.42-7.37 (m, 2H), 7.33 (d, J=8.8 Hz, 1H), 7.25 (br s, 1H), 7.18 (br d, J=7.2 Hz, 1H), 7.12 (d, J=2.0 Hz, 1H), 7.06-7.00 (m, 4H), 6.43 (d, J=2.0 Hz, 1H), 4.19 (t, J=6.8 Hz, 2H), 2.34-2.20 (m, 8H), 2.04-1.98 (m, 2H). LCMS: m/z 431.21 [M+H]⁺.

Other analogues prepared by this method:

Compound 4013, 3-((1-(3-(dimethylamino)propyl)-1H-indol-5-yl)amino)-N-phenylbenzamide (14%).

¹H NMR (400 MHz, DMSO-d₆): δ 10.16 (br s, 1H), 8.08 (br s, 1H). 7.76 (d, J=7.6 Hz, 2H), 7.45-7.43 (m, 2H), 7.35-7.23 (m, 6H), 7.11-7.06 (m, 2H), 6.99 (dd, J=8.8 Hz, 2.0 Hz, 1H), 6.36 (d, J=2.8 Hz, 1H), 4.19 (t, J=6.8 Hz, 2H), 2.35-2.27 (m, 2H), 2.25 (s, 6H), 2.02-1.92 (m, 2H). LCMS: m/z 413.1 [M+H]⁺.

Compound 4068, 3-((1-(3-(dimethylamino)propyl)-1H-indol-5-yl)amino)-N-(4-methoxyphenyl)benzamide (24%).

¹H NMR (400 MHz, DMSO-d₆): δ 9.98 (br s, 1H), 7.99 (br s, 1H), 7.64 (d, J=8.8 Hz, 2H), 7.43-7.40 (m, 2H), 7.33-7.19 (m, 4H), 7.07 (br d, J=7.2 Hz, 1H), 6.98 (br d, J=8.4 Hz, 1H), 6.90 (d, J=8.8 Hz, 2H), 6.35 (d, J=2.8 Hz, 1H), 4.17 (t, J=6.8 Hz, 2H), 3.73 (s, 3H), 2.41-2.30 (m, 8H), 1.94-1.85 (m, 2H). LCMS: m/z 443.52 [M+H]⁺.

Compound 4069, N-(benzo[d][1,3]dioxol-5-yl)-3-((1-(3-(dimethylamino)propyl)-1H-indol-5-yl)amino)benzamide (12%).

¹H NMR (400 MHz, DMSO-d₆): δ 10.02 (br s, 1H), 8.02 (br s, 1H), 7.45-7.40 (m, 3H), 7.33-7.31 (m, 2H), 7.26 (t, J=8.0 Hz, 1H), 7.20-7.15 (m, 2H), 7.07 (br d, J=8.8 Hz, 1H), 6.99 (dd, J=8.8 Hz, 2.0 Hz, 1H), 6.87 (d, J=8.4 Hz, 1H), 6.35 (d, J=2.8 Hz, 1H), 5.99 (s, 2H), 4.18 (t, J=6.8 Hz, 2H), 2.25-2.12 (m, 8H), 1.99-1.90 (m, 2H). LCMS: m/z 457.0 [M+H]⁺.

Compound 4070, 3-((1-(3-(dimethylamino)propyl)-1H-indol-5-yl)amino)-N-(3- fluorophenyl)benzamide (20%).

¹H NMR (400 MHz, DMSO-d₆): δ 10.30 (br s, 1H), 8.03 (br s, 1H), 7.72 (dt, J=11.6 Hz, 2.1 Hz, 1H), 7.54 (br d, J=8.0 Hz, 1H), 7.44-7.20 (m, 7H), 7.10 (d, J=8.0 Hz, 1H), 6.98 (dd, J=8.8 Hz, 2.0 Hz, 1H), 6.90 (td, J=8.4 Hz, 2.4 Hz, 1H), 6.35 (d, J=2.8 Hz, 1H), 4.16 (t, J=6.8 Hz, 2H), 2.18-2.10 (m, 8H), 1.87 (quintet, J=6.8 Hz, 2H). LCMS: m/z 431.47 [M+H]⁺.

Compound 4071, 34(1-(3-(dimethylamino)propyl)-1H-indol-5-yl)amino)-N-(3-methoxyphenyl)benzamide (21%).

¹H NMR (300 MHz, DMSO-d₆): δ 10.09 (br s, 1H), 8.03 (br s, 1H), 7.46-7.39 (m, 3H), 7.36-7.18 (m, 6H), 7.09 (dd, J=8.4 Hz, 1.8 Hz, 1H), 6.98 (dd, J=8.4 Hz, 2.4 Hz, 1H), 6.66 (dd, J=8.4 Hz, 1.8 Hz, 1H), 6.35 (d, J=3.0 Hz, 1H), 4.17 (t, J=6.9 Hz, 2H), 3.74 (s, 3H), 2.23-2.07 (m, 8H), 1.94-1.83 (m, 2H). LCMS: m/z 443.10 [M+H]⁺.

### Preparation of Compound 4072, 3-((1-(3-(dimethylamino)propyl)-2,3-dimethyl-1H-indol-5-yl)amino)-N-(4-fluorophenyl)benzamide

To a stirred solution of 3-(5-bromo-2,3-dimethyl-1H-indol-1-yl)-N,N-dimethylpropan-1-amine (100 mg, 0.32 mmol) in 1,4-dioxane (10 mL) was added NaOtBu (92 mg, 0.96 mmol), Pd₂(dba)₃ (44 mg, 0.048 mmol), Dave Phos (38 mg, 0.096 mmol) and 3-amino-N-(4-fluorophenyl)benzamide (94 mg, 0.38 mmol) at room temperature. The reaction mixture was heated to 100° C. for 16 hours. After complete consumption of the starting material, the reaction mixture was filtered and extracted with ethyl acetate. The organic layer was washed with brine solution, dried over anhydrous Na₂SO₄ and concentrated under reduced pressure to afford the crude product. The crude compound was purified by flash column chromatography, eluting with 5% MeOH in DCM to afford a white solid (15 mg, 12%).

(10%). ¹H NMR (400 MHz, DMSO-d₆): δ 10.19 (br s, 1H), 7.98 (br s, 1H), 7.56 (dd, J=8.8 Hz, 4.8 Hz, 2H), 7.40 (br s, 1H), 7.31 (d, J=8.4 Hz, 1H), 7.26 (t, J=8.0 Hz, 1H), 7.22-7.14 (m, 4H), 7.06 (br d, J=7.6 Hz, 1H), 6.91 (dd, J=8.0 Hz, 1.6 Hz, 1H), 4.09 (t, J=6.8 Hz, 2H), 2.39-2.31 (m, 5H), 2.25 (br s, 6H), 2.14 (s, 3H), 1.80 (quintet, J=6.8 Hz, 2H). LCMS: m/z 459.11 [M+H]⁺.

Compound 4011, 3-((1-(3-(dimethylamino)propyl)-2,3-dimethyl-1H-indol-5-yl)amino)-N-phenylbenzamide (8%).

¹H NMR (400 MHz, CD₃OD): δ 7.67 (d, J=8.0 Hz, 2H), 7.45 (br s, 1H), 7.35 (t, J=8.0 Hz, 2H), 7.29-7.21 (m, 4H), 7.16-7.10 (m, 2H), 7.00 (dd, J=8.4 Hz, 1.6 Hz, 1H), 4.16 (t, J=6.8 Hz, 2H), 2.57 (t, J=7.6 Hz, 2H), 2.39 (s, 6H), 2.37 (s, 3H), 2.20 (2, 3H), 2.00-1.96 (m, 2H). LCMS: m/z 441.3 [M+H]⁺.

Compound 4073, 3-((1-(3-(dimethylamino)propyl)-2,3-dimethyl-1H-indol-5-yl)amino)-N-(4-methoxyphenyl)benzamide (29%).

¹H NMR (400 MHz, DMSO-d₆): δ 9.97 (br s, 1H), 7.93 (br s, 1H), 7.64 (d, J=8.8 Hz, 2H), 7.40 (br s, 1H), 7.30 (d, J=8.8 Hz, 1H), 7.24 (t, J=8.0 Hz, 1H), 7.20-7.16 (m, 2H), 7.04 (br d, J=7.6 Hz, 1H), 6.92-6.85 (m, 3H), 4.08 (t, J=7.2 Hz, 2H), 3.73 (s, 3H), 2.32 (s, 3H), 2.20 (t, J=6.8 Hz, 2H), 2.14 (br s, 9H), 1.76 (quintet, J=6.8 Hz, 2H). LCMS: m/z 470.78 [M+H]⁺.

Compound 4074, N-(benzo[d][1,3]dioxol-5-yl)-3-((1-(3-(dimethylamino)propyl)-2,3-dimethyl-1H-indol-5-yl)amino)benzamide (10%).

¹H NMR (400 MHz, DMSO-d₆): δ 10.00 (br s, 1H), 7.94 (br s, 1H), 7.41-7.37 (m, 2H), 7.31 (d, J=8.4 Hz, 1H), 7.24 (t, J=8.0 Hz, 1H), 7.20-7.14 (m, 3H), 7.05 (br d, J=8.4 Hz, 1H), 6.91 (br d, J=8.8 Hz, 1H), 6.86 (d, J=8.8 Hz, 1H), 5.99 (s, 2H), 4.10 (t, J=6.8 Hz, 2H), 2.39-2.20 (m, 11H), 2.14 (s, 3H), 1.81 (quintet, J=6.8 Hz, 2H). LCMS: m/z 485.05 [M+H]⁺.

Compound 4075, 3-((1-(3-(dimethylamino)propyl)-2,3-dimethyl-1H-indol-5-yl)amino)-N-(3-fluorophenyl)benzamide (10%).

¹H NMR (400 MHz, DMSO-d₆): δ 10.29 (br s, 1H), 7.97 (br s, 1H), 7.72 (br d, J=11.6 Hz, 1H), 7.54 (d, J=8.8 Hz, 1H), 7.44-7.20 (m, 6H), 7.07 (br d, J=8.4 Hz, 1H), 6.93-6.87 (m, 2H), 4.09 (t, J=6.8 Hz, 2H), 2.32 (s, 3H), 2.27-2.08 (m, 11H), 1.77 (quintet, J=6.8 Hz, 2H). LCMS: m/z 459.0 [M+H]⁺.

Compound 4076, 3-((1-(3-(dimethylamino)propyl)-2,3-dimethyl-1H-indol-5-yl)amino)-N-(3-methoxyphenyl)benzamide (18%).

¹H NMR (300 MHz, DMSO-d₆): δ 10.08 (br s, 1H), 7.97 (br s, 1H), 7.46-7.17 (m, 8H), 7.06 (br d, J=7.8 Hz, 1H), 6.91 (br d, J=9.0 Hz, 1H), 6.66 (br d, J=7.2 Hz, 1H), 4.09 (t, J=7.2 Hz, 2H), 3.74 (s, 3H), 2.32 (s, 3H), 2.23-2.08 (m, 11H), 1.81-1.70 (m, 2H). LCMS: m/z 471.54 [M+H]⁺.

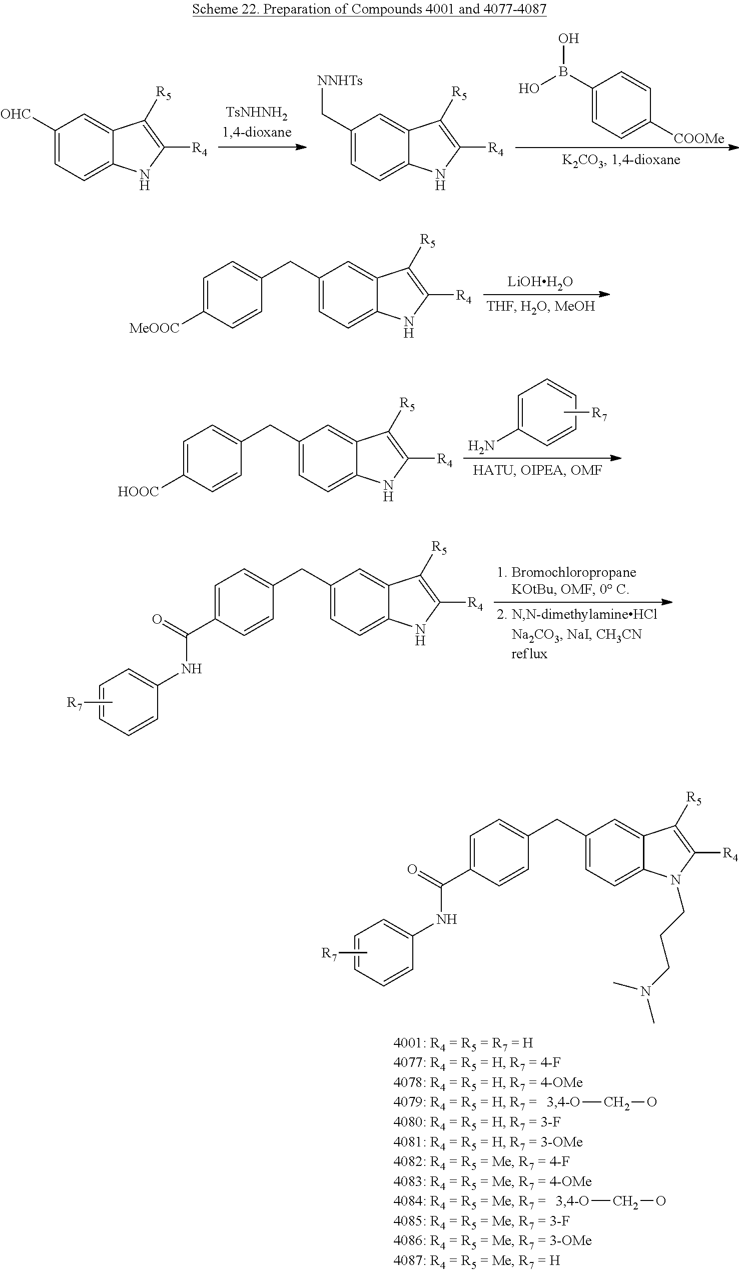

### Preparation of methyl 4-((2,3-dimethyl-1H-indol-5-yl)methyl)benzoate

Tosyl hydrazine (2.14 g, 11.5 mmol) was added to a stirred solution of 2,3-dimethyl-1H-indole-5-carbaldehyde (2.0 g, 11.5 mmol) in dry 1, 4-dioxane (50 mL) at room temperature. The temperature was raised to 80° C. and maintained for 2 hours.

To the crude 2,3-dimethyl-5-((1-tosyl-2λ²-diazanyl)methyl)-1H-indole in the reaction mass, K₂CO₃ (7.13 g, 51.6 mmol) and (4-(methoxycarbonyl)phenyl)boronic acid (6.19 g, 34.4 mmol) were added after cooling to 0° C. The reaction temperature was raised to 110° C. and maintained for 4 hours. After complete consumption of the starting material, the reaction mass was concentrated, diluted with water and extracted with ethyl acetate. The combined organic layers were washed with water and brine, dried over anhydrous Na₂SO₄ and concentrated under reduced pressure to afford the crude product. The crude compound was purified by flash column chromatography using 20-25% EtOAc in petroleum ether as an eluent to obtain a brown solid (1.5 g, 45%).

¹H NMR (300 MHz, DMSO-d₆): δ 10.55 (br s, 1H), 7.86 (d, J=8.7 Hz, 2H), 7.36 (d, J=8.4 Hz, 2H), 7.21 (br s, 1H), 7.12 (d, J=8.4 Hz, 1H), 6.83 (dd, J=8.1 Hz, 1.5 Hz, 1H), 4.05 (s, 2H), 3.82 (s, 3H), 2.27 (s, 3H), 2.11 (s, 3H). LCMS: m/z 294.41 [M+H]⁺.

Other analogues prepared by this method:

methyl 4-((1H-indol-5-yl)methyl)benzoate (40%).

¹H NMR (400 MHz, DMSO-d₆): δ 10.98 (br s, 1H), 7.86 (d, J=8.4 Hz, 2H), 7.39-7.36 (m, 3H), 7.31-7.27 (m, 2H), 6.94 (dd, J=8.4 Hz, 1.2 Hz, 1H), 6.34 (br s, 1H), 4.06 (s, 2H), 3.82 (s, 3H). LCMS: m/z 265.90 [M+H]⁺.

### Preparation of 4-((2,3-dimethyl-1H-indol-5-yl)methyl)benzoic acid

To a solution of methyl 4-((2,3-dimethyl-1H-indol-5-yl)methyl)benzoate (3.0 g, 10.2 mmol) in THF/H₂O/MeOH (10 mL, 6:2:2) was added LiOH.H₂O (1.28 g, 30.7 mmol) at 0° C. The reaction mixture was allowed to stir at room temperature for 16 hours. After complete consumption of the starting material, the reaction mass was concentrated and then partitioned between EtOAc and water. The aqueous layer was collected and acidified with saturated citric acid solution at 0° C. The precipitate thus obtained was collected by filtration and dried over vacuum to afford a brown solid (1.8 g, 63%).

¹H NMR (400 MHz, DMSO-d₆): δ 12.76 (br s, 1H), 10.54 (br s, 1H), 7.83 (d, J=8.0 Hz, 2H), 7.33 (d, J=8.4 Hz, 2H), 7.21 (br s, 1H), 7.12 (d, J=8.4 Hz, 1H), 6.83 (dd, J=8.4 Hz, 1.6 Hz, 1H), 4.04 (s, 2H), 2.30 (s, 3H), 2.14 (s, 3H). LCMS: m/z 280.39 [M+H]⁺.

Other analogues prepared by this method:

4-((1H-indol-5-yl)methyl)benzoic acid (80%).

¹H NMR (400 MHz, DMSO-d₆): δ 12.73 (br s, 1H), 10.94 (br s, 1H), 7.84 (d, J=8.8 Hz, 2H), 7.39-7.26 (m, 5H), 6.95 (dd, J=1.2 Hz, 8.4 Hz, 1H), 6.34 (br s, 1H), 4.05 (s, 2H). LCMS: m/z 252.18 [M+H]⁺.

### Preparation of 4-((2,3-dimethyl-1H-indol-5-yl)methyl)-N-(4-methoxyphenyl)benzamide

To a stirred solution of 4-((2,3-dimethyl-1H-indol-5-yl)oxy)benzoic acid (300 mg, 1.07 mmol) in DMF (5 mL), DIPEA (0.32 mL, 2.14 mmol) was added. After stirring for 10 minutes, HATU (612 mg, 1.61 mmol) was added and the reaction mixture was stirred for a further 30 minutes. The reaction mass was cooled to 0° C., 4-methoxyaniline (145 mg, 1.18 mmol) was added and the reaction mixture was stirred at room temperature overnight. After complete consumption of the starting material, the reaction mixture was poured into ice water. The precipitate thus obtained was collected by filtration and dried to give an off white solid (300 mg, 73%).

¹H NMR (300 MHz, DMSO-d₆): δ 10.54 (br s, 1H), 10.00 (br s, 1H), 7.84 (d, J=8.1 Hz, 2H), 7.64 (d, J=9.0 Hz, 2H), 7.36 (d, J=8.4 Hz, 2H), 7.22 (br s, 1H), 7.13 (d, J=8.1 Hz, 1H), 6.91 (d, J=9.0 Hz, 2H), 6.84 (dd, J=8.4 Hz, 1.5 Hz, 1H), 4.05 (br s, 2H), 3.74 (s, 3H), 2.28 (s, 3H), 2.11 (s, 3H). LCMS: m/z 385.43 [M+H]⁺.

Other analogues prepared by this method:

4-((1H-indol-5-yl)methyl)-N-phenylbenzamide (61%).

4-((1H-indol-5-yl)methyl)-N-(4-fluorophenyl)benzamide (99%).

4-((1H-indol-5-yl)methyl)-N-(4-methoxyphenyl)benzamide (98%).

4-((1H-indol-5-yl)methyl)-N-(benzo[d][1,3]dioxol-5-yl)benzamide (94%).

4-((1H-indol-5-yl)methyl)-N-(3-fluorophenyl)benzamide (94%).

4-((1H-indol-5-yl)methyl)-N-(3-methoxyphenyl)benzamide (95%).

4-((2,3-dimethyl-1H-indol-5-yl)methyl)-N-(4-fluorophenyl)benzamide (75%).

N-(benzo[d][1,3]dioxol-5-yl)-4-((2,3-dimethyl-1H-indol-5-yl)methyl)benzamide (70%).

4-((2,3-dimethyl-1H-indol-5-yl)methyl)-N-(3-fluorophenyl)benzamide (75%).

4-((2,3-dimethyl-1H-indol-5-yl)methyl)-N-(3-methoxyphenyl)benzamide (73%).

4-((2,3-dimethyl-1H-indol-5-yl)methyl)-N-phenylbenzamide (90%).

### Preparation of 4-((1-(3-chloropropyl)-2,3-dimethyl-1H-indol-5-yl)methyl)-N-(4-methoxyphenyl)benzamide

Potassium tert-butoxide (169 mg, 1.51 mmol) was added portionwise to a stirred solution of 4-((1H-indol-5-yl)methyl)-N-(4-methoxyphenyl)benzamide (300 mg, 0.78 mmol) in dry DMF (5 mL) at 0° C. The reaction mixture was allowed to warm to room temperature for 30 minutes. Bromochloropropane (0.39 mL, 3.9 mmol) was added dropwise at 0° C. and the mixture was allowed to stir at room temperature for 3 hours. After complete consumption of the starting material, ice cold water was added into the reaction mixture, which was then extracted with ethyl acetate. The organic layer was washed with brine solution, dried over anhydrous Na₂SO₄ and concentrated under reduced pressure to afford the crude product. The crude compound was purified by flash column chromatography on silica gel using EtOAc as an eluent to give a brown gummy solid (150 mg, 42%). LCMS: m/z 461.43 [M+H]⁺.

Other analogues prepared by this method:

4-((1-(3-chloropropyl)-1H-indol-5-yl)methyl)-N-phenylbenzamide (99%).

4-((1-(3-chloropropyl)-1H-indol-5-yl)methyl)-N-(4-fluorophenyl)benzamide (87%).

4-((1-(3-chloropropyl)-1H-indol-5-yl)methyl)-N-(4-methoxyphenyl)benzamide (92%).

N-(benzo[d][1,3]dioxol-5-yl)-4-((1-(3-chloropropyl)-1H-indol-5-yl)methyl)benzamide (95%).

4-((1-(3-chloropropyl)-1H-indol-5-yl)methyl)-N-(3-fluorophenyl)benzamide (89%).

4-((1-(3-chloropropyl)-1H-indol-5-yl)methyl)-N-(3-methoxyphenyl)benzamide (92%).

4-((1-(3-chloropropyl)-2,3-dimethyl-1H-indol-5-yl)methyl)-N-(4-fluorophenyl)benzamide (42%).

N-(benzo[d][1,3]dioxol-5-yl)-4-((1-(3-chloropropyl)-2,3-dimethyl-1H-indol-5-yl)methyl)benzamide (42%).

4-((1-(3-chloropropyl)-2,3-dimethyl-1H-indol-5-yl)methyl)-N-(3-fluorophenyl)benzamide (42%).

4-((1-(3-chloropropyl)-2,3-dimethyl-1H-indol-5-yl)methyl)-N-(3-methoxyphenyl)benzamide (42%).

4-((1-(3-chloropropyl)-2,3-dimethyl-1H-indol-5-yl)methyl)-N-phenylbenzamide (31%).

### Preparation of Compound 4083, 4-((1-(3-(dimethylamino)propyl)-2,3-dimethyl-1H-indol-5-yl)methyl)-N-(4-methoxyphenyl)benzamide

To a stirred solution of 4-((1-(3-chloropropyl)-2,3-dimethyl-1H-indol-5₁1)methyl)-N-(4-methoxyphenyl)benzamide (150 mg, 0.325 mmol) in acetonitrile (5 mL) at room temperature, sodium iodide (50 mg, 0.32 mmol) and sodium carbonate (138 mg, 1.30 mmol) were added, followed by N,N-dimethylamine hydrochloride (80 mg, 0.97 mmol). The reaction mixture was heated to reflux for 16 hours. After complete consumption of the starting material, the reaction mixture was cooled to room temperature, diluted with ethyl acetate, washed with water and brine solution, dried over anhydrous Na₂SO₄ and concentrated under reduced pressure to afford the crude product. The crude compound was purified by flash column chromatography using 5% MeOH-DCM as an eluent to afford an off white solid (6 mg, 4%).

¹H NMR (400 MHz, CD₃OD): δ 7.82 (d, J=8.4 Hz, 2H), 7.54 (d, J=8.8 Hz, 2H), 7.35 (d, J=8.0 Hz, 2H), 7.26 (br s, 1H), 7.22 (d, J=8.0 Hz, 1H), 6.95 (dd, J=8.0 Hz, 1.2 Hz, 1H), 6.91 (d, J=8.8 Hz, 2H), 4.16 (t, J=7.2 Hz, 2H), 4.11 (s, 2H), 3.79 (s, 3H), 2.59 (t, J=7.6 Hz, 2H), 2.42 (s, 6H), 2.36 (s, 3H), 2.19 (s, 3H), 1.97 (quintet, J=7.6 Hz, 2H). LCMS: m/z 470.54 [M+H]⁺.

Other analogues prepared by this method:

Compound 4001, 4-((1-(3-(dimethylamino)propyl)-1H-indol-5-yl)methyl)-N-phenylbenzamide (25%).

¹H NMR (300 MHz, DMSO-d₆): δ 10.14 (br s, 1H), 7.87 (d, J=8.1 Hz, 2H)), 7.75 (d, J=8.0 Hz, 1H), 7.75 (d, J=7.2 Hz, 2H), 7.43-7.31 (m, 7H), 7.11-7.03 (m, 2H), 6.39 (d, J=2.7 Hz, 1H), 4.21 (t, J=6.9 Hz, 2H), 4.08 (s, 2H), 2.87-2.68 (m, 2H), 2.57 (s, 6H), 2.10-1.97 (m, 2H). LCMS: m/z 412.5 [M+H]⁺.

Compound 4077, 4-((1-(3-(dimethylamino)propyl)-1H-indol-5-yl)methyl)-N-(4-fluorophenyl)benzamide (22%).

¹H NMR (300 MHz, DMSO-d₆): δ 10.20 (br s, 1H), 7.86 (d, J=8.1 Hz, 2H), 7.76 (dd, J=9.0 Hz, 4.8 Hz, 2H), 7.43-7.37 (m, 4H), 7.34 (d, J=3.0 Hz, 1H), 7.18 (t, J=9.0 Hz, 2H), 7.05 (dd, J=1.5 Hz, 8.7 Hz, 1H), 6.39 (d, J=2.7 Hz, 1H), 4.20 (t, J=6.9 Hz, 2H), 4.09 (s, 2H), 2.84-2.69 (m, 2H), 2.57 (br s, 6H), 2.10-1.97 (m, 2H). LCMS: m/z 430.54 [M+H]⁺.

Compound 4078, 4-((1-(3-(dimethylamino)propyl)-1H-indol-5-yl)methyl)-N-(4-methoxyphenyl)benzamide (30%).

¹H NMR (300 MHz, DMSO-d₆): δ 10.02 (br s, 1H), 7.85 (d, J=8.1 Hz, 2H), 7.64 (d, J=8.7 Hz, 2H), 7.43-7.32 (m, 5H), 7.04 (dd, J=8.7 Hz, 1.2 Hz, 1H), 6.91 (d, J=8.7 Hz, 2H), 6.38 (d, J=3.0 Hz, 1H), 4.20 (t, J=6.9 Hz, 2H), 4.07 (s, 2H), 3.73 (s, 3H), 2.75-2.61 (m, 2H), 2.56 (br s, 6H), 2.07-1.93 (m, 2H). LCMS: m/z 442.53 [M+H]⁺.

Compound 4079, N-(benzo[d][1, 3]dioxol-5-yl)-4-((1-(3-(dimethylamino)propyl)-1H-indol-5-yl)methyl)benzamide (27%).

¹H NMR (300 MHz, DMSO-d₆): δ 10.05 (br s, 1H), 7.84 (d, J=8.1 Hz, 2H), 7.45-7.32 (m, 6H), 7.16 (dd, J=8.4 Hz, 1.8 Hz, 1H), 7.04 (br d, J=8.4 Hz, 1H), 6.88 (d, J=8.4 Hz, 1H), 6.38 (d, J=3.0 Hz, 1H), 5.99 (s, 2H), 4.20 (t, J=6.9 Hz, 2H), 4.08 (s, 2H), 2.84-2.60 (m, 2H), 2.56 (br s, 6H), 2.09-1.96 (m, 2H). LCMS: m/z 456.51 [M+H]⁺.

Compound 4080, 4-((1-(3-(dimethylamino)propyl)-1H-indol-5-yl)methyl)-N-(3-fluorophenyl)benzamide (28%).

¹ H NMR (300 MHz, DMSO-d₆): δ 10.33 (br s, 1H), 7.87 (d, J=8.1 Hz, 2H), 7.74 (br d, J=11.7 Hz, 1H), 7.54 (br d, J=8.7 Hz, 1H), 7.43-7.32 (m, 6H), 7.05 (br d, J=8.7 Hz, 1H), 6.92 (br t, J=8.4 Hz, 1H), 6.38 (d, J=3.0 Hz, 1H), 4.20 (t, J=6.9 Hz, 2H), 4.09 (s, 2H), 2.78-2.62 (m, 2H), 2.51 (br s, 6H), 2.09-1.94 (m, 2H). LCMS: m/z 430.48 [M+H]⁺.

Compound 4081, 4-((1-(3-(dimethylamino)propyl)-1H-indol-5-yl)methyl)-N-(3-methoxyphenyl)benzamide (22%).

¹H NMR (300 MHz, DMSO-d₆): δ 10.10 (br s, 1H), 7.86 (d, J=8.1 Hz, 2H), 7.46-7.32 (m, 7H), 7.23 (t, J=8.1 Hz, 1H), 7.04 (dd, J=8.7 Hz, 0.9 Hz, 1H), 6.67 (dd, J=8.1 Hz, 2.7 Hz, 1H), 6.38 (d, J=3.0 Hz, 1H), 4.19 (t, J=6.9 Hz, 2H), 4.08 (s, 2H), 3.74 (s, 3H), 2.72-2.55 (m, 2H), 2.46 (br s, 6H), 2.06-1.92 (m, 2H). LCMS: m/z 442.55 [M+H]⁺.

Compound 4082, 4-((1-(3-(dimethylamino)propyl)-2,3-dimethyl-1H-indol-5-yl)methyl)-N-(4-fluorophenyl)benzamide (22%).

¹H NMR (400 MHz, CD₃OD): δ 7.83 (d, J=8.4 Hz, 2H), 7.66 (dd, J=9.2 Hz, 4.8 Hz, 2H), 7.36 (d, J=8.4 Hz, 2H), 7.25 (d, J=1.2 Hz, 1H), 7.21 (d, J=8.4 Hz, 1H), 7.08 (t, J=8.8 Hz, 2H), 6.94 (dd, J=8.4 Hz, 1.6 Hz, 1H), 4.15-4.11 (m, 4H), 2.40-2.32 (m, 5H), 2.25 (s, 6H), 2.19 (s, 3H), 1.94-1.85 (m, 2H). LCMS: m/z 458.49 [M+H]⁺.

Compound 4084, N-(benzo[d][1,3]dioxol-5-yl)-4-((1-(3-(dimethylamino)propyl)-2,3-dimethyl-1H-indol-5-yl)methyl)benzamide (5%).

¹H NMR (400 MHz, CD₃OD): δ 7.81 (d, J=8.4 Hz, 2H), 7.35 (d, J=8.4 Hz, 2H), 7.30-7.23 (m, 3H), 7.03 (dd, J=8.4 Hz, 2.0 Hz, 1H), 6.98 (dd, J=8.0 Hz, 1.2 Hz, 1H), 6.79 (d, J=8.4 Hz, 1H), 5.94 (s, 2H), 4.21 (t, J=6.8 Hz, 2H), 4.11 (s, 2H), 3.06-3.02 (m, 2H), 2.78 (s, 6H), 2.37 (s, 3H), 2.19 (s, 3H), 2.15-2.07 (m, 2H). LCMS: m/z 484.50 [M+H]⁺.

Compound 4085, 4-((1-(3-(dimethylamino)propyl)-2,3-dimethyl-1H-indol-5-yl)methyl)-N-(3-fluorophenyl)benzamide (8%).

¹H NMR (300 MHz, CD₃OD): δ 7.83 (d, J=8.7 Hz, 2H), 7.63 (dt, J=11.4 Hz, 2.4 Hz, 1H), 7.45-7.29 (m, 4H), 7.27 (br s, 1H), 7.23 (d, J=8.7 Hz, 1H), 6.95 (dd, J=8.4 Hz, 1.8 Hz, 1H), 6.85 (td, J=8.4 Hz, 2.7 Hz, 1H), 4.16 (t, J=6.9 Hz, 2H), 4.12 (s, 2H), 2.65-2.59 (m, 2H), 2.44 (s, 6H), 2.36 (s, 3H), 2.19 (s, 3H), 2.03-1.92 (m, 2H). LCMS: m/z 458.40 [M+H]⁺.

Compound 4086, 4-((1-(3-(dimethylamino)propyl)-2,3-dimethyl-1H-indol-5-yl)methyl)-N-(3-methoxyphenyl)benzamide (26%).

¹H NMR (400 MHz, CD₃OD): δ 7.82 (d, J=8.0 Hz, 2H), 7.39-7.34 (m, 3H), 7.25 (br s, 1H), 7.24-7.19 (m, 3H), 6.94 (dd, J=8.4 Hz, 1.2 Hz, 1H), 6.71 (dt, J=7.2 Hz, 2.0 Hz, 1H), 4.16-4.10 (m, 4H), 3.80 (s, 3H), 2.43 (t, J=7.6 Hz, 2H), 2.35 (s, 3H), 2.29 (s, 6H), 2.19 (s, 3H), 1.91 (quintet, J=7.2 Hz, 2H). LCMS: m/z 470.57 [M+H]⁺.

Compound 4087, 4-((1-(3-(dimethylamino)propyl)-2,3-dimethyl-1H-indol-5-yl)methyl)-N-phenylbenzamide (7%).

¹H NMR (400 MHz, CD₃OD): δ 7.84 (d, J=8.0 Hz, 2H), 7.66 (d, J=7.6 Hz, 2H), 7.38-7.30 (m, 4H), 7.26 (br s, 1H), 7.24 (d, J=8.0 Hz, 1H), 7.13 (t, J=8.0 Hz, 1H), 6.98 (dd, J=8.0 Hz, 1.2 Hz, 1H), 4.20 (t, J=6.8 Hz, 2H), 4.12 (s, 2H), 2.95 (t, J=7.2 Hz, 2H), 2.71 (s, 6H), 2.37 (s, 3H), 2.19 (s, 3H), 2.19-2.04 (m, 2H). LCMS: m/z 440.54 [M+H]⁺.

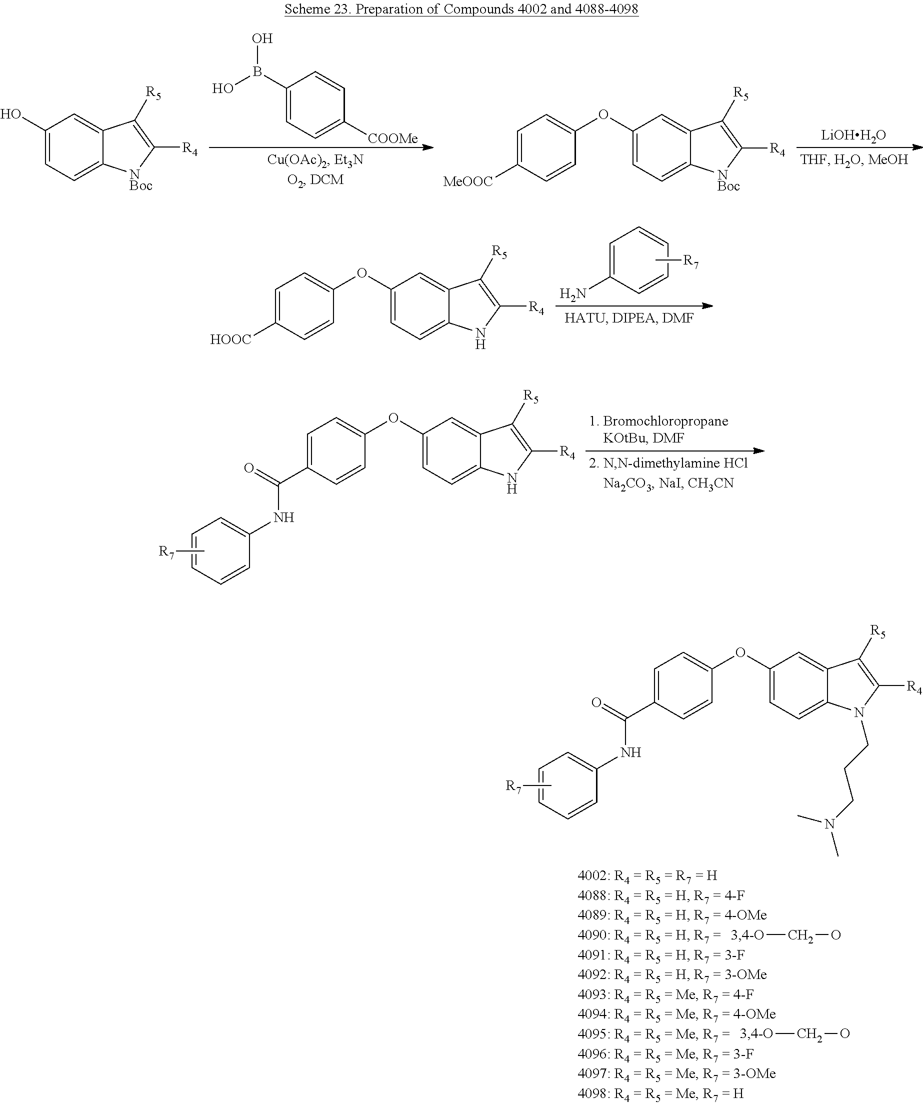

### Preparation of tert-butyl 5-(4-(methoxycarbonyl)phenoxy)-2,3-dimethyl-1H-indole-1-carboxylate

To a stirred solution of tent-butyl 5-hydroxy-2,3-dimethyl-1H-indole-1-carboxylate (5.45 g, 20.9 mmol) in DCM (55 mL) was added (4-(methoxycarbonyl)phenyl)boronic acid (11.3 g, 62.6 mmol). Cu(OAc)₂ (11.36 g, 52.12 mmol) was added, followed by Et₃N (30 mL, 208 mmol) and the system was purged with oxygen gas for 4 hours. The whole reaction mass was stirred under an oxygen atmosphere overnight. After complete consumption of the starting material, the reaction mass was filtered through a bed of Celite. The filtrate was diluted with water and extracted with DCM. The organic layer was washed with brine, dried over anhydrous Na₂SO₄ and concentrated under reduced pressure to afford the crude product. The crude product was purified on 100-200 mesh silica gel eluting with 10% EtOAc in petroleum ether to afford an off white solid (3.7 g, 45%).

¹ H NMR (400 MHz, CDCl₃): δ 8.11 (d, J=8.1 Hz, 1H), 7.98 (d, J=8.7 Hz, 2H), 7.10 (d, J=1,5 Hz, 1H), 7.00-6.94 (m, 3H), 3.87 (s, 3H), 2.54 (s, 3H), 2.14 (s, 3H), 1.64 (s, 9H). LCMS: m/z 395.9 [M+H]⁺.

Other analogues prepared by this method:

tert-butyl 5-(4-(methoxycarbonyl)phenoxy)-1H-indole-1-carboxylate (50%).

¹H NMR (400 MHz, CDCl₃): δ 8.18 (d, J=8.1 Hz, 1H), 7.97 (d, J=8.7 Hz, 2H), 7.62 (d, J=3.0 Hz, 1H), 7.25 (d, J=1.5 Hz, 1H), 7.07 (dd, J=8.1 Hz, 1.5 Hz, 1H), 6.96 (d, J=8.7 Hz, 2H), 6.53 (d, J=3.0 Hz, 1H), 3.87 (s, 3H), 1.68 (s, 9H). LCMS: m/z 368.41

[M+H]⁺.

### Preparation of 4-((2,3-dimethyl-1H-indol-5-yl)oxy)benzoic acid

To a stirred solution of tert-butyl 5-(4-(methoxycarbonyl)phenoxy)-2,3-dimethyl-1H-indole-1-carboxylate (3.7 g, 9.35 mmol) in THF (40 mL) methanol (40 mL) and water (40 mL), was added LiOH.H₂O (7.85 g, 187 mmol). The mixture was stirred at room temperature for 48 hours. After complete consumption of the starting material, THF was evaporated under reduced pressure and the reaction mass was cooled to 0° C., acidified (to pH 1) with 1 N HCl, and then extracted with ethyl acetate. The organic layers were dried over anhydrous Na₂SO₄ and concentrated under reduced pressure to afford the crude product. Trituration with n-pentane afforded a brown solid (2.3 g, 88%).

¹H NMR (300 MHz, DMSO-d₆): δ 12.65 (br s, 1H), 10.79 (br s, 1H), 7.88 (d, J=8.7 Hz, 2H), 7.27 (d, J=8.4 Hz, 1H), 7.09 (d, J=1.5 Hz, 1H), 6.94 (d, J=8.7 Hz, 2H), 6.76 (dd, J=8.4 Hz, 1.5 Hz, 1H), 2.56 (s, 3H), 2.12 (s, 3H). LCMS: m/z 282.0 [M+H]⁺.

Other analogues prepared by this method:

4-((1H-indol-5-yl)oxy)benzoic acid (90%).

¹H NMR (400 MHz, DMSO-d₆): δ 12.65 (br s, 1H), 11.10 (br s, 1H), 7.89 (d, J=8.8 Hz, 2H), 7.52-7.40 (m, 2H), 7.29 (d, J=1.6 Hz, 1H), 6.97 (d, J=8.8 Hz, 2H), 6.88 (dd, J=8.4 Hz, 1.6 Hz, 1H), 6.42 (d, J=3.2 Hz, 1H). LCMS: m/z 254.2 [M+H]⁺.

Preparation of 4-((1H-indol-5-yl)oxy)-N-(4-fluorophenyl)benzamide

To a stirred solution of 4-((1H-indol-5-yl)oxy)benzoic acid (330 mg, 1.31 mmol) in DMF (4 mL), DIPEA (1.1 mL, 6.6 mmol) was added. After 10 minutes stirring, HATU (0.75 g, 2.0 mmol) was added and the mixture was stirred for a further 30 minutes at room temperature. The reaction mass was cooled to 0° C., 4-fluoroaniline (0.20 mL, 2.0 mmol) was added and the reaction mixture was stirred at room temperature overnight. After complete consumption of the starting material, the reaction mixture was poured into ice water and extracted with EtOAc. The organic layer was washed with water and brine, dried over anhydrous Na₂SO₄ and concentrated under reduced pressure to afford the crude product. The crude product was purified on 100-200 mesh silica eluting with 30% EtOAc in petroleum ether to obtain an off white solid, 200 mg (44%).

¹H NMR (400 MHz, DMSO-d₆): δ 11.11 (br s, 1H), 10.26 (br s, 1H), 7.98 (d, J=8.8 Hz, 2H), 7.78 (dd, J=8.8 Hz, 4.8 Hz, 2H), 7.48 (d, J=8.4 Hz, 1H), 7.41 (d, J=3.2 Hz, 1H), 7.27 (d, J=1.2 Hz, 1H), 7.09 (t, J=8.8 Hz, 2H), 6.99 (d, J=8.8 Hz, 2H), 6.86 (dd, J=8.4 Hz, 1.2 Hz, 1H), 6.42 (d, J=3.2 Hz, 1H). LCMS: m/z 347.36 [M+H]⁺.

Other analogues prepared by this method:

4-((1H-indol-5-yl)oxy)-N-phenylbenzamide (72%).

4-((1H-indol-5-yl)oxy)-N-(4-methoxyphenyl)benzamide (92%).

4-((1H-indol-5-yl)oxy)-N-(benzo[d][1,3]dioxol-5-yl)benzamide (67%).

4-((1H-indol-5-yl)oxy)-N-(3-fluorophenyl)benzamide (92%).

4-((1H-indol-5-yl)oxy)-N-(3-methoxyphenyl)benzamide (92%).

4-((2,3-dimethyl-1H-indol-5-yl)oxy)-N-(4-fluorophenyl)benzamide (77%).

4-((2,3-dimethyl-1H-indol-5-yl)oxy)-N-(4-methoxyphenyl)benzamide (34%).

N-(benzo[d][1,3]dioxol-5-yl)-4-((2,3-dimethyl-1H-indol-5-yl)oxy)benzamide (92%).

4-((2,3-dimethyl-1H-indol-5-yl)oxy)-N-(3-fluorophenyl)benzamide (95%).

4-((2,3-dimethyl-1H-indol-5-yl)oxy)-N-(3-methoxyphenyl)benzamide (97%).

4-((2,3-dimethyl-1H-indol-5-yl)oxy)-N-phenylbenzamide (97%).

### Preparation of 4-((1-(3-chloropropyl)-1H-indol-5-yl)oxy)-N-(4-fluorophenyl) benzamide

Potassium tert-butoxide (199 mg, 1.77 mmol) was added portionwise to a stirred solution of 4-((1H-indol-5-yl)oxy)-N-(4-fluorophenyl)benzamide (200 mg, 0.58 mmol) in dry DMF (5 mL) at 0° C. The reaction mixture was allowed to warm to room temperature for 30 minutes. Bromochloropropane (0.12 mL, 1.2 mmol) was added drop wise at 0° C. and the mixture was allowed to stir at room temperature for 3 hours. After complete consumption of the starting material, ice cold water was added into the reaction mixture, which was then extracted with ethyl acetate. The organic layer was washed with brine solution, dried over anhydrous Na₂SO₄ and concentrated under reduced pressure to afford the crude product. The crude product was purified by flash column chromatography on silica gel using EtOAc as an eluent to afford a brown semi solid (190 mg, 78%). LCMS: m/z 423.36 [M+H]⁺.

Other analogues prepared via this method:

4-((1-(3-chloropropyl)-1H-indol-5-yl)oxy)-N-phenylbenzamide (30%).

4-((1-(3-chloropropyl)-1H-indol-5-yl)oxy)-N-(4-methoxyphenyl)benzamide (97%).

N-(benzo[d][1,3]dioxol-5-yl)-4-((1-(3-chloropropyl)-1H-indol-5-yl)oxy)benzamide (66%).

4-((1-(3-chloropropyl)-1H-indol-5-yl)oxy)-N-(3-fluorophenyl)benzamide (71%).

4-((1-(3-chloropropyl)-1H-indol-5-yl)oxy)-N-(3-methoxyphenyl)benzamide (46%).

4-((1-(3-chloropropyl)-2,3-dimethyl-1H-indol-5-yl)oxy)-N-(4-fluorophenyl)benzamide (83%).

4-((1-(3-chloropropyl)-2,3-dimethyl-1H-indol-5-yl)oxy)-N-(4-methoxyphenyl)benzamide (60%).

N-(benzo[d][1,3]dioxol-5-yl)-4-((1-(3-chloropropyl)-2,3-dimethyl-1H-indol-5-yl)oxy)benzamide (66%).

4-((1-(3-chloropropyl)-2,3-dimethyl-1H-indol-5-yl)oxy)-N-(3-fluorophenyl)benzamide (61%).

4-((1-(3-chloropropyl)-2,3-dimethyl-1H-indol-5-yl)oxy)-N-(3-methoxyphenyl)benzamide (69%).

4-((1-(3-chloropropyl)-2,3-dimethyl-1H-indol-5-yl)oxy)-N-phenylbenzamide (48%).

### Preparation of Compound 4088, 4-((1-(3-(dimethylamino)propyl)-1H-indol-5-yl)oxy)-N-(4-fluorophenyl)benzamide

To a stirred solution of 4-((1-(3-chloropropyl)-1H-indol-5-yl)oxy)-N-(4-fluorophenyl)benzamide (190 mg, 0.44 mmol) in acetonitrile (5 mL) at room temperature, sodium iodide (164 mg, 1.1 mmol) and sodium carbonate (234 mg, 2.2 mmol) were added, followed by N,N-dimethylamine hydrochloride (72 mg, 0.88 mmol). The reaction mixture was heated to 75° C. for 16 hours. After complete consumption of the starting material, the reaction mixture was cooled to room temperature, diluted with ethyl acetate, washed with water and brine solution, dried over anhydrous Na₂SO₄ and concentrated under reduced pressure to afford the crude product. The crude product was purified by flash column chromatography using 5% MeOH-DCM as an eluent to afford an off white solid (49 mg, 25%).

¹H NMR (400 MHz, DMSO-d₆): δ 10.18 (br s, 1H), 7.94 (d, J=8.8 Hz, 2H), 7.77 (dd, J=8.8 Hz, 4.8 Hz, 2H), 7.55 (d, J=8.8 Hz, 1H), 7.43 (d, J=2.8 Hz, 1H), 7.29 (d, J=2.4 Hz, 1H), 7.18 (t, J=8.8 Hz, 2H), 7.00 (d, J=8.8 Hz, 2H), 6.93 (dd, J=8.8 Hz, 2.4 Hz, 1H), 6.43 (d, J=2.8 Hz, 1H), 4.22 (t, J=6.8 Hz, 2H), 2.23-2.14 (m, 8H), 1.90 (quintet, J=6.8 Hz, 2H). LCMS: m/z 432.49 [M+H]⁺.

Other analogues prepared by this method:

Compound 4002, 4-((1-(3-(dimethylamino)propyl)-1H-indol-5-yl)oxy)-N-phenylbenzamide (71%).

¹H NMR (300 MHz, DMSO-d₆): δ 10.16 (br s, 1H), 7.95 (d, J=9.0 Hz, 2H), 7.75 (d, J=7.8 Hz, 2H), 7.57 (d, J=8.7 Hz, 1H), 7.45 (d, J=3.3 Hz, 1H), 7.37-7.30 (m, 3H), 7.08 (t, J=7.5 Hz, 1H), 7.00 (d, J=8.7 Hz, 2H), 6.95 (dd, J=8.7 Hz, 2.1 Hz, 1H), 6.46 (d, J=3.0 Hz, 1H), 4.24 (t, J=7.2 Hz, 2H), 2.41-2.33 (m, 8H), 2.08-1.97 (m, 2H). LCMS: m/z 414.50 [M+H]⁺.

Compound 4089, 4-((1-(3-(dimethylamino)propyl)-1H-indol-5-yl)oxy)-N-(4-methoxyphenyl)benzamide (11%).

¹H NMR (300 MHz, DMSO-d₆): δ 10.01 (br s, 1H), 7.94 (d, J=9.0 Hz, 2H), 7.65 (d, J=9.3 Hz, 2H), 7.56 (d, J=9.0 Hz, 1H), 7.44 (d, J=3.0 Hz, 1H), 7.29 (d, J=2.1 Hz, 1H), 7.01-6.89 (m, 5H), 6.44 (d, J=3.0 Hz, 1H), 4.23 (t, J=6.9 Hz, 2H), 3.74 (s, 3H), 2.40-2.15 (m, 8H), 2.00-1.88 (m, 2H). LCMS: m/z 444.47 [M+H]⁺.

Compound 4090, N-(benzo[d][1,3]dioxol-5-yl)-4-((1-(3-(dimethylamino)propyl)-1H-indol-5-yl)oxy)benzamide (53%).

¹H NMR (300 MHz, DMSO-d₆): δ 10.03 (br s, 1H), 7.94 (d, J=8.7 Hz, 2H), 7.59 (br d, J=9.0 Hz, 1H), 7.47 (d, J=8.7 Hz, 1H), 7.42 (d, J=1.8 Hz, 1H), 7.31 (d, J=1.8 Hz, 1H), 7.16 (dd, J=7.8 Hz, 2.1 Hz, 1H), 7.00-6.94 (m, 3H), 6.89 (d, J=8.1 Hz, 1H), 6.47 (d, J=3.0 Hz, 1H), 6.00 (s, 2H), 4.26 (t, J=6.9 Hz, 2H), 2.93-2.65 (m, 2H), 2.65 (br s, 6H), 2.16-2.04 (m, 2H). LCMS: m/z 458.49 [M+H]⁺.

Compound 4091, 4-((1-(3-(dimethylamino)propyl)-1H-indol-5-yl)oxy)-N-(3-fluorophenyl)benzamide (8%).

¹H NMR (400 MHz, DMSO-d₆): δ 10.32 (br s, 1H), 7.95 (d, J=8.4 Hz, 2H), 7.74 (br d, J=12.0 Hz, 1H), 7.57-7.53 (m, 2H), 7.44 (d, J=3.2 Hz, 1H), 7.37 (q, J=8.1 Hz, 1H), 7.30 (d, J=2.8 Hz, 1H), 7.01 (d, J=8.8 Hz, 2H), 6.95-6.88 (m, 2H), 6.44 (d, J=2.8 Hz, 1H), 4.22 (t, J=6.8 Hz, 2H), 2.32-2.14 (m, 8H), 1.96-1.88 (m, 2H). LCMS: m/z 432.46 [M+H]⁺.

Compound 4092, 4-((1-(3-(dimethylamino)propyl)-1H-indol-5-yl)oxy)-N-(3-methoxyphenyl)benzamide (10%).

¹H NMR (400 MHz, DMSO-d₆): δ 10.09 (br s, 1H), 7.94 (d, J=8.8 Hz, 2H), 7.55 (d, J=8.8 Hz, 1H), 7.46 (d, J=2.0 Hz, 1H), 7.44 (d, J=2.8 HZ, 1H), 7.35 (d, J=8.4 Hz, 1H), 7.29 (d, J=2.4 Hz, 1H), 7.23 (t, J=8.4 Hz, 1H), 7.00 (d, J=8.8 Hz, 2H), 6.93 (dd, J=8.4 Hz, 2.4 Hz, 1H), 6.67 (dd, J=8.4 Hz, 2.4 Hz, 1H), 6.43 (d, J=2.8 Hz, 1H), 4.22 (t, J=6.8 Hz, 2H), 3.75 (s, 3H), 2.20-2.14 (m, 8H), 1.93-1.86 (m, 2H). LCMS: m/z 444.50 [M+H]⁺.

Compound 4093, 4-((1-(3-(dimethylamino)propyl)-2,3-dimethyl-1H-indol-5-yl)oxy)-N-(4-fluorophenyl)benzamide (23%).

¹ H NMR (300 MHz, DMSO-d₆): δ 10.17 (br s, 1H), 7.94 (d, J=9.0 Hz, 2H), 7.77 (dd, J=9.3 Hz, 5.1 Hz, 2H), 7.44 (d, J=9.0 Hz, 1H), 7.21-7.15 (m, 3H), 6.98 (d, J=8.7 Hz, 2H), 6.85 (dd, J=8.7 Hz, 2.1 Hz, 1H), 4.15 (t, J=7.2 Hz, 2H), 2.42-2.25 (m, 11H), 2.15 (s, 3H), 1.92-1.78 (m, 2H). LCMS: m/z 460.51 [M+H]⁺.

Compound 4094, 4-((1-(3-(dimethylamino)propyl)-2,3-dimethyl-1H-indol-5-yl)oxy)-N-(4-methoxyphenyl)benzamide (24%).

¹H NMR (300 MHz, CD₃OD): δ 7.87 (d, J=8.7 Hz, 2H), 7.53 (d, J=9.3 Hz, 2H), 7.36 (d, J=8.7 Hz, 1H), 7.12 (d, J=2.4 Hz, 1H), 6.96 (d, J=9.3 Hz, 2H), 6.91 (d, J=8.7 Hz, 2H), 6.84 (dd, J=8.7 Hz, 2.1 Hz, 1H), 4.20 (t, J=6.9 Hz, 2H), 3.79 (s, 3H), 2.50-2.45 (m, 2H), 2.39 (s, 3H), 2.33 (s, 6H), 2.19 (s, 3H), 2.02-1.90 (m, 2H). LCMS: m/z 472.4 [M+H]⁺.

Compound 4095, N-(benzo[d][1,3]dioxol-5-yl)-4-((1-(3-(dimethylamino)propyl)-2,3-dimethyl-1H-indol-5-yl)oxy)benzamide (10%).

¹H NMR (400 MHz, DMSO-d₆): δ 10.02 (br s, 1H), 7.91 (d, J=8.8 Hz, 2H), 7.45 (d, J=8.8 Hz, 1H), 7.42 (d, J=1.6 Hz, 1H), 7.17-7.14 (m, 2H), 6.97 (d, J=8.8 Hz, 2H), 6.88 (d, J=8.4 Hz, 1H), 6.85 (dd, J=8.8 Hz, 2.0 Hz, 1H), 6.00 (s, 2H), 4.15 (t, J=7.2 Hz, 2H), 2.43-2.30 (m, 11H), 2.15 (s, 3H), 1.94-1.83 (m, 2H). LCMS: m/z 486.45 [M+H]⁺.

Compound 4096, 4-((1-(3-(dimethylamino)propyl)-2,3-dimethyl-1H-indol-5-yl)oxy)-N-(3-fluorophenyl)benzamide (15%).

¹ H NMR (400 MHz, DMSO-d₆): δ 10.29 (br s, 1H), 7.94 (d, J=8.8 Hz, 2H), 7.74 (br d, J=12.0 Hz, 1H), 7.54 (d, J=8.8 Hz, 1H), 7.43 (d, J=8.4 Hz, 1H), 7.37 (q, J=8.4 Hz, 1H), 7.15 (d, J=2.0 Hz, 1H), 6.99 (d, J=8.8 Hz, 2H), 6.91 (td, J=8.4 Hz, 1.8 Hz, 1H), 6.84 (dd, J=8.8 Hz, 2.4 Hz, 1H), 4.13 (t, J=7.2 Hz, 2H), 2.35 (s, 3H), 2.27-2.11 (m, 11H), 1.96-1.82 (m, 2H). LCMS: m/z 460.54 [M+H]⁺.

Compound 4097, 4-((1-(3-(dimethylamino)propyl)-2,3-dimethyl-1H-indol-5-yl)oxy)-N-(3-methoxyphenyl)benzamide (7%).

¹H NMR (300 MHz, DMSO-d₆): δ 10.08 (br s, 1H), 7.94 (d, J=8.1 Hz, 2H), 7.51-7.42 (m, 2H), 7.35 (br d, J=7.5 Hz, 1H), 7.26-7.17 (m, 2H), 6.97 (d, J=8.1 Hz, 2H), 6.88 (br d, J=8.1 Hz, 1H), 6.67 (br d, J=7.5 Hz, 1H), 4.18 (t, J=6.8 Hz, 2H), 3.75 (s, 3H), 2.85-2.64 (m, 8H), 2.37 (s, 3H), 2.15 (s, 3H), 2.08-1.91 (m, 2H). LCMS: m/z 472.55 [M+H]⁺.

Compound 4098, 4-((1-(3-(dimethylamino)propyl)-2,3-dimethyl-1H-indol-5-yl)oxy)-N-phenylbenzamide (35%).

¹H NMR (400 MHz, DMSO-d₆): δ 10.10 (br s, 1H), 7.94 (d, J=8.8 Hz, 2H), 7.75 (d, J=7.6 Hz, 2H), 7.43 (d, J=8.8 Hz, 1H), 7.34 (t, J=7.2 Hz, 2H), 7.15 (d, J=2.0 Hz, 1H), 7.08 (t, J=7.6 Hz, 1H), 6.98 (d, J=8.8 Hz, 2H), 6.84 (dd, J=8.8 Hz, 2.4 Hz, 1H), 4.13 (t, J=6.8 Hz, 2H), 2.35 (s, 3H), 2.20 (t, J=6.8 Hz, 2H), 2.14 (br s, 9H), 1.78 (quintet, J=6.8 Hz, 2H). LCMS: m/z 442.52 [M+H]⁺.

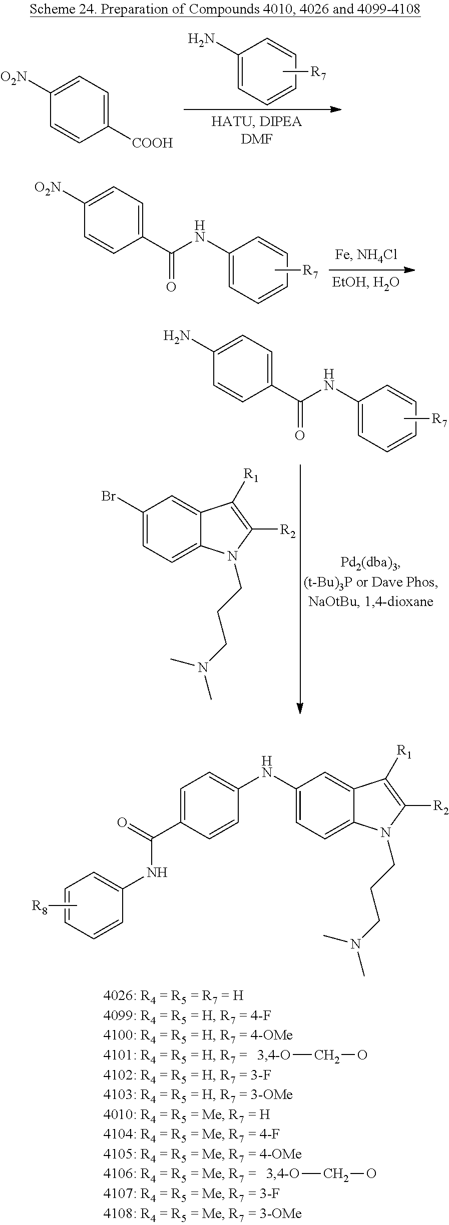

### Preparation of N-(3-fluorophenyl)-4-nitrobenzamide

To a stirred solution of 4-nitrobenzoic acid (1.0 g, 5.9 mmol) in DMF (10 mL), DIPEA (1.97 mL,) was added. After stirring for 10 minutes, HATU (4.55 g, 11.97 mmol) was added and the mixture was stirred for a further 30 minutes at room temperature. The reaction mass was cooled to 0° C., 3-fluoroaniline (665 mg, 5.9 mmol) was added and the mixture was stirred at room temperature overnight. The progress of the reaction was monitored by TLC. After complete consumption of the starting material, the reaction mixture was poured into ice water; the resulting precipitate was, filtered off and dried. The crude compound was washed with n-pentane to give a yellow solid (1.6 g, quant.).

¹H NMR (400 MHz, DMSO-d₆): δ 10.72 (br s, 1H), 8.18 (d, J=8.8 Hz, 2H), 8.09 (d, J=8.8 Hz, 2H), 7.75 (dt, J=11.2 Hz, 2.4 Hz), 7.56 (br d, J=8.8 Hz, 1H), 7.42 (q, J=8.8 Hz, 1H), 6.97 (td, J=8.8 Hz, 2.0 Hz, 1H). LCMS: m/z 260.89 [M+H]⁺.

Other analogues prepared by this method:

4-nitro-N-phenylbenzamide (42%).

4-nitro-N-(4-fluorophenyl)benzamide (87%).

4-nitro-N-(4-methoxyphenyl)benzamide (67%).

N-(benzo[d][1,3]dioxol-5-yl)-4-nitrobenzamide (99%).

4-nitro-N-(3-methoxyphenyl)benzamide (7%).

### Preparation of 4-amino-N-(3-fluorophenyl)benzamide

To a stirred solution of compound N-(3-fluorophenyl)-4-nitrobenzamide (1.3 g, 5.0 mmol) in EtOH (20 mL), H₂O (20 mL), Fe powder (1.39 g, 25 mmol) and NH₄Cl (0.53 g, 10 mmol) were added at room temperature. The reaction mixture was heated to 70° C. for 2 hours. The reaction mixture was filtered off and washed with ethyl acetate. The mother liquor was concentrated to remove EtOH. The crude compound was dissolved in ethyl acetate (100 mL) and washed with water, followed by brine solution. 10, The solution was concentrated under reduced pressure to to obtain a yellow solid. (1.0 g, 91%).

¹H NMR (400 MHz, DMSO-d₆): δ 10.90 (br s, 1H), 7.77-7.68 (m, 3H), 7.54 (d, J=8.8 Hz, 1H), 7.37 (q, J=8.8 Hz, 1H), 6.84 (td, J=8.8 Hz, 2.0 Hz, 1H), 6.59 (d, J=8.8 Hz, 2H), 5.78 (br s, 2H). LCMS: m/z 230.97 [M+H]⁺.

Other analogues prepared by this method:

4-amino-N-phenylbenzamide (91%).

4-amino-N-(4-fluorophenyl)benzamide (100%).

4-amino-N-(4-methoxyphenyl)benzamide (61%).

4-amino-N-(benzo[d][1,3]dioxol-5-yl)benzamide (73%).

4-amino-N-(3-methoxyphenyl)benzamide (90%).

### Preparation of Compound 4099, 3-((1-(3-(dimethylamino)propyl)-1H-indol-5-yl)amino)-N-(4-fluorophenyl)benzamide

To a stirred solution of 3-(5-bromo-1H-indol-1-yl)-N,N-dimethylpropan-1-amine (100 mg, 0.356 mmol) in 1,4-dioxane (5 mL) was added NaOtBu (86 mg, 0.89 mmol), Pd₂(dba)₃ (49 mg, 0.054 mmol), (t-Bu)₃P (22 mg, 0.11 mmol) and 4-amino-N-(4-fluorophenyl)benzamide (125 mg, 0.543 mmol) at room temperature. The reaction mixture was heated to 100° C. for 16 hours. After complete consumption of the starting material, the reaction mixture was filtered and extracted with ethyl acetate. The organic layer was washed with brine solution, dried over anhydrous Na₂SO₄ and concentrated under reduced pressure to afford the crude product. The crude product was purified by flash column chromatography, eluting with 5% MeOH in DCM to afford a brown solid (15 mg, 9%).

¹H NMR (400 MHz, DMSO-d₆): δ 9.90 (br s, 1H), 8.35 (br s, 1H), 7.81-7.72 (m, 4H), 7.45 (br d, J=8.4 Hz, 1H), 7.38-7.32 (m, 2H), 7.15 (t, J=8.8 Hz, 2H), 7.00 (br d, J=8.0 Hz, 1H), 6.93 (d, J=8.0 Hz, 2H), 6.38 (br s, 1H), 4.18 (t, J=6.8 Hz, 2H), 2.18-2.09 (m, 8H), 1.92-1.81 (m, 2H). LCMS: m/z 431.4 [M+H]⁺.

Other analogues prepared by this method:

Compound 4026, 4-((1-(3-(dimethylamino)propyl)-1H-indol-5-yl)amino)-N-phenylbenzamide (51%).

¹H NMR (400 MHz, DMSO-d₆): δ 9.84 (br s, 1H), 8.36 (br s, 1H), 7.81 (d, J=8.8 Hz, 2H), 7.75 (d, J=7.6 Hz, 2H), 7.45 (d, J=8.8 Hz, 1H), 7.36-7.29 (m, 4H), 7.05 (t, J=7.2 Hz, 1H), 7.00 (dd, J=8.8 Hz, 2.0 Hz, 1H), 6.94 (d, J=8.8 Hz, 2H), 6.37 (d, J=2.8 Hz, 1H), 4.24 (t, J=6.8 Hz, 2H), 2.30-2.26 (m, 8H), 2.06-1.99 (m, 2H). LCMS: m/z 413.21 [M+H]⁺.

Compound 4100, 4-((1-(3-(dimethylamino)propyl)-1H-indol-5-yl)amino)-N-(4-methoxyphenyl)benzamide (10%).

¹H NMR (400 MHz, DMSO-d₆): δ 10.00 (br s, 1H), 8.01 (br s, 1H), 7.64 (d, J=9.2 Hz, 2H), 7.42 (d, J=8.4 Hz, 2H), 7.33-7.19 (m, 4H), 7.08 (br d, J=8.0 Hz, 1H), 6.98 (d, J=8.8 Hz, 1H), 6.90 (d, J=8.8 Hz, 2H), 6.34 (d, J=2.8 Hz, 1H), 4.16 (t, J=6.8 Hz, 2H), 3.73 (s, 3H), 2.18-2.10 (m, 8H), 1.92-1.83 (m, 2H). LCMS: m/z 443.06 [M+H]⁺.

Compound 4101, N-(benzo[d][1,3]dioxol-5-yl)-4-((1-(3-(dimethylamino)propyl)-1H-indol-5-yl)amino)benzamide (21%).

¹H NMR (400 MHz, DMSO-d₆): δ 9.75 (br s, 1H), 8.33 (br s, 1H), 7.78 (d, J=8.4 Hz, 2H), 7.46-7.40 (m, 2H), 7.37-7.29 (m, 2H), 7.16 (br d, J=6.8 Hz, 1H), 6.99 (br d, J=8.0 Hz, 1H), 6.93 (d, J=8.8 Hz, 2H), 6.86 (d, J=8.4 Hz, 1H), 6.37 (d, J=2.4 Hz, 1H), 5.98 (s, 2H), 4.18 (t, J=6.8 Hz, 2H), 2.18-2.07 (m, 8H), 1.92-1.83 (m, 2H). LCMS: m/z 457.1 [M+H]⁺.

Compound 4102, 4-((1-(3-(dimethylamino)propyl)-1H-indol-5-yl)amino)-N-(3-fluorophenyl)benzamide (20%).

¹H NMR (400 MHz, DMSO-d₆): δ 10.02 (br s, 1H), 8.40 (br s, 1H), 7.80 (d, J=8.8 Hz, 2H), 7.75 (dt, J=9.0 Hz, 2.4 Hz, 1H), 7.54 (br d, J=8.4 Hz, 1H), 7.46 (d, J=8.8 Hz, 1H), 7.37-7.31 (m, 3H), 7.00 (dd, J=8.8 Hz, 2.0 Hz, 1H), 6.94 (d, J=8.4 Hz, 2H), 6.87 (td, J=8.0 Hz, 2.0 Hz, 1H), 6.38 (d, J=2.1 Hz, 1H), 4.18 (t, J=6.8 Hz, 2H), 2.18-2.10 (m, 8H), 1.88 (quintet, J=6.8 Hz, 2H). LCMS: m/z 431.25 [M+H]⁺.

Compound 4103, 4-((1-(3-(dimethylamino)propyl)-1H-indol-5-yl)amino)-N-(3-methoxyphenyl)benzamide (51%).

¹H NMR (400 MHz, DMSO-d₆): δ 9.79 (br s, 1H), 8.34 (br s, 1H), 7.80 (d, J=8.8 Hz, 2H), 7.48-7.44 (m, 2H), 7.36-7.33 (m, 3H), 7.21 (t, J=8.0 Hz, 1H), 7.00 (dd, J=8.4 Hz, 2.0 Hz, 1H), 6.93 (d, J=8.8 Hz, 2H), 6.63 (dd, J=8.0 Hz, 2.0 Hz, 1H), 6.37 (d, J=2.8 Hz, 1 H), 4.18 (t, J=6.8 Hz, 2H), 3.74 (s, 3H), 2.21-2.12 (m, 8H), 1.89 (quintet, J=6.8 Hz, 2H). LCMS: m/z 443.0 [M+H]⁺.

### Preparation of Compound 4104, 4-((1-(3-(dimethylamino)propyl)-2,3-dimethyl-1H-indol-5-yl)amino)-N-(4-fluorophenyl)benzamide

To a stirred solution of 3-(5-bromo-2,3-dimethyl-1H-indol-1-yl)-N,N-dimethylpropan-1-amine (100 mg, 0.32 mmol) in 1,4-dioxane (10 mL) was added NaOtBu (92 mg, 0.90 mmol), Pd₂(dba)₃ (49 mg, 0.054 mmol), Dave Phos (35 mg, 0.01 mmol) and 4-amino-N-(4-fluorophenyl)benzamide (135 mg, 0.59 mmol) at room temperature. The reaction mixture was heated to 90° C. for 16 hours. After complete consumption of the starting material, the reaction mixture was filtered and extracted with ethyl acetate. The organic layer was washed with brine solution, dried over anhydrous Na₂SO₄ and concentrated under reduced pressure to afford the crude product. The crude product was purified by silica column chromatography in 100-200 mesh silica, eluting with 10%-100% ethyl acetate in petroleum ether to afford an off white solid (70 mg, 16%).

¹H NMR (300 MHz, DMSO-d₆): δ 9.98 (br s, 1H), 8.35 (br s, 1H), 7.82-7.74 (m, 4H), 7.37 (d, J=8.7 Hz, 1H), 7.21 (d, J=1.8 Hz, 1H), 7.15 (t, J=9.3 Hz, 2H), 6.95-6.89 (m, 3H), 4.13 (t, J=6.9 Hz, 2H), 2.73-2.60 (m, 2H), 2.44 (br s, 6H), 2.34 (s, 3H), 2.15 (s, 3H), 1.96-1.80 (m, 2H). LCMS: m/z 458.24 [M+H]⁺.

Other analogues prepared by this method:

Compound 4010, 4-((1-(3-(dimethylamino)propyl)-2,3-dimethyl-1H-indol-5-yl)amino)-N-phenylbenzamide (13%).

¹H NMR (400 MHz, CD₃OD): δ 7.79 (d, J=8.8 Hz, 2H), 7.67 (d, J=7.6 Hz, 2H), 7.37-7.27 (m, 4H), 7.12 (t, J=7.6 Hz, 1H), 7.00 (dd, J=8.4 Hz, 2.0 Hz, 1H), 6.95 (d, J=8.8 Hz, 2H), 4.18 (t, J=7.2 Hz, 2H), 2.56 (t, J=7.8 Hz, 2H), 2.39 (s, 9H), 2.21 (s, 3H), 2.02-1.95 (m, 2H). LCMS: m/z 441.1 [M+H]⁺.

Compound 4105, 4-((1-(3-(dimethylamino)propyl)-2,3-dimethyl-1H-indol-5-yl)amino)-N-(4-methoxyphenyl)benzamide (37%).

¹H NMR (400 MHz, CDCl₃): δ 7.71 (d, J=8.4 Hz, 2H), 7.58-7.50 (m, 3H), 7.33 (d, J=1.6 Hz, 1H), 7.00 (br d, J=8.4 Hz, 1H), 6.91-6.85 (m, 4H), 4.16 (t, J=7.2 Hz, 2H), 3.81 (s, 3H), 2.45-2.30 (m, 11H), 2.22 (s, 3H), 2.08-1.90 (m, 2H). LCMS: m/z 471.6 [M+H]⁺.

Compound 4106, N-(benzo[d][1,3]dioxol-5-yl)-4-((1-(3-(dimethylamino)propyl)-2,3-dimethyl-1H-indol-5-yl)amino)benzamide (31%).

1_{H NMR} (400 MHz, DMSO-d₆): δ 9.74 (br s, 1H), 8.31 (br s, 1H), 7.77 (d, J=8.8 Hz, 2H), 7.43 (d, J=2.4 Hz, 1H), 7.34 (d, J=8.4 Hz, 1H), 7.20 (d, J=2.0 Hz, 1H), 7.16 (dd, J=8.4 Hz, 2.0 Hz, 1H), 6.93-6.89 (m, 3H), 6.86 (d, J=8.4 Hz, 1H), 5.98 (s, 2H), 4.11 (t, J=6.8 Hz, 2H), 2.38-2.30 (m, 5H), 2.24 (br s, 6H), 2.15 (s, 3H), 1.87-1.74 (m, 2H). LCMS: m/z 485.0 [M+H]⁺.

Compound 4107, 4-((1-(3-(dimethylamino)propyl)-2,3-dimethyl-1H-indol-5-yl)amino)-N-(3-fluorophenyl)benzamide (14%).

¹H NMR (400 MHz, CDCl₃): δ 7.72-7.68 (m, 3H), 7.33 (d, J=2.0 Hz, 1H), 7.31-7.22 (m, 3H), 7.00 (dd, J=8.8 Hz, 2.4 Hz, 1H), 6.88 (d, J=8.4 Hz, 2H), 6.83-6.78 (m, 1H), 4.16 (t, J=6.8 Hz, 2H), 2.49-2.29 (m, 11H), 2.22 (s, 3H), 2.07-1.92 (m, 2H). LCMS: m/z 459.1 [M+H]⁺.

Compound 4108, 4-((1-(3-(dimethylamino)propyl)-2,3-dimethyl-1H-indol-5-yl)amino)-N-(3-methoxyphenyl)benzamide (14%).

¹H NMR (300 MHz, DMSO-d₆): δ 9.80 (br s, 1H), 8.34 (br s, 1H), 7.80 (d, J=8.4 Hz, 2H), 7.47 (br s, 1H), 7.38-7.33 (m, 2H), 7.23-7.17 (m, 2H), 6.94-6.88 (m, 3H), 6.63 (dd, J=8.1 Hz, 1.8 Hz, 1H), 4.12 (t, J=6.9 Hz, 2H), 3.74 (s, 3H), 2.40-2.22 (m, 11H), 2.15 (s, 3H), 1.89-1.78 (m, 2H). LCMS: m/z 471.50 [M+H]⁺.

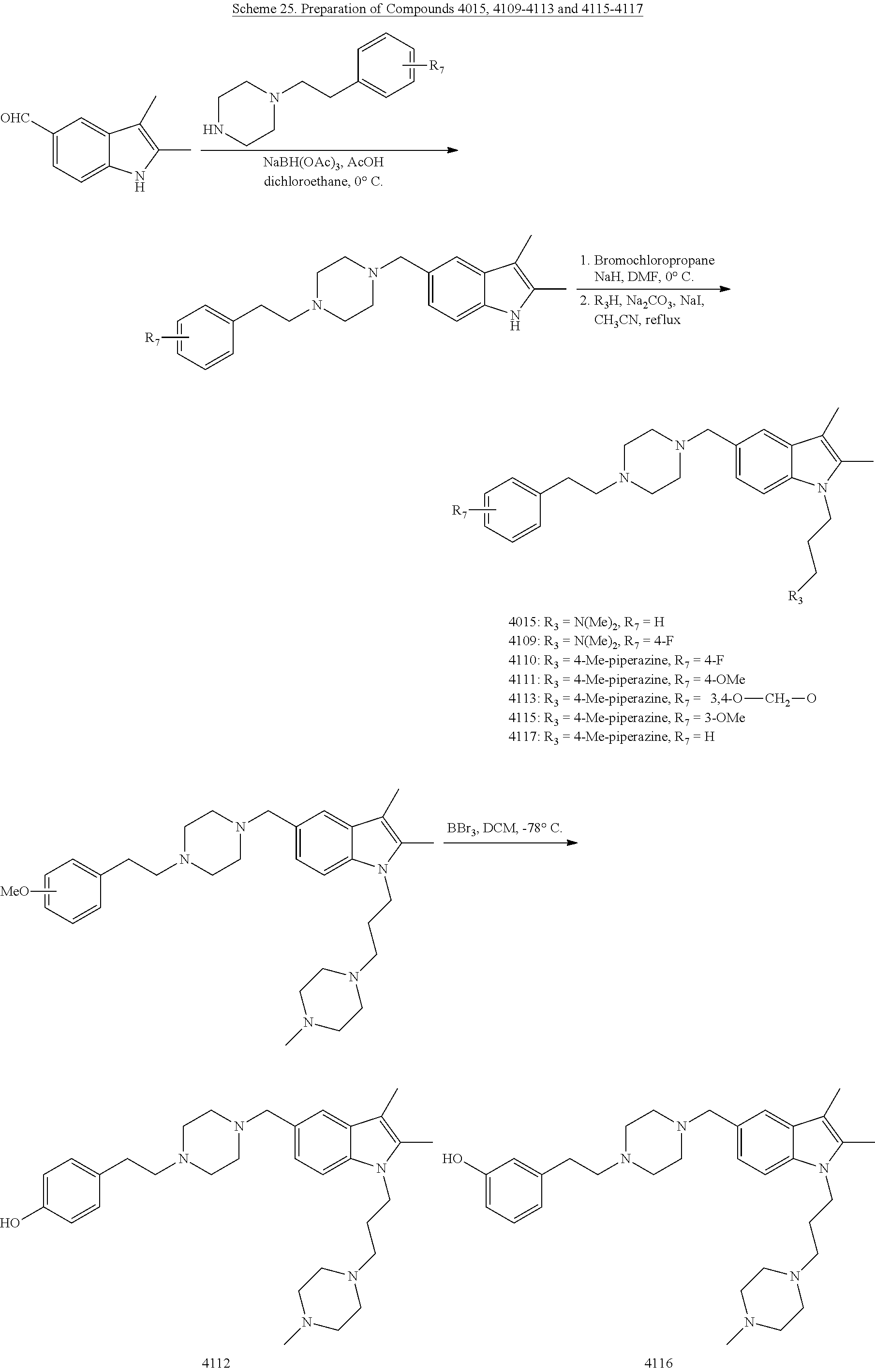

### Preparation of 5-((4-(4-methoxyphenethyl)piperazin-1-yl)methyl)-2,3-dimethyl-1H-indole

To a stirred solution of 2,3-dimethyl-1H-indole-5-carbaldehyde (900 mg, 5.20 mmol) in dichloroethane (5 mL) was added 1-(4-methoxyphenethyl)piperazine (1.14 g, 5.20 mmol), AcOH (1 mL) and STABH (1.21 g, 5.72 mmol) at 0° C., then stirred for 10 minutes. The reaction temperature was raised to room temperature and maintained for 12 hours. After complete consumption of the starting material, the reaction mass was concentrated, diluted with water and extracted with ethyl acetate. The combined organic layers were washed with water and brine, dried over anhydrous Na₂SO₄ and concentrated under reduced pressure to afford the crude product. The crude compound was purified by flash column using 2-5% MeOH in DCM as an eluent to obtain a brown gummy solid (0.77 g, 39%).

¹H NMR (300 MHz, DMSO-d₆): δ 10.76 (br s, 1H), 7.22 (br s, 1H), 7.17-7.04 (m, 3H), 6.92 (br d, J=7.8 Hz, 1H), 6.82 (d, J=8.4 Hz, 2H), 3.70 (s, 3H), 3.47 (br s, 2H), 2.66-2.60 (m, 2H), 2.52-2.31 (m, 10H), 2.28 (s, 3H), 2.13 (s, 3H). LCMS: m/z 378.53 [M+H]⁺.

Other analogues prepared by this method:

5-((4-(4-fluorophenethyl)piperazin-1-yl)methyl)-2,3-dimethyl-1H-indole (32%).

5-((4-(2-(benzo[d][1,3]dioxol-5-yl)ethyl)piperazin-1-yl)methyl)-2,3-dimethyl-1H-indole (23%).

5-((4-(3-methoxyphenethyl)piperazin-1-yl)methyl)-2,3-dimethyl-1H-indole (36%).

2,3-dimethyl-5-((4-phenethylpiperazin-1-yl)methyl)-1H-indole (50%).

### Preparation of 1-(3-chloropropyl)-5-((4-(4-methoxyphenethyl)piperazin-1-yl)methyl)-2,3-dimethyl-1H-indole

NaH (163.1 mg, 4.07 mmol) was added portionwise to a stirred solution of 5-((4-(4-methoxyphenethyl)piperazin-1-yl)methyl)-2,3-dimethyl-1H-indole (770 mg, 2.03 mmol) in DMF (5 mL) at 0° C. The mixture was allowed to warm to room temperature for 30 minutes. Bromochloropropane (0.45 mL, 4.07 mmol) was added dropwise at 0° C. and the resulting mixture was stirred at room temperature for 3 hours. After complete consumption of the starting material, ice cold water was added and the mixture was extracted with ethyl acetate. The organic layer was washed with brine solution, dried over anhydrous Na₂SO₄ and concentrated under reduced pressure to afford the crude product. The crude compound was purified by flash column chromatography using 2-5% MeOH in DCM as an eluent to obtain a brown gummy solid (600 mg, 65%).

¹H NMR (300 MHz, DMSO-d₆): δ 7.32-7.25 (m, 2H), 7.11 (d, J=9.0 Hz, 2H), 7.00 (br d, J=8.1 Hz, 1H), 6.82 (d, J=8.7 Hz, 2H), 4.19 (t, J=7.2 Hz, 2H), 3.71 (s, 3H), 3.64 (t, J=6.3 Hz, 2H) 3.47 (br s, 2H), 2.66-2.57 (m, 2H), 2.55-2.28 (m, 13H), 2.16 (s, 3H), 2.11-2.04 (m, 2H). LCMS: m/z 454.50 [M+H]⁺.

Other analogues prepared by this method:

1-(3-chloropropyl)-5-((4-(4-fluorophenethyl)piperazin-1-yl)methyl)-2,3-dimethyl-1 H-indole (66%).

5-((4-(2-(benzo[d][1,3]dioxol-5-yl)ethyl)piperazin-1-yl)methyl)-1-(3-chloropropyl)-2,3-dimethyl-1H-indole (58%).

1-(3-chloropropyl)-5-((4-(3-methoxyphenethyl)piperazin-1-yl)methyl)-2,3-dimethyl-1H-indole (18%).

1-(3-chloropropyl)-5-((4-phenethylpiperazin-1-yl)methyl)-2,3-dimethyl-1H-indole (46%).

### Preparation of Compound 4109, 3-(5-((4-(4-fluorophenethyl)piperazin-1-yl)methyl)-2,3-dimethyl-1H-indol-1-yl)-N,N-dimethylpropan-1-amine

To a stirred solution of the intermediate 1-(3-chloropropyl)-5-((4-(4-fluorophenethyl)piperazin-1-yl)methyl)-2,3-dimethyl-1H-indole (210 mg, 0.475 mmol) in acetonitrile (10 mL) at room temperature, sodium iodide (142 mg, 0.95 mmol) and sodium carbonate (151 mg, 1.43 mmol) were added, followed by N,N-dimethylamine hydrochloride (97 mg, 1.19 mmol). The reaction mixture was heated at reflux for 12 hours. After complete consumption of the starting material, the reaction mixture was cooled to room temperature, diluted with EtOAc, washed with water and brine solution, dried over anhydrous Na₂SO₄ and concentrated under reduced pressure to afford the crude product. The crude product was purified by flash column chromatography using 7% MeOH-DCM as an eluent to afford the target compound as a brown gummy solid (15 mg, 7%).

¹H NMR (300 MHz, CD₃OD): δ 7.37 (br s, 1H), 7.28-7.15 (m, 3H), 7.09-6.93 (m, 3H), 4.14 (t, J=6.6 Hz, 2H), 3.64 (s, 2H), 2.81-2.45 (m, 12H), 2.39-2.30 (m, 5H), 2.27-2.19 (m, 9H), 1.95-1.85 (m, 2H). LCMS: m/z 451.56 [M+H]⁺.

Other analogues prepared by this method:

Compound 4015, 3-(2,3-dimethyl-5-((4-phenethylpiperazin-1-yl)methyl)-1H-indol-1-yl)-N,N-dimethylpropan-1-amine (16%).

¹H NMR (400 MHz, CD₃OD): δ 7.37 (d, J=0.8 Hz, 1H), 7.27-7.22 (m, 3H), 7.21-7.13 (m, 3H), 7.07 (dd, J=8.0 Hz, 1.5 Hz, 1H), 4.14 (t, J=7.2 Hz, 2H), 3.65 (s, 2H), 2.81-2.77 (m, 2H), 2.69-2.48 (m, 10H), 2.37-2.33 (m, 5H), 2.24 (s, 6H), 2.22 (s, 3H), 1.90 (quintet, J=7.2 Hz, 2H). LCMS: m/z 433.52 [M+H]⁺.

Compound 4110, 5-((4-(4-fluorophenethyl)piperazin-1-yl)methyl)-2,3-dimethyl-1-(3-(4-methylpiperazin-1-yl)propyl)-1H-indole (19%).

¹H NMR (300 MHz, CD₃OD): δ 7.36 (br s, 1H), 7.27 (d, J=8.4 Hz, 1H), 7.20 (dd, J=8.7 Hz, 5.4 Hz, 2H), 7.05 (dd, J=8.4 Hz, 1.5 Hz, 1H), 6.98 (t, J=8.7 Hz, 2H), 4.16 (t, J=6.9 Hz, 2H), 3.64 (s, 2H), 2.81-2.74 (m, 2H), 2.72-2.28 (m, 23H), 2.27 (s, 3H), 2.22 (s, 3H), 1.90 (quintet, J=6.9 Hz, 2H). LCMS: m/z 506.58 [M+H]⁺.

Compound 4111, 5-((4-(4-methoxyphenethyl)piperazin-1-yl)methyl)-2,3-dimethyl-1-(3-(4-methylpiperazin-1-yl)propyl)-1H-indole (34%).

¹H NMR (300 MHz, CD₃OD): δ 7.37 (br s, 1H), 7.28 (d, J=8.4 Hz, 1H), 7.13-7.02 (m, 3H), 6.82 (d, J=8.4 Hz, 2H), 4.16 (t, J=6.9 Hz, 2H), 3.75 (s, 3H), 3.67 (s, 2H), 2.81-2.74 (m, 2H), 2.79-2.26 (m, 26H), 2.22 (s, 3H), 1.97-1.82 (m, 2H). LCMS: m/z 518.59 [M+H]⁺.

Compound 4113, 5-((4-(2-(benzo[d][1,3]dioxol-5-yl)ethyl)piperazin-1-yl)methyl)-2,3-dimethyl-1-(3-(4-methylpiperazin-1-yl)propyl)-1H-indole (32%).

¹H NMR (300 MHz, CD₃OD): δ 7.37 (br s, 1H), 7.27 (d, J=8.1 Hz, 1H), 7.06 (dd, J=8.4 Hz, 1.5 Hz, 1H), 6.72-6.28 (m, 3H), 5.88 (s, 2H), 4.16 (t, J=6.9 Hz, 2H), 3.65 (s, 2H), 2.74-2.29 (m, 25H), 2.28 (s, 3H), 2.22 (s, 3H), 1.96-1.87 (m, 2H). LCMS: m/z 532.55 [M+H]⁺.

Compound 4115, 5-((4-(3-methoxyphenethyl)piperazin-1-yl)methyl)-2,3-dimethyl-1-(3-(4-methylpiperazin-1-yl)propyl)-1H-indole (16%).

¹H NMR (400 MHz, CD₃OD): δ 7.37 (br s, 1H), 7.27 (d, J=8.0 Hz, 1H), 7.16 (t, J=8.0 Hz, 1H), 7.06 (br d, J=8.0 Hz, 1H), 6.78-6.72 (m, 3H), 4.16 (t, J=6.8 Hz, 2H), 3.76 (s, 3H), 3.65 (s, 2H), 2.82-2.29 (m, 25H), 2.28 (s, 3H), 2.22 (s, 3H), 1.94-1.87 (m, 2H). LCMS: m/z 518.56 [M+H]⁺.

Compound 4117, 2,3-dimethyl-1-(3-(4-methylpiperazin-1-yl)propyl)-5-((4-phenethylpiperazin-1-yl)methyl)-1H-indole (7%).

¹H NMR (400 MHz, CD₃OD): δ 7.37 (br s, 1H), 7.29-7.11 (m, 6H), 7.06 (br d, J=8.4 Hz, 1H), 4.16 (t, J=6.6 Hz, 2H), 3.65 (s, 2H), 2.74-2.24 (m, 28H), 2.22 (s, 3H), 1.96-1.83 (m, 2H). LCMS: m/z 488.59 [M+H]⁺.

### Preparation of Compound 4112, 4-(2-(4-((2,3-dimethyl-1-(3-(4-methylpiperazin-1-yl)propyl)-1H-indol-5-yl)methyl)piperazin-1-yl)ethyl)phenol

To a stirred solution of Compound 4111 (100 mg, 0.19 mmol) in dichloromethane (5 mL) at −78° C. was added boron tribromide (1 M in DCM, 1 mL). The mixture was stirred at this temperature for 10 minutes, then allowed to warm to room temperature and stirred for 2 hours. The reaction mixture was quenched with saturated NaHCO₃, then extracted with 10% Methanol in DCM, dried over Na₂SO₄. The organic layer was concentrated under reduced pressure and the crude compound was purified by preparative-HPLC to obtain a brown solid (15 mg, 11%).

¹H NMR (400 MHz, CD₃OD): δ 7.42 (br s, 1H), 7.31 (d, J=8.0 Hz, 1H), 7.09 (dd, J=8.4 Hz, 1.6 Hz, 1H), 7.02 (d, J=8.8 Hz, 2H), 6.69 (d, J=8.8 Hz, 2H), 4.18 (t, J=6.8 Hz, 2H), 3.84 (s, 2H), 2.91-2.42 (m, 20H), 2.41-2.32 (m, 8H), 2.23 (s, 3H), 1.91 (quintet, J=6.8 Hz, 2H). LCMS: m/z 504.56 [M+H]⁺.

Other analogues prepared by this method:

Compound 4116, 3-(2-(4-((2,3-dimethyl-1-(3-(4-methylpiperazin-1-yl)propyl)-1H-indol-5-yl)methyl)piperazin-1-yl)ethyl)phenol, (32%).

¹H NMR (400 MHz, CD₃OD): δ 7.41 (br s, 1H), 7.31 (d, J=8.4 Hz, 1H), 7.10-7.05 (m, 2H), 6.66 (br d, J=8.0 Hz, 1H), 6.68-6.59 (m, 2H), 4.18 (t, J=6.8 Hz, 2H), 3.80 (s, 2H), 2.90-2.41 (m, 20H), 2.38-2.31 (m, 8H), 2.23 (s, 3H), 1.91 (quintet, J=6.8 Hz, 2H). LCMS: m/z 504.56 [M+H]⁺.

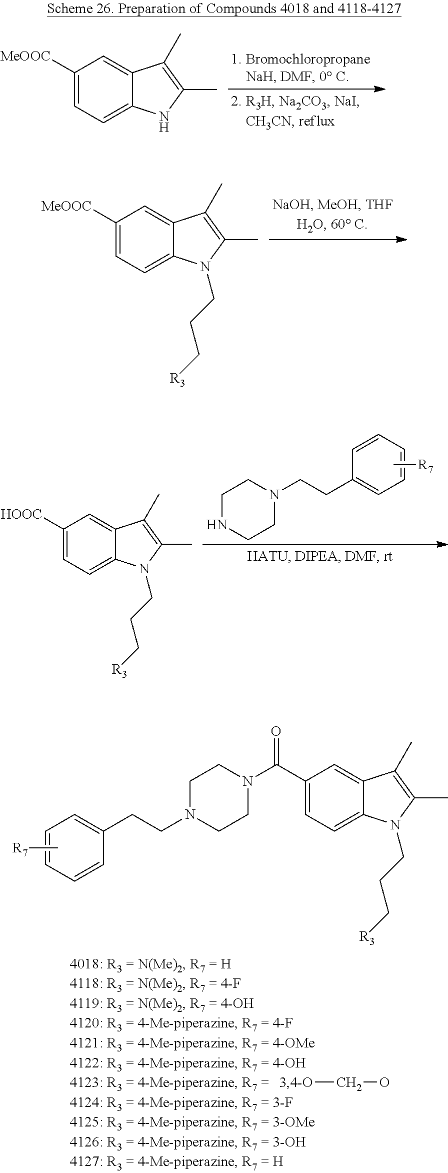

### Preparation of methyl 1-(3-chloropropyl)-2,3-dimethyl-1H-indole-5-carboxylate

NaH (394 mg, 9.70 mmol) was added portionwise to a stirred solution of methyl 2,3-dimethyl-1H-indole-5-carboxylate (1.0 g, 4.97 mmol) in DMF (10 mL) at 0° C. The reaction mixture was allowed to warm to room temperature for 30 minutes. Bromochloropropane (2.5 mL, 156 mmol) was added dropwise at 0° C. and the mixture was allowed to stir at room temperature for 3 hours. After complete consumption of the starting material, ice cold water was added into the reaction mixture, which was then extracted with ethyl acetate. The organic layer was washed with brine solution, dried over anhydrous Na₂SO₄ and concentrated under reduced pressure to afford the crude product. The crude product was purified by flash column chromatography on silica gel, using 10-15% EtOAc in petroleum ether as an eluent, to afford an off-white solid (1.0 g, 76%).

¹H NMR (400 MHz, CDCl₃): δ 8.24 (d, J=2.0 Hz, 1H), 7.85 (dd, J=8.8 Hz, 2.0 Hz, 1H), 7.28 (d, J=8.8 Hz, 1H), 4.24 (t, J=7.2 Hz, 2H), 3.93 (s, 3H), 3.52 (t, J=7.2 Hz, 2H), 2.39 (s, 3H), 2.26 (s, 3H), 2.21 (quintet, J=7.2 Hz, 2H). LCMS: m/z 279.76 [M+H]⁺.

### Preparation of methyl 2,3-dimethyl-1-(3-(4-methylpiperazin-1-yl)propyl)-1H-indole-5-carboxylate

To a stirred solution of methyl 1-(3-chloropropyl)-2,3-dimethyl-1H-indole-5-carboxylate (300 mg, 1.06 mmol) in acetonitrile (40 mL) at room temperature, sodium iodide (398 mg, 2.00 mmol) and sodium carbonate (338 mg, 3.00 mmol) were added, followed by N-methylpiperazine (0.3 mL 2.65 mmol). The reaction mixture was heated to 75° C. for 16 hours. After complete consumption of the starting material, the reaction mass was concentrated, diluted with water, extracted with ethyl acetate. The combined organic layers were washed with water and brine, dried over anhydrous Na₂SO₄ and concentrated under reduced pressure to afford the crude product. The crude compound was purified by flash column chromatography using 4-5% MeOH in DCM as an eluent to obtain a yellow liquid (250 mg, 68%).

¹H NMR (400 MHz, DMSO-d₆): δ 8.09 (d, J=1.2 Hz, 1H), 7.73 (dd, J=8.4 Hz, 1.2 Hz, 1H), 7.47 (d, J=8.4 Hz, 1H), 4.18 (t, J=6.9 Hz, 2H), 3.84 (s, 3H), 2.46-2.17 (m, 19H), 1.79 (quintet, J=6.9 Hz, 2H). LCMS: m/z 343.46 [M+H]⁺.

Other analogues prepared by this method:

methyl 1-(3-(dimethylamino)propyl)-2,3-dimethyl-1H-indole-5-carboxylate (100%).

### Preparation of 2,3-dimethyl-1-(3-(4-methylpiperazin-1-yl)propyl)-1H-indole-5-carboxylic acid

To a stirred solution of methyl 2,3-dimethyl-1-(3-(4-methylpiperazin-1-yl)propyl)-1H-indole-5-carboxylate (250 mg, 0.72 mmol) in THF/H₂O/MeOH (10 mL, 6:2:2), was added NaOH.H₂O (174 mg, 4.36 mmol) at 0° C. The reaction mixture was heated to 60° C. for 16 hours. After complete consumption of the starting material, the reaction mass was concentrated and then partitioned between ethyl acetate and water. The aqueous layer was collected and acidified with conc. HCl at 0° C. The resulting precipitate was filtered off and dried over vacuum to afford an off white solid (70 mg, 29%).

¹H NMR (400 MHz, DMSO-d₆): δ 12.21 (br s, 1H), 8.07 (d, J=1.6 Hz, 1H), 7.64 (dd, J=8.4 Hz, 1.6 Hz, 1H), 7.43 (d, J=8.4 Hz, 1H), 4.18 (t, J=6.9 Hz, 2H), 2.50-2.09 (m, 19H), 1.89 (quintet, J=6.9 Hz, 2H). LCMS: m/z 329.44 [M+H]⁺.

Other analogues prepared by this method:

1-(3-(dimethylamino)propyl)-2,3-dimethyl-1H-indole-5-carboxylic acid (53%).

### Preparation of Compound 4120, (2,3-dimethyl-1-(3-(4-methylpiperazin-1-yl)propyl)-1H-indol-5-yl)(4-(4-fluorophenethyl)piperazin-1-yl)methanone

To a stirred solution of 2,3-dimethyl-1-(3-(4-methylpiperazin-1-yl)propyl)-1H-indole-5-carboxylic acid (245 mg, 0.74 mmol) in DMF (5 mL), DIPEA (0.28 mL, 1.6 mmol) was added. The mixture was stirred for 10 minutes, followed by the addition of HATU (422 mg, 1.11 mmol) and stirring for a further 30 minutes. The reaction mass was cooled to 0° C., 1-(4-fluorophenethyl)piperazine (154 mg, 0.74 mmol) was added and the reaction mixture was stirred at room temperature for 12 hours. After complete consumption of the starting material, the reaction mass was concentrated, diluted with water and extracted with ethyl acetate. The combined organic layers were washed with water and brine, dried over anhydrous Na₂SO₄ and concentrated under reduced pressure to afford the crude product. The crude compound was purified by flash column chromatography using 5-6% MeOH in DCM as an eluent to give an off-white solid (54 mg, 14%).

¹H NMR (400 MHz, CD₃OD): δ 7.53 (br s, 1H), 7.42 (d, J=8.4 Hz, 1H), 7.24 (dd, J=8.4 Hz, 5.6 Hz, 2H), 7.17 (dd, J=8.4 Hz, 1.2 Hz, 1H), 6.99 (t, J=8.8 Hz, 2H), 4.24 (t, J=6.8 Hz, 2H), 3.72 (br s, 4H), 3.12-2.81 (m, 6H), 2.75-2.57 (m, 12H), 2.40 (br s, 6H), 2.24 (s, 3H), 1.94 (quintet, J=6.8 Hz, 2H). LCMS: m/z 520.50 [M+H]⁺.

Other analogues prepared by this method:

Compound 4018, (1-(3-(dimethylamino)propyl)-2,3-dimethyl-1H-indol-5-yl)(4-phenethylpiperazin-1-yl)methanone (3%).

¹H NMR (400 MHz, CD₃OD): δ 7.53 (d, J=1.2 Hz, 1H), 7.39 (d, J=8.8 Hz, 1H), 7.28-7.15 (m, 6H), 4.20 (t, J=7.2 Hz, 2H), 3.70 (br s, 4H), 2.86-2.81 (m, 2H), 2.67-2.52 (m, 6H), 2.39-2.35 (m, 5H), 2.25 (br s, 9H), 1.91 (quintet, J=7.2 Hz, 2H). LCMS: m/z 447.5 [M+H]⁺.

Compound 4118, (1-(3-(dimethylamino)propyl)-2,3-dimethyl-1H-indol-5-yl)(4-(4-fluorophenethyl)piperazin-1-yl)methanone (10%).

¹H NMR (300 MHz, CD₃OD): δ 7.55 (d, J=1.2 Hz, 1H), 7.42 (d, J=8.4 Hz, 1H), 7.24 (dd, J=8.7 Hz, 5.4 Hz, 2H), 7.20 (dd, J=8.4 Hz, 2.0 Hz, 1H), 7.00 (t, J=8.7 Hz, 2H), 4.27 (t, J=7.2 Hz, 2H), 3.73 (br s, 4H), 3.14-3.07 (m, 2H), 2.88-2.80 (m, 8H), 2.78-2.54 (m, 6H), 2.41 (s, 3H), 2.25 (s, 3H), 2.19-2.10 (m, 2H). LCMS: m/z 465.5 [M+H]⁺.

Compound 4119, (1-(3-(dimethylamino)propyl)-2,3-dimethyl-1H-indol-5-yl)(4-(4-hydroxyphenethyl)piperazin-1-yl)methanone (6%).

¹H NMR (300 MHz, CD₃OD): δ 7.53 (d, J=1.2 Hz, 1H), 7.38 (d, J=8.4 Hz, 1H), 7.16 (br d, 8.4 Hz, 1H), 7.03 (d, J=8.1 Hz, 2H), 6.69 (d, J=8.4 Hz, 2H), 4.19 (t, J=6.9 Hz, 2H), 3.70 (br s, 4H), 2.76-2.70 (m, 2H), 2.63-2.52 (m, 6H), 2.39 (s, 3H), 2.36-2.31 (m, 2H), 2.24 (s, 3H), 2.22 (s, 6H), 1.95-1.86 (m, 2H). LCMS: m/z 462.62 [M+H]⁺.

Compound 4121, (2,3-dimethyl-1-(3-(4-methylpiperazin-1-yl)propyl)-1H-indol-5-yl)(4-(4-methoxyphenethyl)piperazin-1-yl)methanone (5%).

¹H NMR (300 MHz, CD₃OD): δ 7.55 (br s, 1H), 7.44 (d, J=8.4 Hz, 1H), 7.22-7.14 (m, 3H), 6.85 (d, J=8.7 Hz, 2H), 4.25 (t, J=6.6 Hz, 2H), 3.81-3.68 (m, 7H), 3.30-2.69 (m, 18H), 2.40 (br s, 6H), 2.25 (s, 3H), 2.00-1.91 (m, 2H). LCMS: m/z 532.5 [M+H]⁺.

Compound 4122, (2,3-dimethyl-1-(3-(4-methylpiperazin-1-yl)propyl)-1H-indol-5-yl)(4-(4-hydroxyphenethyl)piperazin-1-yl)methanone (11%).

¹H NMR (300 MHz, CD₃OD): δ 7.52 (br s, 1H), 7.40 (d, J=8.4 Hz, 1H), 7.15 (dd, J=8.4 Hz, 1.5 Hz, 1H), 7.03 (d, J=8.4 Hz, 2H), 6.69 (d, J=8.4 Hz, 2H), 4.21 (t, J=6.9 Hz, 2H), 3.71 (br s, 4H), 2.74-2.28 (m, 21H), 2.27 (s, 3H), 2.24 (s, 3H), 1.95-1.87 (m, 2H). LCMS: m/z 517.7 [M+H]⁺.

Compound 4123, (4-(2-(benzo[d][1,3]dioxol-5-yl)ethyl)piperazin-1-yl)(2,3-dimethyl-1-(3-(4-methylpiperazin-1-yl)propyl)-1H-indol-5-yl)methanone (20%).

¹H NMR (400 MHz, CD₃OD): δ 7.54 (br s, 1H), 7.42 (d, J=8.4 Hz, 1H), 7.17 (dd, J=8.4 Hz, 1.2 Hz, 1H), 6.74-6.67 (m, 3H), 5.89 (s, 2H), 4.25 (t, J=6.8 Hz, 2H), 3.73 (br s, 4H), 2.99-2.57 (m, 18H), 2.40 (br s, 6H), 2.25 (s, 3H), 1.95 (quintet, J=6.8 Hz, 2H). LCMS: m/z 546.5 [M+H]⁺.

Compound 4124, (2, 3-dimethyl-1-(3-(4-methyl piperazin-1-yl)propyl)-1H-indol-5-yl)(4-(3-fluorophenethyl)piperazin-1-yl)methanone (37%).

¹H NMR (400 MHz, CD₃OD): δ 7.55 (br s, 1H), 7.43 (d, J=8.0 Hz, 1H), 7.29 (q, J=8.0 Hz, 1H), 7.18 (dd, J=8.4 Hz, 1.2 Hz, 1H), 7.06 (d, J=7.6 Hz, 1H), 7.00 (br d, J=9.6 Hz, 1H), 6.93 (td, J=8.4 Hz, 2.4 Hz, 1H), 4.25 (t, J=6.8 Hz, 2H), 3.75 (br s, 4H), 3.13-2.67 (m, 18H), 2.40 (br s, 6H), 2.25 (s, 3H), 1.99-1.92 (m, 2H). LCMS: m/z 520.5 [M+H]⁺.

Compound 4125, (2,3-dimethyl-1-(3-(4-methylpiperazin-1-yl)propyl)-1H-indol-5-yl)(4-(3-methoxyphenethyl)piperazin-1-yl)methanone (5%).

¹H NMR (400 MHz, CD₃OD): δ 7.54 (d, J=1.2 Hz, 1H), 7.43 (d, J=8.4 Hz, 1H), 7.21-7.16 (m, 2H), 6.83-6.74 (m, 3H), 4.25 (t, J=6.8 Hz, 2H), 3.81-3.64 (m, 7H), 2.86-2.57 (m, 18H), 2.40 (br s, 6H), 2.25 (s, 3H), 1.99-1.91 (m, 2H). LCMS: m/z 532.5 [M+H]⁺.

Compound 4126, (2,3-dimethyl-1-(3-(4-methylpiperazin-1-yl)propyl)-1H-indol-5-yl)(4-(3-hydroxyphenethyl)piperazin-1-yl)methanone (7%).

¹H NMR (300 MHz, CD₃OD): δ 7.52 (br s, 1H), 7.40 (d, J=8.4 Hz, 1H), 7.15 (br d, J=8.1 Hz, 1H), 7.07 (t, J=7.5 Hz, 1H), 6.70-6.58 (m, 3H), 4.21 (t, J=6.6 Hz, 2H), 3.71 (br s, 4H), 2.86-2.31 (m, 21H), 2.27 (s, 3H), 2.24 (s, 3H), 1.98-1.87 (m, 2H). LCMS: m/z 517.7 [M+H]⁺.

Compound 4127, (2,3-dimethyl-1-(3-(4-methylpiperazin-1-yl)propyl)-1H-indol-5-yl)(4-phenethylpiperazin-1-yl)methanone (48%).

¹H NMR (400 MHz, CD₃OD): δ 7.56 (br s, 1H), 7.43 (d, J=8.4 Hz, 1H), 7.31-7.17 (m, 6H), 4.25 (t, J=6.8 Hz, 2H), 3.77 (br s, 4H), 3.13-2.65 (m, 18H), 2.40 (br s, 6H), 2.25 (s, 3H), 1.99-1.91 (m, 2H). LCMS: m/z 502.57 [M+H]⁺.

### Activity of Anti-tropomyosin Compounds as Monotherapy

Anti-proliferative Activity of Compounds of the Invention

In silico modelling has identified binding sites on tropomyosin Tm5NM1, yielding the series of tropomyosin inhibitors the subject of the present invention. Inhibition of Tm5NM1 in tumour cells results in disruption of the actin cytoskeleton and ultimately cell death. The activity of compounds 4001-4015 was assessed in vitro using the Microfilament disruption assay. Briefly, cells were seeded (5×10³/well) into 8-well chamber slides (NUNC) and treated with the concentrations of anti-tropomyosin compounds nominated in Table 1 for 24 hours using DMSO as vehicle control. Actin was visualized with Alexa 555 conjugated phalloidin (Molecular probes). Random fields were imaged using an Olympus IX81 microscope. Cells (n≧50) were scored on the basis of positive filament staining from n=3 independent experiments.

Cell viability assays were also conducted to assess the anti-proliferative effects of the anti-tropomyosin compounds 4001-4015. Briefly, cells (1×10³/well) were plated (96-well) and treated (48 hr) with anti-tropomyosin drug and viability measured using 3-(4,5-dimethylthiazol-2-yl)-2,5-diphenylterazolium bromide MTT. Cell viability was normalized to control (vehicle alone) and dose-response curves, and half maximal effective concentration (EC₅₀) values were determined using Graph Pad Prism 5 (nonlinear regression sigmoidal dose-response variable slope).

Data demonstrate that those anti-tropomyosin compounds that effectively disrupt the actin microfilament also have a strong anti-proliferative effect on neuroblastoma (SH-EP) and melanoma (SK-Mel-28) cells (Table 1).

TABLE 1
Biological activity of compounds of the invention

| Compound | IC50 (μM) | | Microfilament disruption | | |
|---|---|---|---|---|---|
| ID | SKMEL28 | SHEP | 2.5 μM | 5 μM | 10 μM |
| 4001 | 4.3 | 4.63 | NT | NT | +++ |
| 4002 | 7.04 | 9.24 | NT | NT | + |
| 4003 | 25.9 | NT | NT | NT | NT |
| 4004 | 9.8 | 6.7 | | | + |
| 4005 | 53.8 | 5.4 | NT | NT | + |
| 4006 | ND | 107 | NT | NT | − |
| 4007 | ND | ND | NT | NT | − |
| 4008 | 3.4 | 2.7 | NT | NT | +++ |
| 4009 | 5.4 | 3 | NT | NT | +++ |
| 4010 | 3.4 | 6.4 | NT | NT | +++ |
| 4011 | 9.7 | 8.7 | NT | NT | ++ |
| 4012 | ND | ND | NT | NT | − |
| 4013 | 15.8 | 14.5 | NT | NT | + |
| 4014 | 4 | 4.3 | NT | NT | +++ |
| 4015 | 6.4 | 4.6 | NT | NT | +++ |

Compounds 4001, 4010, 4014, 4008 and 4015 were then screened against non-transfected and Tm5NM1 stably transfected MEFs. Briefly, MEFs were transfected with pEYFP-C1/Tm5NM1 constructs using the Amaxa nucleofector apparatus (program A-023) and MEF nucleofector kit 1 (Lonza) according to the manufacturer's instructions. Cell viability assays were also conducted to assess the anti-proliferative effects of the anti-tropomyosin compounds. Briefly, cells (1×10³/well) were plated (96-well) and treated (48 hr) with anti-tropomyosin compounds and viability measured using 3-(4,5-dimethylthiazol-2-yl)-2, 5-diphenylterazolium bromide MTT. Cell viability was normalized to control (vehicle alone) and dose-response curves, and half maximal effective concentration (EC50) values were determined using Graph Pad Prism 5 (nonlinear regression sigmoidal dose-response variable slope).

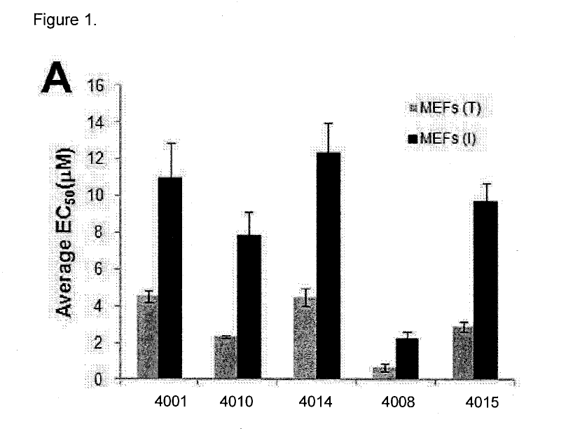

Data generated show that the selectivity of Tm5NM1/2 inhibitors was improved with less toxicity noted against primary mouse embryonic fibroblasts (MEFs) compared with MEFs stably transfected with Tm5NM1/2, particularly compounds 4001, 4010, 4014 and 4015 (FIG. 1).

The ability of compounds 4001, 4002, 4008, 4010, 4011, 4013-4015, 4018, 4026, 4045-4113 and 4115-4127 to inhibit the proliferation of cancer cells representative of neuroblastoma, melanoma, prostate cancer, colorectal cancer, non-small cell lung carcinoma, and triple negative breast cancer was assessed. These studies were conducted by contract research (GVK-BIO). Briefly, a pre-determined number of cells as calculated from cell growth assays for each of the cell lines employed were seeded into their respective culture medium (using ATCC culture parameters—http://www.atcc.org) and cultured for 24 h at 37° C. and 5% CO₂ in 96-well culture plates. Once attached, each cell line was then exposed to various concentrations of each respective analogue (30, 3, 0.3 and 0.03 μM for compounds in Tables 2, 3 and 4; 30, 10, 3, 1, 0.3 and 0.1 μM for compounds in Table 5), cultured for a further 72 h and exposed to cell-titre luminescent reagent (100 μL/well) for a further 30 min). Luminescence was captured using an EnVision multilabel reader and the data for each analogue concentration compared against no treatment control. For compounds in Tables 2-4, semi-log plots of Percent of Control versus concentration were prepared and IC₅₀ determined using linear regression analysis. For compounds in Table 5, cell viability was normalized to control (vehicle alone) and dose-response curves, and half maximal effective concentration (EC₅₀) values were determined using Graph Pad Prism 6 (nonlinear regression sigmoidal dose-response variable slope).

TABLE 2
Anti-proliferative activity of compounds of the invention
against a range of somatic cancer cells.

| | IC₅₀/μM | | | | | | |
|---|---|---|---|---|---|---|---|
| | | | | | | Lung | Breast |
| Compound | Neuroblastoma | Melanoma | Prostate | | Colorectal | (NSLC) | MDA-MB- |
| ID | SK-N-SH | SK-Mel-28 | DU145 | PC3 | CaCo2 | A549 | 231 |
| 4001 | 4.1 | 3.6 | 3.2 | 2.8 | 3.0 | 2.7 | 3.1 |
| 4008 | 9.3 | 3.3 | 3.3 | 4.3 | 5.8 | 3.8 | 3.9 |
| 4010 | 3.6 | 3.4 | 3.4 | 4.0 | 3.5 | 2.8 | 2.6 |
| 4011 | 2.5 | 6.1 | 3.3 | 4.3 | 2.8 | 3.0 | 2.5 |
| 4013 | 4.2 | 9.2 | >30 | >30 | >30 | 7.6 | 3.7 |
| 4014 | 3.0 | 3.2 | 3.6 | 3.7 | 3.9 | 2.9 | 2.5 |
| 4015 | 4.6 | 3.9 | 3.7 | 2.9 | 2.9 | 1.8 | 3.0 |
| 4018 | >30 | 67.3 | >30 | >30 | >30 | >30 | >30 |
| 4026 | 3.7 | 5.9 | 5.0 | 6.1 | 15.4 | 3.8 | 3.5 |
| 4045 | 3.8 | 3.7 | 5.5 | 5.7 | 5.2 | 3.7 | 2.6 |
| 4046 | 3.2 | 1.9 | 2.6 | 1.1 | 3.2 | 1.7 | 1.9 |
| 4047 | 3.1 | 2.5 | 3.4 | 3.1 | 3.0 | 3.0 | 3.0 |
| 4048 | 3.9 | 3.1 | 3.3 | 3.8 | 6.9 | 3.0 | 3.2 |

TABLE 3
Anti-proliferative activity of compounds of the invention
against a range of somatic cancer cells.

| | IC₅₀/μM | | | | | | |
|---|---|---|---|---|---|---|---|
| | | | | | | Lung | Breast |
| Compound | Neuroblastoma | Melanoma | Prostate | | Colorectal | (NSLC) | MDA-MB- |
| ID | SK-N-SH | SK-Mel-28 | DU145 | PC3 | CaCo2 | A549 | 231 |
| 4049 | 3.3 | 3.1 | 3.3 | 3.4 | 3.2 | 3.0 | 3.4 |
| 4050 | 4.0 | 2.6 | 4.3 | 4.2 | 4.2 | 3.1 | 3.7 |
| 4051 | 4.3 | 3.4 | 4.9 | 17.3 | 16.1 | 4.1 | 4.5 |
| 4052 | 31.9 | 6.2 | 13.8 | 11.1 | 27.1 | 8.0 | 30.0 |
| 4053 | 3.8 | 2.5 | 4.3 | 3.7 | 4.6 | 2.0 | 2.5 |
| 4054 | 4.5 | 4.1 | 4.5 | 3.9 | 5.1 | 3.9 | 4.6 |
| 4055 | 4.6 | 5.1 | 4.5 | 7.9 | 6.8 | 4.7 | 4.7 |
| 4056 | 3.4 | 3.4 | 4.3 | 7.0 | 4.7 | 4.0 | 3.0 |
| 4057 | 4.9 | 2.7 | 6.4 | 8.7 | 10.3 | 4.4 | 3.0 |
| 4058 | 3.4 | 2.7 | 4.7 | 7.2 | 9.7 | 4.7 | 3.8 |
| 4059 | 11.3 | 15.5 | >30 | >30 | >30 | 13.4 | 13.2 |
| 4060 | 3.6 | 2.8 | 4.0 | 5.0 | 4.0 | 4.2 | 2.8 |
| 4061 | 2.3 | 2.5 | 3.0 | 2.2 | 3.7 | 3.0 | 3.2 |
| 4062 | 4.8 | 4.3 | 4.4 | 9.0 | 6.0 | 2.3 | 3.7 |
| 4063 | 4.2 | 3.8 | 3.6 | 4.9 | 9.6 | 3.0 | 3.7 |
| 4064 | 2.4 | 2.7 | 3.4 | 2.6 | 3.8 | 3.5 | 3.3 |
| 4067 | 3.3 | 2.5 | 4.3 | 4.3 | 4.3 | 4.0 | 3.8 |
| 4068 | 3.5 | 2.7 | 5.4 | 14.6 | >30 | 2.9 | 2.8 |
| 4069 | 3.4 | 2.5 | 4.4 | 9.9 | >30 | 4.2 | 4.2 |
| 4070 | 2.2 | 2.5 | 3.1 | 2.9 | 1.7 | 2.3 | 1.7 |
| 4071 | 3.7 | 4.9 | 4.7 | 7.6 | 12.0 | 4.0 | 3.4 |
| 4072 | 1.7 | 3.0 | 2.9 | 3.8 | 2.0 | 1.9 | 1.7 |
| 4073 | 2.6 | 3.1 | 4.0 | 4.2 | 2.8 | 3.9 | 3.4 |
| 4074 | 1.3 | 2.7 | 2.8 | 22.7 | 2.4 | 2.5 | 1.5 |
| 4075 | 2.6 | 2.7 | 3.0 | 4.5 | 3.3 | 2.9 | 2.6 |
| 4076 | 2.8 | 2.1 | 3.2 | 3.7 | 2.9 | 1.8 | 1.7 |
| 4077 | 3.1 | 2.7 | 3.2 | 2.8 | 3.3 | 3.3 | 3.4 |
| 4078 | 6.5 | 1.9 | 1.4 | 1.2 | 3.6 | 1.4 | 1.6 |
| 4079 | 2.9 | 2.8 | 3.1 | 1.8 | >30 | 3.1 | 4.3 |
| 4080 | 2.8 | 3.1 | 3.5 | 3.0 | 3.0 | 3.1 | 3.2 |
| 4081 | 1.6 | 2.3 | 2.9 | 2.1 | 3.1 | 2.9 | 2.5 |
| 4085 | 9.8 | 10.5 | 11.0 | 10.1 | 10.9 | 10.1 | 11.2 |
| 4088 | 3.4 | 4.3 | 3.3 | 3.5 | 4.1 | 3.4 | 3.8 |
| 4093 | 2.5 | 0.9 | 1.7 | 1.2 | 1.8 | 3.0 | 2.1 |
| 4095 | 1.5 | 2.3 | 1.2 | 1.4 | 2.3 | 1.0 | 1.6 |
| 4096 | 3.7 | 3.5 | 3.3 | 2.5 | 2.7 | 2.9 | 2.6 |

TABLE 4
Anti-proliferative activity of compounds of the invention
against a range of somatic cancer cells.

| | IC₅₀/μM | | | | | | |
|---|---|---|---|---|---|---|---|
| | | | | | | Lung | Breast |
| Compound | Neuroblastoma | Melanoma | Prostate | | Colorectal | (NSLC) | MDA-MB- |
| ID | SK-N-SH | SK-Mel-28 | DU145 | PC3 | CaCo2 | A549 | 231 |
| 4097 | 2.9 | 3.6 | 3.5 | 4.9 | 3.5 | 3.0 | 2.5 |
| 4098 | 3.4 | 3.8 | 3.4 | 3.3 | 4.0 | 2.9 | 3.7 |
| 4099 | 3.9 | 3.7 | 5.5 | 4.9 | >30 | 4.1 | 3.4 |
| 4100 | 3.6 | 2.5 | >30 | >30 | >30 | 4.5 | 5.2 |
| 4101 | 3.4 | 3.3 | 5.2 | 7.8 | >30 | 4.0 | 3.9 |
| 4102 | 3.5 | 3.5 | 6.0 | 5.5 | >30 | 4.0 | 3.6 |
| 4103 | 2.9 | 5.4 | 3.7 | 5.4 | 5.0 | 3.0 | 2.7 |
| 4104 | 3.4 | 3.6 | 4.1 | 4.1 | 3.2 | 4.0 | 4.2 |
| 4105 | 3.4 | 2.6 | 3.6 | 3.7 | 2.6 | 3.6 | 2.7 |
| 4106 | 1.8 | 2.6 | 3.1 | 3.6 | 2.8 | 2.4 | 1.9 |
| 4107 | 3.6 | 2.8 | 3.5 | 3.5 | 3.6 | 2.9 | 2.8 |
| 4108 | 2.9 | 2.9 | 3.5 | 3.6 | 2.1 | 3.4 | 2.2 |
| 4118 | 31.9 | 18.1 | >30 | >30 | >30 | >30 | >30 |
| 4120 | 13.8 | 17.7 | >30 | >30 | >30 | 23.0 | >30 |
| 4121 | 13.6 | 14.7 | >30 | >30 | >30 | 15.7 | 13.2 |
| 4123 | 12.3 | 16.8 | >30 | >30 | >30 | 15.6 | 16.3 |
| 4124 | 10.9 | 15.9 | >30 | 21.8 | 29.6 | 15.0 | 13.9 |
| 4125 | 14.9 | 25.0 | >30 | >30 | >30 | 19.4 | 33.2 |
| 4127 | 10.5 | 14.4 | >30 | 12.9 | 10.7 | 13.1 | 13.5 |

TABLE 5
Anti-proliferative activity of compounds of the invention
against a range of somatic cancer cells.

| | IC₅₀/μM | | | | | | |
|---|---|---|---|---|---|---|---|
| | | | | | | Lung | Breast |
| Compound | Neuroblastoma | Melanoma | Prostate | | Colorectal | (NSLC) | MDA-MB- |
| ID | SK-N-SH | SK-Mel-28 | DU145 | PC3 | CaCo2 | A549 | 231 |
| 4002 | 3.2 | 9.3 | 37.4 | 12.4 | 13.1 | 16.3 | 15.7 |
| 4065 | 5.2 | 5.0 | 4.2 | 6.5 | 6.5 | 4.6 | 5.0 |
| 4066 | 5.4 | 4.9 | 6.9 | 10.0 | 14.8 | 8.5 | 11.4 |
| 4082 | 4.8 | 4.6 | 2.6 | 3.9 | 4.0 | 4.9 | 4.2 |
| 4083 | 3.3 | 4.7 | 5.1 | 13.2 | 11.9 | 11.2 | 13.1 |
| 4084 | 10.6 | 5.5 | 4.8 | 3.5 | 7.1 | 6.0 | 6.5 |
| 4086 | 5.7 | 5.2 | 4.3 | 4.8 | 7.6 | 5.0 | 6.2 |
| 4087 | 5.6 | 4.8 | 7.1 | 7.4 | 23.1 | 8.9 | 7.5 |
| 4089 | 4.4 | 4.3 | 4.4 | 4.4 | 13.2 | 7.7 | 5.2 |
| 4090 | 6.3 | 7.2 | 4.6 | 2.5 | 5.3 | 8.4 | 9.6 |
| 4091 | 9.7 | 15.4 | 10.6 | 19.6 | 10.2 | 9.5 | 28.9 |
| 4092 | 13.8 | 13.3 | 16.7 | 38.3 | 17.1 | 8.3 | 22.6 |
| 4094 | 1.8 | 3.8 | 2.0 | 1.5 | 4.2 | 2.7 | 1.7 |
| 4109 | 3.7 | 1.0 | 5.3 | 9.5 | 9.1 | 6.5 | 3.6 |
| 4110 | 4.2 | 4.4 | 22.7 | 17.7 | 12.9 | 33.2 | 6.9 |
| 4111 | 3.4 | 2.4 | 11.3 | 7.5 | 7.9 | 5.5 | 3.9 |
| 4112 | 7.0 | 13.0 | 8.3 | 8.4 | 17.5 | 5.9 | 11.8 |
| 4113 | 1.7 | 0.9 | 5.4 | 11.4 | 4.6 | 5.2 | 2.8 |
| 4115 | 4.9 | 1.1 | 6.1 | 9.4 | 6.2 | 8.8 | 3.4 |
| 4116 | 4.6 | 3.2 | 7.6 | 13.2 | 6.0 | 10.1 | 4.1 |
| 4117 | 4.6 | 2.0 | 5.6 | 10.2 | 8.2 | 8.6 | 3.5 |
| 4119 | >30 | >30 | >30 | >30 | >30 | >30 | >30 |
| 4122 | >30 | >30 | >30 | >30 | >30 | >30 | >30 |
| 4126 | >30 | >30 | >30 | >30 | >30 | >30 | >30 |

The anti-proliferative activity of compound 4093 against ovarian cancer (SKOV3) and glioblastoma (U251) cells was also evaluated (Table 6). Briefly, cells were seeded at 2000 cells/well in a 96 well plate. 24 hrs post plating, each cell line was exposed to various concentrations of ATM-4093 (10, 5, 3,1, 0.7, 0.5, 0.3, 0.1 μM) for 72 hours. Cell viability was determined using CellTiter 96 AQᵤₑₒᵤₛ (Promega) and absorbance measured at 490 nm on the SpectraMax M2 plate reader. Cell viability was normalized to control (vehicle alone) and dose-response curves, and half maximal effective concentration (EC₅₀) values were determined using Graph Pad Prism 6 (nonlinear regression sigmoidal dose-response variable slope).

TABLE 6
Anti-proliferative activity of compound 4093 against
ovarian cancer and glioblastoma cells.

| Cell line | IC₅₀/μM | 95% CI |
|---|---|---|
| SKOV3 (Ovarian) | 0.6 | 0.2-0.6 |
| U251 (Glioblastoma) | 0.5 | 0.3-1.8 |

Impact of Compounds of the Invention on the Actin Cytoskeleton

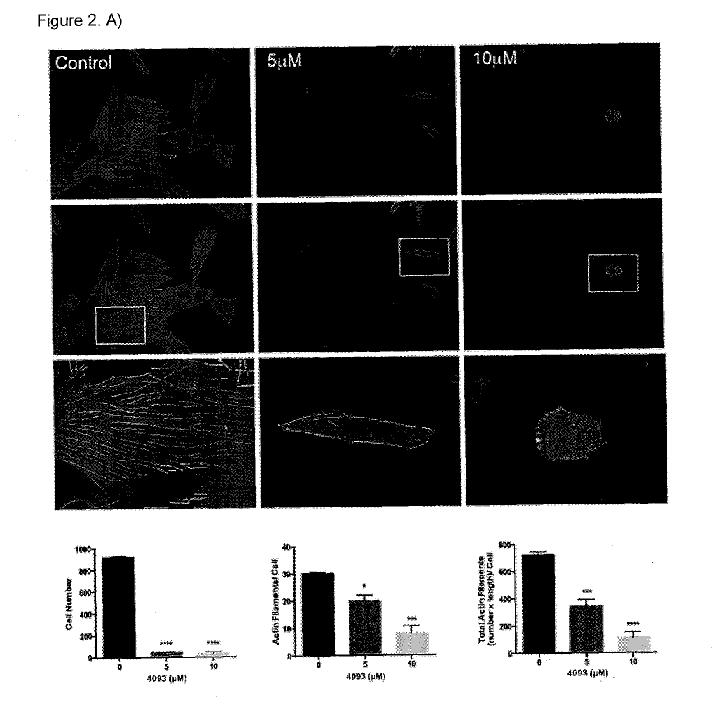

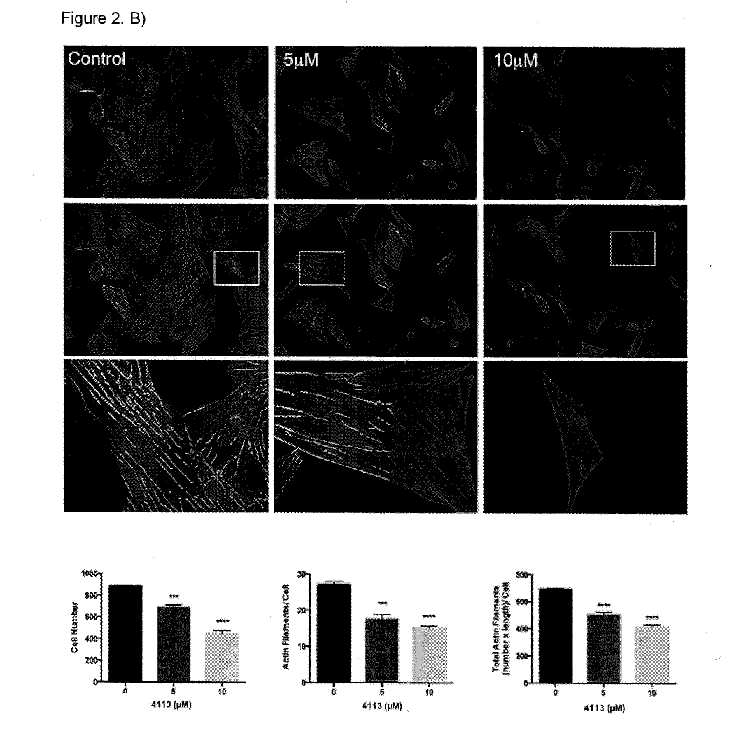

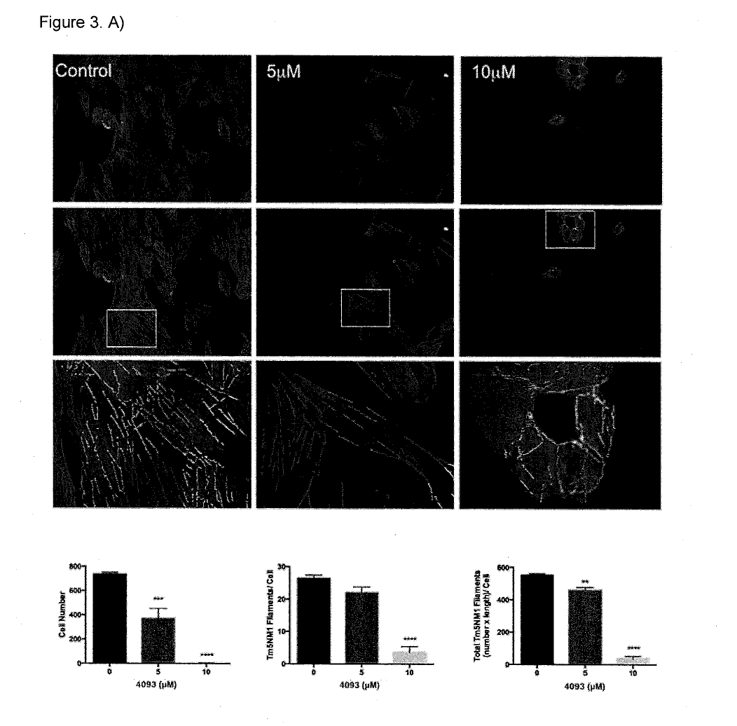

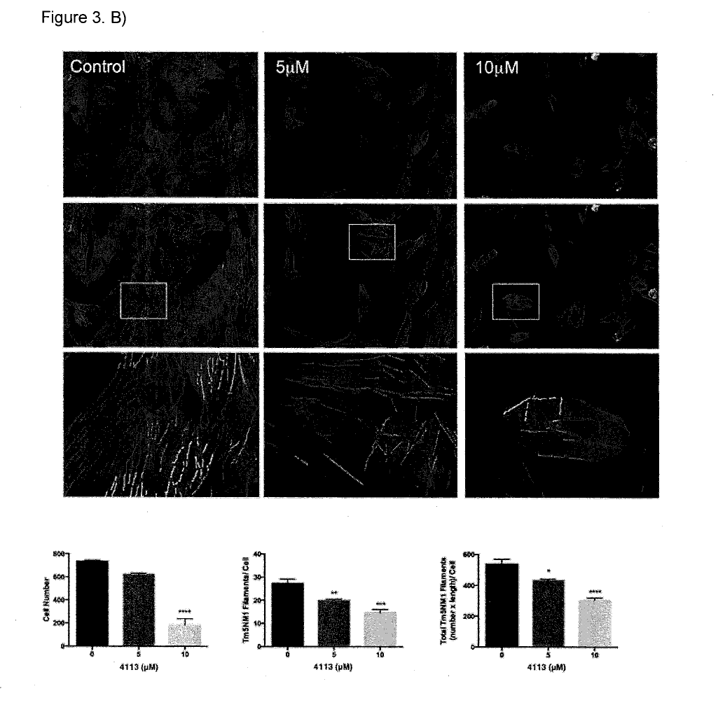

The ability of compounds 4093 and 4113 to disrupt the total actin cytoskeleton (FIG. 2) and to specifically target Tm5NM1-containing actin filaments (FIG. 3) was assessed in vitro using the microfilament disruption assay.

Briefly, SK-N-SH neuroblastoma cells were seeded at 1800 cells/well in a 384 Perkin Elmer High Content Imaging “View” plate and left to plate down 24 h prior to treatment. Cells were then treated with 0-40 μM of the test compounds (1:2 serial dilution in a 10 point dose response). 24 h post treatment, cells were fixed with 4% paraformaldehyde (PBS), permeabilized with Triton-X-100 and stained with 488-Atto-Phallodin and DAPI to visualize the actin filament bundles and the nucleus, or with γ9d (sheep polycolonal, 1:100) followed by 488-conjugated secondary (1:1000) and DAPI to visualize the Tm5NM1 containing filament bundles and the nucleus, respectively. Single plane images were obtained on the Perkin Elmer Opera confocal microscope using a 20× objective. Twelve fields of view per condition were imaged. Images were then exported and changes in the organization and numbers of actin filaments within the cell were quantitated using a linear feature detection algorithm developed by the CSIRO. This algorithm detects the “ridge lines” or “peaks” in local pixel intensity in the cell image. It is these “ridge lines” that correspond to actin filament bundles and allow us to quantitate the number of filaments per cell.

Data demonstrate that compounds 4093 and 4113 disrupt both the total actin cytoskeleton and Tm5NM1-containing actin filaments in a dose-dependent manner.

In order to demonstrate that the compounds of the invention impaired Tm5NM1 function the impact of compounds 4015 and 4093 on Tm5NM1-regulated actin filament depolymerization was assessed using a well-characterized pyrene-based actin filament depolymerization assay (Broschat, 1990; Kostyukova and Hitchcock, 2004). A brief overview and rationale of the assay is as follows: to promote depolymerization, pyrene-labelled actin filaments were diluted below the critical concentration of the pointed end (0.5 μM, as defined by Pollard et al., 1986). A decline in fluorescence was measured over time as actin monomers dissociate. It is well established that in the presence of Tm5NM1 the rate of actin depolymerization is significantly reduced (Bonello 2013). Therefore, any compound, which interacts with, and impacts Tm5NM1 function, would nullify the protective effect of Tm5NM1 on actin depolymerization.

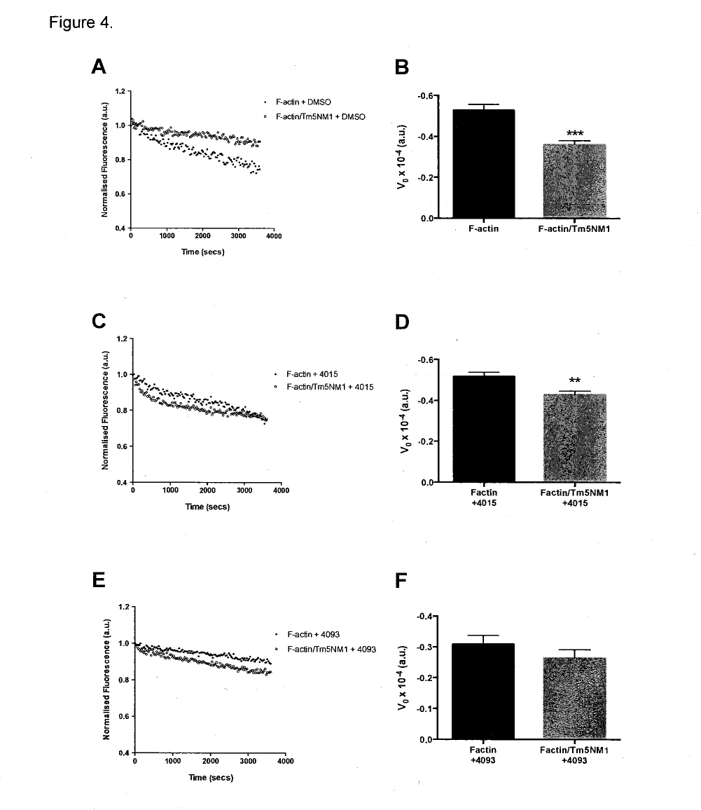

For all assays the depolymerization of F-actin alone and F-actin-coated with the human homologue of Tm5NM1 was used as a comparative control. Briefly, Tm5NM1 was pre-incubated with F-actin for 20 minutes prior to diluting the filaments, to allow for proper assembly of the Tm5NM1 polymer. As expected, in the presence of saturating amounts of Tm5NM1, the initial rate (V₀) of F-actin depolymerization was significantly slower for Tm5NM1-containing actin filaments (−0.36±0.02×10⁻⁴) when compared to actin filaments alone (−0.53±0.027×10⁻⁴; FIG. 4A and B, p<0.0001).

The depolymerization of F-actin alone and F-actin-coated with Tm5NM1 was then measured in the presence of test compound and initial rates of depolymerization were compared. Tm5NM1 was pre-incubated with 50 μM 4015 and 4093 prior to being added to the actin filaments as previously described. In the presence of compound 4015, the rate of depolymerization of Tm5NM1-containing actin filaments was increased. With Compound 4093, no significant difference was observed between the rates of depolymerization of Tm5NM1-containing actin compared to F-actin alone (−0.26±0.028×10⁻⁴ vs −0.30±0.029×10⁻⁴; FIG. 4E and F, p=0.2772). These data demonstrate that both compounds 4015 and 4093 interact with and impair Tm5NM1 function.

Tolerance and in vivo Efficacy of Compound 4015

In vivo efficacy studies for compound 4015 hydrochloride salt were conducted using an A375 melanoma xenograft model in Foxn-1 nu/nu athymic mice. The human melanoma cell line A375 (sourced from American Type Culture Collection (ATCC), USA) was used for developing the flank xenograft model. Briefly, five million cells were injected subcutaneously in the right flank region of the animal. When the tumors reached 130-150 mm³ the animals were randomized into two groups, each group with 8 animals in them, so that the average tumor volume of all the groups was same.

First group received vehicle (sodium bicarbonate buffer) intravenously once a day (QD). The second group received compound 4015 at 20 mg/kg, intravenously (QD) (Table 7). The volume of dosing to each animal was calculated and adjusted every day based on individual body weight measured prior to dosing.

TABLE 7
Drug dosing regimen for compound 4015 efficacy study

| | | | Route and | | |
|---|---|---|---|---|---|
| Group | No. | | frequency of | Dose | |
| No. | Animals | Treatment | administration | volume | [Dose] |
| 1 | 8 | Bicarbonate | Intra- | 10 mL/kg | NA |
| | | vehicle | venous/QD | | |
| 2 | 8 | Compound | Intra- | 10 mL/kg | 20 mg/kg |
| | | 4015 | venous/QD | | |

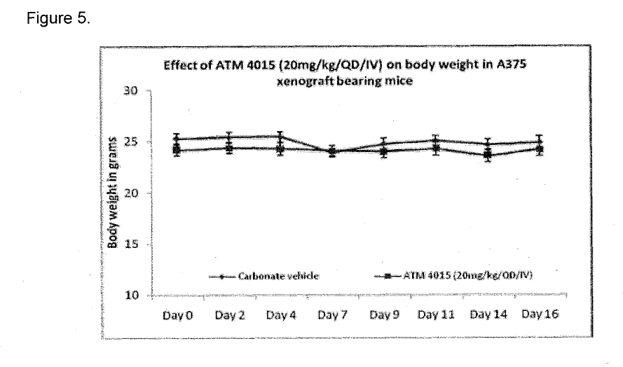

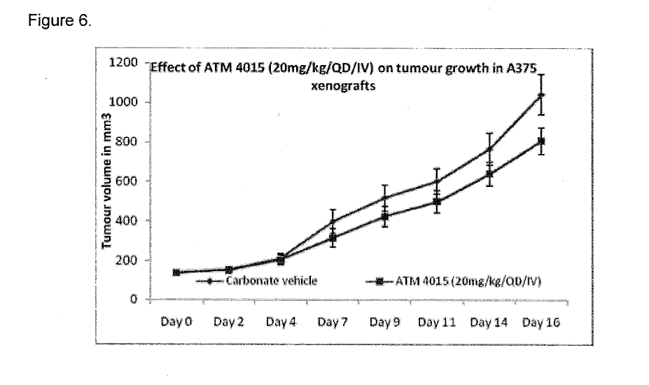

Animals were treated from the day of randomization (Day 1) for 15 days. Body weight and tumor dimensions (length and diameter) were measured three times in a week including the termination day of the study. Throughout the study period, mice were monitored daily for clinical condition. Animals were healthy in terms of body weight (FIG. 5) and clinical observations throughout the study period demonstrating that at this dose compound 4015 was well tolerated. Further to this, 4015 treatment resulted in a 26% reduction in tumor growth compared to control (FIG. 6).

These studies demonstrate that 4015 was well tolerated and effective in reducing tumor growth in vivo.

### SELECTED REFERENCE ARTICLES

Broschat, K. O. (1990). Tropomyosin prevents depolymerization of actin filaments from the pointed end. J Biol Chem 265, 21323-21329.

Kostyukova, A. S., and Hitchcock-DeGregori, S. E. (2004). Effect of the structure of the N terminus of tropomyosin on tropomodulin function. J Biol Chem 279, 5066-5071.

Pollard, T. D. (1986). Rate constants for the reactions of ATP- and ADP-actin with the ends of actin filaments. J Cell Biol 103, 2747-2754.

Bonello, T. B (2013). Characterising the impact of tropomyosin targeting compounds in the actin cytoskeleton. Ph.D thesis, School of Medical Sciences, University of New South Wales, Australia

It will be understood that the invention disclosed and defined in this specification extends to all alternative combinations of two or more of the individual features mentioned or evident from the text or drawings. All of these different combinations constitute various alternative aspects of the invention.

1. A compound of formula (I) or a pharmaceutically acceptable drug or prodrug thereof, wherein: 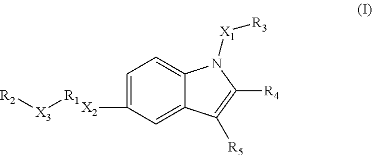 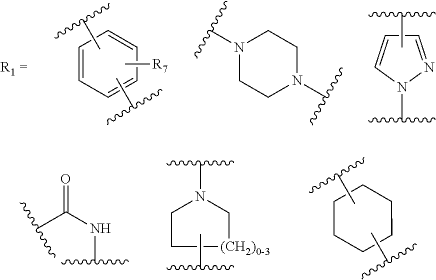
X₁═(CH₂)₀₋₅
X₂ and X₃═O, NH, NHR_{5′}, SO₂, C(O), C(O)NH, (CH₂)₀₋₅, C(O)(CH₂)₁₋₅, NH(CH₂)₁₋₅, CHR_{5′}, CHR(R_{5′})C(O), pyrazole, isooxazole
X₄═O, NH, NR₆
R₂═N(R₆)₂, CH(R₆)₂, indole 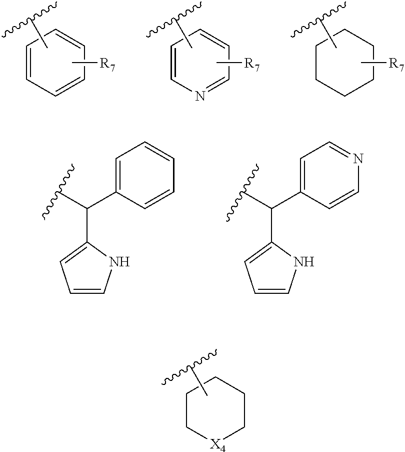
R₄, R₅ and R_{5′}═H, CH₃, CH₂CH₃
R₆═H, CH₃, (CH₂)₁₋₅CH₃, (CH₂)₁₋₅OCH₃, CF₃, CN, OCF₃
R₇═H, OH, alkyl, halo, alkoxy, hydroxyalkyl, amino, aminoalkyl, diaminoalkyl, or a dioxolane ring fused to 2 adjacent carbon atoms of R₁ of R₂
R₃═H, NH₂, N(R₆)₂, OR₄,

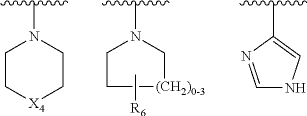

2. A compound according to claim 1, wherein X₁ is CH₂, (CH₂)₂ ^{or} (CH₂)₃.

3. A compound according to claim 1, wherein R₃ is N(R₆)₂.

4. A compound according to claim 3, wherein R₆ is CH₃.

5. A compound according to claim 1, wherein R₃ is H.

6. A compound according to claim 1, wherein R₃ is

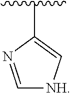 [SMILES: *c1c[nH]cn1]

7. A compound according to claim 1 or 2, wherein R₃ is

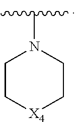

8. A compound according to claim 7, wherein X₄ is NR_{S}.

9. A compound according to claim 8, wherein R₅ is CH₃.

10. A compound according to claim 1, wherein R₄ is CH₃ or H.

11. A compound according to claim 1, wherein R₅ is CH₃ or H.

12. A compound according to claim 1, wherein X₂ is CH₂, O, (CH₂)₀, NH or C(O).

13. A compound according to claim 1, wherein R₁ is

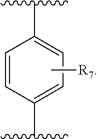

14. A compound according to claim 1 , wherein R₁ is

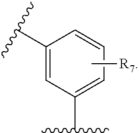

15. A compound according to claim 13, wherein R₇ is H.

16. A compound according to claim 1, wherein R₁ is

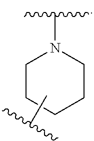

17. A compound according to claim 1, wherein X₃ is (CH₂)₂, C(O)NH, CH₂, (CH₂)₀, O or CHR₅.

18. A compound according to claim 17, wherein R_{5′} is CH₃.

19. A compound according to claim 1, wherein R₂ is 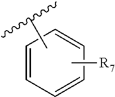
and R₇ is H, OH, halo, alkoxy or the dioxolane ring.

20. A compound according to claim 19, wherein halo is F.

21. A compound according to claim 19, wherein alkoxy is OCH₃.

22. A compound according to claim 1, wherein R₂ is

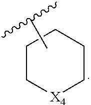

23. A compound according to claim 22, wherein X₄ is O.

24. A compound according to claim 22, wherein X₄ is NR₆.

25. A compound according to claim 24, wherein R₆ is CH₃.

26. A compound according to claim 1, wherein R₂ is 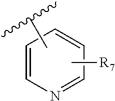
and R₇ is H.

27. A compound according to claim 1, wherein R₂ is CH(R₆)₂.

28. A compound according to claim 27, wherein R₆ is CH₃.

29. A compound according to claim 1, wherein R₂ is N(R₆)₂.

30. A compound according to claim 29, wherein R₆ is CH₃.

31. A compound according to claim 1, wherein R₂ is 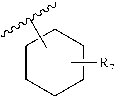
and R₇ is hydroxyalkyl.

32. A compound according to claim 31, wherein hydroxyalkyl is CH₂OH.

33. A compound according to claim 1, selected from the group consisting of:

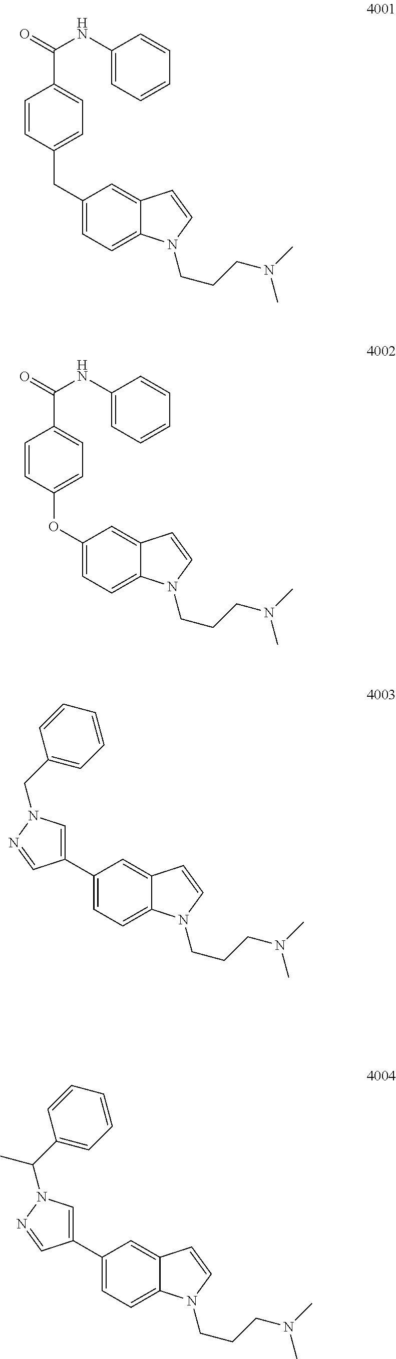

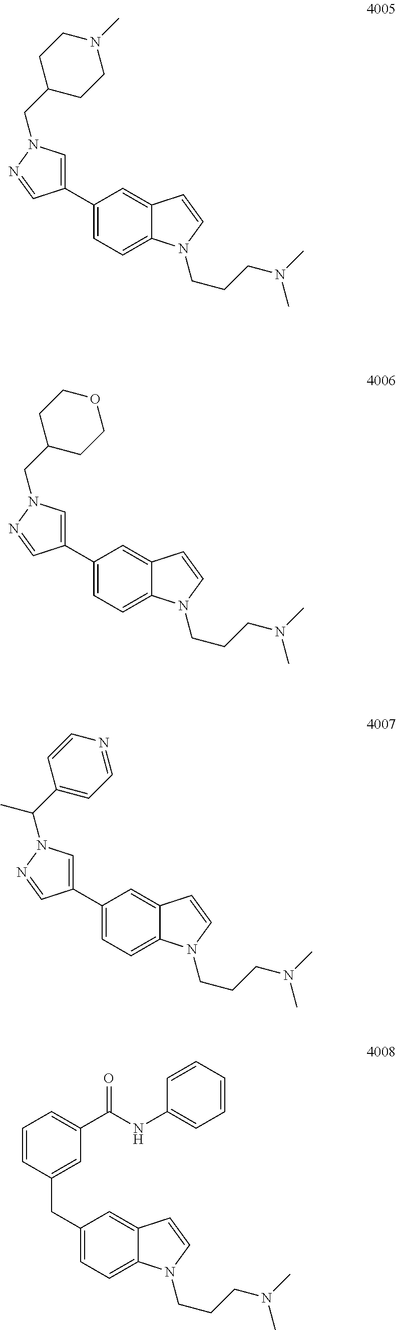

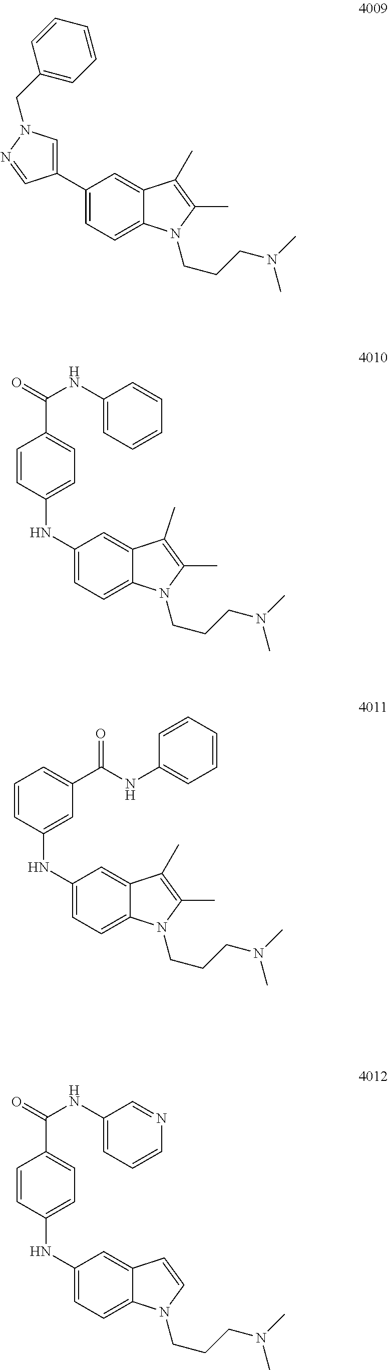 [SMILES: Cc1c(C)n(CCCN(C)C)c2ccc(-c3cnn(Cc4ccccc4)c3)cc12 ; Cc1c(C)n(CCCN(C)C)c2ccc(Nc3ccc(C(=O)Nc4ccccc4)cc3)cc12 ; Cc1c(C)n(CCCN(C)C)c2ccc(Nc3cccc(C(=O)Nc4ccccc4)c3)cc12 ; CN(C)CCCn1ccc2cc(Nc3ccc(C(=O)Nc4cccnc4)cc3)ccc21]

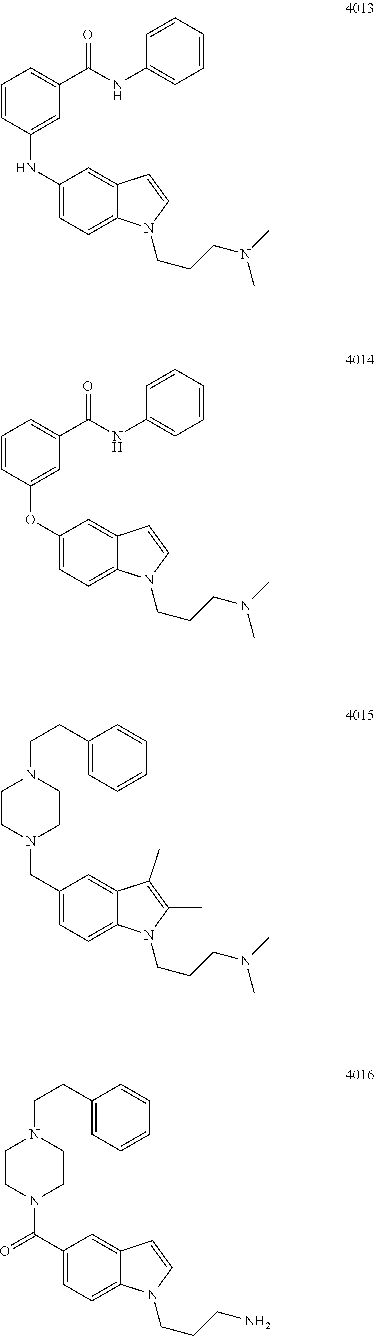 [SMILES: CN(C)CCCn1ccc2cc(Nc3cccc(C(=O)Nc4ccccc4)c3)ccc21 ; CN(C)CCCn1ccc2cc(Oc3cccc(C(=O)Nc4ccccc4)c3)ccc21 ; Cc1c(C)n(CCCN(C)C)c2ccc(CN3CCN(CCc4ccccc4)CC3)cc12 ; NCCCn1ccc2cc(C(=O)N3CCN(CCc4ccccc4)CC3)ccc21]

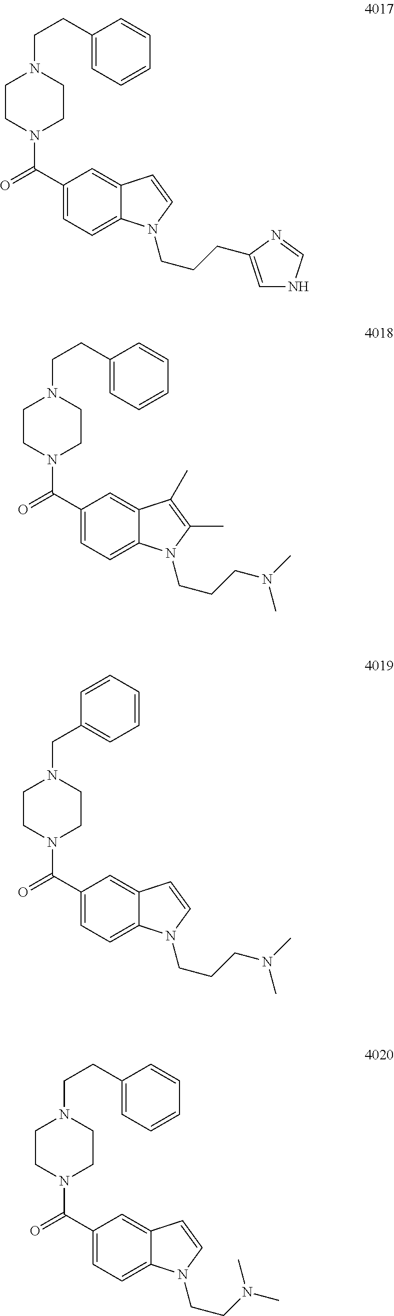 [SMILES: O=C(c1ccc2c(ccn2CCCc2c[nH]cn2)c1)N1CCN(CCc2ccccc2)CC1 ; Cc1c(C)n(CCCN(C)C)c2ccc(C(=O)N3CCN(CCc4ccccc4)CC3)cc12 ; CN(C)CCCn1ccc2cc(C(=O)N3CCN(Cc4ccccc4)CC3)ccc21 ; CN(C)CCn1ccc2cc(C(=O)N3CCN(CCc4ccccc4)CC3)ccc21]

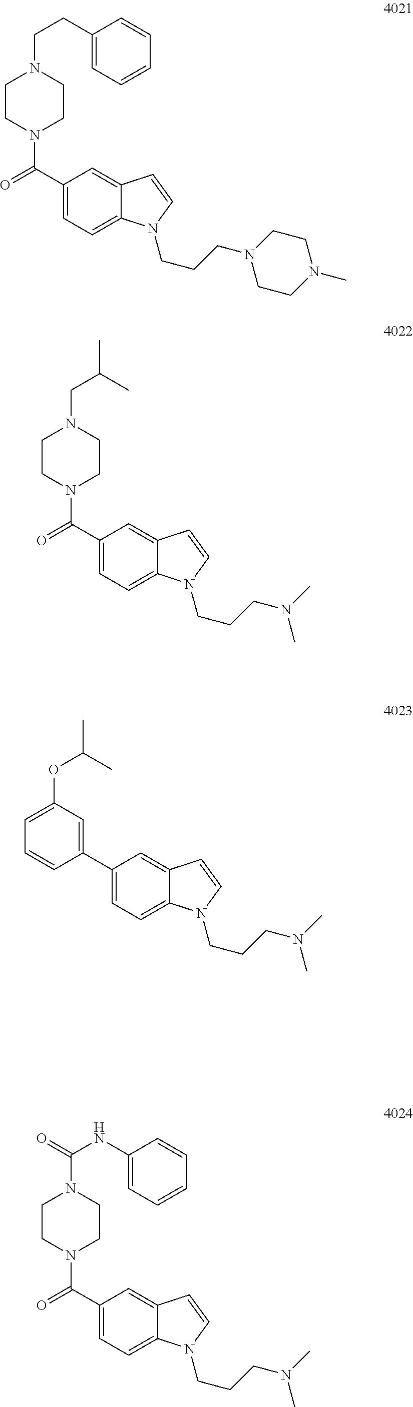 [SMILES: CN1CCN(CCCn2ccc3cc(C(=O)N4CCN(CCc5ccccc5)CC4)ccc32)CC1 ; CC(C)CN1CCN(C(=O)c2ccc3c(ccn3CCCN(C)C)c2)CC1 ; CC(C)Oc1cccc(-c2ccc3c(ccn3CCCN(C)C)c2)c1 ; CN(C)CCCn1ccc2cc(C(=O)N3CCN(C(=O)Nc4ccccc4)CC3)ccc21]

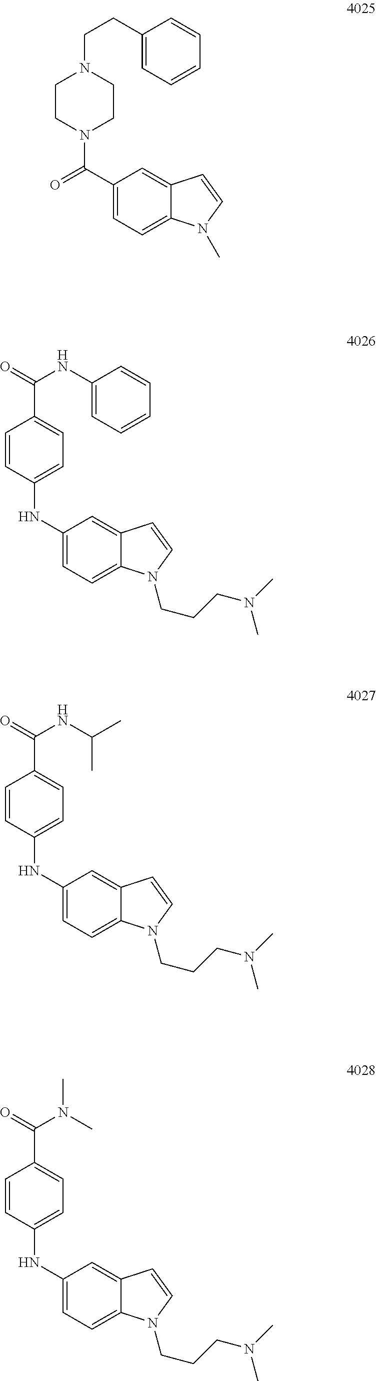 [SMILES: Cn1ccc2cc(C(=O)N3CCN(CCc4ccccc4)CC3)ccc21 ; CN(C)CCCn1ccc2cc(Nc3ccc(C(=O)Nc4ccccc4)cc3)ccc21 ; CC(C)NC(=O)c1ccc(Nc2ccc3c(ccn3CCCN(C)C)c2)cc1 ; CN(C)CCCn1ccc2cc(Nc3ccc(C(=O)N(C)C)cc3)ccc21]

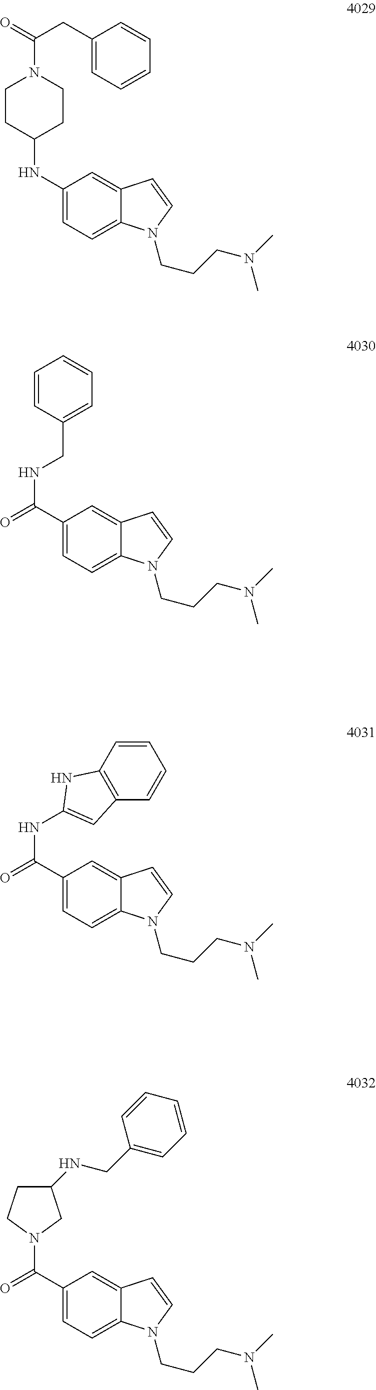 [SMILES: CN(C)CCCn1ccc2cc(NC3CCN(C(=O)Cc4ccccc4)CC3)ccc21 ; CN(C)CCCn1ccc2cc(C(=O)NCc3ccccc3)ccc21 ; CN(C)CCCn1ccc2cc(C(=O)Nc3cc4ccccc4[nH]3)ccc21 ; CN(C)CCCn1ccc2cc(C(=O)N3CCC(NCc4ccccc4)C3)ccc21]

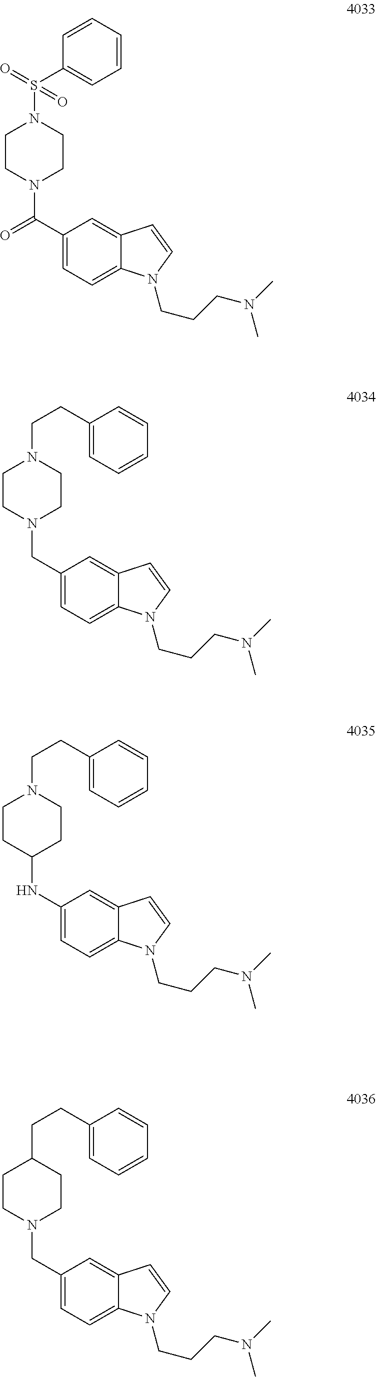 [SMILES: CN(C)CCCn1ccc2cc(C(=O)N3CCN(S(=O)(=O)c4ccccc4)CC3)ccc21 ; CN(C)CCCn1ccc2cc(CN3CCN(CCc4ccccc4)CC3)ccc21 ; CN(C)CCCn1ccc2cc(NC3CCN(CCc4ccccc4)CC3)ccc21 ; CN(C)CCCn1ccc2cc(CN3CCC(CCc4ccccc4)CC3)ccc21]

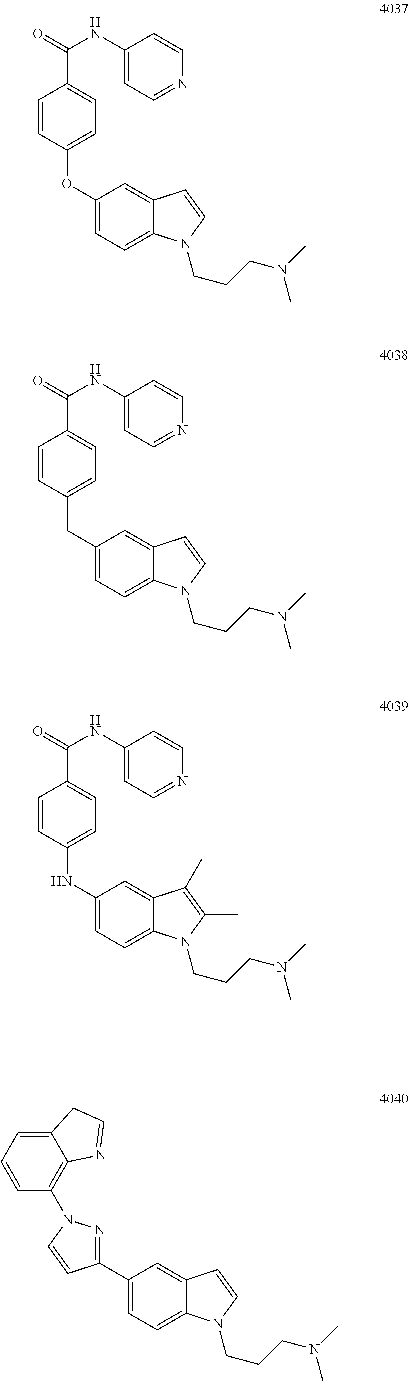 [SMILES: CN(C)CCCn1ccc2cc(Oc3ccc(C(=O)Nc4ccncc4)cc3)ccc21 ; CN(C)CCCn1ccc2cc(Cc3ccc(C(=O)Nc4ccncc4)cc3)ccc21 ; Cc1c(C)n(CCCN(C)C)c2ccc(Nc3ccc(C(=O)Nc4ccncc4)cc3)cc12 ; CN(C)CCCn1ccc2cc(-c3ccn(-c4cccc5c4N=CC5)n3)ccc21]

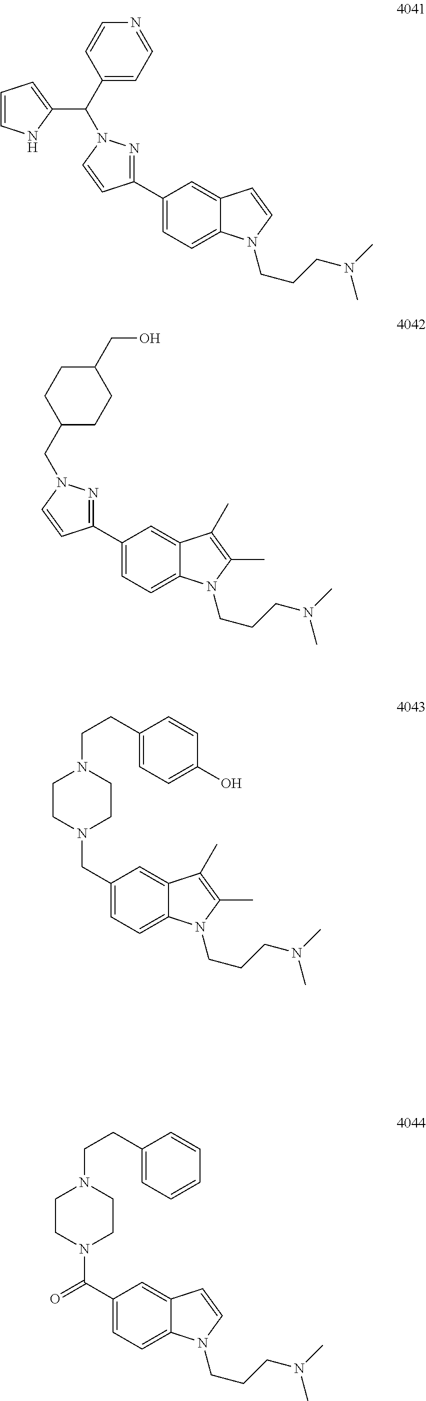 [SMILES: CN(C)CCCn1ccc2cc(-c3ccn(C(c4ccncc4)c4ccc[nH]4)n3)ccc21 ; Cc1c(C)n(CCCN(C)C)c2ccc(-c3ccn(CC4CCC(CO)CC4)n3)cc12 ; Cc1c(C)n(CCCN(C)C)c2ccc(CN3CCN(CCc4ccc(O)cc4)CC3)cc12 ; CN(C)CCCn1ccc2cc(C(=O)N3CCN(CCc4ccccc4)CC3)ccc21]

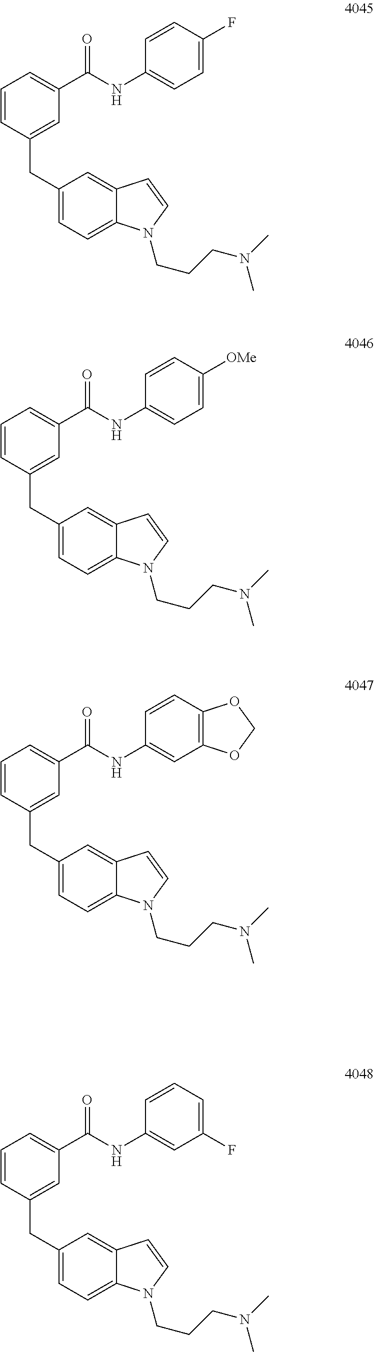 [SMILES: CN(C)CCCn1ccc2cc(Cc3cccc(C(=O)Nc4ccc(F)cc4)c3)ccc21 ; COc1ccc(NC(=O)c2cccc(Cc3ccc4c(ccn4CCCN(C)C)c3)c2)cc1 ; CN(C)CCCn1ccc2cc(Cc3cccc(C(=O)Nc4ccc5c(c4)OCO5)c3)ccc21 ; CN(C)CCCn1ccc2cc(Cc3cccc(C(=O)Nc4cccc(F)c4)c3)ccc21]

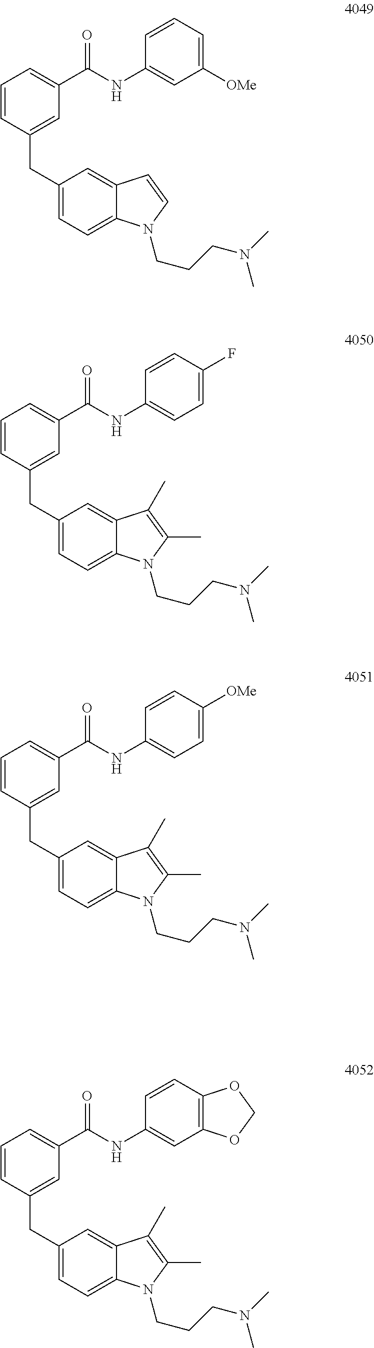 [SMILES: COc1cccc(NC(=O)c2cccc(Cc3ccc4c(ccn4CCCN(C)C)c3)c2)c1 ; Cc1c(C)n(CCCN(C)C)c2ccc(Cc3cccc(C(=O)Nc4ccc(F)cc4)c3)cc12 ; COc1ccc(NC(=O)c2cccc(Cc3ccc4c(c3)c(C)c(C)n4CCCN(C)C)c2)cc1 ; Cc1c(C)n(CCCN(C)C)c2ccc(Cc3cccc(C(=O)Nc4ccc5c(c4)OCO5)c3)cc12]

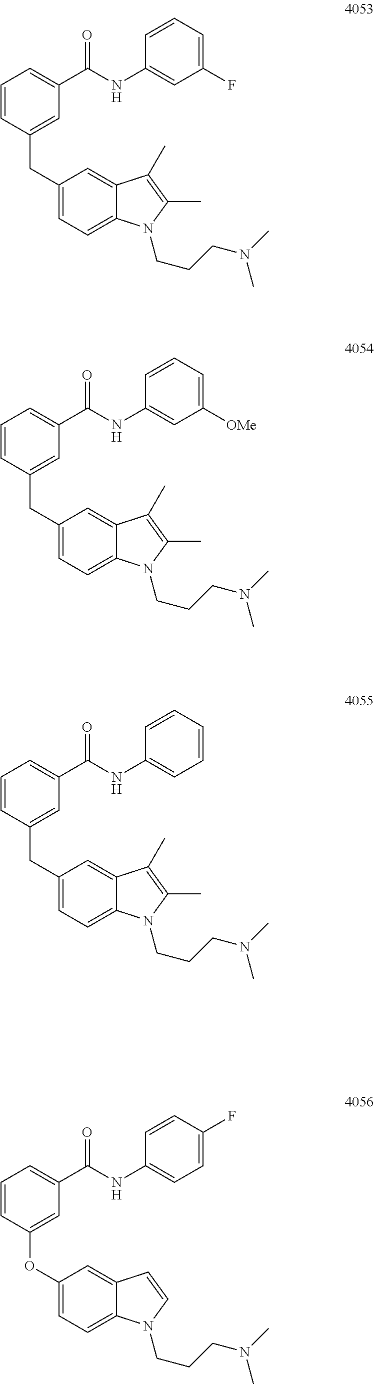 [SMILES: Cc1c(C)n(CCCN(C)C)c2ccc(Cc3cccc(C(=O)Nc4cccc(F)c4)c3)cc12 ; COc1cccc(NC(=O)c2cccc(Cc3ccc4c(c3)c(C)c(C)n4CCCN(C)C)c2)c1 ; Cc1c(C)n(CCCN(C)C)c2ccc(Cc3cccc(C(=O)Nc4ccccc4)c3)cc12 ; CN(C)CCCn1ccc2cc(Oc3cccc(C(=O)Nc4ccc(F)cc4)c3)ccc21]

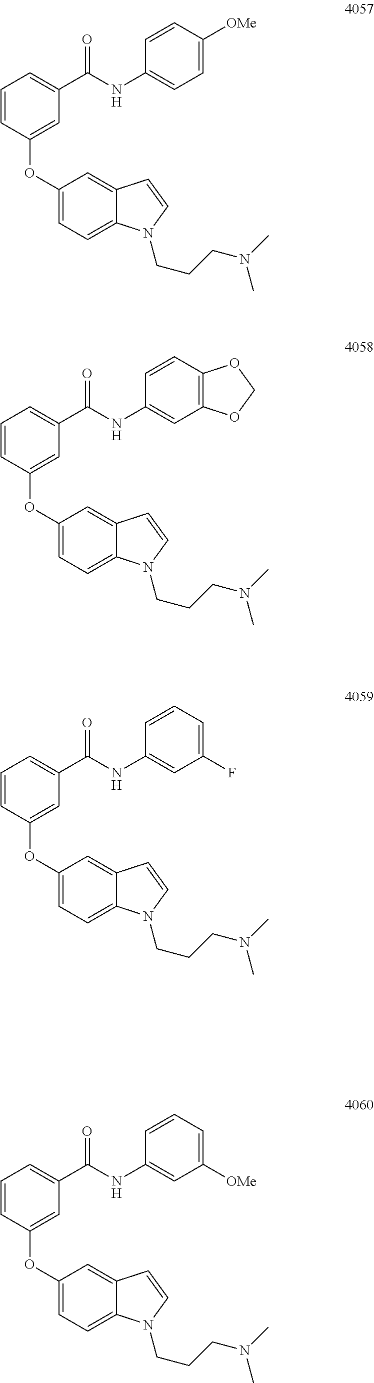 [SMILES: COc1ccc(NC(=O)c2cccc(Oc3ccc4c(ccn4CCCN(C)C)c3)c2)cc1 ; CN(C)CCCn1ccc2cc(Oc3cccc(C(=O)Nc4ccc5c(c4)OCO5)c3)ccc21 ; CN(C)CCCn1ccc2cc(Oc3cccc(C(=O)Nc4cccc(F)c4)c3)ccc21 ; COc1cccc(NC(=O)c2cccc(Oc3ccc4c(ccn4CCCN(C)C)c3)c2)c1]

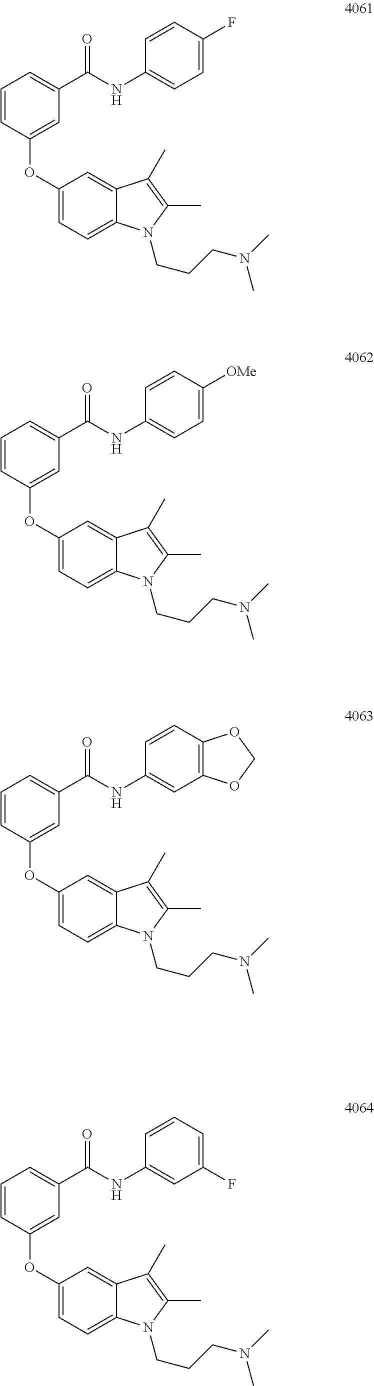 [SMILES: Cc1c(C)n(CCCN(C)C)c2ccc(Oc3cccc(C(=O)Nc4ccc(F)cc4)c3)cc12 ; COc1ccc(NC(=O)c2cccc(Oc3ccc4c(c3)c(C)c(C)n4CCCN(C)C)c2)cc1 ; Cc1c(C)n(CCCN(C)C)c2ccc(Oc3cccc(C(=O)Nc4ccc5c(c4)OCO5)c3)cc12 ; Cc1c(C)n(CCCN(C)C)c2ccc(Oc3cccc(C(=O)Nc4cccc(F)c4)c3)cc12]

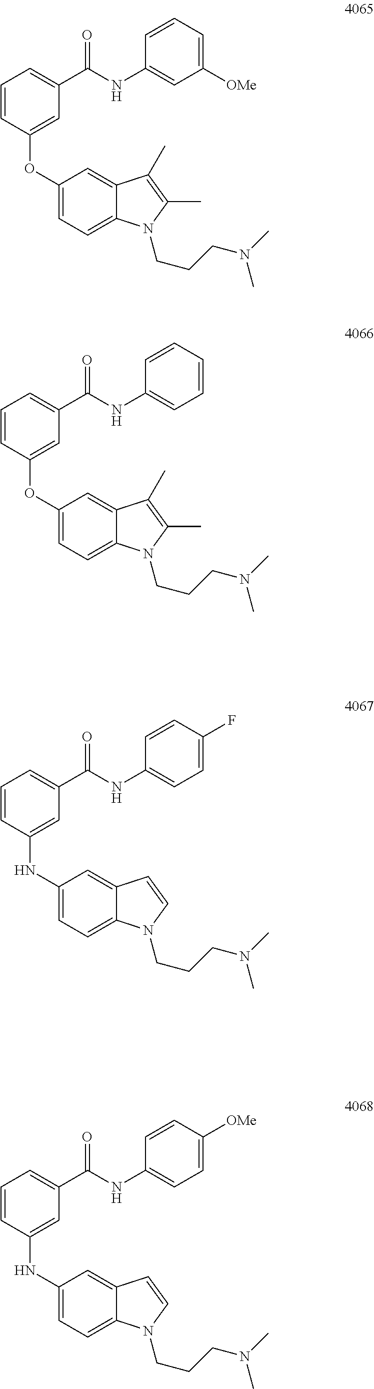 [SMILES: COc1cccc(NC(=O)c2cccc(Oc3ccc4c(c3)c(C)c(C)n4CCCN(C)C)c2)c1 ; Cc1c(C)n(CCCN(C)C)c2ccc(Oc3cccc(C(=O)Nc4ccccc4)c3)cc12 ; CN(C)CCCn1ccc2cc(Nc3cccc(C(=O)Nc4ccc(F)cc4)c3)ccc21 ; COc1ccc(NC(=O)c2cccc(Nc3ccc4c(ccn4CCCN(C)C)c3)c2)cc1]

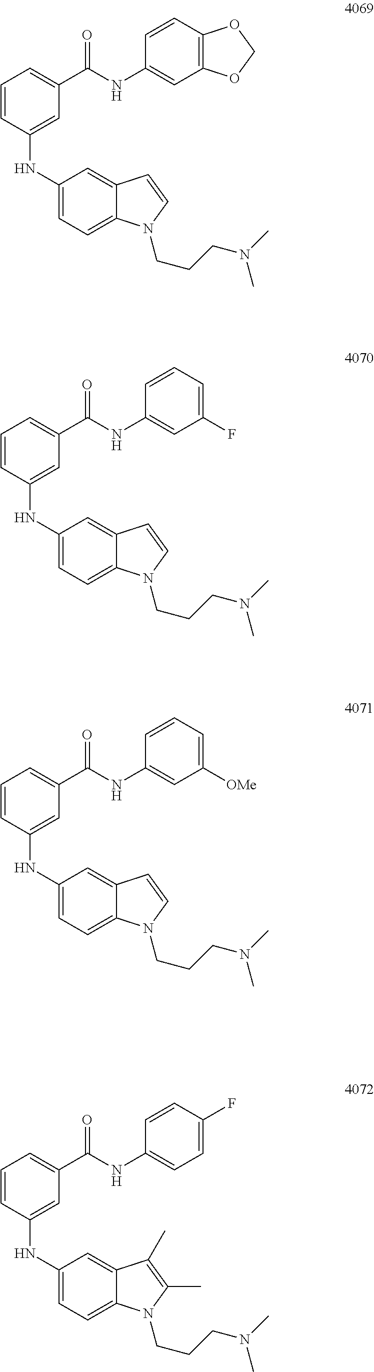 [SMILES: CN(C)CCCn1ccc2cc(Nc3cccc(C(=O)Nc4ccc5c(c4)OCO5)c3)ccc21 ; CN(C)CCCn1ccc2cc(Nc3cccc(C(=O)Nc4cccc(F)c4)c3)ccc21 ; COc1cccc(NC(=O)c2cccc(Nc3ccc4c(ccn4CCCN(C)C)c3)c2)c1 ; Cc1c(C)n(CCCN(C)C)c2ccc(Nc3cccc(C(=O)Nc4ccc(F)cc4)c3)cc12]

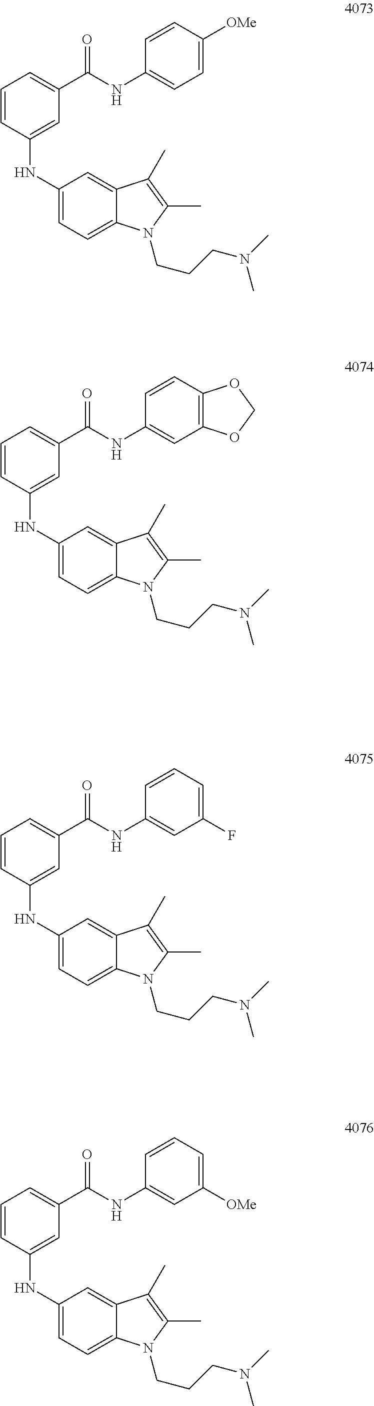 [SMILES: COc1ccc(NC(=O)c2cccc(Nc3ccc4c(c3)c(C)c(C)n4CCCN(C)C)c2)cc1 ; Cc1c(C)n(CCCN(C)C)c2ccc(Nc3cccc(C(=O)Nc4ccc5c(c4)OCO5)c3)cc12 ; Cc1c(C)n(CCCN(C)C)c2ccc(Nc3cccc(C(=O)Nc4cccc(F)c4)c3)cc12 ; COc1cccc(NC(=O)c2cccc(Nc3ccc4c(c3)c(C)c(C)n4CCCN(C)C)c2)c1]

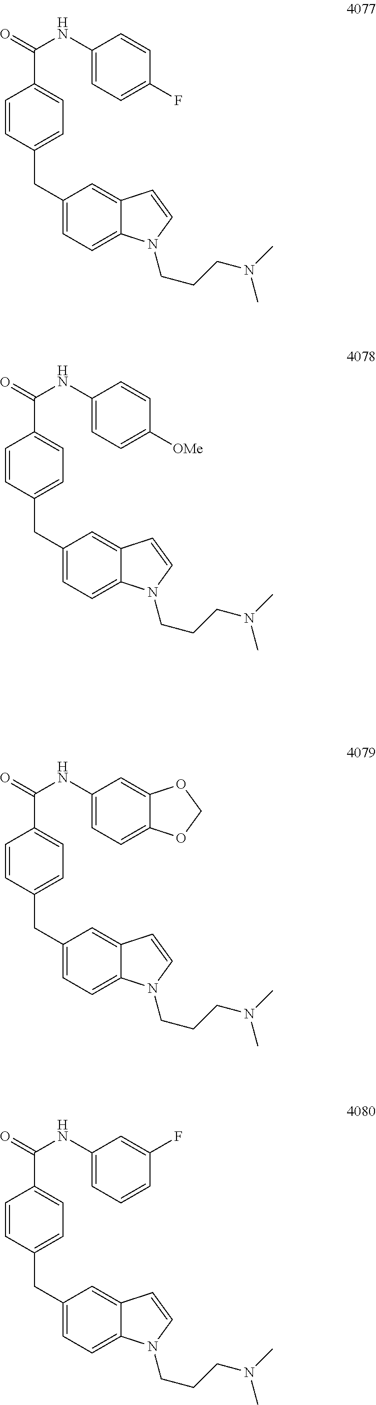 [SMILES: CN(C)CCCn1ccc2cc(Cc3ccc(C(=O)Nc4ccc(F)cc4)cc3)ccc21 ; COc1ccc(NC(=O)c2ccc(Cc3ccc4c(ccn4CCCN(C)C)c3)cc2)cc1 ; CN(C)CCCn1ccc2cc(Cc3ccc(C(=O)Nc4ccc5c(c4)OCO5)cc3)ccc21 ; CN(C)CCCn1ccc2cc(Cc3ccc(C(=O)Nc4cccc(F)c4)cc3)ccc21]

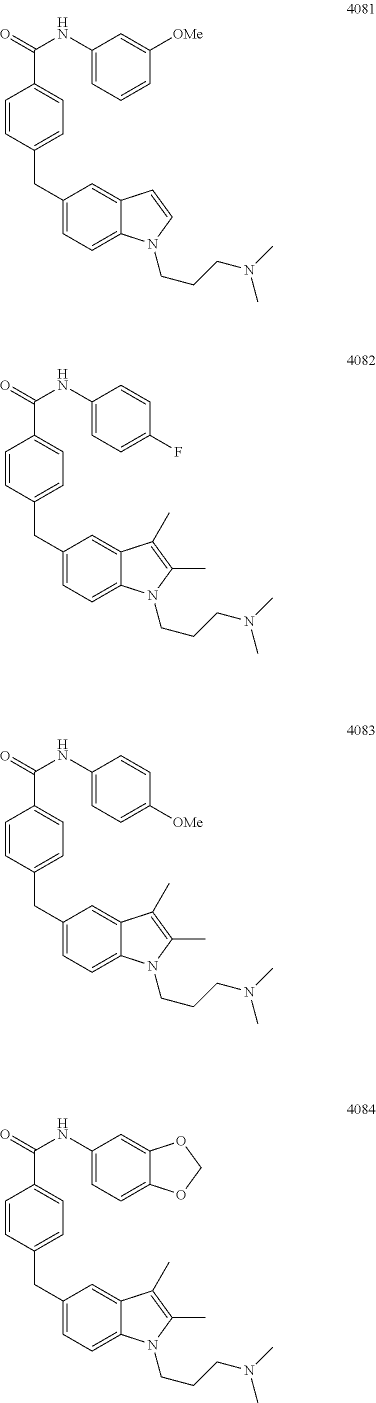 [SMILES: COc1cccc(NC(=O)c2ccc(Cc3ccc4c(ccn4CCCN(C)C)c3)cc2)c1 ; Cc1c(C)n(CCCN(C)C)c2ccc(Cc3ccc(C(=O)Nc4ccc(F)cc4)cc3)cc12 ; COc1ccc(NC(=O)c2ccc(Cc3ccc4c(c3)c(C)c(C)n4CCCN(C)C)cc2)cc1 ; Cc1c(C)n(CCCN(C)C)c2ccc(Cc3ccc(C(=O)Nc4ccc5c(c4)OCO5)cc3)cc12]

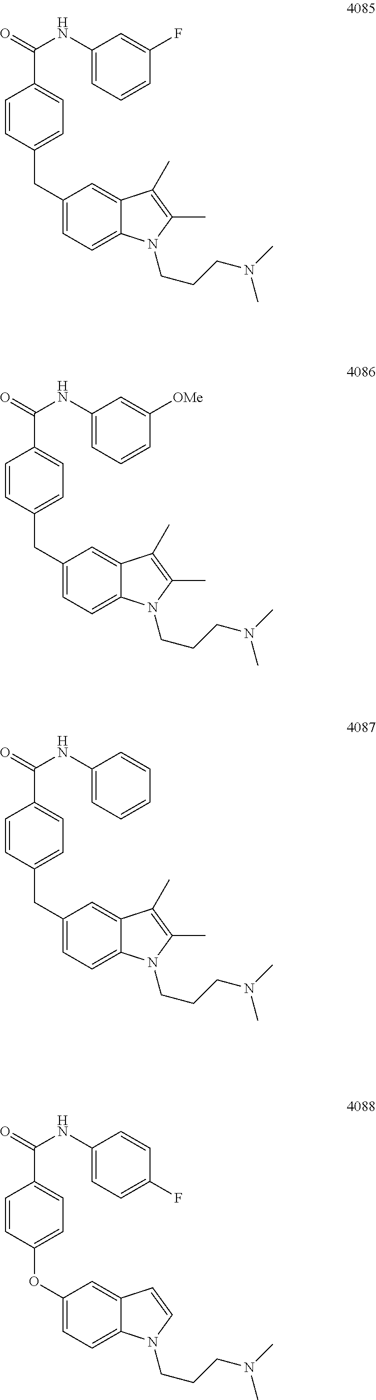 [SMILES: Cc1c(C)n(CCCN(C)C)c2ccc(Cc3ccc(C(=O)Nc4cccc(F)c4)cc3)cc12 ; COc1cccc(NC(=O)c2ccc(Cc3ccc4c(c3)c(C)c(C)n4CCCN(C)C)cc2)c1 ; Cc1c(C)n(CCCN(C)C)c2ccc(Cc3ccc(C(=O)Nc4ccccc4)cc3)cc12 ; CN(C)CCCn1ccc2cc(Oc3ccc(C(=O)Nc4ccc(F)cc4)cc3)ccc21]

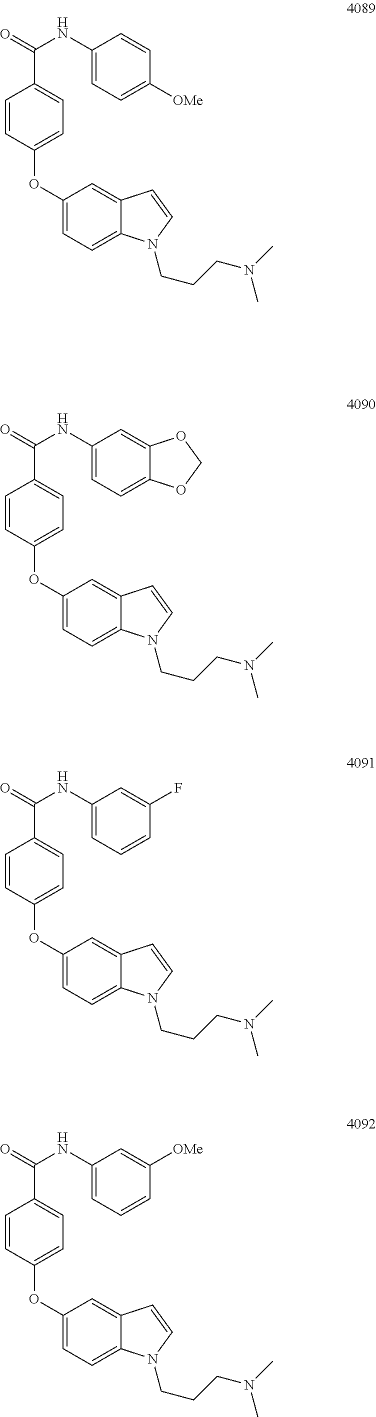 [SMILES: COc1ccc(NC(=O)c2ccc(Oc3ccc4c(ccn4CCCN(C)C)c3)cc2)cc1 ; CN(C)CCCn1ccc2cc(Oc3ccc(C(=O)Nc4ccc5c(c4)OCO5)cc3)ccc21 ; CN(C)CCCn1ccc2cc(Oc3ccc(C(=O)Nc4cccc(F)c4)cc3)ccc21 ; COc1cccc(NC(=O)c2ccc(Oc3ccc4c(ccn4CCCN(C)C)c3)cc2)c1]

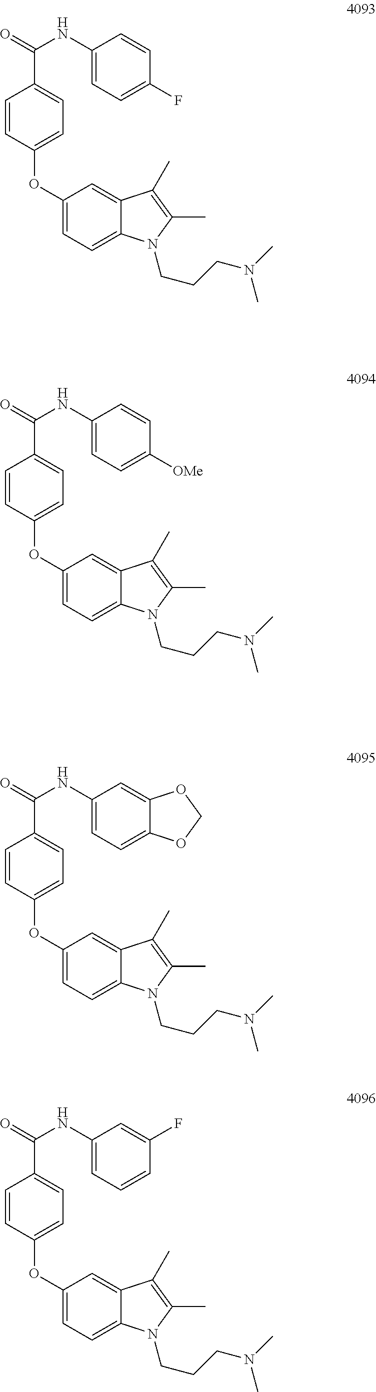 [SMILES: Cc1c(C)n(CCCN(C)C)c2ccc(Oc3ccc(C(=O)Nc4ccc(F)cc4)cc3)cc12 ; COc1ccc(NC(=O)c2ccc(Oc3ccc4c(c3)c(C)c(C)n4CCCN(C)C)cc2)cc1 ; Cc1c(C)n(CCCN(C)C)c2ccc(Oc3ccc(C(=O)Nc4ccc5c(c4)OCO5)cc3)cc12 ; Cc1c(C)n(CCCN(C)C)c2ccc(Oc3ccc(C(=O)Nc4cccc(F)c4)cc3)cc12]

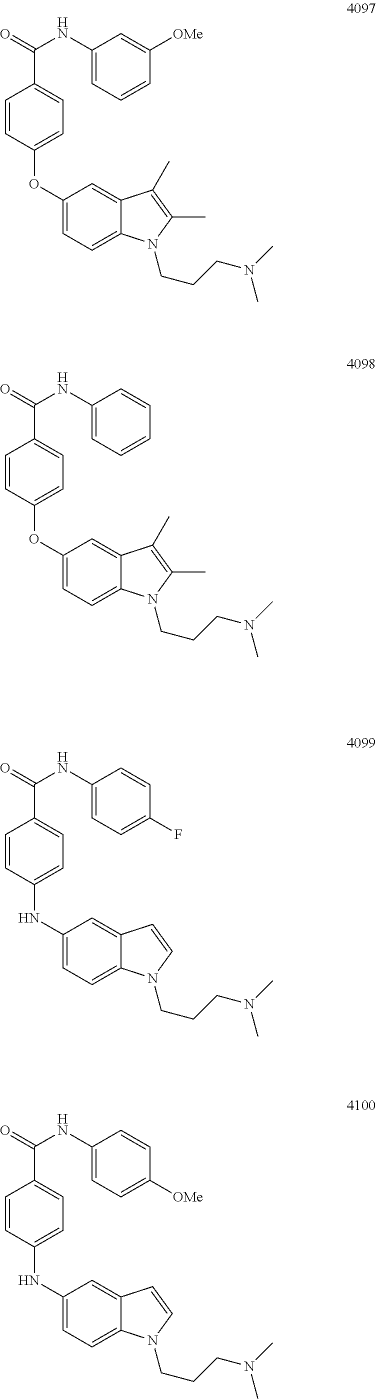 [SMILES: COc1cccc(NC(=O)c2ccc(Oc3ccc4c(c3)c(C)c(C)n4CCCN(C)C)cc2)c1 ; Cc1c(C)n(CCCN(C)C)c2ccc(Oc3ccc(C(=O)Nc4ccccc4)cc3)cc12 ; CN(C)CCCn1ccc2cc(Nc3ccc(C(=O)Nc4ccc(F)cc4)cc3)ccc21 ; COc1ccc(NC(=O)c2ccc(Nc3ccc4c(ccn4CCCN(C)C)c3)cc2)cc1]

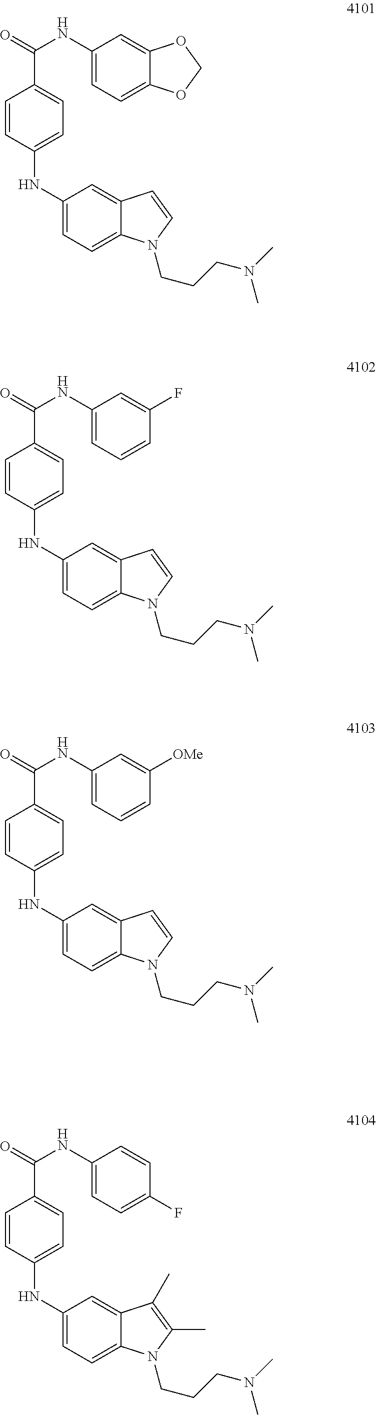 [SMILES: CN(C)CCCn1ccc2cc(Nc3ccc(C(=O)Nc4ccc5c(c4)OCO5)cc3)ccc21 ; CN(C)CCCn1ccc2cc(Nc3ccc(C(=O)Nc4cccc(F)c4)cc3)ccc21 ; COc1cccc(NC(=O)c2ccc(Nc3ccc4c(ccn4CCCN(C)C)c3)cc2)c1 ; Cc1c(C)n(CCCN(C)C)c2ccc(Nc3ccc(C(=O)Nc4ccc(F)cc4)cc3)cc12]

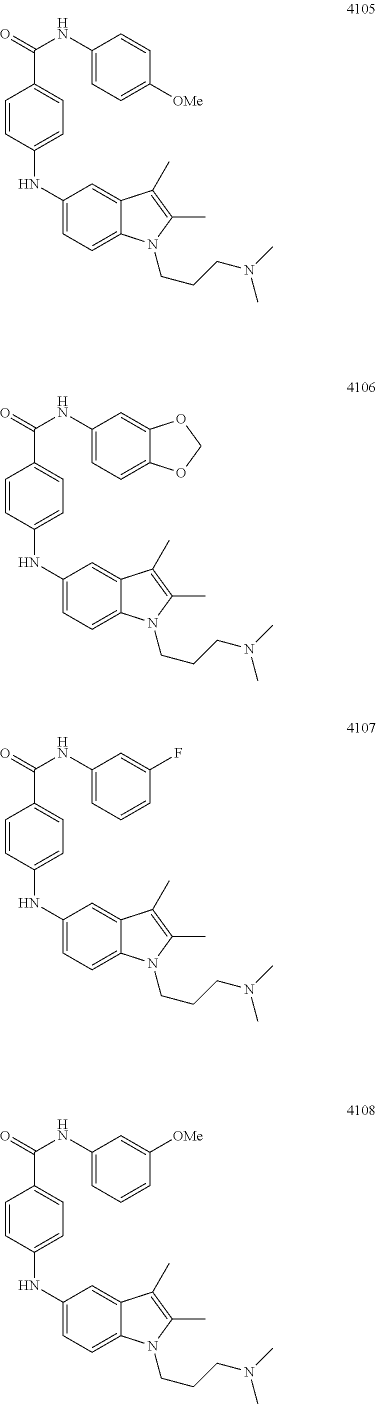 [SMILES: COc1ccc(NC(=O)c2ccc(Nc3ccc4c(c3)c(C)c(C)n4CCCN(C)C)cc2)cc1 ; Cc1c(C)n(CCCN(C)C)c2ccc(Nc3ccc(C(=O)Nc4ccc5c(c4)OCO5)cc3)cc12 ; Cc1c(C)n(CCCN(C)C)c2ccc(Nc3ccc(C(=O)Nc4cccc(F)c4)cc3)cc12 ; COc1cccc(NC(=O)c2ccc(Nc3ccc4c(c3)c(C)c(C)n4CCCN(C)C)cc2)c1]

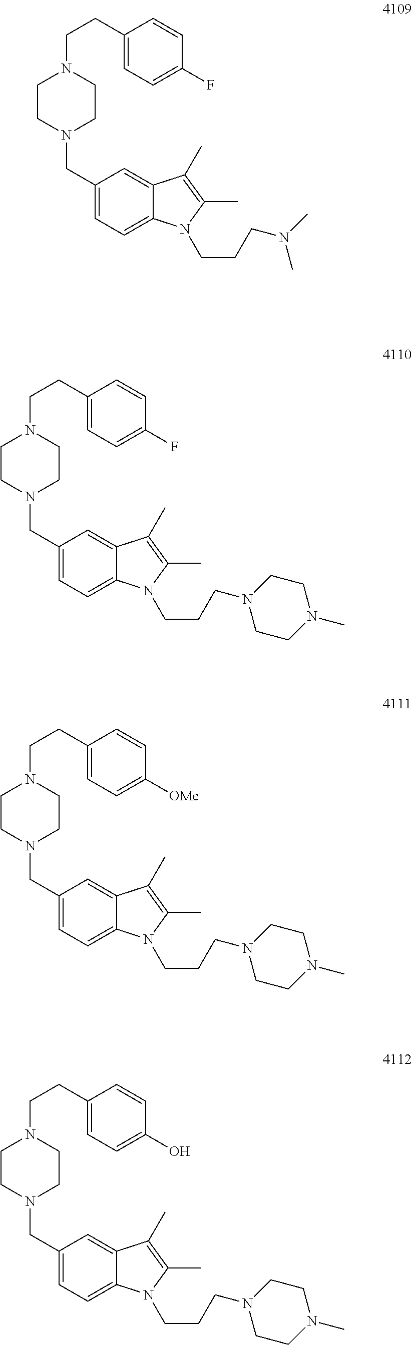 [SMILES: Cc1c(C)n(CCCN(C)C)c2ccc(CN3CCN(CCc4ccc(F)cc4)CC3)cc12 ; Cc1c(C)n(CCCN2CCN(C)CC2)c2ccc(CN3CCN(CCc4ccc(F)cc4)CC3)cc12 ; COc1ccc(CCN2CCN(Cc3ccc4c(c3)c(C)c(C)n4CCCN3CCN(C)CC3)CC2)cc1 ; Cc1c(C)n(CCCN2CCN(C)CC2)c2ccc(CN3CCN(CCc4ccc(O)cc4)CC3)cc12]

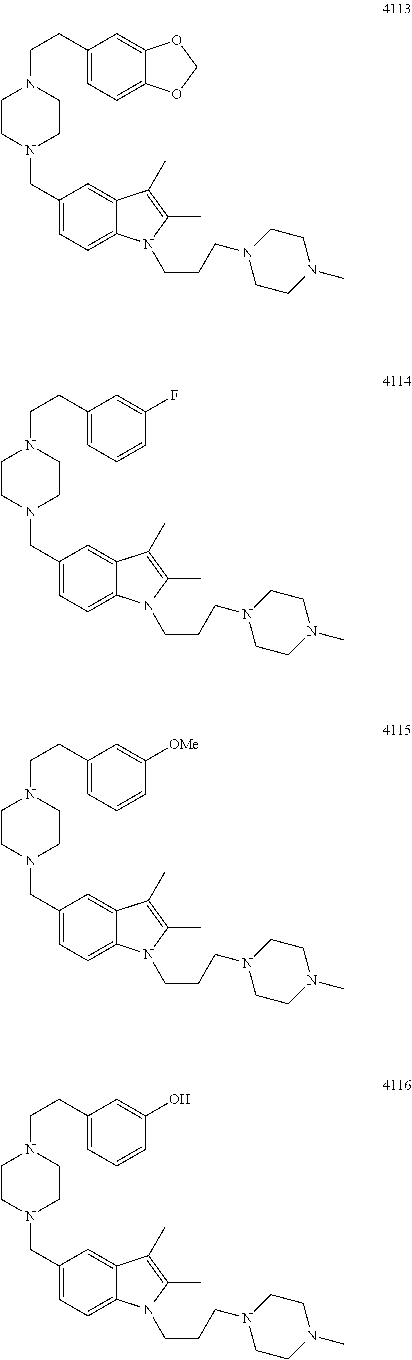 [SMILES: Cc1c(C)n(CCCN2CCN(C)CC2)c2ccc(CN3CCN(CCc4ccc5c(c4)OCO5)CC3)cc12 ; Cc1c(C)n(CCCN2CCN(C)CC2)c2ccc(CN3CCN(CCc4cccc(F)c4)CC3)cc12 ; COc1cccc(CCN2CCN(Cc3ccc4c(c3)c(C)c(C)n4CCCN3CCN(C)CC3)CC2)c1 ; Cc1c(C)n(CCCN2CCN(C)CC2)c2ccc(CN3CCN(CCc4cccc(O)c4)CC3)cc12]

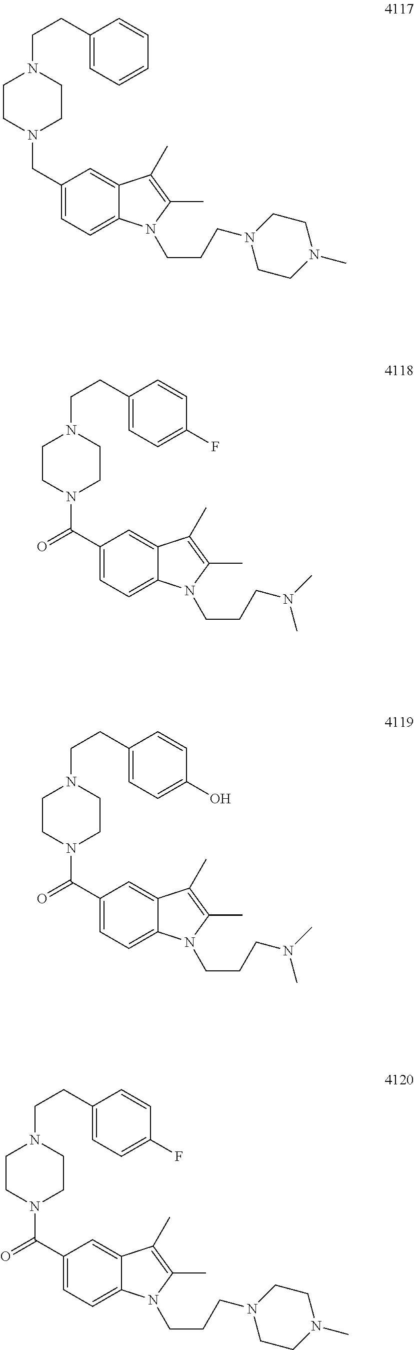 [SMILES: Cc1c(C)n(CCCN2CCN(C)CC2)c2ccc(CN3CCN(CCc4ccccc4)CC3)cc12 ; Cc1c(C)n(CCCN(C)C)c2ccc(C(=O)N3CCN(CCc4ccc(F)cc4)CC3)cc12 ; Cc1c(C)n(CCCN(C)C)c2ccc(C(=O)N3CCN(CCc4ccc(O)cc4)CC3)cc12 ; Cc1c(C)n(CCCN2CCN(C)CC2)c2ccc(C(=O)N3CCN(CCc4ccc(F)cc4)CC3)cc12]

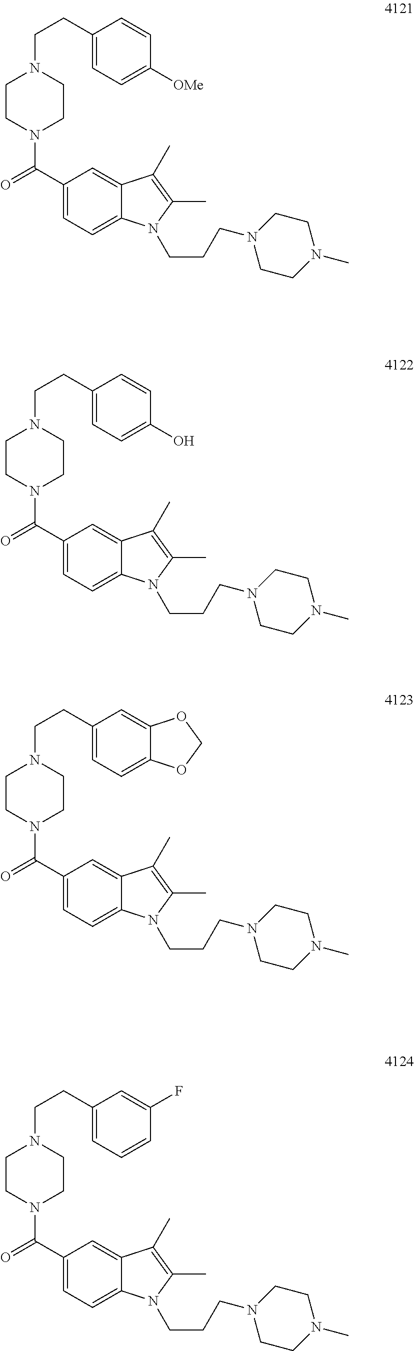 [SMILES: COc1ccc(CCN2CCN(C(=O)c3ccc4c(c3)c(C)c(C)n4CCCN3CCN(C)CC3)CC2)cc1 ; Cc1c(C)n(CCCN2CCN(C)CC2)c2ccc(C(=O)N3CCN(CCc4ccc(O)cc4)CC3)cc12 ; Cc1c(C)n(CCCN2CCN(C)CC2)c2ccc(C(=O)N3CCN(CCc4ccc5c(c4)OCO5)CC3)cc12 ; Cc1c(C)n(CCCN2CCN(C)CC2)c2ccc(C(=O)N3CCN(CCc4cccc(F)c4)CC3)cc12]

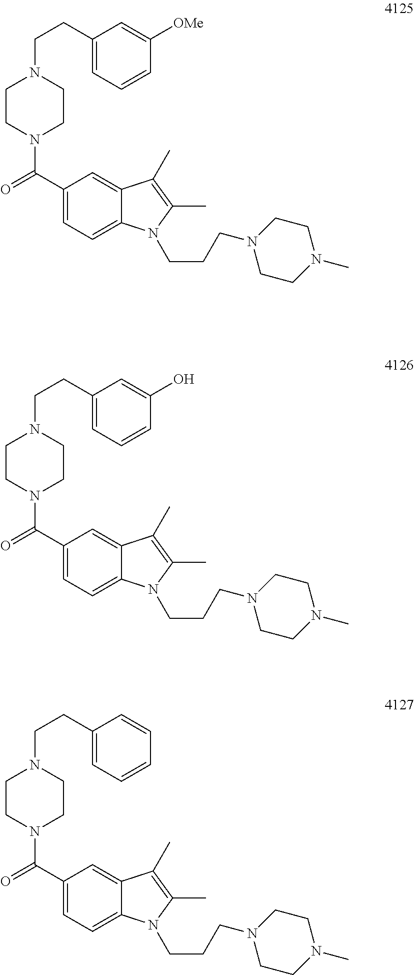 [SMILES: COc1cccc(CCN2CCN(C(=O)c3ccc4c(c3)c(C)c(C)n4CCCN3CCN(C)CC3)CC2)c1 ; Cc1c(C)n(CCCN2CCN(C)CC2)c2ccc(C(=O)N3CCN(CCc4cccc(O)c4)CC3)cc12 ; Cc1c(C)n(CCCN2CCN(C)CC2)c2ccc(C(=O)N3CCN(CCc4ccccc4)CC3)cc12]

34. A pharmaceutical composition comprising a compound according to claim 1.

35. A method for the treatment of a proliferative disease in a subject in need thereof, the method comprising administering to the subject a therapeutically effective amount of a compound according to claim 1.

36.-37. (canceled)

38. A method according to claim 35, wherein the proliferative disease is cancer.

39. (canceled)

40. A method for preventing recurrence of a solid tumor in a subject, the method comprising administering to the subject a compound according to claim 1.

41.-42. (canceled)
